(19) United States
(12) Patent Application Publication
(10) Pub. No.: US 2022/0378822 A1
(43) Pub. Date: Dec. 1, 2022

(54) COMBINATORIAL CANCER IMMUNOTHERAPY

(71) Applicant: Senti Bioscience, Inc., South San Francisco, CA (US)

(72) Inventors: Timothy Kuan-Ta Lu, Cambridge, MA (US); Russell Morrison Gordley, San Francisco, CA (US); Jack Tzu-Chiao Lin, Redwood City, CA (US); Brian Scott Garrison, San Jose, CA (US); Philip Janmin Lee, Alameda, CA (US); Alba Gonzalez-Junca, San Francisco, CA (US); Don-Hong Wang, South San Francisco, CA (US)

(21) Appl. No.: 17/636,302

(22) PCT Filed: Aug. 28, 2020
(86) PCT No.: PCT/US2020/048557

§ 371 Date: Feb. 17, 2022

Related U.S. Application Data
(60) Provisional application No. 62/893,060, filed on Aug. 28, 2019.

(51) Int. Cl. A61K 35/13; A61K 48/00; A61K 35/28; A61K 38/20; A61P 35/00

(52) U.S. Cl. CPC A61K 35/13; A61K 48/005; A61K 35/28; A61K 38/20; A61P 35/00

(57) ABSTRACT

Provided herein are methods and compositions for dynamically controlling and targeting multiple immunosuppressive mechanisms in cancer. Some aspects provide cells engineered to produce multiple effector molecules, each of which modulates a different immunosuppressive mechanisms of a tumor, as well as methods of using the cells to treat cancer, such as ovarian, breast, or colon cancer.

### CROSS REFERENCE TO RELATED APPLICATIONS

This application claims the benefit of U.S. Provisional Application No. 62/893,060, filed Aug. 28, 2019, which is hereby incorporated by reference in its entirety for all purposes.

### SEQUENCE LISTING

The instant application contains a Sequence Listing which has been submitted via EFS-Web and is hereby incorporated by reference in its entirety. Said ASCII copy, created on Aug. 28, 2020, is named STB_017WO_sequencelisting.txt, and is 142,438 bytes in size.

### BACKGROUND

There are more than 22,000 new cases of ovarian cancer and more than 14,000 deaths each year in the United States (Siegel R L, et al. (2016) CA Cancer J Clin 66(1):7-30), with an estimated annual healthcare burden of greater than $600M (Dizon D M J (2010) Gynecol Oncol 116(3)). Conventional approaches, such as chemotherapy (e.g., carboplatin/cisplatin and/or paclitaxel), are often unable to cure ovarian cancer. Approximately 70% of patients do not achieve remission on first-line chemotherapy, and 40-50% of patients that do have a remission will relapse within three years.

Treatment of other cancers, such as breast cancer and colon cancer, is associated with five-year survival rates of 85% and 65%, respectively. Therapies often include a combination of invasive surgeries and chemotherapies.

### SUMMARY

Provided herein, in some embodiments, is a combinatorial cell-based immunotherapy for the targeted treatment of cancer, such as ovarian cancer, breast cancer, colon cancer, lung cancer, and pancreatic cancer. This combinatorial immunotherapy relies on engineered cell circuits that enable multifactorial modulation within and/or near a tumor (a “tumor microenvironment (TME)”). Despite exciting advancements in combinatorial immunotherapy, its efficacy against cancer has been limited due in part to the following challenges. It is difficult to deliver multiple therapies simultaneously to achieve maximal efficacy without triggering significant side effects. It is also difficult in clinical trials to determine the appropriate dosing and timing of multiple systemically-administered and/or locally-injected therapies.

The combinatorial immunotherapy provided herein, however, is tumor-specific and effective yet limits systemic toxicity. This combinatorial immunotherapy delivers to a tumor microenvironment multiple immunomodulatory effector molecules from a single delivery vehicle. The design of the delivery vehicle is optimized to improve overall function in cancer therapy, including, but not limited to, optimization of the promoters, linkers, signal peptides, and order of the multiple immunomodulatory effector molecules.

It has been increasingly recognized that tumors are a complex interplay between the tumor cells and the surrounding stroma, which includes the extracellular matrix, cancer-associated stromal cells (MSCs and fibroblasts), tumor vasculature, and the immune system. The TME suppresses anti-tumor immune responses through multiple mechanisms that target both the innate and adaptive immune system of the patient. For example, tumors can recruit and induce regulatory T cells that suppress the anti-tumor activity of conventional T cells by elaborating specific chemokines such as CCL22. Tumors can also express molecules that inhibit the activity of T cells and NK cells, such as immune checkpoints such as PD-L1. Thus, targeting a single pathway is likely insufficient for achieving robust efficacy against solid tumors.

Non-limiting examples of effector molecules encompassed by the present disclosure include cytokines, antibodies, chemokines, nucleotides, peptides, enzymes, and oncolytic viruses. For example, cells may be engineered to express (and typically secrete) at least one, two, three or more of the following effector molecules: IL-12, IL-16, IFN-β, IFN-γ, IL-2, IL-15, IL-7, IL-36γ, IL-18, IL-10, IL-21, OX40-ligand, CD40L, anti-PD-1 antibodies, anti-PD-L1 antibodies, anti-CTLA-4 antibodies, anti-TGFβ antibodies, anti-TNFR2, MIP1α (CCL3), MIP1β (CCL5), CCL21, CpG oligodeoxynucleotides, and anti-tumor peptides (e.g., anti-microbial peptides having anti-tumor activity, see, e.g., Gaspar, D. et al. Front Microbiol. 2013; 4: 294; Chu, H. et al. PLoS One. 2015; 10(5): e0126390, and website:aps.unmc.edu/AP/main.php).

Provided herein is a tumor cell engineered to produce two or more effector molecules. In some aspects, the tumor cell is selected from the group consisting of a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, an ovarian tumor cell, a pancreatic tumor cell, a prostate tumor cell, a skin tumor cell, a thyroid tumor cell, and a uterine tumor cell. In some aspects, the cell was engineered via transduction with an oncolytic virus. In some aspects, the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some aspects, the oncolytic virus is a recombinant oncolytic virus comprising one more transgenes encoding one or more of the two or more effector molecules.

Also provided herein is an erythrocyte engineered to produce two or more effector molecules.

Also provided herein is a platelet cell engineered to produce two or more effector molecules.

Also provided herein is a bacterial cell engineered to produce two or more mammalian effector molecules. In some aspects, the bacterial cell is selected from the group consisting of Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

In some aspects, each of the two or more effector molecules comprises a secretion signal. In some aspects, each of the two or more effector molecules is secreted from the cell. In some aspects, the cell was engineered via transfection with an isolated nucleic acid. In some aspects, the isolated nucleic acid is a cDNA comprising a polynucleotide sequence encoding one or more of the two or more effector molecules. In some aspects, the isolated nucleic acid is an mRNA comprising a polynucleotide sequence encoding one or more of the two or more effector molecules. In some aspects, the isolated nucleic acid is a naked plasmid comprising a polynucleotide sequence encoding one or more of the two or more effector molecules.

In some aspects, the two or more effector molecules are encoded by a polynucleotide sequence. In some aspects, the polynucleotide sequence comprises a promoter. In some aspects, the promoter comprises an exogenous promoter polynucleotide sequence. In some aspects, the promoter comprises an endogenous promoter. In some aspects, the polynucleotide sequence further comprises a linker polynucleotide sequence. In some aspects, the promoter is operably linked to the polynucleotide sequence such that the two or more effector molecules are capable of being transcribed as a single polynucleotide comprising the formula (L-E)x, wherein

L comprises a linker polynucleotide sequence,

E comprises a polynucleotide encoding one of the two or more effector molecules,

X=2 to 20, and

wherein for the first iteration of the (L-E) unit L is absent.

In some aspects, the linker polynucleotide sequence is operably associated with the translation of the two or more effector molecules as separate polypeptides. In some aspects, the linker polynucleotide sequence encodes a 2A ribosome skipping tag. In some aspects, the 2A ribosome skipping tag is selected from the group consisting of P2A, T2A, E2A, and F2A. In some aspects, the linker polynucleotide sequence encodes a cleavable polypeptide. In some aspects, the cleavable polypeptide comprises a furin polypeptide sequence. In some aspects, the linker polynucleotide sequence encodes an Internal Ribosome Entry Site (IRES). In some aspects, the linker polynucleotide sequence encodes an additional promoter. In some aspects, the promoter is operably linked to the polynucleotide sequence such that a first polynucleotide encoding one of the two or more effector molecules is capable of being transcribed, wherein the additional promoter is operably linked to the polynucleotide sequence such that a second polynucleotide encoding a second of the two or more effector molecules is capable of being transcribed, and wherein the first and the second polynucleotides are separate polynucleotides. In some aspects, the promoter and the additional promoter are identical. In some aspects, the promoter and the additional promoter are different.

In some aspects, the engineered cell is a human cell. In some aspects, the human cell is an isolated cell from a subject. In some aspects, the engineered cell is a cultured cell.

In some aspects, the promoter and/or the additional promoter comprises a constitutive promoter. In some aspects, the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb. In some aspects, the promoter and/or the additional promoter comprises an inducible promoter. In some aspects, the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, one secretion signal peptide comprises a native secretion signal peptide native to a corresponding effector molecule of the two or more effector molecules. In some aspects, one secretion signal peptide comprises a non-native secretion signal peptide non-native to at least one of the two or more effector molecules. In some aspects, the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12. In some aspects, each secretion signal peptide is identical.

In some aspects, a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.

In some aspects, a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme. In some aspects, the therapeutic class of the first effector molecule and the second effector molecule are different. In some aspects, the cytokine is selected from the group consisting of IL12, IL7, IL21, IL18, IL15, Type I interferons, and Interferon-gamma. In some aspects, the IL12 cytokine is an IL12p70 fusion protein. In some aspects, the chemokine is selected from the group consisting of CCL21a, CXCL10, CXCL11, CXCL13, CXCL10-11 fusion, CCL19, CXCL9, and XCL1. In some aspects, the growth factor is selected from the group consisting of FLT3L and GM-CSF. In some aspects, the co-activation molecule is selected from the group consisting of 4-1BBL and CD40L. In some aspects, the tumor microenvironment modifier is selected from the group consisting of adenosine deaminase, TGFbeta inhibitors, immune checkpoint inhibitors, VEGF inhibitors, and HPGE2. In some aspects, the TGFbeta inhibitors are selected from the group consisting of an anti-TGFbeta peptide, an anti-TGFbeta antibody, a TGFb-TRAP, and combinations thereof. In some aspects, the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies. In some aspects, the VEGF inhibitors comprise anti-VEGF antibodies, anti-VEGF peptides, or combinations thereof.

In some aspects, at least one of the two or more effector molecules is a human-derived effector molecule. In some aspects, one effector molecule comprises IL12. In some aspects, a second effector molecule comprises CCL21a, IL7, IL15, or IL21.

In some aspects, the polynucleotide sequence comprises:

a) an SFFV promoter; and

b) a polynucleotide described in a formula, oriented from 5′ to 3′, comprising

S1-E1-L-S2-E2

wherein

S1 comprises a polynucleotide sequence encoding a first secretion signal peptide, wherein the first secretion signal peptide is a human IL12 secretion signal peptide;

E1 comprises a polynucleotide sequence encoding a first effector molecule, wherein the first effector molecule is a human IL12p70 fusion protein;

L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;

S2 comprises a polynucleotide sequence encoding a second secretion signal peptide, wherein the second secretion signal peptide is a human IL21 secretion signal peptide;

E2 comprises a polynucleotide sequence encoding a second effector molecule, wherein the second effector molecule is human IL21; and

wherein the SFFV promoter is operably linked to the polynucleotide, the first secretion signal peptide is operably linked to the first effector molecule, and the second secretion signal peptide is operably linked to the second effector molecule. In some aspects, the polynucleotide sequence comprises the polynucleotide sequence of SEQ ID NO: 144.

In some aspects, the polynucleotide sequence is integrated into the genome of the engineered cell. In some aspects, the polynucleotide sequence comprises one or more viral vector polynucleotide sequences. In some aspects, the one or more viral vector polynucleotide sequences comprise lentiviral, retroviral, retrotransposon, adenoviral, or adeno-associated viral polynucleotide sequences.

Also provide herein is a population of cells comprising one or more of the engineered cells described herein.

Also provide herein is a composition comprising one or more of the engineered cells described herein or any of the population of cells described herein, and a pharmaceutically acceptable carrier.

Also provide herein is a method of treating a subject having cancer, the method comprising administering a therapeutically effective dose of any one or more of the engineered cells described herein, any of the population of cells described herein, or any one or more of the engineered cells described herein or population of cells described here comprising a pharmaceutically acceptable carrier.

Also provide herein is a method of reducing tumor volume in a subject, the method comprising administering to a subject having a tumor a composition comprising any one or more of the engineered cells described herein, any of the population of cells described herein, or any one or more of the engineered cells described herein or population of cells described here comprising a pharmaceutically acceptable carrier.

In some aspects, the administering comprises systemic administration. In some aspects, the administering comprises intratumoral administration or intraperitoneal administration. In some aspects, the engineered cell is derived from the subject. In some aspects, the engineered cell is allogeneic with reference to the subject.

In some aspects, the method further comprises administering a checkpoint inhibitor. In some aspects, the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody. In some aspects, the method further comprises administering an anti-CD40 antibody.

In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor. In some aspects, the tumor is a tumor located in a peritoneal space.

Also provided herein is a lipid structure delivery system comprising a lipid-based structure comprising two or more effector molecules. In some aspects, the two or more effector molecules are encoded by a polynucleotide sequence.

Also provided herein is a lipid-based structure comprising an engineered nucleic acid, wherein the engineered nucleic acid comprises a polynucleotide sequence encoding two or more effector molecules. In some aspects, the engineered nucleic acid is a cDNA. In some aspects, the engineered nucleic acid is an mRNA. In some aspects, the engineered nucleic acid is a naked plasmid.

In some aspects, the polynucleotide sequence comprises a promoter. In some aspects, the promoter comprises an exogenous promoter polynucleotide sequence. In some aspects, the promoter comprises an endogenous promoter.

In some aspects, the polynucleotide sequence further comprises a linker polynucleotide sequence. In some aspects, the promoter is operably linked to the polynucleotide sequence such that the two or more effector molecules are capable of being transcribed as a single polynucleotide comprising the formula (L-E)x, wherein

L comprises a linker polynucleotide sequence,

E comprises a polynucleotide encoding one of the two or more effector molecules,

X=2 to 20, and

wherein for the first iteration of the (L-E) unit L is absent.

In some aspects, the linker polynucleotide sequence is operably associated with the translation of the two or more effector molecules as separate polypeptides. In some aspects, the linker polynucleotide sequence encodes a 2A ribosome skipping tag. In some aspects, the 2A ribosome skipping tag is selected from the group consisting of P2A, T2A, E2A, and F2A. In some aspects, the linker polynucleotide sequence encodes a cleavable polypeptide. In some aspects, the cleavable polypeptide comprises a furin polypeptide sequence. In some aspects, the linker polynucleotide sequence encodes an Internal Ribosome Entry Site (IRES). In some aspects, the linker polynucleotide sequence encodes an additional promoter. In some aspects, the promoter is operably linked to the polynucleotide sequence such that a first polynucleotide encoding one of the two or more effector molecules is capable of being transcribed, wherein the additional promoter is operably linked to the polynucleotide sequence such that a second polynucleotide encoding a second of the two or more effector molecules is capable of being transcribed, and wherein the first and the second polynucleotides are separate polynucleotides. In some aspects, the promoter and the additional promoter are identical. In some aspects, the promoter and the additional promoter are different.

In some aspects, the promoter and/or the additional promoter comprises a constitutive promoter. In some aspects, the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb. In some aspects, the promoter and/or the additional promoter comprises an inducible promoter. In some aspects, the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, each of the two or more effector molecules comprises a secretion signal. In some aspects, one secretion signal peptide comprises a native secretion signal peptide native to a corresponding effector molecule of the two or more effector molecules. In some aspects, one secretion signal peptide comprises a non-native secretion signal peptide non-native to at least one of the two or more effector molecules. In some aspects, the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12. In some aspects, each secretion signal peptide is identical.

In some aspects, a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme. In some aspects, a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme. In some aspects, the therapeutic class of the first effector molecule and the second effector molecule are different.

In some aspects, the cytokine is selected from the group consisting of IL12, IL7, IL21, IL18, IL15, Type I interferons, and Interferon-gamma. In some aspects, the IL12 cytokine is an IL12p70 fusion protein. In some aspects, the chemokine is selected from the group consisting of CCL21a, CXCL10, CXCL11, CXCL13, CXCL10-11 fusion, CCL19, CXCL9, and XCL1. In some aspects, the growth factor is selected from the group consisting of FLT3L and GM-CSF. In some aspects, the co-activation molecule is selected from the group consisting of 4-1BBL and CD40L. In some aspects, the tumor microenvironment modifier is selected from the group consisting of adenosine deaminase, TGFbeta inhibitors, immune checkpoint inhibitors, VEGF inhibitors, and HPGE2. In some aspects, the TGFbeta inhibitors are selected from the group consisting of an anti-TGFbeta peptide, an anti-TGFbeta antibody, a TGFb-TRAP, and combinations thereof. In some aspects, the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies. In some aspects, the VEGF inhibitors comprise anti-VEGF antibodies, anti-VEGF peptides, or combinations thereof.

In some aspects, at least one of the two or more effector molecules is a human-derived effector molecule. In some aspects, one effector molecule comprises IL12. In some aspects, a second effector molecule comprises CCL21a, IL7, IL15, or IL21.

In some aspects, the polynucleotide sequence comprises:

a) an SFFV promoter; and

b) a polynucleotide described in a formula, oriented from 5′ to 3′, comprising

S1-E1-L-S2-E2

wherein S1 comprises a polynucleotide sequence encoding a first secretion signal peptide, wherein the first secretion signal peptide is a human IL12 secretion signal peptide;

E1 comprises a polynucleotide sequence encoding a first effector molecule, wherein the first effector molecule is a human IL12p70 fusion protein;

L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;

S2 comprises a polynucleotide sequence encoding a second secretion signal peptide, wherein the second secretion signal peptide is a human IL21 secretion signal peptide;

E2 comprises a polynucleotide sequence encoding a second effector molecule, wherein the second effector molecule is human IL21; and

wherein the SFFV promoter is operably linked to the polynucleotide, the first secretion signal peptide is operably linked to the first effector molecule, and the second secretion signal peptide is operably linked to the second effector molecule. In some aspects, the polynucleotide sequence comprises the polynucleotide sequence of SEQ ID NO: 144.

In some aspects, the lipid-based structure comprises a extracellular vesicle. In some aspects, the extracellular vesicle is selected from the group consisting of a nanovesicle and an exosome. In some aspects, the lipid-based structure comprises a lipid nanoparticle or a micelle. In some aspects, the lipid-based structure comprises a liposome.

Also provided herein is a composition comprising the lipid-based structure of any one of the lipid-based structures described herein and a pharmaceutically acceptable carrier.

Also provided herein is a method of treating a subject having cancer, the method comprising administering a therapeutically effective dose of any of the lipid-based structures described herein or any one of the lipid-based structures described herein and a pharmaceutically acceptable carrier.

Also provided herein is a method of reducing tumor volume in a subject, the method comprising administering to a subject having a tumor a composition comprising any of the lipid-based structures described herein and a pharmaceutically acceptable carrier.

In some aspects, the administering comprises systemic administration. In some aspects, the administering comprises intratumoral administration or intraperitoneal administration. In some aspects, the lipid-based structure is capable of engineering a cell in the in the subject to produce two or more effector molecules that modulate tumor-mediated immunosuppressive mechanisms. In some aspects, the cell is a tumor cell, an immune cell, an erythrocyte, or a platelet cell.

In some aspects, the method further comprises administering a checkpoint inhibitor. In some aspects, the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody. In some aspects, the method further comprises administering an anti-CD40 antibody.

In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor. In some aspects, the tumor is a tumor located in a peritoneal space.

Also provided herein is a nanoparticle comprising two or more effector molecules. In some aspects, the two or more effector molecules are encoded by a polynucleotide sequence.

Also provided herein is a nanoparticle comprising an engineered nucleic acid, wherein the engineered nucleic acid comprises a polynucleotide sequence encoding two or more effector molecules. In some aspects, the engineered nucleic acid is a cDNA. In some aspects, the engineered nucleic acid is an mRNA. In some aspects, the engineered nucleic acid is a naked plasmid.

In some aspects, the polynucleotide sequence comprises a promoter. In some aspects, the promoter comprises an exogenous promoter polynucleotide sequence. In some aspects, the promoter comprises an endogenous promoter. In some aspects, the polynucleotide sequence further comprises a linker polynucleotide sequence. In some aspects, the promoter is operably linked to the polynucleotide sequence such that the two or more effector molecules are capable of being transcribed as a single polynucleotide comprising the formula (L-E)x, wherein

L comprises a linker polynucleotide sequence,

E comprises a polynucleotide encoding one of the two or more effector molecules,

X=2 to 20, and

wherein for the first iteration of the (L-E) unit L is absent.

In some aspects, the linker polynucleotide sequence is operably associated with the translation of the two or more effector molecules as separate polypeptides. In some aspects, the linker polynucleotide sequence encodes a 2A ribosome skipping tag. In some aspects, the 2A ribosome skipping tag is selected from the group consisting of P2A, T2A, E2A, and F2A. In some aspects, the linker polynucleotide sequence encodes a cleavable polypeptide. In some aspects, the cleavable polypeptide comprises a furin polypeptide sequence. In some aspects, the linker polynucleotide sequence encodes an Internal Ribosome Entry Site (IRES). In some aspects, the linker polynucleotide sequence encodes an additional promoter.

In some aspects, the promoter is operably linked to the polynucleotide sequence such that a first polynucleotide encoding one of the two or more effector molecules is capable of being transcribed, wherein the additional promoter is operably linked to the polynucleotide sequence such that a second polynucleotide encoding a second of the two or more effector molecules is capable of being transcribed, and wherein the first and the second polynucleotides are separate polynucleotides. In some aspects, the promoter and the additional promoter are identical. In some aspects, the promoter and the additional promoter are different.

In some aspects, the promoter and/or the additional promoter comprises a constitutive promoter. In some aspects, the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb. In some aspects, the promoter and/or the additional promoter comprises an inducible promoter. In some aspects, the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, each of the two or more effector molecules comprises a secretion signal. In some aspects, one secretion signal peptide comprises a native secretion signal peptide native to at least one of the two or more effector molecules. In some aspects, one secretion signal peptide comprises a non-native secretion signal peptide non-native to at least one of the two or more effector molecules. In some aspects, the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12. In some aspects, each secretion signal peptide is identical.

In some aspects, a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme. In some aspects, a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme. In some aspects, the therapeutic class of the first effector molecule and the second effector molecule are different.

In some aspects, the cytokine is selected from the group consisting of IL12, IL7, IL21, IL18, IL15, Type I interferons, and Interferon-gamma. In some aspects, the IL12 cytokine is an IL12p70 fusion protein. In some aspects, the chemokine is selected from the group consisting of CCL21a, CXCL10, CXCL11, CXCL13, CXCL10-11 fusion, CCL19, CXCL9, and XCL1. In some aspects, the growth factor is selected from the group consisting of FLT3L and GM-CSF. In some aspects, the co-activation molecule is selected from the group consisting of 4-1BBL and CD40L. In some aspects, the tumor microenvironment modifier is selected from the group consisting of adenosine deaminase, TGFbeta inhibitors, immune checkpoint inhibitors, VEGF inhibitors, and HPGE2. In some aspects, the TGFbeta inhibitors are selected from the group consisting of an anti-TGFbeta peptide, an anti-TGFbeta antibody, a TGFb-TRAP, and combinations thereof. In some aspects, the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies. In some aspects, the VEGF inhibitors comprise anti-VEGF antibodies, anti-VEGF peptides, or combinations thereof.

In some aspects, at least one of the two or more effector molecules is a human-derived effector molecule. In some aspects, one effector molecule comprises IL12. In some aspects, a second effector molecule comprises CCL21a, IL7, IL15, or IL21. In some aspects, the polynucleotide sequence comprises:

a) an SFFV promoter; and

b) a polynucleotide described in a formula, oriented from 5′ to 3′, comprising

S1-E1-L-S2-E2

wherein S1 comprises a polynucleotide sequence encoding a first secretion signal peptide, wherein the first secretion signal peptide is a human IL12 secretion signal peptide;

E1 comprises a polynucleotide sequence encoding a first effector molecule, wherein the first effector molecule is a human IL12p70 fusion protein;

L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;

S2 comprises a polynucleotide sequence encoding a second secretion signal peptide, wherein the second secretion signal peptide is a human IL21 secretion signal peptide;

E2 comprises a polynucleotide sequence encoding a second effector molecule, wherein the second effector molecule is human IL21; and

wherein the SFFV promoter is operably linked to the polynucleotide, the first secretion signal peptide is operably linked to the first effector molecule, and the second secretion signal peptide is operably linked to the second effector molecule. In some aspects, the polynucleotide sequence comprises the polynucleotide sequence of SEQ ID NO: 144.

In some aspects, the nanoparticle comprises an inorganic material.

Also provided herein is a composition comprising any of the nanoparticles described herein and a pharmaceutically acceptable carrier.

Also provided herein is a method of treating a subject having cancer, the method comprising administering a therapeutically effective dose of any of the nanoparticles described herein or any of the nanoparticles described herein and a pharmaceutically acceptable carrier.

Also provided herein is a method of reducing tumor volume in a subject, the method comprising administering to a subject having a tumor a composition comprising any of the nanoparticles described herein and a pharmaceutically acceptable carrier.

In some aspects, the administering comprises systemic administration. In some aspects, the administering comprises intratumoral administration or intraperitoneal administration. In some aspects, the nanoparticle is capable of engineering a cell in the subject to produce two or more effector molecules that modulate tumor-mediated immunosuppressive mechanisms. In some aspects, the cell is a tumor cell, an immune cell, an erythrocyte, or a platelet cell.

In some aspects, the method further comprises administering a checkpoint inhibitor. In some aspects, the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody. In some aspects, the method further comprises administering an anti-CD40 antibody.

In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor. In some aspects, the tumor is a tumor located in a peritoneal space.

Also provided herein is a virus engineered to comprise a heterologous nucleic acid, wherein the heterologous nucleic acid comprises a polynucleotide sequence encoding two or more effector molecules. In some aspects, the virus is selected from the group consisting of a lentivirus, a retrovirus, an oncolytic virus, an adenovirus, an adeno-associated virus (AAV), and a virus-like particle (VLP).

Also provided herein is an oncolytic virus engineered to comprise a heterologous nucleic acid, wherein the heterologous nucleic acid comprises a polynucleotide sequence encoding two or more effector molecules. In some aspects, the engineered nucleic acid comprises DNA. In some aspects, the engineered nucleic acid comprises RNA.

In some aspects, the polynucleotide sequence comprises a promoter. In some aspects, the promoter comprises an exogenous promoter In some aspects, the polynucleotide sequence further comprises a linker polynucleotide sequence. In some aspects, the promoter is operably linked to the polynucleotide sequence such that the two or more effector molecules are capable of being transcribed as a single polynucleotide comprising the formula (L-E)x, wherein

L comprises a linker polynucleotide sequence,

E comprises a polynucleotide encoding one of the two or more effector molecules,

X=2 to 20, and

wherein for the first iteration of the (L-E) unit L is absent.

In some aspects, the linker polynucleotide sequence is operably associated with the translation of the two or more effector molecules as separate polypeptides. In some aspects, the linker polynucleotide sequence encodes a 2A ribosome skipping tag. In some aspects, the 2A ribosome skipping tag is selected from the group consisting of P2A, T2A, E2A, and F2A. In some aspects, the linker polynucleotide sequence encodes a cleavable polypeptide. In some aspects, the cleavable polypeptide comprises a furin polypeptide sequence. In some aspects, the linker polynucleotide sequence encodes an Internal Ribosome Entry Site (IRES). In some aspects, the linker polynucleotide sequence encodes an additional promoter. In some aspects, the promoter is operably linked to the polynucleotide sequence such that a first polynucleotide encoding one of the two or more effector molecules is capable of being transcribed, wherein the additional promoter is operably linked to the polynucleotide sequence such that a second polynucleotide encoding a second of the two or more effector molecules is capable of being transcribed, and wherein the first and the second polynucleotides are separate polynucleotides. In some aspects, the promoter and the additional promoter are identical. In some aspects, the promoter and the additional promoter are different.

In some aspects, the promoter and/or the additional promoter comprises a constitutive promoter. In some aspects, the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb. In some aspects, the promoter and/or the additional promoter comprises an inducible promoter. In some aspects, the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, each of the two or more effector molecules comprises a secretion signal. In some aspects, one secretion signal peptide comprises a native secretion signal peptide native to a corresponding effector molecule of the two or more effector molecules. In some aspects, one secretion signal peptide comprises a non-native secretion signal peptide non-native to at least one of the two or more effector molecules. In some aspects, the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12. In some aspects, each secretion signal peptide is identical.

In some aspects, a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme. In some aspects, a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme. In some aspects, the therapeutic class of the first effector molecule and the second effector molecule are different.

In some aspects, the cytokine is selected from the group consisting of IL12, IL7, IL21, IL18, IL15, Type I interferons, and Interferon-gamma. In some aspects, the IL12 cytokine is an IL12p70 fusion protein. In some aspects, the chemokine is selected from the group consisting of CCL21a, CXCL10, CXCL11, CXCL13, CXCL10-11 fusion, CCL19, CXCL9, and XCL1. In some aspects, the growth factor is selected from the group consisting of FLT3L and GM-CSF. In some aspects, the co-activation molecule is selected from the group consisting of 4-1BBL and CD40L. In some aspects, the tumor microenvironment modifier is selected from the group consisting of adenosine deaminase, TGFbeta inhibitors, immune checkpoint inhibitors, VEGF inhibitors, and HPGE2. In some aspects, the TGFbeta inhibitors are selected from the group consisting of an anti-TGFbeta peptide, an anti-TGFbeta antibody, a TGFb-TRAP, and combinations thereof. In some aspects, the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies. In some aspects, the VEGF inhibitors comprise anti-VEGF antibodies, anti-VEGF peptides, or combinations thereof.

In some aspects, at least one of the two or more effector molecules is a human-derived effector molecule. In some aspects, one effector molecule comprises IL12. In some aspects, a second effector molecule comprises CCL21a, IL7, IL15, or IL21. In some aspects, the polynucleotide sequence comprises:

a) an SFFV promoter; and

b) a polynucleotide described in a formula, oriented from 5′ to 3′, comprising

S1-E1-L-S2-E2

wherein S1 comprises a polynucleotide sequence encoding a first secretion signal peptide, wherein the first secretion signal peptide is a human IL12 secretion signal peptide;

E1 comprises a polynucleotide sequence encoding a first effector molecule, wherein the first effector molecule is a human IL12p70 fusion protein;

L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;

S2 comprises a polynucleotide sequence encoding a second secretion signal peptide, wherein the second secretion signal peptide is a human IL21 secretion signal peptide;

E2 comprises a polynucleotide sequence encoding a second effector molecule, wherein the second effector molecule is human IL21; and wherein the SFFV promoter is operably linked to the polynucleotide, the first secretion signal peptide is operably linked to the first effector molecule, and the second secretion signal peptide is operably linked to the second effector molecule. In some aspects, the polynucleotide sequence comprises the polynucleotide sequence of SEQ ID NO: 144.

In some aspects, the two or more effector molecules are capable of being transcribed and/or translated in a tumor cell. In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.

In some aspects, the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

Also provided herein is a pharmaceutical composition comprising any of the engineered viruses described herein and a pharmaceutically acceptable carrier.

Also provided herein is a method of treating a subject having cancer, the method comprising administering a therapeutically effective dose of any of the engineered viruses described herein or any of the engineered viruses described herein and a pharmaceutically acceptable carrier.

Also provided herein is a method of reducing tumor volume in a subject, the method comprising administering to a subject having a tumor a composition comprising any of the engineered viruses described herein and a pharmaceutically acceptable carrier.

In some aspects, the administering comprises systemic administration. In some aspects, the administering comprises intratumoral administration or intraperitoneal administration. In some aspects, the engineered virus infects a cell in the subject and produces two or more effector molecules that modulate tumor-mediated immunosuppressive mechanisms. In some aspects, the cell is a tumor cell.

In some aspects, the method further comprises administering a checkpoint inhibitor. In some aspects, the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GALS antibody, an anti-A2AR antibody, an anti-phosphatidylserine antibody, an anti-CD27 antibody, an anti-TNFa antibody, an anti-TREM1 antibody, and an anti-TREM2 antibody. In some aspects, the method further comprises administering an anti-CD40 antibody.

In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor. In some aspects, the tumor is a tumor located in a peritoneal space.

Also provided herein is a method of treating a subject having cancer, the method comprising administering a therapeutically effective dose of a composition, wherein the composition comprises two or more effector molecules.

Also provided herein is a method of reducing tumor volume in a subject, the method comprising administering to a subject having a tumor a therapeutically effective dose of a composition, wherein the composition comprises two or more effector molecules.

Also provided herein is a method of treating a subject having cancer, the method comprising administering a therapeutically effective dose of a composition, wherein the composition comprises an engineered nucleic acid, and wherein the engineered nucleic acid comprises a polynucleotide sequence encoding two or more effector molecules.

Also provided herein is a method of reducing tumor volume in a subject, the method comprising administering to a subject having a tumor a therapeutically effective dose of a composition, wherein the composition comprises an engineered nucleic acid, and wherein the engineered nucleic acid comprises a polynucleotide sequence encoding two or more effector molecules.

In some aspects, the administering comprises one or more intraperitoneal injections. In some aspects, the administering comprises one or more intratumoral injections. In some aspects, the administering comprises systemic administration.

In some aspects, the engineered nucleic acid is an mRNA. In some aspects, the engineered nucleic acid is a cDNA. In some aspects, the composition comprises naked mRNA. In some aspects, the composition comprises a naked plasmid.

In some aspects, the composition comprises a delivery system selected from the group consisting of a viral system, a transposon system, and a nuclease genomic editing system. In some aspects, the viral system is selected from the group consisting of a lentivirus, a retrovirus, a retrotransposon, an oncolytic virus, an adenovirus, an adeno-associated virus (AAV), and a virus-like particle (VLP). In some aspects, the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some aspects, the nuclease genomic editing system is selected from the group consisting of a zinc-finger system, a TALEN system, and a CRISPR system.

In some aspects, the composition comprises an erythrocyte or a platelet cell.

In some aspects, the composition comprises a lipid structure delivery system comprising a lipid-based structure. In some aspects, the lipid-based structure is selected from the group consisting of an extracellular vesicle, a lipid nanoparticle, a micelle, nanovesicle, an exosome, and a liposome.

In some aspects, the composition comprises a nanoparticle. In some aspects, the nanoparticle comprises an inorganic material. In some aspects, the nanoparticle encapsulates the engineered nucleic acid or encapsulates the two or more effector molecules.

In some aspects, the polynucleotide sequence comprises a promoter. In some aspects, the promoter comprises an exogenous promoter polynucleotide sequence. In some aspects, the promoter comprises an endogenous promoter. In some aspects, the polynucleotide sequence further comprises a linker polynucleotide sequence. In some aspects, the promoter is operably linked to the polynucleotide sequence such that the two or more effector molecules are capable of being transcribed as a single polynucleotide comprising the formula (L-E)x, wherein

L comprises a linker polynucleotide sequence,

E comprises a polynucleotide encoding one of the two or more effector molecules,

X=2 to 20, and

wherein for the first iteration of the (L-E) unit L is absent.

In some aspects, the linker polynucleotide sequence is operably associated with the translation of the two or more effector molecules as separate polypeptides. In some aspects, the linker polynucleotide sequence encodes a 2A ribosome skipping tag. In some aspects, the 2A ribosome skipping tag is selected from the group consisting of P2A, T2A, E2A, and F2A. In some aspects, the linker polynucleotide sequence encodes a cleavable polypeptide. In some aspects, the cleavable polypeptide comprises a furin polypeptide sequence. In some aspects, the linker polynucleotide sequence encodes an Internal Ribosome Entry Site (IRES). In some aspects, the linker polynucleotide sequence encodes an additional promoter. In some aspects, the promoter is operably linked to the polynucleotide sequence such that a first polynucleotide encoding one of the two or more effector molecules is capable of being transcribed, wherein the additional promoter is operably linked to the polynucleotide sequence such that a second polynucleotide encoding a second of the two or more effector molecules is capable of being transcribed, and wherein the first and the second polynucleotides are separate polynucleotides. In some aspects, the promoter and the additional promoter are identical. In some aspects, the promoter and the additional promoter are different.

In some aspects, the promoter and/or the additional promoter comprises a constitutive promoter. In some aspects, the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb. In some aspects, the promoter and/or the additional promoter comprises an inducible promoter. In some aspects, the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, each of the two or more effector molecules comprises a secretion signal. In some aspects, one secretion signal peptide comprises a native secretion signal peptide native to a corresponding effector molecule of the two or more effector molecules. In some aspects, one secretion signal peptide comprises a non-native secretion signal peptide non-native to at least one of the two or more effector molecules. In some aspects, the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12. In some aspects, each secretion signal peptide is identical.

In some aspects, a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme. In some aspects, a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme. In some aspects, the therapeutic class of the first effector molecule and the second effector molecule are different.

In some aspects, the cytokine is selected from the group consisting of IL12, IL7, IL21, IL18, IL15, Type I interferons, and Interferon-gamma. In some aspects, the IL12 cytokine is an IL12p70 fusion protein. In some aspects, the chemokine is selected from the group consisting of CCL21a, CXCL10, CXCL11, CXCL13, CXCL10-11 fusion, CCL19, CXCL9, and XCL1. In some aspects, the growth factor is selected from the group consisting of FLT3L and GM-CSF. In some aspects, the co-activation molecule is selected from the group consisting of 4-1BBL and CD40L. In some aspects, the tumor microenvironment modifier is selected from the group consisting of adenosine deaminase, TGFbeta inhibitors, immune checkpoint inhibitors, VEGF inhibitors, and HPGE2. In some aspects, the TGFbeta inhibitors are selected from the group consisting of an anti-TGFbeta peptide, an anti-TGFbeta antibody, a TGFb-TRAP, and combinations thereof. In some aspects, the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies. In some aspects, the VEGF inhibitors comprise anti-VEGF antibodies, anti-VEGF peptides, or combinations thereof.

In some aspects, at least one of the two or more effector molecules is a human-derived effector molecule. In some aspects, one effector molecule comprises IL12. In some aspects, a second effector molecule comprises CCL21a, IL7, IL15, or IL21. In some aspects, the polynucleotide sequence comprises:

a) an SFFV promoter; and

b) an expression cassette described in a formula, oriented from 5′ to 3′, comprising

S1-E1-L-S2-E2

wherein S1 comprises a polynucleotide sequence encoding a first secretion signal peptide, wherein the first secretion signal peptide is a human IL12 secretion signal peptide;

E1 comprises a polynucleotide sequence encoding a first effector molecule, wherein the first effector molecule is a human IL12p70 fusion protein;

L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;

S2 comprises a polynucleotide sequence encoding a second secretion signal peptide, wherein the second secretion signal peptide is a human IL21 secretion signal peptide;

E2 comprises a polynucleotide sequence encoding a second effector molecule, wherein the second effector molecule is human IL21; and

wherein the SFFV promoter is operably linked to the expression cassette, the first secretion signal peptide is operably linked to the first effector molecule, and the second secretion signal peptide is operably linked to the second effector molecule. In some aspects, the polynucleotide sequence comprises the polynucleotide sequence of SEQ ID NO: 144.

In some aspects, the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.

### BRIEF DESCRIPTION OF THE DRAWINGS

FIG. 1 shows treatment using syngeneic and allogeneic MSCs expressing IL12p70/CCL21a in a CT26 model.

FIG. 2A shows rechallenge of tumor free mice with CT26 tumors previously treated using syngeneic and allogeneic MSCs expressing IL12p70/CCL21a in a CT26 model. FIG. 2A shows the treatment schematic.

FIG. 2B shows rechallenge of tumor free mice with CT26 tumors previously treated using syngeneic and allogeneic MSCs expressing IL12p70/CCL21a in a CT26 model. FIG. 2B shows tumor free mice rejecting the tumor implant in contrast to naïve control mice where the tumor became established.

FIG. 3 shows data indicating that intraperitoneally injected murine BM-derived MSCs (BM-MSCs) home to the tumor site of 4T1 breast cancer cells in vivo. Fluorescently labeled BM-MSCs (therapeutic cells) were injected into mice bearing 4T1 breast tumor cells. The breast tumor cells express a luciferase reporter. The first top two panels on the left show imaging of therapeutic cells (BM-MSCs) in mice bearing tumors on day 1 and on day 7 after injection as indicated. The third top panel on the left shows imaging of tumor cells in mice bearing tumors on day 7 after injection. The bottom two panels on the left show imaging of therapeutic cells in normal mice not bearing tumors on day 1 and on day 7 after injection as indicated. A schematic showing the effect of tumors on homing of therapeutic cells is provided on the far right.

FIG. 4 shows data indicating that engineered MSCs expressing IL-12 and CCL21a induced significant tumor growth delay in an orthotopic mouse model of breast cancer. The chart on the left shows the effects of engineered MSCs on 4T1 breast tumor growth in mice (n=8). Each line in the chart represents tumor volume in mice receiving intraperitoneal injection of either control MSC growth media or engineered MSCs on day 0 and day 7. Mice received intraperitoneal injection of engineered MSCs expressing IL-12 and engineered MSCs expressing CCL21a. Tumor volume was determined by caliper measurements every other day. Data represent mean±SEM. *p<0.05, **p<0.005 as compared to control media group. The schematic on the right shows a timeline of treatment and the effect of engineered MSCs expressed combinatorial genes IL-12 and CCL21a on tumor burden in treated mice.

FIG. 5A includes data indicating that engineered MSCs expressing IFN-β, IFN-γ, IL-12, CCL21a, or combinations thereof inhibit tumor growth in an orthotopic mouse model of breast cancer (4T1 triple negative breast carcinoma). Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 5A represents an individual mouse.

FIG. 5B includes data indicating that engineered MSCs expressing IFN-β, IFN-γ, IL-12, CCL21a, or combinations thereof inhibit tumor growth in an orthotopic mouse model of breast cancer (4T1 triple negative breast carcinoma). Each effector was expressed by a different MSC, and the MSCs were combined (at a 1:1 ratio) for combinatorial treatment. The left graph of FIG. 5B shows the tumor weight for individual mice in each treatment on day 14, and the mean±SEM for each treatment group. The right graph of FIG. 5B shows the tumor volume represented as mean±SEM for mice receiving each treatment over time.

FIG. 6A includes data indicating that engineered MSCs expressing OX40L, TRAIL, IL15, cGAS, or combinations thereof do not inhibit tumor growth significantly in an orthotopic mouse model of breast cancer (4T1 triple negative breast carcinoma). Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 6A represents an individual mouse.

FIG. 6B includes data indicating that engineered MSCs expressing OX40L, TRAIL, IL15, cGAS, or combinations thereof do not inhibit tumor growth significantly in an orthotopic mouse model of breast cancer (4T1 triple negative breast carcinoma). Each effector was expressed by a different MSC, and the MSCs were combined (at a 1:1 ratio) for combinatorial treatment. The left graph of FIG. 6B shows the tumor weight for individual mice in each treatment, and the mean±SEM for each treatment group. The right graph of FIG. 6B shows tumor volume represented as mean±SEM for mice receiving each treatment over time.

FIG. 7A includes data indicating that engineered MSCs expressing IL-12 and CCL21a inhibit tumor growth in an orthotopic mouse model of breast cancer (4T1 triple negative breast carcinoma); however the addition of anti-CD40 antibody does not reduce tumor growth. Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 7A represents an individual mouse.

FIG. 7B includes data indicating that engineered MSCs expressing IL-12 and CCL21a inhibit tumor growth in an orthotopic mouse model of breast cancer (4T1 triple negative breast carcinoma); however the addition of anti-CD40 antibody does not reduce tumor growth. Each effector was expressed by a different MSC, and the MSCs were combined (at a 1:1 ratio) for combinatorial treatment. FIG. 7B shows the tumor weight for individual mice in each treatment, and the mean±SEM for each treatment group.

FIG. 8A includes data indicating that engineered MSCs expressing OX40L, TRAIL, IL15, HACvPD-1, or combinations thereof do not inhibit tumor growth significantly in an subcutaneous mouse model of breast cancer (4T1 triple negative breast carcinoma). Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 8A represents an individual mouse.

FIG. 8B includes data indicating that engineered MSCs expressing OX40L, TRAIL, IL15, HACvPD-1, or combinations thereof do not inhibit tumor growth significantly in an subcutaneous mouse model of breast cancer (4T1 triple negative breast carcinoma). Each effector was expressed by a different MSC, and the MSCs were combined (at a 1:1 ratio) for combinatorial treatment. The left graph of FIG. 8B shows the tumor weight for individual mice in each treatment, and the mean±SEM for each treatment group. The right graph of FIG. 8B shows body weight represented as mean±SEM for mice receiving each treatment over time.

FIG. 9A includes data indicating that engineered MSCs expressing IL-12 and CCL21a inhibit tumor growth in an orthotopic mouse model of breast cancer (4T1 triple negative breast carcinoma); however the combination of MSCs expressing CCL21a, IL-36 gamma and IL-7 does not reduce tumor growth. Some of the effector combinations tested, however, may cause toxicity. Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 9A represents an individual mouse.

FIG. 9B includes data indicating that engineered MSCs expressing IL-12 and CCL21a inhibit tumor growth in an orthotopic mouse model of breast cancer (4T1 triple negative breast carcinoma); however the combination of MSCs expressing CCL21a, IL-36 gamma and IL-7 does not reduce tumor growth. Some of the effector combinations tested, however, may cause toxicity. Each effector was expressed by a different MSC, and the MSCs were combined (at a 1:1 ratio) for combinatorial treatment. FIG. 9B shows the tumor weight for individual mice in each treatment, and the mean±SEM for each treatment group.

FIG. 10A includes data from a GFP dose escalation study for toxicity and screening. FIG. 10A shows that engineered MSCs expressing GFP do not elicit toxicity. Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 10A represents an individual mouse.

FIG. 10B includes data from a GFP dose escalation study for toxicity and screening. Each effector was expressed by a different MSC, and the MSCs were combined (at a 1:1 ratio) for combinatorial treatment. FIG. 10B shows the tumor weight for individual mice in each treatment, and the mean±SEM for each treatment group.

FIG. 11A shows that engineered human MSCs do not home to mouse 4T1 tumors. Each line of FIG. 11A represents an individual mouse.

FIG. 11B shows that engineered human MSCs do not home to mouse 4T1 tumors. FIG. 11B shows the tumor weight for individual mice in each treatment, and the mean±SEM for each treatment group.

FIG. 12 includes data showing that IL-12 and CCL21a can reduce tumor expansion. Each line of FIG. 12 represents an individual mouse.

FIG. 13A includes data indicating that engineered MSCs expressing IL-12 and CCL21 are sufficient to inhibit tumor growth in an orthotopic mouse model of breast cancer (4T1 triple negative breast carcinoma), and the addition of a checkpoint inhibitor (anti-PD-1 antibody or anti-CTLA-4 antibody) did not increase efficacy. Each effector was expressed by a different MSC, and the MSCs were combined (at a 1:1 ratio) for combinatorial treatment, and the checkpoint inhibitor was injected separately. Each chart shows the effect of engineered MSCs expressing the indicated immunotherapies alone or in combination on the growth of 4T1 breast tumors in mice (n=6-8). Each line of FIG. 13A represents an individual mouse.

FIG. 13B includes data indicating that engineered MSCs expressing IL-12 and CCL21 are sufficient to inhibit tumor growth in an orthotopic mouse model of breast cancer (4T1 triple negative breast carcinoma), and the addition of a checkpoint inhibitor (anti-PD-1 antibody or anti-CTLA-4 antibody) did not increase efficacy. Each effector was expressed by a different MSC, and the MSCs were combined (at a 1:1 ratio) for combinatorial treatment, and the checkpoint inhibitor was injected separately. FIG. 13B shows the tumor weight for individual mice in each treatment, and the mean±SEM for each treatment group.

FIG. 14 shows data indicating that engineered MSCs expressing IL-12 and CCL21a induced significant tumor growth delay in a mouse model of colorectal cancer. The graph on the left shows the effects of engineered MSCs on CT26 colorectal tumor growth in mice (n=8). Each line in the chart represents tumor volume in mice receiving intraperitoneal injection of either control MSC growth media or engineered MSCs on day 0 and day 7. Mice received intraperitoneal injection of engineered MSCs expressing IL-12 and engineered MSCs expressing CCL21a. Tumor volume was determined by caliper measurements every other day. Data represent mean±SEM. *p<0.05, **p<0.005 as compared to control media group. The schematic on the right shows a timeline of treatment and the effect of engineered MSCs expressed combinatorial genes IL-12 and CCL21a on tumor burden in treated mice.

FIG. 15 is a graph showing tumor growth kinetics in the CT26 mouse model to determine optimal time for dosing the engineered MSC cells.

FIG. 16A includes data indicating the effects of engineered MSCs expressing IL-12 and CCL21a combined with anti-CD40 or anti-CTLA4 antibodies on average tumor growth in a syngeneic mouse model of colon cancer. Mice bearing CT26 colon tumors were treated with one of seven treatments (n=5-6 per treatment group). MSC-IL-12+MSC-CCL21a indicates treatment with engineered cells expressing IL-12 and with engineered cells expressing CCL21a (at a 1:1 ratio) for combinatorial treatment. Each line of FIG. 16A represents an individual mouse.

FIG. 16B includes data indicating the effects of engineered MSCs expressing IL-12 and CCL21a combined with anti-CD40 or anti-CTLA4 antibodies on average tumor growth in a syngeneic mouse model of colon cancer. Mice bearing CT26 colon tumors were treated with one of seven treatments (n=5-6 per treatment group). MSC-IL-12+MSC-CCL21a indicates treatment with engineered cells expressing IL-12 and with engineered cells expressing CCL21a (at a 1:1 ratio) for combinatorial treatment. The left graph of FIG. 16B shows the tumor weight for individual mice in each treatment, and the mean±SEM for each treatment group. The right graph of FIG. 16B shows the tumor volume represented as mean±SEM for mice receiving each treatment over time.

FIG. 17A includes data from a dose-dependent long-term survival study. FIG. 17A shows the tumor volume of the individual group. Each line of FIG. 17A represents an individual mouse.

FIG. 17B includes data from a dose-dependent long-term survival study. FIG. 17B shows body weight represented as mean±SEM (top left), tumor volume represented as mean±SEM (bottom left), and survival rate (right).

FIG. 18A includes data indicating that engineered MSCs expressing IL-12, CCL21a, and either IL15 or HACvPD-1 inhibit tumor growth significantly in a mouse model of colorectal cancer. Each effector was expressed by a different MSC, and the MSCs were combined (at a 1:1 ratio) for combinatorial treatment. Each chart shows the effect of engineered MSCs expressing the indicated immunotherapies alone or in combination on the growth of CT26 colorectal tumors in mice (n=6-8). Each line of FIG. 18A represents an individual mouse.

FIG. 18B includes data indicating that engineered MSCs expressing IL-12, CCL21a, and either IL15 or HACvPD-1 inhibit tumor growth significantly in a mouse model of colorectal cancer. Each effector was expressed by a different MSC, and the MSCs were combined (at a 1:1 ratio) for combinatorial treatment. FIG. 18B shows the tumor weight for individual mice in each treatment, and the mean±SEM for each treatment group.

FIG. 18C includes data indicating that engineered MSCs expressing IL-12, CCL21a, and either IL15 or HACvPD-1 inhibit tumor growth significantly in a mouse model of colorectal cancer. Each effector was expressed by a different MSC, and the MSCs were combined (at a 1:1 ratio) for combinatorial treatment. FIG. 18C is a representative graph of the infiltrating immune population within the tumor microenvironment for individual mice in each treatment, and the mean±SEM for each treatment group.

FIG. 18D includes data indicating that engineered MSCs expressing IL-12, CCL21a, and either IL15 or HACvPD-1 inhibit tumor growth significantly in a mouse model of colorectal cancer. Each effector was expressed by a different MSC, and the MSCs were combined (at a 1:1 ratio) for combinatorial treatment. FIG. 18D shows the percentage of regulatory T cells (Treg) in the total CD3 population for individual mice in each treatment, and the mean±SEM for each treatment group. There was a significant decrease in the numbers of Tregs in the tumor microenvironment treated with engineered MSC-IL2 and CCL21a.

FIG. 18E includes data indicating that engineered MSCs expressing IL-12, CCL21a, and either IL15 or HACvPD-1 inhibit tumor growth significantly in a mouse model of colorectal cancer. Each effector was expressed by a different MSC, and the MSCs were combined (at a 1:1 ratio) for combinatorial treatment. FIG. 18E correlates the percentage of immune infiltration with tumor weight. Samples with high lymphocytes (CD3+) were found to correlate with low tumor weight, while samples with high myeloid (CD11b+) infiltration were correlated with higher tumor burden.

FIG. 19 shows the tumor volume for individual mice in each treatment. Efficacy was determined by tumor volume from caliper measurement every other day.

FIG. 20 shows the tumor weight for individual mice in each treatment, and the mean±SEM for each treatment group. Efficacy was determined by tumor weight.

FIG. 21A shows the kinetics of CT26-LUC (luciferase) tumor growth in the intraperitoneal space. A CT26 cell line was injected at day 0 and three (3) mice were harvested at day 7, day 10, day 14, and day 18 to determine the kinetics of tumor growth. The first row of FIG. 21A measures the mice body weight (left panel) and ROI (right panel) with an IVIS imager to monitor tumor burden. The second row monitors the tumor weight (left panel) and the ROI (right panel) of the tumor of individual mice in each group. The third row correlates the tumor weight with either whole body ROI (left panel) or tumor ROI (right panel).

FIG. 21B shows the kinetics of CT26-LUC (luciferase) tumor growth in the intraperitoneal space. A CT26 cell line was injected at day 0 and three (3) mice were harvested at day 7, day 10, day 14, and day 18 to determine the kinetics of tumor growth. FIG. 21B shows the immune profile of three (3) mice in the day 18 group to better characterize the tumor microenvironment.

FIG. 22A includes data indicating that engineered MSCs expressing IL-12 and CCL21a inhibit tumor growth in a subcutaneous mouse model of colorectal cancer; however the combination of MSCs expressing CCL21a and IL-36 gamma or IL-7 does not reduce tumor growth. Each effector was expressed by a different MSC, and the MSCs were combined (at a 1:1 ratio) for combinatorial treatment. Each chart shows the effect of engineered MSCs expressing the indicated immunotherapies alone or in combination on the growth of CT26 colon tumors in mice (n=6-8). Each line of FIG. 22A represents an individual mouse.

FIG. 22B includes data indicating that engineered MSCs expressing IL-12 and CCL21a inhibit tumor growth in a subcutaneous mouse model of colorectal cancer; however the combination of MSCs expressing CCL21a and IL-36 gamma or IL-7 does not reduce tumor growth. Each effector was expressed by a different MSC, and the MSCs were combined (at a 1:1 ratio) for combinatorial treatment. FIG. 22B shows the tumor weight for individual mice in each treatment group, and the mean±SEM for each treatment group.

FIG. 23A includes tumor immune infiltrate statistics from the experiment represented by FIGS. 22A-22B. Three mice were selected from PBS, Naïve MSC, and MSC-IL12+MSC-CCL21a (combo) group to run flow cytometry to immune profile tumor microenvironment. FIG. 23A shows a significant increase in infiltrating CD3 and CD8 cytotoxic T population in the combo group compared to the group dosed with naïve MSC.

FIG. 23B includes tumor immune infiltrate statistics from the experiment represented by FIGS. 22A-22B. Three mice were selected from PBS, Naïve MSC, and MSC-IL12+MSC-CCL21a (combo) group to run flow cytometry to immune profile tumor microenvironment. FIG. 23B shows a significant reduction in granulocytic myeloid-derived suppressor cells (gMDSCs) and macrophage population in the combo group compared to group treated with Naïve MSC.

FIG. 24A includes data relating to immune percentage and tumor weight, relating to the experiments represented by FIGS. 22A-22B. FIG. 24A shows that samples with more CD3+ and CD8+ T cells (top left and top center graph) correlate strongly with a decrease in tumor weight. These figures also show that samples with fewer CD11b myeloid cells, including macrophage, dendritic cells, and MDSC, display lower tumor burden (lower center and lower right graph).

FIG. 24B includes data relating to immune percentage and tumor weight, relating to the experiments represented by FIGS. 22A-22B. FIG. 24B shows that samples with fewer CD11b myeloid cells, including macrophage, dendritic cells, and MDSC, display lower tumor burden (upper row).

FIG. 25A includes data from MSC-IL-12+CCL21a therapy in intraperitoneal and subcutaneous colorectal cancer mouse models. Three different lots of a lentiviral transduced line was tested for MSC-IL12 and CCL21a (TLOO8-3/4, TL019-01/02, and TL022-01/02; each TL number represents one lot). FIG. 25A shows that all three lots of MSC-IL12+MSC-CCL21a can reduce tumor burden in both subcutaneous and intraperitoneal model (first 5 graphs are from the SC model and last 3 are from the IP model). Tumors from all mice were collected on day 11. Each line of FIG. 25A represents an individual mouse.

FIG. 25B includes data from MSC-IL-12+CCL21a therapy in intraperitoneal and subcutaneous colorectal cancer mouse models. Three different lots of a lentiviral transduced line was tested for MSC-IL12 and CCL21a (TLOO8-3/4, TL019-01/02, and TL022-01/02; each TL number represents one lot). FIG. 25B shows the average tumor weight from each group, and the mean±SEM for each treatment group.

FIG. 26A includes data indicating that engineered combination treatment MSC-IL-12+MSC-CCL21a, or MSC-CCL21a+MSC-IFN-β, inhibit tumor growth in a subcutaneous mouse model of colorectal cancer; however the combination of MSCs expressing CCL21a and s41BBL does not reduce tumor growth. Each effector was expressed by a different MSC, and the MSCs were combined (at a 1:1 ratio) for combinatorial treatment. Each chart shows the effect of engineered MSCs expressing the indicated immunotherapies alone or in combination on the growth of CT26 tumors in mice (n=6-8). Each line of FIG. 26A represents an individual mouse.

FIG. 26B includes data indicating that engineered combination treatment MSC-IL-12+MSC-CCL21a, or MSC-CCL21a+MSC-IFN-β, inhibit tumor growth in a subcutaneous mouse model of colorectal cancer; however the combination of MSCs expressing CCL21a and s41BBL does not reduce tumor growth. Each effector was expressed by a different MSC, and the MSCs were combined (at a 1:1 ratio) for combinatorial treatment. FIG. 26B shows the tumor weight for individual mice in each treatment, and the mean for each treatment group. MSC-IL12+MSC-CCL21a shows best efficacy compared to mice injected with naïve MSC. Treatment efficacy was also observed in the group treated with MSC-IFNb+MSC-CCL21a.

FIG. 27A provides additional data from the experiment represented by FIGS. 26A-26B. FIG. 27A are graphs that show immune profiles of each group treated with indicated engineered MSC. A consistent decrease in macrophage population was observed after treating with MSC-IL12+MSC-CCL21a. A general trend of increased infiltration in CD3+ population and decreased infiltration in CD11b+ population was also observed when compared to group treated with MSC-IL12+MSC-CCL21a against naïve MSC.

FIG. 27B provides additional data from the experiment represented by FIGS. 26A-26B. FIG. 27B are graphs that show immune profiles of each group treated with indicated engineered MSC. A general trend of increased infiltration in CD3+ population and decreased infiltration in CD11b+ population was also observed when compared to group treated with MSC-IL12+MSC-CCL21a against naïve MSC.

FIG. 28A also provides additional data from the experiment represented by FIGS. 26A-26B. FIG. 28A shows the correlation of immune infiltration with tumor weight.

FIG. 28B also provides additional data from the experiment represented by FIGS. 26A-26B. FIG. 28B shows the correlation of immune infiltration with tumor weight. Samples with low macrophage and dendritic cells have lower tumor burden (top center and top right).

FIG. 29 shows graphs combining the in vivo data from the colorectal cancer models above (FIG. 22A and FIG. 26A). The combined CT26 data from FIG. 22A and FIG. 26A capture three groups: Tumor only (PBS), treated with naïve MSC, and treated with MSC-IL12+MSC-CCL21a.

FIG. 30A also shows combined data from FIG. 22A and FIG. 26A. The graphs show the average number of immune infiltration from the flow cytometry experiment data. Statistical significance was observed in CD8+T, demonstrating the ability of MSC-IL12+MSC-CCL21a to repolarize tumor microenvironment and allow more cytotoxic T cell infiltration.

FIG. 30B also shows combined data from FIG. 22A and FIG. 26A. The graphs show the average number of immune infiltration from the flow cytometry experiment data. There was a reduction in CD11b+ myeloid population infiltration in the groups that were treated by MSC-IL12+MSC-CCL21a. The data collected show that the dendritic cells and the macrophage population was statistical significance.

FIG. 31 shows the vector map of pL17D.

FIG. 32 shows MSCs engineered to express different effector molecules either alone or in combination and their efficacy in reducing CT26 tumor burden in an IP tumor model as assessed by BLI levels.

FIG. 33 shows MSCs engineered to express different effector molecules either alone or in combination and their efficacy in reducing B16F10 tumor burden in an IP tumor model as assessed by BLI levels.

FIG. 34 shows the lentiviral expression vector map for expression of human IL12 (p70) and human CCL21a from a single lentiviral expression vector.

FIG. 35A shows production by engineered hMSCs of hIL12, as assessed by cytokine ELISA.

FIG. 35B shows production by engineered hMSCs of hCCL21a, as assessed by cytokine ELISA.

FIG. 36A shows a schematic of a transwell assay for assessing functional T cell modulation by hIL12 produced from MSCs.

FIG. 36B shows a transwell assay demonstrating functional T cell modulation by hIL12 produced from MSCs as assessed by IFNγ production.

FIG. 37A shows homing to tumors by MSCs in IP tumor-bearing mice tumors as assessed by bioluminescence imaging. FIG. 37A shows homing in a CT26 tumor model (images shown).

FIG. 37B shows homing to tumors by MSCs in IP tumor-bearing mice tumors as assessed by bioluminescence imaging. FIG. 37B shows homing in a CT26 tumor model for individual mice in each treatment, and the mean±SEM for each treatment group (quantification summary of images shown in FIG. 37A).

FIG. 37C shows homing to tumors by MSCs in IP tumor-bearing mice tumors as assessed by bioluminescence imaging. FIG. 37C shows quantitative real time PCR for individual mice in each treatment, and the mean±SEM for each treatment group.

FIG. 37D shows homing to tumors by MSCs in IP tumor-bearing mice tumors as assessed by bioluminescence imaging. FIG. 37D shows fluorescence microscopy against firefly luciferase.

FIG. 37E shows homing to tumors by MSCs in IP tumor-bearing mice tumors as assessed by bioluminescence imaging. FIG. 37E shows homing in a B16F10 tumor model for individual mice in each treatment, and the mean±SEM for each treatment group (quantification summary of images).

FIG. 38 shows IL12p70 expressing MSCs leading to reduction in tumor burden as assessed by BLI (top panels—images; and bottom left panel—individual mice in each treatment and the mean±SEM for each treatment group) and a complete elimination of detectable intraperitoneal tumors by tumor weight for individual mice in each treatment, and the mean±SEM for each treatment group (bottom right panel) in a CT26 IP model.

FIG. 39 shows IL12p70 expressing MSCs leading to reduction in tumor burden as assessed by BLI (top panels—images; and bottom left panel—individual mice in each treatment and the mean±SEM for each treatment group) and a complete elimination of detectable intraperitoneal tumors by tumor weight for individual mice in each treatment, and the mean±SEM for each treatment group (bottom right panel) in a B16F10 IP model.

FIG. 40A shows IL12p70/CCL21a expressing MSCs leading to reduction in tumor burden as assessed by BLI in a CT26 IP model. FIG. 40A shows the mean tumor burden as assessed by BLI for PBS treated (circle), MSC-Flag-Myc (“Naïve MSC” square), and IL12p70/CCL21a expressing MSCs (triangle).

FIG. 40B shows IL12p70/CCL21a expressing MSCs leading to reduction in tumor burden as assessed by BLI in a CT26 IP model. FIG. 40B shows the tumor burden in individual mice as assessed by BLI for PBS treated, MSC-Flag-Myc (“Naïve MSC”), and IL12p70/CCL21a expressing MSCs (left, middle, and right panels, respectively). Each line of FIG. 40B represents an individual mouse.

FIG. 40C shows treatment with IL12p70/CCL21a expressing MSCs led to prolonged survival (100% survival greater than 90 days), while control treated mice all died or were euthanized by Day 20.

FIG. 41 shows treatment with IL12p70 expressing MSCs led to prolonged survival.

FIG. 42A shows relative growth of genetically engineered MSCs across different MOIs (95000, 9500, 950, or uninfected) in Donor 1.

FIG. 42B shows relative growth of genetically engineered MSCs across different MOIs (95000, 9500, 950, or uninfected) in Donor 2.

FIG. 42C shows relative growth of genetically engineered MSCs across different MOIs (95000, 9500, 950, or uninfected) in Donor 3.

FIG. 43 shows two independent human BM-MSC cell lines from 2 different donors (top and bottom row, respectively) that were transduced with constructs containing various promoters driving EGFP expression. Percent GFP (left panels) and MFI (right panels) of engineered cells at day 25 post transduction is shown.

FIG. 44 shows two independent human BM-MSC cell lines from 2 different donors that were transduced with constructs containing various promoters driving EGFP expression. Shown is EGFP MFI tracked over time (day 7 to day 28 post-transduction) for either the two independent human BM-MSC cell lines individually (left panel) or with data from the two independent human BM-MSC cell lines combined (right panel).

FIG. 45 shows secretion of IL-12p70 by engineered MSCs as assessed by ELISA.

FIG. 46 shows secretion of IL-21 by engineered MSCs as assessed by ELISA.

FIG. 47 shows the ratio of secreted IL-12p70 to IL-21 by engineered MSCs as assessed by ELISA.

FIG. 48 shows results of a functional reporter assay for IL-12p70 using HEK-293T cells with a STAT4-SEAP reporter to assess cytokine production and secretion by engineered MSCs.

FIG. 49 shows a results of a functional reporter assay for IL-21 using intracellular phospho-flow to quantify phospho-STAT1 (left panel) and phospho-STAT3 (right panel) in NK-92 human natural killer cells to assess cytokine production and secretion by engineered MSCs.

FIG. 50 shows results of a functional reporter assay for IL-12 using a IL21R-U2OS IL21R/IL2RG dimerization reporter to assess cytokine production and secretion by engineered MSCs.

### DETAILED DESCRIPTION

Provided herein are combinatorial immunotherapy compositions and methods directed to modulating different tumor-mediated immunosuppressive mechanisms within and/or near a tumor (a “tumor microenvironment (TME)”) through the production of effector molecules.

An “effector molecule,” refers to a molecule (e.g., a nucleic acid such as DNA or RNA, or a protein (polypeptide) or peptide) that binds to another molecule and modulates the biological activity of that molecule to which it binds. For example, an effector molecule may act as a ligand to increase or decrease enzymatic activity, gene expression, or cell signaling. Thus, in some embodiments, an effector molecule modulates (activates or inhibits) different immunomodulatory mechanisms. By directly binding to and modulating a molecule, an effector molecule may also indirectly modulate a second, downstream molecule. In some embodiments, an effector molecule is a secreted molecule, while in other embodiments, an effector molecule is bound to the cell surface or remains intracellular. For example, effector molecules include intracellular transcription factors, microRNA, and shRNAs that modify the internal cell state to, for example, enhance immunomodulatory activity, homing properties, or persistence of the cell. Non-limiting examples of effector molecules include cytokines, chemokines, enzymes that modulate metabolite levels, antibodies or decoy molecules that modulate cytokines, homing molecules, and/or integrins.

The term “modulate” encompasses maintenance of a biological activity, inhibition (partial or complete) of a biological activity, and stimulation/activation (partial or complete) of a biological activity. The term also encompasses decreasing or increasing (e.g., enhancing) a biological activity. Two different effector molecules are considered to “modulate different tumor-mediated immunosuppressive mechanisms” when one effector molecule modulates a tumor-mediated immunosuppressive mechanism (e.g., stimulates T cell signaling) that is different from the tumor-mediated immunosuppressive mechanism modulated by the other effector molecule (e.g., stimulates antigen presentation and/or processing).

Modulation by an effector molecule may be direct or indirect. Direct modulation occurs when an effector molecule binds to another molecule and modulates activity of that molecule. Indirect modulation occurs when an effector molecule binds to another molecule, modulates activity of that molecule, and as a result of that modulation, the activity of yet another molecule (to which the effector molecule is not bound) is modulated.

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in an increase in an immunostimulatory and/or anti-tumor immune response by at least 20%, at least 30%, at least 40%, at least 50%, at least 60%, at least 70%, at least 80%, at least 90%, at least 100%. In some embodiments, modulation of a tumor-mediated immunosuppressive mechanism results in an increase in an immunostimulatory and/or anti-tumor immune response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “an increase” in an immunostimulatory and/or anti-tumor immune response, for example, systemically or in a tumor microenvironment, is relative to the immunostimulatory and/or anti-tumor immune response that would otherwise occur, in the absence of the effector molecule(s).

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in an increase in an immunostimulatory and/or anti-tumor immune response by at least 3 fold, at least 5 fold, at least 10 fold, at least 20 fold, at least 50 fold, or at least 100 fold. In some embodiments, modulation of a tumor-mediated immunosuppressive mechanism results in an increase in an immunostimulatory and/or anti-tumor immune response by 2-10, 2-20, 2-30, 2-40, 2-50, 2-60, 2-70, 2-80, 2-90, or 2-100 fold.

Non-limiting examples of immunostimulatory and/or anti-tumor immune mechanisms include T cell signaling, activity and/or recruitment, antigen presentation and/or processing, natural killer cell-mediated cytotoxic signaling, activity and/or recruitment, dendritic cell differentiation and/or maturation, immune cell recruitment, pro-inflammatory macrophage signaling, activity and/or recruitment, stroma degradation, immunostimulatory metabolite production, stimulator of interferon genes (STING) signaling (which increases the secretion of IFN and Th1 polarization, promoting an anti-tumor immune response), and/or Type I interferon signaling. An effector molecule may stimulate at least one (one or more) of the foregoing immunostimulatory mechanisms, thus resulting in an increase in an immunostimulatory response. Changes in the foregoing immunostimulatory and/or anti-tumor immune mechanisms may be assessed, for example, using in vitro assays for T cell proliferation or cytotoxicity, in vitro antigen presentation assays, expression assays (e.g., of particular markers), and/or cell secretion assays (e.g., of cytokines).

In some embodiments, modulation of a tumor-mediated immunosuppressive mechanism by at least one effector molecule results in a decrease in an immunosuppressive response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in a decrease in an immunosuppressive response by at least 20%, at least 30%, at least 40%, at least 50%, at least 60%, at least 70%, at least 80%, at least 90%, at least 100%. In some embodiments, modulation of a tumor-mediated immunosuppressive mechanism results in a decrease in an immunosuppressive response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “a decrease” in an immunosuppressive response, for example, systemically or in a tumor microenvironment, is relative to the immunosuppressive response that would otherwise occur, in the absence of the effector molecule(s).

In some embodiments, modulation of a tumor-mediated immunosuppressive mechanism by at least one effector molecule results in a decrease in an immunosuppressive response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in a decrease in an immunosuppressive response by at least 3 fold, at least 5 fold, at least 10 fold, at least 20 fold, at least 50 fold, or at least 100 fold. In some embodiments, modulation of a tumor-mediated immunosuppressive mechanism results in a decrease in an immunosuppressive response by 2-10, 2-20, 2-30, 2-40, 2-50, 2-60, 2-70, 2-80, 2-90, or 2-100 fold.

Non-limiting examples of immunosuppressive mechanisms include negative costimulatory signaling, pro-apoptotic signaling of cytotoxic cells (e.g., T cells and/or NK cells), T regulatory (Treg) cell signaling, tumor checkpoint molecule production/maintenance, myeloid-derived suppressor cell signaling, activity and/or recruitment, immunosuppressive factor/metabolite production, and/or vascular endothelial growth factor signaling. An effector molecule may inhibit at least one (one or more) of the foregoing immunosuppressive mechanisms, thus resulting in a decrease in an immunosuppressive response. Changes in the foregoing immunosuppressive mechanisms may be assessed, for example, by assaying for an increase in T cell proliferation and/or an increase in IFNγ production (negative co-stimulatory signaling, T_{reg} cell signaling and/or MDSC); Annexin V/PI flow staining (pro-apoptotic signaling); flow staining for expression, e.g., PDL1 expression (tumor checkpoint molecule production/maintenance); ELISA, LUMINEX®, RNA via qPCR, enzymatic assays, e.g., IDO tryptophan catabolism (immunosuppressive factor/metabolite production); and phosphorylation of PI3K, Akt, p38 (VEGF signaling).

In some embodiments, effector molecules function additively: the effect of two effector molecules, for example, may be equal to the sum of the effect of the two effector molecules functioning separately. In other embodiments, effector molecules function synergistically: the effect of two effector molecules, for example, may be greater than the combined function of the two effector molecules.

Effector molecules that modulate tumor-mediated immunosuppressive mechanisms and/or modify tumor microenvironments may be, for example, secreted factors (e.g., cytokines, chemokines, antibodies, and/or decoy receptors that modulate extracellular mechanisms involved in the immune system), inhibitors (e.g., antibodies, antibody fragments, ligand TRAP and/or small blocking peptides), intracellular factors that control cell state (e.g., microRNAs and/or transcription factors that modulate the state of cells to enhance pro-inflammatory properties), factors packaged into exosomes (e.g., microRNAs, cytosolic factors, and/or extracellular factors), surface displayed factors (e.g., checkpoint inhibitors, TRAIL), and and/or metabolic genes (e.g., enzymes that produce/modulate or degrade metabolites or amino acids).

In some embodiments, at least one of the effector molecules stimulates an immunostimulatory mechanism in the tumor microenvironment and/or inhibits an immunosuppressive mechanism in the tumor microenvironment.

In some embodiments, at least one of the effector molecules (a) stimulates T cell signaling, activity and/or recruitment, (b) stimulates antigen presentation and/or processing, (c) stimulates natural killer cell-mediated cytotoxic signaling, activity and/or recruitment, (d) stimulates dendritic cell differentiation and/or maturation, (e) stimulates immune cell recruitment, (f) stimulates pro-inflammatory macrophage signaling, activity and/or recruitment or inhibits anti-inflammatory macrophage signaling, activity and/or recruitment, (g) stimulates stroma degradation, (h) stimulates immunostimulatory metabolite production, (i) stimulates Type I interferon signaling, (j) inhibits negative costimulatory signaling, (k) inhibits pro-apoptotic signaling of anti-tumor immune cells, (l) inhibits T regulatory (T_{reg}) cell signaling, activity and/or recruitment, (m) inhibits tumor checkpoint molecules, (n) stimulates stimulator of interferon genes (STING) signaling, (o) inhibits myeloid-derived suppressor cell signaling, activity and/or recruitment, (p) degrades immunosuppressive factors/metabolites, (q) inhibits vascular endothelial growth factor signaling, and/or (r) directly kills tumor cells.

In some embodiments, effector molecules may be selected from the following non-limiting classes of molecules: cytokines, antibodies, chemokines, nucleotides, peptides, and enzymes. Non-limiting examples of the foregoing classes of effector molecules are listed in Table 1 and specific sequences encoding exemplary effector molecules are listed in Table 6. Effector molecules can be human, such as those listed in Table 1 or Table 6 or human equivalents of murine effector molecules listed in Table 1 or Table 6. Effector molecules can be human-derived, such as the endogenous human effector molecule or an effector molecule modified and/or optimized for function, e.g., codon optimized to improve expression, modified to improve stability, or modified at its signal sequence (see below). Various programs and algorithms for optimizing function are known to those skilled in the art and can be selected based on the improvement desired, such as codon optimization for a specific species (e.g., human, mouse, bacteria, etc.).

TABLE 1
Exemplary Effector Molecules

| Effector name | Category | Function |
|---|---|---|
| anti-CD40 or | Agonist antibody | Stimulates B-cells and antigen |
| CD40 Ligand | | presenting cells. |
| Flt3L | Ligand agonist | Stimulates myeloid cells and |
| | | antigen presenting cells |
| CXCL10-11 | Chemokine | Attracts T-cells |
| fusion | | |
| TGFb blocking | Antagonist peptides | Inhibit TGFb pathway, |
| peptides | | TME modifier |
| Adenosine | TME modifier | Degradation of suppressive |
| deaminase | | adenosine in the TME |
| (ADA) | | |
| Kyneurinase | TME modifier | Degradation of kyneurine |
| HPGE2 | TME modifier | Degradation of PGE2 |
| CXCL13 | Chemokine | Attracts B-cells |
| anti PD-1/PD-L1 | Agonist antibody | Remove checkpoint |
| anti-CTLA-4 | Agonist antibody | Remove checkpoint |
| anti-VEGF | Antagonist | Neutralizes an |
| | antibody | immunosuppressive/ |
| | | angiogenesis factor |
| anti-TNFa | Antagonist | Neutralizes cytokine/ |
| | antibody | pro-tumor factor |
| anti-IL-10 | Antagonist | Neutralizes |
| | antibody | immunosuppressive cytokine |
| anti-SDF1/ | Antagonist | Neutralizes pro-tumor |
| CXCL12 | antibody | chemokine |
| (TβRII)2 trap | Capture trap | Neutralizes an |
| | | immunosuppressive cytokine |
| CCL21 | Chemokine | Attracts leukocytes/NK |
| CCL1 | Chemokine | Attracts leukocytes/NK |
| CCL17 | Chemokine | Attracts leukocytes/NK |
| CCL19 | Chemokine | Attracts leukocytes/NK |
| CCL21 | Chemokine | Attracts leukocytes/NK |
| CCL20 | Chemokine | Attracts leukocytes/NK |
| CCL21a | Chemokine | Attracts leukocytes/NK |
| MIP1b (CCL5) | Chemokine | Attracts leukocytes/NK |
| CXCL10 | Chemokine | Attracts leukocytes/NK |
| CXCL11 | Chemokine | Attracts leukocytes/NK |
| CCL2 | Chemokine | Attracts monocytes |
| MIP-1alpha | Chemokine | Attracts leukocytes/NK |
| (CCL3) | | |
| XCL1 | Chemokine | Attracts leukocytes/NK |
| IFNbeta | Cytokine | T cell response, tumor cell |
| | | killing |
| IFNgamma | Cytokine | T cell response, tumor cell |
| | | killing |
| IL-12 | Cytokine | T cells, NK cells |
| IL-1beta | Cytokine | T cells, NK cells |
| IL-15 | Cytokine | Stimulates T-cells and NK |
| IL-2 | Cytokine | Stimulates T-cells and NK |
| IL-21 | Cytokine | Stimulates T-cells |
| IL-24 | Cytokine | Stimulates T-cells |
| IL36-gamma | Cytokine | Stimulates T-cells |
| IL-7 | Cytokine | Stimulates T-cells |
| IL-22 | Cytokine | Stimulates T-cells |
| IL-18 | Cytokine | Stimulates T-cells |
| Granzymes/ | Enzyme | Direct tumor cell killing |
| Perforin | | |
| OX86 | ligand | Stimulates T-cells |
| (anti-OX40) | | |
| anti-TGFbeta | Neutralizing | Neutralizes an |
| | antibody | Immunosuppressive cytokine |
| TRAIL | Receptor/ligand | Direct tumor cell killing |
| FASL (CD49L) | Receptor/ligand | Direct tumor cell killing |
| OX40-L | Receptor/Ligand | Stimulates T-cells |
| cGAS | secreted molecule | Stimulates antigen-presenting |
| | | cells |
| 41BBL | secreted molecule | Co-activation of T-cells |
| CD40L | secreted molecule | Stimulates T-cells |
| GM-CSF | secreted molecule | Growth factor for monocytes |
| STING | secreted molecule | Stimulates antigen-presenting |
| | | cells |
| HAC-V | Antagonist | inhibits checkpoint |
| ‘microbody’ | antibody | |
| PD1 | | |
| yCD | Pro-drug | Converts to cytotoxic |
| | | molecule upon activation |
| CpG/ | Nucleotides | STING agonist |
| Nucleotides | | |

Engineered Nucleic Acids

Provided herein are engineered nucleic acids encoding at least one effector molecule. Provided herein are engineered nucleic acids encoding two or more effector molecules.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules). Engineered nucleic acids can be an isolated nucleic acid. Isolated nucleic acids include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, and an oligonucleotide.

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5’ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

### Promoters

In some embodiments, an engineered nucleic acid comprises a promoter operably linked to a nucleotide sequence encoding an effector molecule. In some embodiments, an engineered nucleic acid comprises a promoter operably linked to a nucleotide sequence encoding at least 2 effector molecules. For example, the engineered nucleic acid may comprise a promoter operably linked to a nucleotide sequence encoding at least 3, at least 4, at least 5, at least 6, at least 7, at least 8, at least 8, at least 9, or at least 10 effector molecules. In some embodiments, an engineered nucleic acid comprises a promoter operably linked to a nucleotide sequence encoding 1, 2, 3, 4, 5, 6, 7, 8, 9, 10, or more effector molecules.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 2, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Other non-limiting examples of inducible promoters include those presented in Table 3.

TABLE 2
Examples of Responsive Promoters.

| | | | | Response to | |
|---|---|---|---|---|---|
| | | Transcription | | inducer | |
| System | Promoter and operator | factor (TF) | Inducer molecule | TF | T |
| Transcriptional activator-responsive promoters | | | | | |
| AIR | PAIR | AlcR | Acetaldehyde | n.d. | A |
| | (OalcA-PhCMVmin) | | | | |
| ART | PART | ArgR-VP16 | 1-Arginine | B | A |
| | (OARG-PhCMVmin) | | | | |
| BIT | PBIT3 | BIT | Biotin | B | A |
| | (OBirA3-PhCMVmin) | (BirA-VP16) | | | |
| Cumate- | PCR5 | cTA | Cumate | D | DA |
| activator | (OCuO6-PhCMVmin) | (CymR-VP16) | | | |
| Cumate-reverse | PCR5 | rcTA | Cumate | B | A |
| activator | (OCuO6-PhCMVmin) | (rCymR-VP16) | | | |
| E-OFF | PETR | ET | Erythromycin | D | DA |
| | (OETR-PhCMVmin) | (E-VP16) | | | |
| NICE-OFF | PNIC | NT | 6-Hydroxy-nicotine | D | DA |
| | (ONIC-PhCMVmin) | (HdnoR-VP16) | | | |
| PEACE | PTtgR1 | TtgA1 | Phloretin | D | DA |
| | (OTtgR-PhCMVmin) | (TtgR-VP16) | | | |
| PIP-OFF | PPIR | PIT | Pristinamycin I | D | DA |
| | (OPIR-Phsp70min) | (PIP-VP16) | | | |
| QuoRex | PSCA | SCA | SCB1 | D | DA |
| | (OscbR-PhCMVmin) | (ScbR-VP16) | | | |
| | PSPA | | | | |
| | (OpapRI-PhCMVmin) | | | | |
| Redox | PROP (OROP- | REDOX | NADH | D | DA |
| | PhCMVmin) | (REX-VP16) | | | |
| TET-OFF | PhCMV*-1 | tTA | Tetracycline | D | DA |
| | (OtetO7-PhCMVmin) | (TetR-VP16) | | | |
| TET-ON | PhCMV*-1 | rtTA | Doxycycline | B | A |
| | (OtetO7-PhCMVmin) | (rTetR-VP16) | | | |
| TIGR | PCTA | CTA | Heat | D | DA |
| | (OrheO-PhCMVmin) | (RheA-VP16) | | | |
| TraR | O7x(tra box)- | p65-TraR | 3-Oxo-C8-HSL | B | A |
| | PhCMVmin | | | | |
| VAC-OFF | P1VanO2 | VanA1 | Vanillic acid | D | DA |
| | (OVanO2-PhCMVmin) | (VanR-VP16) | | | |
| Transcriptional repressor-responsive promoters | | | | | |
| Cumate - | PCuO | CymR | Cumate | D | DR |
| repressor | (PCMV5-OCuO) | | | | |
| E-ON | PETRON8 | E-KRAB | Erythromycin | D | DR |
| | (PSV40-OETR8) | | | | |
| NICE-ON | PNIC | NS | 6-Hydroxy-nicotine | D | DR |
| | (PSV40-ONIC8) | (HdnoR-KRAB) | | | |
| PIP-ON | PPIRON | PIT3 | Pristinamycin 1 | D | DR |
| | (PSV40-OPIR3) | (PIP-KRAB) | | | |
| Q-ON | PSCAON8 | SCS | SCB1 | D | DR |
| | (PSV40-OscbR8) | (ScbR-KRAB) | | | |
| TET- | OtetO-PHPRT | tTS-H4 | Doxycycline | D | DR |
| ON<comma> | | (TetR-HDAC4) | | | |
| repressor-based | | | | | |
| T-REX | PTetO | TetR | Tetracycline | D | DR |
| | (PhCMV-OtetO2) | | | | |
| UREX | PUREX8 | mUTS | Uric acid | D | DR |
| | (PSV40-OhucO8) | (KRAB-HucR) | | | |
| VAC-ON | PVanON8 | VanA4 | Vanillic acid | D | DR |
| | (PhCMV-OVanO8) | (VanR-KRAB) | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | TetracyclineErythromycin | DD | DADR |
| ON(NOT IF gate) | PhCMVmin | | | | |
| TET-OFFPIP- | OtetO7-OPIR3- | tTAPIT3E-KRAB | TetracyclinePristinamycin | DDD | DADRDR |
| ONE-ON | OETR8-PhCMVmin | | IErythromycin | | |

TABLE 3
Exemplary Inducible Promoters

| Name | DNA SEQUENCE | Source |
|---|---|---|
| minimal promoter; minP | AGAGGGTATATAATGGAAGCTCGACTT | EU581860.1 |
| | CCAG (SEQ ID NO: 1) | (Promega) |
| NFkB response element | GGGAATTTCCGGGGACTTTCCGGGAAT | EU581860.1 |
| protein promoter; 5x | TTCCGGGGACTTTCCGGGAATTTCC | (Promega) |
| NFkB-RE | (SEQ ID NO: 2) | |
| CREB response element | CACCAGACAGTGACGTCAGCTGCCAGA | DQ904461.1 |
| protein promoter; 4x CRE | TCCCATGGCCGTCATACTGTGACGTCTT | (Promega) |
| | TCAGACACCCCATTGACGTCAATGGGA | |
| | GAA (SEQ ID NO: 3) | |
| NF AT response element | GGAGGAAAAACTGTTTCATACAGAAGG | DQ904462.1 |
| protein promoter; 3x | CGTGGAGGAAAAACTGTTTCATACAGA | (Promega) |
| NF AT binding sites | AGGCGTGGAGGAAAAACTGTTTCATAC | |
| | AGAAGGCGT (SEQ ID NO: 4) | |
| SRF response element | AGGATGTCCATATTAGGACATCTAGGA | FJ773212.1 |
| protein promoter; 5x SRE | TGTCCATATTAGGACATCTAGGATGTC | (Promega) |
| | CATATTAGGACATCTAGGATGTCCATA | |
| | TTAGGACATCTAGGATGTCCATATTAG | |
| | GACATCT (SEQ ID NO: 5) | |
| SRF response element | AGTATGTCCATATTAGGACATCTACCA | FJ773213.1 |
| protein promoter 2; 5x | TGTCCATATTAGGACATCTACTATGTCC | (Promega) |
| SRF-RE | ATATTAGGACATCTTGTATGTCCATATT | |
| | AGGACATCTAAAATGTCCATATTAGGA | |
| | CATCT (SEQ ID NO: 6) | |
| API response element | TGAGTCAGTGACTCAGTGAGTCAGTGA | JQ858516.1 |
| protein promoter; 6x AP1- | CTCAGTGAGTCAGTGACTCAG (SEQ ID | (Promega) |
| RE | NO: 7) | |
| TCF-LEF response | AGATCAAAGGGTTTAAGATCAAAGGGC | JX099537.1 |
| element protein promoter; | TTAAGATCAAAGGGTATAAGATCAAAG | (Promega) |
| 8x TCF-LEF-RE | GGCCTAAGATCAAAGGGACTAAGATCA | |
| | AAGGGTTTAAGATCAAAGGGCTTAAGA | |
| | TCAAAGGGCCTA (SEQ ID NO: 8) | |
| SBEx4 | GTCTAGACGTCTAGACGTCTAGACGTC | Addgene Cat No: |
| | TAGAC (SEQ ID NO: 9) | 16495 |
| SMAD2/3-CAGACA x4 | CAGACACAGACACAGACACAGACA | Jonk et al. (J Biol |
| | (SEQ ID NO: 10) | Chem. 1998 Aug |
| | | 14;273(33):21145-52. |
| STAT3 binding site | Ggatccggtactcgagatctgcgatctaagtaagcttggcattc | Addgene Sequencing |
| | cggtactgttggtaaagccac (SEQ ID NO: 11) | Result #211335 |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter (see Table 4).

TABLE 4
Exemplary Constitutive Promoters

| Name | DNA SEQUENCE |
|---|---|
| CMV | GTTGACATTGATTATTGACTAGTTATTAATAGTAATCAATTACGGGGTCATT |
| | AGTTCATAGCCCATATATGGAGTTCCGCGTTACATAACTTACGGTAAATGG |
| | CCCGCCTGGCTGACCGCCCAACGACCCCCGCCCATTGACGTCAATAATGAC |
| | GTATGTTCCCATAGTAACGCCAATAGGGACTTTCCATTGACGTCAATGGGT |
| | GGAGTATTTACGGTAAACTGCCCACTTGGCAGTACATCAAGTGTATCATAT |
| | GCCAAGTACGCCCCCTATTGACGTCAATGACGGTAAATGGCCCGCCTGGCA |
| | TTATGCCCAGTACATGACCTTATGGGACTTTCCTACTTGGCAGTACATCTAC |
| | GTATTAGTCATCGCTATTACCATGGTGATGCGGTTTTGGCAGTACATCAATG |
| | GGCGTGGATAGCGGTTTGACTCACGGGGATTTCCAAGTCTCCACCCCATTG |
| | ACGTCAATGGGAGTTTGTTTTGGCACCAAAATCAACGGGACTTTCCAAAAT |
| | GTCGTAACAACTCCGCCCCATTGACGCAAATGGGCGGTAGGCGTGTACGGT |
| | GGGAGGTCTATATAAGCAGAGCTC (SEQ ID NO: 12) |
| EF1a | GGCTCCGGTGCCCGTCAGTGGGCAGAGCGCACATCGCCCACAGTCCCCGA |
| | GAAGTTGGGGGGAGGGGTCGGCAATTGAACCGGTGCCTAGAGAAGGTGGC |
| | GCGGGGTAAACTGGGAAAGTGATGCCGTGTACTGGCTCCGCCTTTTTCCCG |
| | AGGGTGGGGGAGAACCGTATATAAGTGCAGTAGTCGCCGTGAACGTTCTTT |
| | TTCGCAACGGGTTTGCCGCCAGAACACAGGTAAGTGCCGTGTGTGGTTCCC |
| | GCGGGCCTGGCCTCTTTACGGGTTATGGCCCTTGCGTGCCTTGAATTACTTC |
| | CACCTGGCTGCAGTACGTGATTCTTGATCCCGAGCTTCGGGTTGGAAGTGG |
| | GTGGGAGAGTTCGAGGCCTTGCGCTTAAGGAGCCCCTTCGCCTCGTGCTTG |
| | AGTTGAGGCCTGGCCTGGGCGCTGGGGCCGCCGCGTGCGAATCTGGTGGC |
| | ACCTTCGCGCCTGTCTCGCTGCTTTCGATAAGTCTCTAGCCATTTAAAATTT |
| | TTGATGACCTGCTGCGACGCTTTTTTTCTGGCAAGATAGTCTTGTAAATGCG |
| | GGCCAAGATCTGCACACTGGTATTTCGGTTTTTGGGGCCGCGGGCGGCGAC |
| | GGGGCCCGTGCGTCCCAGCGCACATGTTCGGCGAGGCGGGGCCTGCGAGC |
| | GCGACCACCGAGAATCGGACGGGGGTAGTCTCAAGCTGGCCGGCCTGCTC |
| | TGGTGCCTGTCCTCGCGCCGCCGTGTATCGCCCCGCCCCGGGCGGCAAGGC |
| | TGGCCCGGTCGGCACCAGTTGCGTGAGCGGAAAGATGGCCGCTTCCCGGTC |
| | CTGCTGCAGGGAGCTCAAAATGGAGGACGCGGCGCTCGGGAGAGCGGGCG |
| | GGTGAGTCACCCACACAAAGGAAAAGGGCCTTTCCGTCCTCAGCCGTCGCT |
| | TCATGTGACTCCACGGAGTACCGGGCGCCGTCCAGGCACCTCGATTAGTTC |
| | TCGAGCTTTTGGAGTACGTCGTCTTTAGGTTGGGGGGAGGGGTTTTATGCG |
| | ATGGAGTTTCCCCACACTGAGTGGGTGGAGACTGAAGTTAGGCCAGCTTGG |
| | CACTTGATGTAATTCTCCTTGGAATTTGCCCTTTTTGAGTTTGGATCTTGGT |
| | TCATTCTCAAGCCTCAGACAGTGGTTCAAAGTTTTTTTCTTCCATTTCAGGT |
| | GTCGTGA (SEQ ID NO: 13) |
| EFS | GGATCTGCGATCGCTCCGGTGCCCGTCAGTGGGCAGAGCGCACATCGCCCA |
| | CAGTCCCCGAGAAGTTGGGGGGAGGGGTCGGCAATTGAACCGGTGCCTAG |
| | AGAAGGTGGCGCGGGGTAAACTGGGAAAGTGATGTCGTGTACTGGCTCCG |
| | CCTTTTTCCCGAGGGTGGGGGAGAACCGTATATAAGTGCAGTAGTCGCCGT |
| | GAACGTTCTTTTTCGCAACGGGTTTGCCGCCAGAACACAGCTGAAGCTTCG |
| | AGGGGCTCGCATCTCTCCTTCACGCGCCCGCCGCCCTACCTGAGGCCGCCA |
| | TCCACGCCGGTTGAGTCGCGTTCTGCCGCCTCCCGCCTGTGGTGCCTCCTGA |
| | ACTGCGTCCGCCGTCTAGGTAAGTTTAAAGCTCAGGTCGAGACCGGGCCTT |
| | TGTCCGGCGCTCCCTTGGAGCCTACCTAGACTCAGCCGGCTCTCCACGCTTT |
| | GCCTGACCCTGCTTGCTCAACTCTACGTCTTTGTTTCGTTTTCTGTTCTGCGC |
| | CGTTACAGATCCAAGCTGTGACCGGCGCCTAC (SEQ ID NO: 14) |
| MND | TTTATTTAGTCTCCAGAAAAAGGGGGGAATGAAAGACCCCACCTGTAGGTT |
| | TGGCAAGCTAGGATCAAGGTTAGGAACAGAGAGACAGCAGAATATGGGCC |
| | AAACAGGATATCTGTGGTAAGCAGTTCCTGCCCCGGCTCAGGGCCAAGAA |
| | CAGTTGGAACAGCAGAATATGGGCCAAACAGGATATCTGTGGTAAGCAGT |
| | TCCTGCCCCGGCTCAGGGCCAAGAACAGATGGTCCCCAGATGCGGTCCCGC |
| | CCTCAGCAGTTTCTAGAGAACCATCAGATGTTTCCAGGGTGCCCCAAGGAC |
| | CTGAAATGACCCTGTGCCTTATTTGAACTAACCAATCAGTTCGCTTCTCGCT |
| | TCTGTTCGCGCGCTTCTGCTCCCCGAGCTCAATAAAAGAGCCCA (SEQ ID |
| | NO: 15) |
| PGK | GGGGTTGGGGTTGCGCCTTTTCCAAGGCAGCCCTGGGTTTGCGCAGGGACG |
| | CGGCTGCTCTGGGCGTGGTTCCGGGAAACGCAGCGGCGCCGACCCTGGGT |
| | CTCGCACATTCTTCACGTCCGTTCGCAGCGTCACCCGGATCTTCGCCGCTAC |
| | CCTTGTGGGCCCCCCGGCGACGCTTCCTGCTCCGCCCCTAAGTCGGGAAGG |
| | TTCCTTGCGGTTCGCGGCGTGCCGGACGTGACAAACGGAAGCCGCACGTCT |
| | CACTAGTACCCTCGCAGACGGACAGCGCCAGGGAGCAATGGCAGCGCGCC |
| | GACCGCGATGGGCTGTGGCCAATAGCGGCTGCTCAGCGGGGCGCGCCGAG |
| | AGCAGCGGCCGGGAAGGGGCGGTGCGGGAGGCGGGGTGTGGGGCGGTAG |
| | TGTGGGCCCTGTTCCTGCCCGCGCGGTGTTCCGCATTCTGCAAGCCTCCGG |
| | AGCGCACGTCGGCAGTCGGCTCCCTCGTTGACCGAATCACCGACCTCTCTC |
| | CCCAG (SEQ ID NO: 16) |
| SFFV | GTAACGCCATTTTGCAAGGCATGGAAAAATACCAAACCAAGAATAGAGAA |
| | GTTCAGATCAAGGGCGGGTACATGAAAATAGCTAACGTTGGGCCAAACAG |
| | GATATCTGCGGTGAGCAGTTTCGGCCCCGGCCCGGGGCCAAGAACAGATG |
| | GTCACCGCAGTTTCGGCCCCGGCCCGAGGCCAAGAACAGATGGTCCCCAG |
| | ATATGGCCCAACCCTCAGCAGTTTCTTAAGACCCATCAGATGTTTCCAGGC |
| | TCCCCCAAGGACCTGAAATGACCCTGCGCCTTATTTGAATTAACCAATCAG |
| | CCTGCTTCTCGCTTCTGTTCGCGCGCTTCTGCTTCCCGAGCTCTATAAAAGA |
| | GCTCACAACCCCTCACTCGGCGCGCCAGTCCTCCGACAGACTGAGTCGCCC |
| | GGG (SEQ ID NO: 17) |
| SV40 | CTGTGGAATGTGTGTCAGTTAGGGTGTGGAAAGTCCCCAGGCTCCCCAGCA |
| | GGCAGAAGTATGCAAAGCATGCATCTCAATTAGTCAGCAACCAGGTGTGG |
| | AAAGTCCCCAGGCTCCCCAGCAGGCAGAAGTATGCAAAGCATGCATCTCA |
| | ATTAGTCAGCAACCATAGTCCCGCCCCTAACTCCGCCCATCCCGCCCCTAA |
| | CTCCGCCCAGTTCCGCCCATTCTCCGCCCCATGGCTGACTAATTTTTTTTAT |
| | TTATGCAGAGGCCGAGGCCGCCTCTGCCTCTGAGCTATTCCAGAAGTAGTG |
| | AGGAGGCTTTTTTGGAGGCCTAGGCTTTTGCAAAAAGCT (SEQ ID NO: 18) |
| UbC | GCGCCGGGTTTTGGCGCCTCCCGCGGGCGCCCCCCTCCTCACGGCGAGCGC |
| | TGCCACGTCAGACGAAGGGCGCAGGAGCGTTCCTGATCCTTCCGCCCGGAC |
| | GCTCAGGACAGCGGCCCGCTGCTCATAAGACTCGGCCTTAGAACCCCAGTA |
| | TCAGCAGAAGGACATTTTAGGACGGGACTTGGGTGACTCTAGGGCACTGG |
| | TTTTCTTTCCAGAGAGCGGAACAGGCGAGGAAAAGTAGTCCCTTCTCGGCG |
| | ATTCTGCGGAGGGATCTCCGTGGGGCGGTGAACGCCGATGATTATATAAG |
| | GACGCGCCGGGTGTGGCACAGCTAGTTCCGTCGCAGCCGGGATTTGGGTCG |
| | CGGTTCTTGTTTGTGGATCGCTGTGATCGTCACTTGGTGAGTTGCGGGCTGC |
| | TGGGCTGGCCGGGGCTTTCGTGGCCGCCGGGCCGCTCGGTGGGACGGAAG |
| | CGTGTGGAGAGACCGCCAAGGGCTGTAGTCTGGGTCCGCGAGCAAGGTTG |
| | CCCTGAACTGGGGGTTGGGGGGAGCGCACAAAATGGCGGCTGTTCCCGAG |
| | TCTTGAATGGAAGACGCTTGTAAGGCGGGCTGTGAGGTCGTTGAAACAAG |
| | GTGGGGGGCATGGTGGGCGGCAAGAACCCAAGGTCTTGAGGCCTTCGCTA |
| | ATGCGGGAAAGCTCTTATTCGGGTGAGATGGGCTGGGGCACCATCTGGGG |
| | ACCCTGACGTGAAGTTTGTCACTGACTGGAGAACTCGGGTTTGTCGTCTGG |
| | TTGCGGGGGCGGCAGTTATGCGGTGCCGTTGGGCAGTGCACCCGTACCTTT |
| | GGGAGCGCGCGCCTCGTCGTGTCGTGACGTCACCCGTTCTGTTGGCTTATA |
| | ATGCAGGGTGGGGCCACCTGCCGGTAGGTGTGCGGTAGGCTTTTCTCCGTC |
| | GCAGGACGCAGGGTTCGGGCCTAGGGTAGGCTCTCCTGAATCGACAGGCG |
| | CCGGACCTCTGGTGAGGGGAGGGATAAGTGAGGCGTCAGTTTCTTTGGTCG |
| | GTTTTATGTACCTATCTTCTTAAGTAGCTGAAGCTCCGGTTTTGAACTATGC |
| | GCTCGGGGTTGGCGAGTGTGTTTTGTGAAGTTTTTTAGGCACCTTTTGAAAT |
| | GTAATCATTTGGGTCAATATGTAATTTTCAGTGTTAGACTAGTAAAGCTTCT |
| | GCAGGTCGACTCTAGAAAATTGTCCGCTAAATTCTGGCCGTTTTTGGCTTTT |
| | TTGTTAGAC (SEQ ID NO: 19) |
| hEF1aV1 | GGCTCCGGTGCCCGTCAGTGGGCAGAGCGCACATCGCCCACAGTCCCC |
| | GAGAAGTTGGGGGGAGGGGTCGGCAATTGAACCGGTGCCTAGAGAAGG |
| | TGGCGCGGGGTAAACTGGGAAAGTGATGTCGTGTACTGGCTCCGCCTTT |
| | TTCCCGAGGGTGGGGGAGAACCGTATATAAGTGCAGTAGTCGCCGTGAA |
| | CGTTCTTTTTCGCAACGGGTTTGCCGCCAGAACACAGGTAAGTGCCGTGT |
| | GTGGTTCCCGCGGGCCTGGCCTCTTTACGGGTTATGGCCCTTGCGTGCC |
| | TTGAATTACTTCCACCTGGCTGCAGTACGTGATTCTTGATCCCGAGCTTC |
| | GGGTTGGAAGTGGGTGGGAGAGTTCGAGGCCTTGCGCTTAAGGAGCCC |
| | CTTCGCCTCGTGCTTGAGTTGAGGCCTGGCCTGGGCGCTGGGGCCGCC |
| | GCGTGCGAATCTGGTGGCACCTTCGCGCCTGTCTCGCTGCTTTCGATAA |
| | GTCTCTAGCCATTTAAAATTTTTGATGACCTGCTGCGACGCTTTTTTTCTG |
| | GCAAGATAGTCTTGTAAATGCGGGCCAAGATCTGCACACTGGTATTTCGG |
| | TTTTTGGGGCCGCGGGCGGCGACGGGGCCCGTGCGTCCCAGCGCACAT |
| | GTTCGGCGAGGCGGGGCCTGCGAGCGCGGCCACCGAGAATCGGACGG |
| | GGGTAGTCTCAAGCTGGCCGGCCTGCTCTGGTGCCTGGTCTCGCGCCG |
| | CCGTGTATCGCCCCGCCCTGGGCGGCAAGGCTGGCCCGGTCGGCACCA |
| | GTTGCGTGAGCGGAAAGATGGCCGCTTCCCGGCCCTGCTGCAGGGAGC |
| | TCAAAATGGAGGACGCGGCGCTCGGGAGAGCGGGCGGGTGAGTCACCC |
| | ACACAAAGGAAAAGGGCCTTTCCGTCCTCAGCCGTCGCTTCATGTGACTC |
| | CACGGAGTACCGGGCGCCGTCCAGGCACCTCGATTAGTTCTCGAGCTTT |
| | TGGAGTACGTCGTCTTTAGGTTGGGGGGAGGGGTTTTATGCGATGGAGT |
| | TTCCCCACACTGAGTGGGTGGAGACTGAAGTTAGGCCAGCTTGGCACTT |
| | GATGTAATTCTCCTTGGAATTTGCCCTTTTTGAGTTTGGATCTTGGTTCATT |
| | CTCAAGCCTCAGACAGTGGTTCAAAGTTTTTTTCTTCCATTTCAGGTGTCG |
| | TGA (SEQ ID NO: 20) |
| hCAGG | ACTAGTTATTAATAGTAATCAATTACGGGGTCATTAGTTCATAGCCCATAT |
| | ATGGAGTTCCGCGTTACATAACTTACGGTAAATGGCCCGCCTGGCTGACC |
| | GCCCAACGACCCCCGCCCATTGACGTCAATAATGACGTATGTTCCCATAG |
| | TAACGCCAATAGGGACTTTCCATTGACGTCAATGGGTGGAGTATTTACGG |
| | TAAACTGCCCACTTGGCAGTACATCAAGTGTATCATATGCCAAGTACGCC |
| | CCCTATTGACGTCAATGACGGTAAATGGCCCGCCTGGCATTATGCCCAGT |
| | ACATGACCTTATGGGACTTTCCTACTTGGCAGTACATCTACGTATTAGTCA |
| | TCGCTATTACCATGGTCGAGGTGAGCCCCACGTTCTGCTTCACTCTCCCC |
| | ATCTCCCCCCCCTCCCCACCCCCAATTTGTATTTATTTATTTTTAATTAT |
| | TTTGTGCAGCGATGGGGGCGGGGGGGGGGGGGGGGCGCGCGCCAGGC |
| | GGGGCGGGGCGGGGCGAGGGGCGGGGCGGGGCGAGGCGGAGAGGTG |
| | CGGCGGCAGCCAATCAGAGCGGCGCGCTCCGAAAGTTTCCTTTTATGGC |
| | GAGGCGGCGGCGGCGGCGGCCCTATAAAAAGCGAAGCGCGCGGCGGG |
| | CGGGGAGTCGCTGCGACGCTGCCTTCGCCCCGTGCCCCGCTCCGCCGC |
| | CGCCTCGCGCCGCCCGCCCCGGCTCTGACTGACCGCGTTACTCCCACA |
| | GGTGAGCGGGCGGGACGGCCCTTCTCCTCCGGGCTGTAATTAGCGCTTG |
| | GTTTAATGACGGCTTGTTTCTTTTCTGTGGCTGCGTGAAAGCCTTGAGGG |
| | GCTCCGGGAGGGCCCTTTGTGCGGGGGGAGCGGCTCGGGGGGTGCGT |
| | GCGTGTGTGTGTGCGTGGGGAGCGCCGCGTGCGGCTCCGCGCTGCCCG |
| | GCGGCTGTGAGCGCTGCGGGCGCGGCGCGGGGCTTTGTGCGCTCCGCA |
| | GTGTGCGCGAGGGGAGCGCGGCCGGGGGCGGTGCCCCGCGGTGCGGG |
| | GGGGGCTGCGAGGGGAACAAAGGCTGCGTGCGGGGTGTGTGCGTGGG |
| | GGGGTGAGCAGGGGGTGTGGGCGCGTCGGTCGGGCTGCAACCCCCCCT |
| | GCACCCCCCTCCCCGAGTTGCTGAGCACGGCCCGGCTTCGGGTGCGGG |
| | GCTCCGTACGGGGCGTGGCGCGGGGCTCGCCGTGCCGGGCGGGGGGT |
| | GGCGGCAGGTGGGGGTGCCGGGCGGGGCGGGGCCGCCTCGGGCCGG |
| | GGAGGGCTCGGGGGAGGGGCGCGGCGGCCCCCGGAGCGCCGGCGGC |
| | TGTCGAGGCGCGGCGAGCCGCAGCCATTGCCTTTTATGGTAATCGTGCG |
| | AGAGGGCGCAGGGACTTCCTTTGTCCCAAATCTGTGCGGAGCCGAAATC |
| | TGGGAGGCGCCGCCGCACCCCCTCTAGCGGGCGCGGGGCGAAGCGGT |
| | GCGGCGCCGGCAGGAAGGAAATGGGCGGGGAGGGCCTTCGTGCGTCG |
| | CCGCGCCGCCGTCCCCTTCTCCCTCTCCAGCCTCGGGGCTGTCCGCGG |
| | GGGGACGGCTGCCTTCGGGGGGGACGGGGCAGGGCGGGGTTCGGCTT |
| | CTGGCGTGTGACCGGCGGCTCTAGAGCCTCTGCTAACCATGTTCATGCC |
| | TTCTTCTTTTTCCTACAGCTCCTGGGCAACGTGCTGGTTATTGTGCTGTCT |
| | CATCATTTTGGCAAAGAATTC (SEQ ID NO: 21) |
| hEF1aV2 | Gggcagagcgcacatcgcccacagtccccgagaagttggggggaggggtcggcaattgaaccggtgcc |
| | tagagaaggtggcgcggggtaaactgggaaagtgatgtcgtgtactggctccgcctttttcccgagggtggg |
| | ggagaaccgtatataagtgcagtagtcgccgtgaacgttctttttcgcaacgggtttgccgccagaacacag |
| | (SEQ ID NO: 22) |
| hACTb | CCACTAGTTCCATGTCCTTATATGGACTCATCTTTGCCTATTGCGACACAC |
| | ACTCAATGAACACCTACTACGCGCTGCAAAGAGCCCCGCAGGCCTGAGG |
| | TGCCCCCACCTCACCACTCTTCCTATTTTTGTGTAAAAATCCAGCTTCTTG |
| | TCACCACCTCCAAGGAGGGGGAGGAGGAGGAAGGCAGGTTCCTCTAGG |
| | CTGAGCCGAATGCCCCTCTGTGGTCCCACGCCACTGATCGCTGCATGCC |
| | CACCACCTGGGTACACACAGTCTGTGATTCCCGGAGCAGAACGGACCCT |
| | GCCCACCCGGTCTTGTGTGCTACTCAGTGGACAGACCCAAGGCAAGAAA |
| | GGGTGACAAGGACAGGGTCTTCCCAGGCTGGCTTTGAGTTCCTAGCACC |
| | GCCCCGCCCCCAATCCTCTGTGGCACATGGAGTCTTGGTCCCCAGAGTC |
| | CCCCAGCGGCCTCCAGATGGTCTGGGAGGGCAGTTCAGCTGTGGCTGC |
| | GCATAGCAGACATACAACGGACGGTGGGCCCAGACCCAGGCTGTGTAGA |
| | CCCAGCCCCCCCGCCCCGCAGTGCCTAGGTCACCCACTAACGCCCCAG |
| | GCCTGGTCTTGGCTGGGCGTGACTGTTACCCTCAAAAGCAGGCAGCTCC |
| | AGGGTAAAAGGTGCCCTGCCCTGTAGAGCCCACCTTCCTTCCCAGGGCT |
| | GCGGCTGGGTAGGTTTGTAGCCTTCATCACGGGCCACCTCCAGCCACTG |
| | GACCGCTGGCCCCTGCCCTGTCCTGGGGAGTGTGGTCCTGCGACTTCTA |
| | AGTGGCCGCAAGCCACCTGACTCCCCCAACACCACACTCTACCTCTCAA |
| | GCCCAGGTCTCTCCCTAGTGACCCACCCAGCACATTTAGCTAGCTGAGC |
| | CCCACAGCCAGAGGTCCTCAGGCCCTGCTTTCAGGGCAGTTGCTCTGAA |
| | GTCGGCAAGGGGGAGTGACTGCCTGGCCACTCCATGCCCTCCAAGAGCT |
| | CCTTCTGCAGGAGCGTACAGAACCCAGGGCCCTGGCACCCGTGCAGACC |
| | CTGGCCCACCCCACCTGGGCGCTCAGTGCCCAAGAGATGTCCACACCTA |
| | GGATGTCCCGCGGTGGGTGGGGGGCCCGAGAGACGGGCAGGCCGGGG |
| | GCAGGCCTGGCCATGCGGGGCCGAACCGGGCACTGCCCAGCGTGGGG |
| | CGCGGGGGCCACGGCGCGCGCCCCCAGCCCCCGGGCCCAGCACCCCA |
| | AGGCGGCCAACGCCAAAACTCTCCCTCCTCCTCTTCCTCAATCTCGCTCT |
| | CGCTCTTTTTTTTTTTCGCAAAAGGAGGGGAGAGGGGGTAAAAAAATGCT |
| | GCACTGTGCGGCGAAGCCGGTGAGTGAGCGGCGCGGGGCCAATCAGCG |
| | TGCGCCGTTCCGAAAGTTGCCTTTTATGGCTCGAGCGGCCGCGGCGGCG |
| | CCCTATAAAACCCAGCGGCGCGACGCGCCACCACCGCCGAGACCGCGT |
| | CCGCCCCGCGAGCACAGAGCCTCGCCTTTGCCGATCCGCCGCCCGTCC |
| | ACACCCGCCGCCAGgtaagcccggccagccgaccggggcaggcggctcacggcccggccgc |
| | aggcggccgcggccccttcgcccgtgcagagccgccgtctgggccgcagcggggggcgcatggggggg |
| | gaaccggaccgccgtggggggcgcgggagaagcccctgggcctccggagatgggggacaccccacgc |
| | cagttcggaggcgcgaggccgcgctcgggaggcgcgctccgggggtgccgctctcggggcgggggcaa |
| | ccggcggggtctttgtctgagccgggctcttgccaatggggatcgcagggtgggcgcggcggagcccccgc |
| | caggcccggtgggggctggggcgccattgcgcgtgcgcgctggtcctttgggcgctaactgcgtgcgcgctg |
| | ggaattggcgctaattgcgcgtgcgcgctgggactcaaggcgctaactgcgcgtgcgttctggggcccggg |
| | gtgccgcggcctgggctggggcgaaggcgggctcggccggaaggggtggggtcgccgcggctcccggg |
| | cgcttgcgcgcacttcctgcccgagccgctggccgcccgagggtgtggccgctgcgtgcgcgcgcgccga |
| | cccggcgctgtttgaaccgggcggaggcggggctggcgcccggttgggagggggttggggcctggcttcct |
| | gccgcgcgccgcggggacgcctccgaccagtgtttgccttttatggtaataacgcggccggcccggcttccttt |
| | gtccccaatctgggcgcgcgccggcgccccctggcggcctaaggactcggcgcgccggaagtggccagg |
| | gcgggggcgacctcggctcacagcgcgcccggctat (SEQ ID NO: 23) |
| heIF4A1 | GTTGATTTCCTTCATCCCTGGCACACGTCCAGGCAGTGTCGAATCCATCT |
| | CTGCTACAGGGGAAAACAAATAACATTTGAGTCCAGTGGAGACCGGGAG |
| | CAGAAGTAAAGGGAAGTGATAACCCCCAGAGCCCGGAAGCCTCTGGAGG |
| | CTGAGACCTCGCCCCCCTTGCGTGATAGGGCCTACGGAGCCACATGACC |
| | AAGGCACTGTCGCCTCCGCACGTGTGAGAGTGCAGGGCCCCAAGATGG |
| | CTGCCAGGCCTCGAGGCCTGACTCTTCTATGTCACTTCCGTACCGGCGA |
| | GAAAGGCGGGCCCTCCAGCCAATGAGGCTGCGGGGCGGGCCTTCACCT |
| | TGATAGGCACTCGAGTTATCCAATGGTGCCTGCGGGCCGGAGCGACTAG |
| | GAACTAACGTCATGCCGAGTTGCTGAGCGCCGGCAGGCGGGGCCGGGG |
| | CGGCCAAACCAATGCGATGGCCGGGGCGGAGTCGGGCGCTCTATAAGTT |
| | GTCGATAGGCGGGCACTCCGCCCTAGTTTCTAAGGACCATG (SEQ ID NO: |
| | 24) |
| hGAPDH | AGTTCCCCAACTTTCCCGCCTCTCAGCCTTTGAAAGAAAGAAAGGGGAGG |
| | GGGCAGGCCGCGTGCAGTCGCGAGCGGTGCTGGGCTCCGGCTCCAATT |
| | CCCCATCTCAGTCGCTCCCAAAGTCCTTCTGTTTCATCCAAGCGTGTAAG |
| | GGTCCCCGTCCTTGACTCCCTAGTGTCCTGCTGCCCACAGTCCAGTCCT |
| | GGGAACCAGCACCGATCACCTCCCATCGGGCCAATCTCAGTCCCTTCCC |
| | CCCTACGTCGGGGCCCACACGCTCGGTGCGTGCCCAGTTGAACCAGGC |
| | GGCTGCGGAAAAAAAAAAGCGGGGAGAAAGTAGGGCCCGGCTACTAGC |
| | GGTTTTACGGGCGCACGTAGCTCAGGCCTCAAGACCTTGGGCTGGGACT |
| | GGCTGAGCCTGGCGGGAGGCGGGGTCCGAGTCACCGCCTGCCGCCGC |
| | GCCCCCGGTTTCTATAAATTGAGCCCGCAGCCTCCCGCTTCGCTCTCTGC |
| | TCCTCCTGTTCGACAGTCAGCCGCATCTTCTTTTGCGTCGCCAGgtgaagac |
| | gggcggagagaaacccgggaggctagggacggcctgaaggcggcaggggcgggcgcaggccggat |
| | gtgttcgcgccgctgcggggtgggcccgggcggcctccgcattgcaggggcgggcggaggacgtgatgc |
| | ggcgcgggctgggcatggaggcctggtgggggaggggaggggaggcgtgggtgtcggccggggccact |
| | aggcgctcactgttctctccctccgcgcagCCGAGCCACATCGCTGAGACAC (SEQ ID |
| | NO: 25) |
| hGRP78 | AGTGCGGTTACCAGCGGAAATGCCTCGGGGTCAGAAGTCGCAGGAGAGA |
| | TAGACAGCTGCTGAACCAATGGGACCAGCGGATGGGGCGGATGTTATCT |
| | ACCATTGGTGAACGTTAGAAACGAATAGCAGCCAATGAATCAGCTGGGG |
| | GGGCGGAGCAGTGACGTTTATTGCGGAGGGGGCCGCTTCGAATCGGCG |
| | GCGGCCAGCTTGGTGGCCTGGGCCAATGAACGGCCTCCAACGAGCAGG |
| | GCCTTCACCAATCGGCGGCCTCCACGACGGGGCTGGGGGAGGGTATAT |
| | AAGCCGAGTAGGCGACGGTGAGGTCGACGCCGGCCAAGACAGCACAGA |
| | CAGATTGACCTATTGGGGTGTTTCGCGAGTGTGAGAGGGAAGCGCCGCG |
| | GCCTGTATTTCTAGACCTGCCCTTCGCCTGGTTCGTGGCGCCTTGTGACC |
| | CCGGGCCCCTGCCGCCTGCAAGTCGGAAATTGCGCTGTGCTCCTGTGCT |
| | ACGGCCTGTGGCTGGACTGCCTGCTGCTGCCCAACTGGCTGGCAC (SEQ |
| | ID NO: 26) |
| hGRP94 | TAGTTTCATCACCACCGCCACCCCCCCGCCCCCCCGCCATCTGAAAGGG |
| | TTCTAGGGGATTTGCAACCTCTCTCGTGTGTTTCTTCTTTCCGAGAAGCG |
| | CCGCCACACGAGAAAGCTGGCCGCGAAAGTCGTGCTGGAATCACTTCCA |
| | ACGAAACCCCAGGCATAGATGGGAAAGGGTGAAGAACACGTTGCCATGG |
| | CTACCGTTTCCCCGGTCACGGAATAAACGCTCTCTAGGATCCGGAAGTAG |
| | TTCCGCCGCGACCTCTCTAAAAGGATGGATGTGTTCTCTGCTTACATTCAT |
| | TGGACGTTTTCCCTTAGAGGCCAAGGCCGCCCAGGCAAAGGGGCGGTCC |
| | CACGCGTGAGGGGCCCGCGGAGCCATTTGATTGGAGAAAAGCTGCAAAC |
| | CCTGACCAATCGGAAGGAGCCACGCTTCGGGCATCGGTCACCGCACCTG |
| | GACAGCTCCGATTGGTGGACTTCCGCCCCCCCTCACGAATCCTCATTGG |
| | GTGCCGTGGGTGCGTGGTGCGGCGCGATTGGTGGGTTCATGTTTCCCGT |
| | CCCCCGCCCGCGAGAAGTGGGGGTGAAAAGCGGCCCGACCTGCTTGGG |
| | GTGTAGTGGGCGGACCGCGCGGCTGGAGGTGTGAGGATCCGAACCCAG |
| | GGGTGGGGGGTGGAGGCGGCTCCTGCGATCGAAGGGGACTTGAGACTC |
| | ACCGGCCGCACGTC (SEQ ID NO: 27) |
| hHSP70 | GGGCCGCCCACTCCCCCTTCCTCTCAGGGTCCCTGTCCCCTCCAGTGAA |
| | TCCCAGAAGACTCTGGAGAGTTCTGAGCAGGGGGCGGCACTCTGGCCTC |
| | TGATTGGTCCAAGGAAGGCTGGGGGGCAGGACGGGAGGCGAAAACCCT |
| | GGAATATTCCCGACCTGGCAGCCTCATCGAGCTCGGTGATTGGCTCAGA |
| | AGGGAAAAGGCGGGTCTCCGTGACGACTTATAAAAGCCCAGGGGCAAGC |
| | GGTCCGGATAACGGCTAGCCTGAGGAGCTGCTGCGACAGTCCACTACCT |
| | TTTTCGAGAGTGACTCCCGTTGTCCCAAGGCTTCCCAGAGCGAACCTGTG |
| | CGGCTGCAGGCACCGGCGCGTCGAGTTTCCGGCGTCCGGAAGGACCGA |
| | GCTCTTCTCGCGGATCCAGTGTTCCGTTTCCAGCCCCCAATCTCAGAGCG |
| | GAGCCGACAGAGAGCAGGGAACCC (SEQ ID NO: 28) |
| hKINb | GCCCCACCCCCGTCCGCGTTACAACCGGGAGGCCCGCTGGGTCCTGCA |
| | CCGTCACCCTCCTCCCTGTGACCGCCCACCTGATACCCAAACAACTTTCT |
| | CGCCCCTCCAGTCCCCAGCTCGCCGAGCGCTTGCGGGGAGCCACCCAG |
| | CCTCAGTTTCCCCAGCCCCGGGCGGGGCGAGGGGCGATGACGTCATGC |
| | CGGCGCGCGGCATTGTGGGGCGGGGCGAGGCGGGGCGCCGGGGGGA |
| | GCAACACTGAGACGCCATTTTCGGCGGCGGGAGCGGCGCAGGCGGCCG |
| | AGCGGGACTGGCTGGGTCGGCTGGGCTGCTGGTGCGAGGAGCCGCGG |
| | GGCTGTGCTCGGCGGCCAAGGGGACAGCGCGTGGGTGGCCGAGGATG |
| | CTGCGGGGCGGTAGCTCCGGCGCCCCTCGCTGGTGACTGCTGCGCCGT |
| | GCCTCACACAGCCGAGGCGGGCTCGGCGCACAGTCGCTGCTCCGCGCT |
| | CGCGCCCGGCGGCGCTCCAGGTGCTGACAGCGCGAGAGAGCGCGGCC |
| | TCAGGAGCAACAC (SEQ ID NO: 29) |
| hUBIb | TTCCAGAGCTTTCGAGGAAGGTTTCTTCAACTCAAATTCATCCGCCTGATA |
| | ATTTTCTTATATTTTCCTAAAGAAGGAAGAGAAGCGCATAGAGGAGAAGG |
| | GAAATAATTTTTTAGGAGCCTTTCTTACGGCTATGAGGAATTTGGGGCTCA |
| | GTTGAAAAGCCTAAACTGCCTCTCGGGAGGTTGGGCGCGGCGAACTACT |
| | TTCAGCGGCGCACGGAGACGGCGTCTACGTGAGGGGTGATAAGTGACG |
| | CAACACTCGTTGCATAAATTTGCGCTCCGCCAGCCCGGAGCATTTAGGG |
| | GCGGTTGGCTTTGTTGGGTGAGCTTGTTTGTGTCCCTGTGGGTGGACGT |
| | GGTTGGTGATTGGCAGGATCCTGGTATCCGCTAACAGgtactggcccacagccgt |
| | aaagacctgcgggggcgtgagaggggggaatgggtgaggtcaagctggaggcttcttggggttgggtggg |
| | ccgctgaggggaggggagggcgaggtgacgcgacacccggcctttctgggagagtgggccttgttgacct |
| | aaggggggcgagggcagttggcacgcgcacgcgccgacagaaactaacagacattaaccaacagcga |
| | ttccgtcgcgtttacttgggaggaaggcggaaaagaggtagtttgtgtggcttctggaaaccctaaatttggaat |
| | cccagtatgagaatggtgtcccttcttgtgtttcaatgggatttttacttcgcgagtcttgtgggtttggttttgttttcag |
| | tttgcctaacaccgtgcttaggtttgaggcagattggagttcggtcgggggagtttgaatatccggaacagttag |
| | tggggaaagctgtggacgcttggtaagagagcgctctggattttccgctgttgacgttgaaaccttgaatgacg |
| | aatttcgtattaagtgacttagccttgtaaaattgaggggaggcttgcggaatattaacgtatttaaggcattttga |
| | aggaatagttgctaattttgaagaatattaggtgtaaaagcaagaaatacaatgatcctgaggtgacacgctt |
| | atgttttacttttaaactagGTCACC (SEQ ID NO: 30) |

In some embodiments, a promoter of the present disclosure is modulated by signals within a tumor microenvironment. A tumor microenvironment is considered to modulate a promoter if, in the presence of the tumor microenvironment, the activity of the promoter is increased or decreased by at least 10%, relative to activity of the promoter in the absence of the tumor microenvironment. In some embodiments, the activity of the promoter is increased or decreased by at least 20%, at least 30%, at least 40%, at least 50%, at least 60%, at least 70%, at least 80%, at least 90%, at least 100%, relative to activity of the promoter in the absence of the tumor microenvironment. For example, the activity of the promoter is increased or decreased by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to activity of the promoter in the absence of the tumor microenvironment.

In some embodiments, the activity of the promoter is increased or decreased by at least 2 fold (e.g., 2, 3, 4, 5, 10, 25, 20, 25, 50, or 100 fold), relative to activity of the promoter in the absence of the tumor microenvironment. For example, the activity of the promoter is increased or decreased by at least 3 fold, at least 5 fold, at least 10 fold, at least 20 fold, at least 50 fold, or at least 100 fold, relative to activity of the promoter in the absence of the tumor microenvironment. In some embodiments, the activity of the promoter is increased or decreased by 2-10, 2-20, 2-30, 2-40, 2-50, 2-60, 2-70, 2-80, 2-90, or 2-100 fold, relative to activity of the promoter in the absence of the tumor microenvironment.

In some embodiments, a promoter of the present disclosure is activated under a hypoxic condition. A “hypoxic condition” is a condition where the body or a region of the body is deprived of adequate oxygen supply at the tissue level. Hypoxic conditions can cause inflammation (e.g., the level of inflammatory cytokines increase under hypoxic conditions). In some embodiments, the promoter that is activated under hypoxic condition is operably linked to a nucleotide encoding an effector molecule that decreases the expression of activity of inflammatory cytokines, thus reducing the inflammation caused by the hypoxic condition. In some embodiments, the promoter that is activated under hypoxic conditions comprises a hypoxia responsive element (EIRE). A “hypoxia responsive element (HRE)” is a response element that responds to hypoxia-inducible factor (HIF). The HRE, in some embodiments, comprises a consensus motif NCGTG (where N is either A or G).

### Multicistronic and Multiple Promoter Systems

In some embodiments, engineered nucleic acids are configured to produce multiple effector molecules. For example, nucleic acids may be configured to produce 2-20 different effector molecules. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, nucleic acids are configured to produce 1, 2, 3, 4, 5, 6, 7, 8, 9, 10, 11, 12, 13, 14, 15, 16, 17, 18, 19, or 20 effector molecules.

In some embodiments, engineered nucleic acids can be multicistronic, i.e., more than one separate polypeptide (e.g., multiple effector molecules) can be produced from a single mRNA transcript. Engineered nucleic acids can be multicistronic through the use of various linkers, e.g., a polynucleotide sequence encoding a first effector molecule can be linked to a nucleotide sequence encoding a second effector molecule, such as in a first gene:linker:second gene 5′ to 3′ orientation. A linker can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker of the present disclosure is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

Engineered nucleic acids can use multiple promoters to express genes from multiple ORFs, i.e., more than one separate mRNA transcript can be produced from a single engineered nucleic acid. For example, a first promoter can be operably linked to a polynucleotide sequence encoding a first effector molecule, and a second promoter can be operably linked to a polynucleotide sequence encoding a second effector molecule. In general, any number of promoters can be used to express any number of effector molecules. In some embodiments, at least one of the ORFs expressed from the multiple promoters can be multicistronic.

“Linkers,” as used herein can refer to polypeptides that link a first polypeptide sequence and a second polypeptide sequence, the multicistronic linkers described above, or the additional promoters that are operably linked to additional ORFs described above.

### Homing Molecules

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007).

In some embodiments, engineered nucleic acids are configured to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, and PDGFR.

In some embodiments, a homing molecule is a chemokine receptor (cell surface molecule that binds to a chemokine). Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. In some embodiments, engineered nucleic acids are configured to produce CXCR4, a chemokine receptor which allows engineered cells to home along a chemokine gradient towards a stromal cell-derived factor 1 (also known as SDF1, C-X-C motif chemokine 12, and CXCL12)-expressing cell, tissue, or tumor. Non-limiting examples of chemokine receptors that may be encoded by the engineered nucleic acids of the present disclosure include: CXC chemokine receptors (e.g., CXCR1, CXCR2, CXCR3, CXCR4, CXCR5, CXCR6, and CXCR7), CC chemokine receptors (CCR1, CCR2, CCR3, CCR4, CCR5, CCR6, CCR7, CCR8, CCR9, CCR10, and CCR11), CX3C chemokine receptors (e.g., CX3CR1, which binds to CX3CL1), and XC chemokine receptors (e.g., XCR1). In some embodiments, a chemokine receptor is a G protein-linked transmembrane receptor, or a member of the tumor necrosis factor (TNF) receptor superfamily (including but not limited to TNFRSF1A, TNFRSF1B). In some embodiments, engineered nucleic acids are configured to produce CXCL8, CXCL9, and/or CXCL10 (promote T-cell recruitment), CCL3 and/or CXCL5, CCL21 (Th1 recruitment and polarization).

In some embodiments, engineered nucleic acids are configured to produce G-protein coupled receptors (GPCRs) that detect N-formylated-containing oligopeptides (including but not limited to FPR2 and FPRL1).

In some embodiments, engineered nucleic acids are configured to produce receptors that detect interleukins (including but not limited to IL6R).

In some embodiments, engineered nucleic acids are configured to produce receptors that detect growth factors secreted from other cells, tissues, or tumors (including but not limited to FGFR, PDGFR, EGFR, and receptors of the VEGF family, including but not limited to VEGF-C and VEGF-D).

In some embodiments, a homing molecule is an integrin. Integrins are transmembrane receptors that facilitate cell-extracellular matrix (ECM) adhesion. Integrins are obligate heterodimers having two subunits: α (alpha) and β (beta). The a subunit of an integrin may be, without limitation: ITGA1, ITGA2, ITGA3, ITGA4, ITGA5, ITGA6, IGTA7, ITGA8, ITGA9, IGTA10, IGTA11, ITGAD, ITGAE, ITGAL, ITGAM, ITGAV, ITGA2B, ITGAX. The β subunit of an integrin may be, without limitation: ITGB1, ITGB2, ITGB3, ITGB4, ITGB5, ITGB6, ITGB7, and ITGB8. Engineered nucleic acids can be configured to produce any combination of the integrin α and β subunits.

In some embodiments, a homing molecule is a matrix metalloproteinase (MMP). MMPs are enzymes that cleave components of the basement membrane underlying the endothelial cell wall. Non-limiting examples of MMPs include MMP-2, MMP-9, and MMP. In some embodiments, engineered nucleic acids are configured to produce an inhibitor of a molecule (e.g., protein) that inhibits MMPs. For example, engineered nucleic acids can be configured to express an inhibitor (e.g., an RNAi molecule) of membrane type 1 MMP (MT1-MMP) or TIMP metallopeptidase inhibitor 1 (TIMP-1).

In some embodiments, a homing molecule is a ligand that binds to selectin (e.g., hematopoietic cell E-/L-selectin ligand (HCELL), Dykstra et al., Stem Cells. 2016 October; 34(10):2501-2511) on the endothelium of a target tissue, for example.

The term “homing molecule” also encompasses transcription factors that regulate the production of molecules that improve/enhance homing of cells.

### Secretion Signals

In general, the one or more effector molecules comprise a secretion signal peptide (also referred to as a signal peptide or signal sequence) at the effector molecule's N-terminus that direct newly synthesized proteins destined for secretion or membrane insertion to the proper protein processing pathways. In embodiments with two or more effector molecules, each effector molecule can comprise a secretion signal. In embodiments with two or more effector molecules, each effector molecule can comprise a secretion signal such that each effector molecule is secreted from an engineered cell.

The secretion signal peptide operably associated with a effector molecule can be a native secretion signal peptide native secretion signal peptide (e.g., the secretion signal peptide generally endogenously associated with the given effector molecule). The secretion signal peptide operably associated with a effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 5.

TABLE 5
Exemplary Signal Secretion Peptides

| Name | Protein SEQUENCE | Source (Uniprot) | DNA SEQUENCE |
|---|---|---|---|
| IL-12 | MCHQQLVISWFSL | P29460 | ATGTGTCACCAGCAGCTCGTTATAT |
| | VFLASPLVA (SEQ | | CCTGGTTTAGTTTGGTGTTTCTCGCT |
| | ID NO: 112) | | TCACCCCTGGTGGCA (SEQ ID NO: |
| | | | 31) |
| IL-12 (Codon | MCHQQLVISWFSL | — | ATGTGCCATCAGCAACTCGTCATCT |
| Optimized) | VFLASPLVA (SEQ | | CCTGGTTCTCCCTTGTGTTCCTCGCT |
| | ID NO: 112) | | TCCCCTCTGGTCGCC (SEQ ID NO: |
| | | | 32) |
| IL-2 (Optimized) | MQLLSCIALILALV | — | ATGCAACTGCTGTCATGTATCGCAC |
| | (SEQ ID NO: 113) | | TCATCCTGGCGCTGGTA (SEQ ID |
| | | | NO: 33) |
| IL-2 (Native) | MYRMQLLSCIALS | P60568 | ATGTATCGGATGCAACTTTTGAGCT |
| | LALVTNS (SEQ ID | | GCATCGCATTGTCTCTGGCGCTGGT |
| | NO: 114) | | GACAAATTCC (SEQ ID NO: 34) |
| Trypsinogen-2 | MNLLLILTFVAAA | P07478 | ATGAATCTCTTGCTCATACTTACGT |
| | VA (SEQ ID NO: | | TTGTCGCTGCTGCCGTTGCG (SEQ ID |
| | 115) | | NO: 35) |
| Gaussia | MGVKVLF ALICIA | — | ATGGGCGTGAAGGTCTTGTTTGCCC |
| Luciferase | VAEA (SEQ ID NO: | | TTATCTGCATAGCTGTTGCGGAGGC |
| | 116) | | G (SEQ ID NO: 36) |
| CD5 | MPMGSLQPLATLY | P06127 | ATGCCGATGGGGAGCCTTCAACCTT |
| | LLGMLVASCLG | | TGGCAACGCTTTATCTTCTGGGGAT |
| | (SEQIDNO: 117) | | GTTGGTTGCTAGTTGCCTTGGG |
| | | | (SEQ ID NO: 37) |
| IgKVII (mouse) | METDTLLLWVLLL | | ATGGAAACTGACACGTTGTTGCTGT |
| | WVPGSTGD (SEQ | | GGGTATTGCTCTTGTGGGTCCCAGG |
| | ID NO: 118) | | ATCTACGGGCGAC (SEQ ID NO: 38) |
| IgKVII (human) | MDMRVPAQLLGL | P01597 | ATGGATATGAGGGTTCCCGCCCAGC |
| | LLLWLRGARC | | TTTTGGGGCTGCTTTTGTTGTGGCTT |
| | (SEQ ID NO: 119) | | CGAGGGGCTCGGTGT (SEQ ID NO: |
| | | | 39) |
| VSV-G | MKCLLYLAFLFIG | — | ATGAAGTGTCTGTTGTACCTGGCGT |
| | VNC (SEQ ID NO: | | TTCTGTTCATTGGTGTAAACTGT |
| | 120) | | (SEQ ID NO: 40) |
| Prolactin | MNIKGSPWKGSLL | P01236 | ATGAATATCAAAGGAAGTCCGTGG |
| | LLLVSNLLLCQSV | | AAGGGTAGTCTCCTGCTGCTCCTCG |
| | AP (SEQ ID NO: | | TATCTAACCTTCTCCTTTGTCAATCC |
| | 121) | | GTGGCACCC (SEQ ID NO: 41) |
| Serum albumin | MKWVTFISLLFLFS | P02768 | ATGAAATGGGTAACATTCATATCAC |
| preproprotein | SAYS (SEQ ID NO: | | TTCTCTTTCTGTTCAGCTCTGCGTAT |
| | 122) | | TCT (SEQ ID NO: 42) |
| Azurocidin | MTRLTVLALLAGL | 20160 | ATGACAAGGCTTACTGTTTTGGCTC |
| preproprotein | LASSRA (SEQ ID | | TCCTCGCTGGACTCTTGGCTTCCTCC |
| | NO: 123) | | CGAGCA (SEQ ID NO: 43) |
| Osteonectin | MRAWIFFLLCLAG | P09486 | ATGAGGGCTTGGATTTTTTTTCTGCT |
| (BM40) | RALA (SEQ ID NO: | | CTGCCTTGCCGGTCGAGCCCTGGCG |
| | 124) | | (SEQ ID NO: 44) |
| CD33 | MPLLLLLPLLWAG | P20138 | ATGCCTCTTCTGCTTTTGCTTCCTCT |
| | ALA (SEQ ID NO: | | TTTGTGGGCAGGTGCCCTCGCA |
| | 125) | | (SEQ ID NO: 45) |
| IL-6 | MNSFSTSAFGPVA | P05231 | ATGAACTCTTTCTCAACCTCTGCGT |
| | FSLGLLLVLPAAFP | | TTGGTCCGGTCGCTTTCTCCCTTGG |
| | AP (SEQ ID NO: | | GCTCCTGCTTGTCTTGCCAGCAGCG |
| | 126) | | TTTCCTGCGCCA (SEQ ID NO: 46) |
| IL-8 | MTSKLAVALLAAF | P10145 | ATGACAAGTAAACTGGCGGTAGCC |
| | LISAALC (SEQ ID | | TTGCTCGCGGCCTTTTTGATTTCCGC |
| | NO: 127) | | AGCCCTTTGT (SEQ ID NO: 47) |
| CCL2 | MKVSAALLCLLLI | P13500 | ATGAAGGTAAGTGCAGCGTTGCTTT |
| | AATFIPQGLA (SEQ | | GCCTTCTCCTCATTGCAGCGACCTT |
| | ID NO: 128) | | TATTCCTCAAGGGCTGGCC (SEQ ID |
| | | | NO: 48) |
| TIMP2 | MGAAARTLRLAL | P16035 | ATGGGAGCGGCAGCTAGAACACTT |
| | GLLLLATLLRPAD | | CGACTTGCCCTTGGGCTCTTGCTCC |
| | A (SEQ ID NO: 129) | | TTGCAACCCTCCTTAGACCTGCCGA |
| | | | CGCA (SEQ ID NO: 49) |
| VEGFB | MSPLLRRLLLAAL | P49765 | ATGTCACCGTTGTTGCGGAGATTGC |
| | LQLAPAQA (SEQ | | TGTTGGCCGCACTTTTGCAACTGGC |
| | ID NO: 130) | | TCCTGCTCAAGCC (SEQ ID NO: 50) |
| Osteoprotegerin | MNNLLCCALVFLD | O00300 | ATGAATAACCTGCTCTGTTGTGCGC |
| | ISIKWTTQ (SEQ ID | | TCGTGTTCCTGGACATTTCTATAAA |
| | NO: 131) | | ATGGACAACGCAA (SEQ ID NO: 51) |
| Serpin E1 | MQMSPALTCLVLG | P05121 | ATGCAAATGTCTCCTGCCCTTACCT |
| | LALVFGEGSA | | GTCTCGTACTTGGTCTTGCGCTCGT |
| | (SEQ ID NO: 132) | | ATTTGGAGAGGGATCAGCC (SEQ ID |
| | | | NO: 52) |
| GROalpha | MARAALSAAPSNP | P09341 | ATGGCAAGGGCTGCACTCAGTGCTG |
| | RLLRVALLLLLLV | | CCCCGTCTAATCCCAGATTGCTTCG |
| | AAGRRAAG (SEQ | | AGTTGCATTGCTTCTTCTGTTGCTGG |
| | ID NO: 133) | | TTGCAGCTGGTAGGAGAGCAGCGG |
| | | | GT (SEQ ID NO: 53) |
| CXCL12 | MNAKVVVVLVLV | P48061 | ATGAATGCAAAAGTCGTGGTCGTGC |
| | LTALCLSDG (SEQ | | TGGTTTTGGTTCTGACGGCGTTGTG |
| | ID NO: 134) | | TCTTAGTGATGGG (SEQ ID NO: 54) |
| IL-21 (Codon | MERIVICLMVIFLG | Q9HBE4 | ATGGAACGCATTGTGATCTGCCTGA |
| Optimized) | TLVHKSSS (SEQ ID | | TGGTCATCTTCCTGGGCACCTTAGT |
| | NO: 135) | | GCACAAGTCGAGCAGC (SEQ ID NO: |
| | | | 55) |

Engineered Cells

Provided herein are engineered cells, and methods of producing the engineered cells, that produce effector molecules that modulate different tumor-mediated immunosuppressive mechanisms. These cells are referred to herein as “engineered cells.” These cells, which typically contain engineered nucleic acid, do not occur in nature. In some embodiments, the cells are engineered to include a nucleic acid comprising a promoter operably linked to a nucleotide sequence encoding an effector molecule, for example, one that stimulates an immune response. An engineered cell can comprise an engineered nucleic acid integrated into the cell's genome. An engineered cell can comprise an engineered nucleic acid capable of expression without integrating into the cell's genome, for example, engineered with a transient expression system such as a plasmid or mRNA.

The present disclosure also encompasses additivity and synergy between an effector molecule(s) and the engineered cell from which they are produced. In some embodiments, cells are engineered to produce at least two (e.g., 2, 3, 4, 5, 6, 7, 8, 9, 10 or more) effector molecules, each of which modulates a different tumor-mediated immunosuppressive mechanism. In other embodiments, cells are engineered to produce at least one effector molecule that is not natively produced by the cells. Such an effector molecule may, for example, complement the function of effector molecules natively produced by the cells.

In some embodiments, cells are engineered to express membrane-tethered anti-CD3 and/or anti-CD28 agonist extracellular domains.

In some embodiments, a cell (e.g., a tumor cell, an erythrocyte, a platelet cell, or a bacterial cell) is engineered to produce multiple effector molecules. For example, cells may be engineered to produce 2-20 different effector molecules. In some embodiments, cells engineered to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, cells are engineered to produce 1, 2, 3, 4, 5, 6, 7, 8, 9, 10, 11, 12, 13, 14, 15, 16, 17, 18, 19, or 20 effector molecules.

In some embodiments, engineered cells comprise one or more engineered nucleic acids encoding a promoter operably linked to a nucleotide sequence encoding an effector molecule. In some embodiments, cells are engineered to include a plurality of engineered nucleic acids, e.g., at least two engineered nucleic acids, each encoding a promoter operably linked to a nucleotide sequence encoding at least one (e.g., 1, 2 or 3) effector molecule. For example, cells may be engineered to comprise at least 2, at least 3, at least 4, at least 5, at least 6, at least 7, at least 8, at least 8, at least 9, or at least 10, engineered nucleic acids, each encoding a promoter operably linked to a nucleotide sequence encoding at least one (e.g., 1, 2 or 3) effector molecule. In some embodiments, the cells are engineered to comprise 2, 3, 4, 5, 6, 7, 8, 9, 10, or more engineered nucleic acids, each encoding a promoter operably linked to a nucleotide sequence encoding at least one (e.g., 1, 2 or 3) effector molecule. Engineered cells can comprise an engineered nucleic acid encoding at least one of the linkers described above, such as polypeptides that link a first polypeptide sequence and a second polypeptide sequence, one or more multicistronic linker described above, one or more additional promoters operably linked to additional ORFs, or a combination thereof.

Engineered cells of the present disclosure (e.g., a tumor cell, an erythrocyte, a platelet cell, or a bacterial cell) typically produce multiple effector molecules, at least two of which modulate different tumor-mediated immunosuppressive mechanisms. In some embodiments, at least one of the effector molecules stimulates an inflammatory pathway in the tumor microenvironment, and at least one of the effector molecules inhibits a negative regulator of inflammation in the tumor microenvironment.

In some embodiments, a cell (e.g., a tumor cell, an erythrocyte, a platelet cell, or a bacterial cell) is engineered to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, and PDGFR.

In some embodiments, a homing molecule is a chemokine receptor (cell surface molecule that binds to a chemokine). Non-limiting examples of chemokine receptors that may be produced by the engineered cells of the present disclosure include: CXC chemokine receptors (e.g., CXCR1, CXCR2, CXCR3, CXCR4, CXCR5, CXCR6, and CXCR7), CC chemokine receptors (CCR1, CCR2, CCR3, CCR4, CCR5, CCR6, CCR7, CCR8, CCR9, CCR10, and CCR11), CX3C chemokine receptors (e.g., CX3CR1, which binds to CX3CL1), and XC chemokine receptors (e.g., XCR1). In some embodiments, a chemokine receptor is a G protein-linked transmembrane receptor, or a member of the tumor necrosis factor (TNF) receptor superfamily (including but not limited to TNFRSF1A, TNFRSF1B). In some embodiments, cells are engineered to produce CXCL8, CXCL9, and/or CXCL10 (promote T-cell recruitment), CCL3 and/or CXCL5, CCL21 (Th1 recruitment and polarization). In some embodiments, cells are engineered to produce CXCR4.

In some embodiments, a cell (e.g., a tumor cell, an erythrocyte, a platelet cell, or a bacterial cell) is engineered to produce G-protein coupled receptors (GPCRs) that detect N-formylated-containing oligopeptides (including but not limited to FPR2 and FPRL1).

In some embodiments, a cell (e.g., a tumor cell, an erythrocyte, a platelet cell, or a bacterial cell) is engineered to produce receptors that detect interleukins (including but not limited to IL6R).

In some embodiments, a cell (e.g., a tumor cell, an erythrocyte, a platelet cell, or a bacterial cell) is engineered to produce receptors that detect growth factors secreted from other cells, tissues, or tumors (including but not limited to FGFR, PDGFR, EGFR, and receptors of the VEGF family, including but not limited to VEGF-C and VEGF-D).

In some embodiments, a cell (e.g., a tumor cell, an erythrocyte, a platelet cell, or a bacterial cell) is engineered to produce one or more integrins. Cells of the present disclosure may be engineered to produce any combination of integrin α and β subunits. The a subunit of an integrin may be, without limitation: ITGA1, ITGA2, ITGA3, ITGA4, ITGA5, ITGA6, IGTA7, ITGA8, ITGA9, IGTA10, IGTA11, ITGAD, ITGAE, ITGAL, ITGAM, ITGAV, ITGA2B, ITGAX. The β subunit of an integrin may be, without limitation: ITGB1, ITGB2, ITGB3, ITGB4, ITGB5, ITGB6, ITGB7, and ITGB8.

In some embodiments, a cell (e.g., a tumor cell, an erythrocyte, a platelet cell, or a bacterial cell) is engineered to produce one or more matrix metalloproteinases (MMP). Non-limiting examples of MMPs include MMP-2, MMP-9, and MMP. In some embodiments, cells are engineered to produce an inhibitor of a molecule (e.g., protein) that inhibits MMPs. For example, cells may be engineered to express an inhibitor (e.g., an RNAi molecule) of membrane type 1 MMP (MT1-MMP) or TIMP metallopeptidase inhibitor 1 (TIMP-1).

In some embodiments, a cell (e.g., a tumor cell, an erythrocyte, a platelet cell, or a bacterial cell) is engineered to produce a ligand that binds to selectin (e.g., hematopoietic cell E-/L-selectin ligand (HCELL), Dykstra et al., Stem Cells. 2016 October; 34(10):2501-2511) on the endothelium of a target tissue, for example.

The term “homing molecule” also encompasses transcription factors that regulate the production of molecules that improve/enhance homing of cells.

Also provided herein are engineered cells (e.g., tumor cells, erythrocytes, platelet cells, or bacterial cells) that are engineered to produce multiple effector molecules, at least two of which modulate different tumor-mediated immunosuppressive mechanisms. In some embodiments, at least one (e.g., 1, 2, 3, 4, 5, or more) effector molecule stimulates at least one immunostimulatory mechanism in the tumor microenvironment, or inhibits at least one immunosuppressive mechanism in the tumor microenvironment. In some embodiments, at least one (e.g., 1, 2, 3, 4, 5, or more) effector molecule inhibits at least one immunosuppressive mechanism in the tumor microenvironment, and at least one effector molecule (e.g., 1, 2, 3, 4, 5, or more) inhibits at least one immunosuppressive mechanism in the tumor microenvironment. In yet other embodiments, at least two (e.g., 2, 3, 4, 5, or more) effector molecules stimulate at least one immunostimulatory mechanism in the tumor microenvironment. In still other embodiments, at least two (e.g., 1, 2, 3, 4, 5, or more) effector molecules inhibit at least one immunosuppressive mechanism in the tumor microenvironment.

In some embodiments, a cell (e.g., a tumor cell, an erythrocyte, a platelet cell, or a bacterial cell) is engineered to produce at least one effector molecule that stimulates T cell signaling, activity and/or recruitment. In some embodiments, a cell is engineered to produce at least one effector molecule that stimulates antigen presentation and/or processing. In some embodiments, a cell is engineered to produce at least one effector molecule that stimulates natural killer cell-mediated cytotoxic signaling, activity and/or recruitment. In some embodiments, a cell is engineered to produce at least one effector molecule that stimulates dendritic cell differentiation and/or maturation. In some embodiments, a cell is engineered to produce at least one effector molecule that stimulates immune cell recruitment. In some embodiments, a cell is engineered to produce at least one effector molecule that stimulates M1 macrophage signaling, activity and/or recruitment. In some embodiments, a cell is engineered to produce at least one effector molecule that stimulates Th1 polarization. In some embodiments, a cell is engineered to produce at least one effector molecule that stimulates stroma degradation. In some embodiments, a cell is engineered to produce at least one effector molecule that stimulates immunostimulatory metabolite production. In some embodiments, a cell is engineered to produce at least one effector molecule that stimulates Type I interferon signaling. In some embodiments, a cell is engineered to produce at least one effector molecule that inhibits negative costimulatory signaling. In some embodiments, a cell is engineered to produce at least one effector molecule that inhibits pro-apoptotic signaling (e.g., via TRAIL) of anti-tumor immune cells. In some embodiments, a cell is engineered to produce at least one effector molecule that inhibits T regulatory (T_{reg}) cell signaling, activity and/or recruitment. In some embodiments, a cell is engineered to produce at least one effector molecule that inhibits tumor checkpoint molecules. In some embodiments, a cell is engineered to produce at least one effector molecule that activates stimulator of interferon genes (STING) signaling. In some embodiments, a cell is engineered to produce at least one effector molecule that inhibits myeloid-derived suppressor cell signaling, activity and/or recruitment. In some embodiments, a cell is engineered to produce at least one effector molecule that degrades immunosuppressive factors/metabolites. In some embodiments, a cell is engineered to produce at least one effector molecule that inhibits vascular endothelial growth factor signaling. In some embodiments, a cell is engineered to produce at least one effector molecule that directly kills tumor cells (e.g., granzyme, perforin, oncolytic viruses, cytolytic peptides and enzymes, anti-tumor antibodies, e.g., that trigger ADCC).

In some embodiments, at least one effector molecule: stimulates T cell signaling, activity and/or recruitment, stimulates antigen presentation and/or processing, stimulates natural killer cell-mediated cytotoxic signaling, activity and/or recruitment, stimulates dendritic cell differentiation and/or maturation, stimulates immune cell recruitment, stimulates macrophage signaling, stimulates stroma degradation, stimulates immunostimulatory metabolite production, or stimulates Type I interferon signaling; and at least one effector molecule inhibits negative costimulatory signaling, inhibits pro-apoptotic signaling of anti-tumor immune cells, inhibits T regulatory (Treg) cell signaling, activity and/or recruitment, inhibits tumor checkpoint molecules, activates stimulator of interferon genes (STING) signaling, inhibits myeloid-derived suppressor cell signaling, activity and/or recruitment, degrades immunosuppressive factors/metabolites, inhibits vascular endothelial growth factor signaling, or directly kills tumor cells.

In some embodiments, a cell (e.g., a tumor cell, an erythrocyte, a platelet cell, or a bacterial cell) is engineered to produce at least one effector molecule selected from IL-12, IFN-β, IFN-γ, IL-2, IL-15, IL-7, IL-36γ, IL-18, IL-1β, OX40-ligand, and CD40L; and/or at least one effector molecule selected from a checkpoint inhibitor. Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Exemplary checkpoint inhibitors include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies. Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MEDI4736/Imfinzi®—Medimmune/AstraZeneca), atezolizumab (anti-PD-L1; Tecentriq®-Roche/Genentech), BMS-936559 (anti-PD-L1—BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca).

In some embodiments, a cell (e.g., a tumor cell, an erythrocyte, a platelet cell, or a bacterial cell) is engineered to produce at least one effector molecule selected from IL-12, IFN-β, IFN-γ, IL-2, IL-15, IL-7, IL-36γ, IL-18, IL-1β, OX40-ligand, and CD40L; and/or at least one effector molecule selected from anti-PD-1 antibodies, anti-PD-L1 antibodies, anti-CTLA-4 antibodies, and anti-IL-35 antibodies; and/or at least one effector molecule selected from MIP1α (CCL3), MIP1β (CCL5), and CCL21; and/or at least one effector molecule selected from CpG oligodeoxynucleotides; and/or at least one effector molecule selected from microbial peptides.

In some embodiments, a cell (e.g., a tumor cell, an erythrocyte, a platelet cell, or a bacterial cell) is engineered to produce IFN-β and at least one effector molecule selected from cytokines, antibodies, chemokines, nucleotides, peptides, enzymes, and stimulators of interferon genes (STINGs). In some embodiments, a cell is engineered to produce IFN-β and at least one cytokine or receptor/ligand (e.g., IL-12, IFN-γ, IL-2, IL-15, IL-7, IL-36γ, IL-18, IL-1β, OX40-ligand, and/or CD40L).

In some embodiments, a cell (e.g., a tumor cell, an erythrocyte, a platelet cell, or a bacterial cell) is engineered to produce IFN-β and at least one cytokine or receptor/ligand (e.g., IL-12, IFN-γ, IL-2, IL-15, IL-7, IL-36γ, IL-18, IL-1β, OX40-ligand, and/or CD40L).

In some embodiments the cytokine is produced as an engineered fusion protein with an antibody, antibody-fragment, or receptor that self-binds to the cytokine to induce cell-specific targeted binding such as with IL-2 fused to an antibody fragment preventing it from binding to Treg cells and preferentially binding to CD8 and NK cells. In some embodiments, a cell (e.g., a tumor cell, an erythrocyte, a platelet cell, or a bacterial cell) is engineered to produce IFN-β and at least one antibody (e.g., anti-PD-1 antibody, anti-PD-L1 antibody, anti-CTLA-4 antibody, anti-VEGF, anti-TGF-β, anti-IL-10, anti-TNF-α, and/or anti-IL-35 antibody). In some embodiments, a cell is engineered to produce IFN-β and at least one chemokine (MIP1α (CCL3), MIP1β (CCL5), and/or CCL21). In some embodiments, a cell is engineered to produce IFN-β and at least one nucleotide (e.g., a CpG oligodeoxynucleotide). In some embodiments, a cell is engineered to produce IFN-β and at least one peptide (e.g., an anti-tumor peptide). In some embodiments, a cell is engineered to produce IFN-β and at least one enzyme. In some embodiments, a cell is engineered to produce IFN-β and at least one STING activator. In some embodiments, a cell is engineered to produce IFN-β and at least one effector with direct anti-tumor activity (e.g., oncolytic virus).

In some embodiments, a cell (e.g., a tumor cell, an erythrocyte, a platelet cell, or a bacterial cell) is engineered to produce IFN-α and MIP1-α. In some embodiments, a cell is engineered to produce IFN-α and MIP1-β. In some embodiments, a cell is engineered to produce IFN-α and CXCL9. In some embodiments, a cell is engineered to produce IFN-α and CXCL10. In some embodiments, a cell is engineered to produce IFN-α and CXCL11. In some embodiments, a cell is engineered to produce IFN-α and CCL21. In some embodiments, the cell is engineered to further produce IL-12, IFN-γ, IL-2, IL-7, IL-15, IL36-γ, IL-18, CD40L and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce IFN-β and MIP1-α. In some embodiments, a cell is engineered to produce IFN-β and MIP1-β. In some embodiments, a cell is engineered to produce IFN-β and CXCL9. In some embodiments, a cell is engineered to produce IFN-β and CXCL10. In some embodiments, a cell is engineered to produce IFN-β and CXCL11. In some embodiments, a cell is engineered to produce IFN-β and CCL21. In some embodiments, the cell is engineered to further produce IL-12, IFN-γ, IL-2, IL-7, IL-15, IL36-γ, IL-18, CD40L and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce IL-12 and MIP1-α. In some embodiments, a cell is engineered to produce IL-12 and MIP1-β. In some embodiments, a cell is engineered to produce IL-12 and CXCL9. In some embodiments, a cell is engineered to produce IL-12 and CXCL10. In some embodiments, a cell is engineered to produce IL-12 and CXCL11. In some embodiments, a cell is engineered to produce IL-12 and CCL21. In some embodiments, the cell is engineered to further produce IFN-β, IFN-γ, IL-2, IL-7, IL-15, IL36-γ, IL-18, CD40L and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce TNF-related apoptosis-inducing ligand (TRAIL) and MIP1-α. In some embodiments, a cell is engineered to produce TRAIL and MIP1-β. In some embodiments, a cell is engineered to produce TRAIL and CXCL9. In some embodiments, a cell is engineered to produce TRAIL and CXCL10. In some embodiments, a cell is engineered to produce TRAIL and CXCL11. In some embodiments, a cell is engineered to produce TRAIL and CCL21. In some embodiments, the cell is engineered to further produce IL-12, IFN-γ, IL-2, IL-7, IL-15, IL36-γ, IL-18, CD40L and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce a stimulator of interferon gene (STING) and MIP1-α. In some embodiments, a cell is engineered to produce STING and MIP1-β. In some embodiments, a cell is engineered to produce STING and CXCL9. In some embodiments, a cell is engineered to produce STING and CXCL10. In some embodiments, a cell is engineered to produce STING and CXCL11. In some embodiments, a cell is engineered to produce STING and CCL21. In some embodiments, the cell is engineered to further produce IL-12, IFN-γ, IL-2, IL-7, IL-15, IL36-γ, IL-18, CD40L and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce CD40L and MIP1-α. In some embodiments, a cell is engineered to produce CD40L and MIP1-β. In some embodiments, a cell is engineered to produce CD40L and CXCL9. In some embodiments, a cell is engineered to produce CD40L and CXCL10. In some embodiments, a cell is engineered to produce CD40L and CXCL11. In some embodiments, a cell is engineered to produce CD40L and CCL21. In some embodiments, the cell is engineered to further produce IL-12, IFN-γ, IL-2, IL-7, IL-15, IL36-γ, IL-18, and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce cytosine deaminase and MIP1-α. In some embodiments, a cell is engineered to produce cytosine deaminase and MIP1-β. In some embodiments, a cell is engineered to produce cytosine deaminase and CXCL9. In some embodiments, a cell is engineered to produce cytosine deaminase and CXCL10. In some embodiments, a cell is engineered to produce cytosine deaminase and CXCL11. In some embodiments, a cell is engineered to produce cytosine deaminase and CCL21. In some embodiments, the cell is engineered to further produce IL-12, IFN-γ, IL-2, IL-7, IL-15, IL36-γ, IL-18, CD40L, and/or 41BB-L. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce IFN-α and IL-12. In some embodiments, a cell is engineered to produce IFN-α and IFN-γ. In some embodiments, a cell is engineered to produce IFN-α and IL-2. In some embodiments, a cell is engineered to produce IFN-α and IL-7. In some embodiments, a cell is engineered to produce IFN-α and IL-15. In some embodiments, a cell is engineered to produce IFN-α and IL-36γ. In some embodiments, a cell is engineered to produce IFN-α and IL-18. In some embodiments, a cell is engineered to produce IFN-α and CD40L. In some embodiments, a cell is engineered to produce IFN-α and 41BB-L. In some embodiments, the cell is engineered to further produce MIP1-α, MIP1-β, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce IFN-β and IL-12. In some embodiments, a cell is engineered to produce IFN-β and IFN-γ. In some embodiments, a cell is engineered to produce IFN-β and IL-2. In some embodiments, a cell is engineered to produce IFN-β and IL-7. In some embodiments, a cell is engineered to produce IFN-β and IL-15. In some embodiments, a cell is engineered to produce IFN-β and IL-36γ. In some embodiments, a cell is engineered to produce IFN-β and IL-18. In some embodiments, a cell is engineered to produce IFN-β and CD40L. In some embodiments, a cell is engineered to produce IFN-β and 41BB-L. In some embodiments, the cell is engineered to further produce MIP1-α, MIP1-β, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce TNF-related apoptosis-inducing ligand (TRAIL) and IL-12. In some embodiments, a cell is engineered to produce TRAIL and IFN-γ. In some embodiments, a cell is engineered to produce TRAIL and IL-2. In some embodiments, a cell is engineered to produce TRAIL and IL-7. In some embodiments, a cell is engineered to produce TRAIL and IL-15. In some embodiments, a cell is engineered to produce TRAIL and IL-36γ. In some embodiments, a cell is engineered to produce TRAIL and IL-18. In some embodiments, a cell is engineered to produce TRAIL and CD40L. In some embodiments, a cell is engineered to produce TRAIL and 41BB-L. In some embodiments, the cell is engineered to further produce MIP1-α, MIP1-13, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce a stimulator of interferon gene (STING) and IL-12. In some embodiments, a cell is engineered to produce STING and IFN-γ. In some embodiments, a cell is engineered to produce STING and IL-2. In some embodiments, a cell is engineered to produce STING and IL-7. In some embodiments, a cell is engineered to produce STING and IL-15. In some embodiments, a cell is engineered to produce STING and IL-36γ. In some embodiments, a cell is engineered to produce STING and IL-18. In some embodiments, a cell is engineered to produce STING and CD40L. In some embodiments, a cell is engineered to produce STING and 41BB-L. In some embodiments, the cell is engineered to further produce MIP1-α, MIP1-13, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce CD40L and IL-12. In some embodiments, a cell is engineered to produce CD40L and IFN-γ. In some embodiments, a cell is engineered to produce CD40L and IL-2. In some embodiments, a cell is engineered to produce CD40L and IL-7. In some embodiments, a cell is engineered to produce CD40L and IL-15. In some embodiments, a cell is engineered to produce CD40L and IL-36γ. In some embodiments, a cell is engineered to produce CD40L and IL-18. In some embodiments, a cell is engineered to produce CD40L and 41BB-L. In some embodiments, the cell is engineered to further produce MIP1-α, MIP1-β, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce cytosine deaminase and IL-12. In some embodiments, a cell is engineered to produce cytosine deaminase and IFN-γ. In some embodiments, a cell is engineered to produce cytosine deaminase and IL-2. In some embodiments, a cell is engineered to produce cytosine deaminase and IL-7. In some embodiments, a cell is engineered to produce cytosine deaminase and IL-15. In some embodiments, a cell is engineered to produce cytosine deaminase and IL-36γ. In some embodiments, a cell is engineered to produce cytosine deaminase and IL-18. In some embodiments, a cell is engineered to produce cytosine deaminase and CD40L. In some embodiments, a cell is engineered to produce cytosine deaminase and 41BB-L. In some embodiments, the cell is engineered to further produce MIP1-α, MIP1-β, CXCL9, CXCL10, CXCL11, and/or CCL21. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce MIP1-α and IL-12. In some embodiments, a cell is engineered to produce MIP1-α and MIP1-γ. In some embodiments, a cell is engineered to produce MIP1-α and IL-2. In some embodiments, a cell is engineered to produce MIP1-α and IL-7. In some embodiments, a cell is engineered to produce MIP1-α and IL-15. In some embodiments, a cell is engineered to produce MIP1-α and IL-36γ. In some embodiments, a cell is engineered to produce MIP1-α and IL-18. In some embodiments, a cell is engineered to produce MIP1-α and CD40L. In some embodiments, a cell is engineered to produce MIP1-α and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce MIP1-β and IL-12. In some embodiments, a cell is engineered to produce MIP1-β and MIP1-γ. In some embodiments, a cell is engineered to produce MIP1-β and IL-2. In some embodiments, a cell is engineered to produce MIP1-β and IL-7. In some embodiments, a cell is engineered to produce MIP1-β and IL-15. In some embodiments, a cell is engineered to produce MIP1-β and IL-36γ. In some embodiments, a cell is engineered to produce MIP1-β and IL-18. In some embodiments, a cell is engineered to produce MIP1-β and CD40L. In some embodiments, a cell is engineered to produce MIP1- and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce CXCL9 and IL-12. In some embodiments, a cell is engineered to produce CXCL9 and IFN-γ. In some embodiments, a cell is engineered to produce CXCL9 and IL-2. In some embodiments, a cell is engineered to produce CXCL9 and IL-7. In some embodiments, a cell is engineered to produce CXCL9 and IL-15. In some embodiments, a cell is engineered to produce CXCL9 and IL-36γ. In some embodiments, a cell is engineered to produce CXCL9 and IL-18. In some embodiments, a cell is engineered to produce CXCL9 and CD40L. In some embodiments, a cell is engineered to produce CXCL9 and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce a CXCL10 and IL-12. In some embodiments, a cell is engineered to produce CXCL10 and IFN-γ. In some embodiments, a cell is engineered to produce CXCL10 and IL-2. In some embodiments, a cell is engineered to produce CXCL10 and IL-7. In some embodiments, a cell is engineered to produce CXCL10 and IL-15. In some embodiments, a cell is engineered to produce CXCL10 and IL-36γ. In some embodiments, a cell is engineered to produce CXCL10 and IL-18. In some embodiments, a cell is engineered to produce CXCL10 and CD40L. In some embodiments, a cell is engineered to produce CXCL10 and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce CXCL11 and IL-12. In some embodiments, a cell is engineered to produce CXCL11 and IFN-γ. In some embodiments, a cell is engineered to produce CXCL11 and IL-2. In some embodiments, a cell is engineered to produce CXCL11 and IL-7. In some embodiments, a cell is engineered to produce CXCL11 and IL-15. In some embodiments, a cell is engineered to produce CXCL11 and IL-36γ. In some embodiments, a cell is engineered to produce CXCL11 and IL-18. In some embodiments, a cell is engineered to produce CXCL11 and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce CCL21 and IL-12. In some embodiments, a cell is engineered to produce CCL21 and IFN-γ. In some embodiments, a cell is engineered to produce CCL21 and IL-2. In some embodiments, a cell is engineered to produce CCL21 and IL-7. In some embodiments, a cell is engineered to produce CCL21 and IL-15. In some embodiments, a cell is engineered to produce CCL21 and IL-36γ. In some embodiments, a cell is engineered to produce CCL21 and IL-18. In some embodiments, a cell is engineered to produce CCL21 and CD40L. In some embodiments, a cell is engineered to produce CCL21 and 41BB-L. In some embodiments, the cell is engineered to further produce IFN-α, IFN-β, TRAIL, STING, CD40L, and/or cytosine deaminase. In some embodiments, the cell is engineered to further produce anti-CD40 antibody, anti-CTLA4 antibody, anti-PD-L1 antibody, and/or OX40L.

In some embodiments, a cell (e.g., a tumor cell, an erythrocyte, a platelet cell, or a bacterial cell) is engineered to produce IFN-α and anti-PD-L1 antibody. In some embodiments, a cell is engineered to produce IFN-α and OX40L. In some embodiments, a cell is engineered to produce IFN-α and anti-CTLA4 antibody. In some embodiments, a cell is engineered to produce IFN-α and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce IFN-β and anti-PD-L1 antibody. In some embodiments, a cell is engineered to produce IFN-β and OX40L. In some embodiments, a cell is engineered to produce IFN-β and anti-CTLA4 antibody. In some embodiments, a cell is engineered to produce IFN-β and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce TRAIL and anti-PD-L1 antibody. In some embodiments, a cell is engineered to produce TRAIL and OX40L. In some embodiments, a cell is engineered to produce TRAIL and anti-CTLA4 antibody. In some embodiments, a cell is engineered to produce TRAIL and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce STING and anti-PD-L1 antibody. In some embodiments, a cell is engineered to produce STING and OX40L. In some embodiments, a cell is engineered to produce STING and anti-CTLA4 antibody. In some embodiments, a cell is engineered to produce STING and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce CD40L and anti-PD-L1 antibody. In some embodiments, a cell is engineered to produce CD40L and OX40L. In some embodiments, a cell is engineered to produce CD40L and anti-CTLA4 antibody. In some embodiments, a cell is engineered to produce CD40L and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce cytosine deaminase and anti-PD-L1 antibody. In some embodiments, a cell is engineered to produce cytosine deaminase and OX40L. In some embodiments, a cell is engineered to produce cytosine deaminase and anti-CTLA4 antibody. In some embodiments, a cell is engineered to produce cytosine deaminase and anti-CD47 antibody. In some embodiments, the cell is engineered to further produce MIP1-α, MIP1-β, CXCL9, CXCL10, CXCL11, and/or CXCL21. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce MIP1-α and anti-PD-L1 antibody. In some embodiments, a cell is engineered to produce MIP1-α and OX40L. In some embodiments, a cell is engineered to produce MIP1-α and anti-CTLA4 antibody. In some embodiments, a cell is engineered to produce MIP1-α and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce MIP1-β and anti-PD-L1 antibody. In some embodiments, a cell is engineered to produce MIP1-β and OX40L. In some embodiments, a cell is engineered to produce MIP1-β and anti-CTLA4 antibody. In some embodiments, a cell is engineered to produce MIP1-β and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce CXCL9 and anti-PD-L1 antibody. In some embodiments, a cell is engineered to produce CXCL9 and OX40L. In some embodiments, a cell is engineered to produce CXCL9 and anti-CTLA4 antibody. In some embodiments, a cell is engineered to produce CXCL9 and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce CXCL10 and anti-PD-L1 antibody. In some embodiments, a cell is engineered to produce CXCL10 and OX40L. In some embodiments, a cell is engineered to produce CXCL10 and anti-CTLA4 antibody. In some embodiments, a cell is engineered to produce CXCL10 and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce CXCL11 and anti-PD-L1 antibody. In some embodiments, a cell is engineered to produce CXCL11 and OX40L. In some embodiments, a cell is engineered to produce CXCL11 and anti-CTLA4 antibody. In some embodiments, a cell is engineered to produce CXCL11 and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce CCL21 and anti-PD-L1 antibody. In some embodiments, a cell is engineered to produce CCL21 and OX40L. In some embodiments, a cell is engineered to produce CCL21 and anti-CTLA4 antibody. In some embodiments, a cell is engineered to produce CCL21 and anti-CD47 antibody. In some embodiments, the cell is engineered to further produce IFN-α, IFN-β, TRAIL, STING, CD40L, and/or cytosine deaminase. In some embodiments, the cell is engineered to further produce IL-12, IFN-γ, IL-2, IL-7, IL-15, IL-36γ, IL-18, CD40L, and/or 41BB-L.

In some embodiments, a cell (e.g., a tumor cell, an erythrocyte, a platelet cell, or a bacterial cell) is engineered to produce IL-12 and anti-PD-L1 antibody. In some embodiments, a cell is engineered to produce IL-12 and OX40L. In some embodiments, a cell is engineered to produce IL-12 and anti-CTLA4 antibody. In some embodiments, a cell is engineered to produce IL-12 and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IFN-γ and anti-PD-L1 antibody. In some embodiments, a cell is engineered to produce IFN-γ and OX40L. In some embodiments, a cell is engineered to produce IFN-γ and anti-CTLA4 antibody. In some embodiments, a cell is engineered to produce IFN-γ and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IL-2 and anti-PD-L1 antibody. In some embodiments, a cell is engineered to produce IL-2 and OX40L. In some embodiments, a cell is engineered to produce IL-2 and anti-CTLA4 antibody. In some embodiments, a cell is engineered to produce IL-2 and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IL-7 and anti-PD-L1 antibody. In some embodiments, a cell is engineered to produce IL-7 and OX40L. In some embodiments, a cell is engineered to produce IL-7 and anti-CTLA4 antibody. In some embodiments, a cell is engineered to produce IL-7 and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IL-15 and anti-PD-L1 antibody. In some embodiments, a cell is engineered to produce IL-15 and OX40L. In some embodiments, a cell is engineered to produce IL-15 and anti-CTLA4 antibody. In some embodiments, a cell is engineered to produce IL-15 and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IL-36-γ and anti-PD-L1 antibody. In some embodiments, a cell is engineered to produce IL-36-γ and OX40L. In some embodiments, a cell is engineered to produce IL-36-γ and anti-CTLA4 antibody. In some embodiments, a cell is engineered to produce IL-36-γ and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce IL-18 and anti-PD-L1 antibody. In some embodiments, a cell is engineered to produce IL-18 and OX40L. In some embodiments, a cell is engineered to produce IL-18 and anti-CTLA4 antibody. In some embodiments, a cell is engineered to produce IL-18 and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce CD40L and anti-PD-L1 antibody. In some embodiments, a cell is engineered to produce CD40L and OX40L. In some embodiments, a cell is engineered to produce CD40L and anti-CTLA4 antibody. In some embodiments, a cell is engineered to produce CD40L and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

In some embodiments, a cell (e.g., a tumor cell, an erythrocyte, a platelet cell, or a bacterial cell) is engineered to produce 41BB-L and anti-PD-L1 antibody. In some embodiments, a cell is engineered to produce 41BB-L and OX40L. In some embodiments, a cell is engineered to produce 41BB-L and anti-CTLA4 antibody. In some embodiments, a cell is engineered to produce 41BB-L and anti-CD47 antibody. In some embodiments, the cell is engineered to further produce IFN-α, IFN-β, TRAIL, STING, CD40L, and/or cytosine deaminase. In some embodiments, the cell is engineered to further produce MIP1-α, MIP1-β, CXCL9, CXCL10, CXCL11, and/or CCL21.

### Engineered Cell Types

Also provided herein are engineered tumor cells. Tumor cells can be engineered to comprise any of the engineered nucleic acids described herein. Tumor cells can be engineered to possess any of the features of any of the engineered cells described herein. In a particular aspect, provided herein are tumor cells engineered to produce one or more effector molecules. In a particular aspect, provided herein are tumor cells engineered to produce two or more effector molecules.

Examples of tumor cells include, but are not limited to, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, an ovarian tumor cell, a pancreatic tumor cell, a prostate tumor cell, a skin tumor cell, a thyroid tumor cell, and a uterine tumor cell.

A tumor cell can be engineered to produce the effector molecules using methods known to those skilled in the art. For example, tumor cells can be transduced to engineer the tumor. In an embodiment, the tumor cell is transduced using a virus.

In a particular embodiment, the tumor cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

The virus, including any of the oncolytic viruses described herein, can be a recombinant virus that encodes one more transgenes encoding one or more effector molecules, such as any of the engineered nucleic acids described herein. The virus, including any of the oncolytic viruses described herein, can be a recombinant virus that encodes one more transgenes encoding one or more of the two or more effector molecules, such as any of the engineered nucleic acids described herein.

Also provided herein are engineered erythrocytes. Erythrocytes can be engineered to comprise any of the engineered nucleic acids described herein. Erythrocytes can be engineered to possess any of the features of any of the engineered cells described herein. In a particular aspect, provided herein are erythrocytes engineered to produce one or more effector molecules. In a particular aspect, provided herein are erythrocytes engineered to produce two or more effector molecules.

Also provided herein are engineered platelet cells. Platelet cells can be engineered to comprise any of the engineered nucleic acids described herein. Platelet cells can be engineered to possess any of the features of any of the engineered cells described herein. In a particular aspect, provided herein are platelet cells engineered to produce one or more effector molecules. In a particular aspect, provided herein are platelet cells engineered to produce two or more effector molecules.

Also provided herein are engineered bacterial cells. Bacterial cells can be engineered to comprise any of the engineered nucleic acids described herein. Bacterial cells can be engineered to possess any of the features of any of the engineered cells described herein. In a particular aspect, provided herein are bacterial cells engineered to produce two or more effector molecules. Bacterial cells can be engineered to produce one or more mammalian effector molecules. Bacterial cells can be engineered to produce two or more mammalian effector molecules. Examples of bacterial cells include, but are not limited to, Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

An engineered cell can be a human cell. An engineered cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer cell. An engineered primary cell can be any somatic cell. An engineered primary cell can be a MSC.

An engineered cell can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

TABLE 6
Sequences encoding exemplary effector molecules

IL 12 (Human) (SEQ ID NO: 56)
ATGTGCCATCAGCAGCTTGTCATATCTTGGTTTTCACTTGTATTCCTGGCCAGCCCTTTGGTTGCG
ATCTGGGAGCTCAAGAAGGATGTGTACGTTGTAGAGCTGGACTGGTACCCCGATGCTCCCGGTG
AGATGGTCGTTTTGACATGTGACACTCCAGAAGAGGACGGTATTACGTGGACTCTGGACCAGTC
CTCCGAAGTTCTTGGTTCTGGTAAGACTCTGACTATCCAGGTGAAAGAATTTGGGGATGCGGGA
CAATACACATGCCACAAGGGAGGCGAGGTGTTGTCTCATAGTTTGCTGCTTCTCCACAAGAAAG
AGGATGGAATCTGGAGCACCGACATACTCAAGGATCAAAAGGAACCCAAAAATAAGACATTTC
TGCGATGTGAGGCTAAGAACTATAGTGGCCGCTTCACTTGTTGGTGGCTGACTACCATCAGCAC
AGATCTCACGTTTTCAGTAAAAAGTAGTAGAGGTTCAAGTGATCCTCAAGGGGTAACGTGCGGT
GCTGCAACACTGTCTGCTGAACGCGTAAGAGGAGATAATAAGGAGTACGAGTATTCCGTAGAAT
GCCAAGAGGACAGTGCTTGTCCTGCGGCCGAGGAGTCTCTCCCAATAGAAGTGATGGTGGACGC
GGTGCATAAACTGAAATATGAGAACTACACAAGCAGTTTTTTTATAAGAGATATCATCAAGCCC
GATCCGCCGAAGAATTTGCAACTTAAACCGCTTAAAAACTCACGCCAGGTTGAAGTATCCTGGG
AGTATCCGGATACATGGTCAACACCACACAGCTATTTTTCCCTTACCTTCTGTGTGCAGGTCCAA
GGGAAGAGCAAAAGGGAGAAGAAGGACAGGGTATTCACTGATAAAACTTCCGCGACGGTCATC
TGCCGAAAAAACGCTAGTATATCTGTACGGGCGCAGGATAGGTACTATAGTTCTTCTTGGTCTG
AGTGGGCCTCAGTTCCGTGCTCTGGGGGAGGAAGTGGAGGAGGGTCCGGCGGTGGAAGCGGGG
GAGGGAGTCGCAACTTGCCAGTGGCTACACCAGATCCAGGCATGTTTCCATGTCTGCATCATTC
CCAGAATCTCCTGAGAGCGGTGTCAAATATGCTCCAAAAAGCGAGACAAACACTGGAATTTTAC
CCGTGTACCAGTGAGGAGATTGATCACGAGGACATAACCAAGGACAAGACCTCAACTGTAGAA
GCGTGTTTGCCGCTGGAGTTGACTAAGAATGAGTCCTGCCTCAATTCCAGAGAAACTTCATTCAT
TACTAACGGCAGTTGTCTTGCATCCCGGAAAACGTCCTTTATGATGGCCCTTTGCCTTAGTTCAA
TTTACGAGGATCTTAAAATGTATCAAGTGGAGTTTAAAACCATGAATGCTAAACTTCTTATGGA
CCCCAAACGACAAATTTTTCTGGATCAGAATATGCTTGCCGTGATAGACGAACTCATGCAGGCG
CTTAATTTTAACTCCGAAACAGTTCCACAAAAATCTAGCCTTGAAGAACCTGATTTTTATAAAAC
GAAGATTAAACTGTGTATCCTGCTGCATGCCTTTCGCATCCGAGCTGTCACAATCGATAGGGTTA
TGTCCTACCTTAACGCGAGCtaG
IL12p70 (Human; codon optimized; bold denotes signal sequence)
(SEQ ID NO: 57)
ATGTGCCATCAGCAACTCGTCATCTCCTGGTTCTCCCTTGTGTTCCTCGCTTCCCCTCTGGTC
GCCATTTGGGAACTGAAGAAGGACGTCTACGTGGTCGAGCTGGATTGGTACCCGGACGCCCC
TGGAGAAATGGTCGTGCTGACTTGCGATACGCCAGAAGAGGACGGCATAACCTGGACCCTGG
ATCAGAGCTCCGAGGTGCTCGGAAGCGGAAAGACCCTGACCATTCAAGTCAAGGAGTTCGGC
GACGCGGGCCAGTACACTTGCCACAAGGGTGGCGAAGTGCTGTCCCACTCCCTGCTGCTGCT
GCACAAGAAAGAGGATGGAATCTGGTCCACTGACATCCTCAAGGACCAAAAAGAACCGAAGA
ACAAGACCTTCCTCCGCTGCGAAGCCAAGAACTACAGCGGTCGGTTCACCTGTTGGTGGCTG
ACGACAATCTCCACCGACCTGACTTTCTCCGTGAAGTCGTCACGGGGATCAAGCGATCCTCAG
GGCGTGACCTGTGGAGCCGCCACTCTGTCCGCCGAGAGAGTCAGGGGAGACAACAAGGAAT
ATGAGTACTCCGTGGAATGCCAGGAGGACAGCGCCTGCCCTGCCGCGGAAGAGTCCCTGCC
TATCGAGGTCATGGTCGATGCCGTGCATAAGCTGAAATACGAGAACTACACTTCCTCCTTCTTT
ATCCGCGACATCATCAAGCCTGACCCCCCCAAGAACTTGCAGCTGAAGCCACTCAAGAACTCC
CGCCAAGTGGAAGTGTCTTGGGAATATCCAGACACTTGGAGCACCCCGCACTCATACTTCTCG
CTCACTTTCTGTGTGCAAGTGCAGGGAAAGTCCAAACGGGAGAAGAAAGACCGGGTGTTCAC
CGACAAAACCTCCGCCACTGTGATTTGTCGGAAGAACGCGTCAATCAGCGTCCGGGCGCAGG
ATAGATACTACTCGTCCTCCTGGAGCGAATGGGCCAGCGTGCCTTGTTCCGGTGGCGGATCA
GGCGGAGGTTCAGGAGGAGGCTCCGGAGGAGGTTCCCGGAACCTCCCTGTGGCAACCCCCG
ACCCTGGAATGTTCCCGTGCCTACACCACTCCCAAAACCTCCTGAGGGCTGTGTCGAACATGT
TGCAGAAGGCCCGCCAGACCCTTGAGTTCTACCCCTGCACCTCGGAAGAAATTGATCACGAG
GACATCACCAAGGACAAGACCTCGACCGTGGAAGCCTGCCTGCCGCTGGAACTGACCAAGAA
CGAATCGTGTCTGAACTCCCGCGAGACAAGCTTTATCACTAACGGCAGCTGCCTGGCGTCGA
GAAAGACCTCATTCATGATGGCGCTCTGTCTTTCCTCGATCTACGAAGATCTGAAGATGTATCA
GGTCGAGTTCAAGACCATGAACGCCAAGCTGCTCATGGACCCGAAGCGGCAGATCTTCCTGG
ACCAGAATATGCTCGCCGTGATTGATGAACTGATGCAGGCCCTGAATTTCAACTCCGAGACTG
TGCCTCAAAAGTCCAGCCTGGAAGAACCGGACTTCTACAAGACCAAGATCAAGCTGTGCATCC
TGTTGCACGCTTTCCGCATTCGAGCCGTGACCATTGACCGCGTGATGTCCTACCTGAACGCCA
GT
IL 12 (Mouse) (SEQ ID NO: 58)
ATGTGTCCACAGAAGCTGACAATAAGTTGGTTTGCCATTGTCCTCCTGGTGAGCCCACTCATGGC
AATGTGGGAACTCGAAAAGGATGTCTACGTGGTAGAAGTAGATTGGACTCCAGACGCGCCAGG
GGAGACAGTGAATTTGACATGTGACACACCAGAAGAAGATGACATTACATGGACATCTGACCA
ACGCCATGGCGTAATAGGGAGTGGGAAAACACTCACGATCACAGTTAAAGAGTTCTTGGATGCT
GGTCAATATACTTGCCATAAAGGCGGCGAGACACTCAGCCACTCACATTTGCTTTTGCATAAAA
AAGAGAATGGCATTTGGAGCACTGAAATACTTAAGAACTTTAAGAACAAGACATTTCTCAAGTG
TGAGGCCCCTAATTACAGCGGCAGGTTCACGTGCTCATGGCTGGTCCAGCGCAACATGGACCTC
AAGTTTAACATAAAATCTTCTTCCTCTTCACCTGACTCCAGAGCTGTTACTTGCGGCATGGCTTC
TCTGAGCGCAGAAAAAGTAACGTTGGATCAAAGAGACTACGAAAAGTACTCTGTTTCTTGTCAA
GAGGATGTTACGTGCCCGACGGCCGAAGAAACGCTTCCAATTGAACTCGCGTTGGAAGCTCGCC
AACAAAACAAGTATGAAAACTACAGTACAAGCTTCTTTATACGGGATATAATTAAACCCGATCC
CCCCAAGAACTTGCAAATGAAACCACTTAAGAACAGCCAGGTGGAAGTTTCCTGGGAGTATCCA
GACTCATGGAGTACTCCTCACAGCTATTTTTCTCTGAAATTCTTTGTAAGGATACAACGGAAGAA
AGAGAAGATGAAAGAGACCGAGGAGGGTTGTAATCAGAAGGGAGCGTTTCTCGTGGAGAAAAC
GTCTACCGAAGTCCAATGTAAAGGTGGCAATGTGTGCGTCCAAGCTCAGGATAGATACTATAAT
TCAAGTTGCTCCAAGTGGGCCTGTGTTCCATGCCGCGTTCGGAGCGGGGGAGGTAGCGGAGGAG
GTAGTGGGGGTGGGTCAGGAGGAGGGAGTCGAGTTATCCCGGTGTCAGGCCCCGCACGCTGCTT
GAGCCAGAGTCGCAACCTCCTTAAGACAACAGATGACATGGTGAAAACAGCACGCGAAAAGCT
TAAACACTACTCTTGTACGGCGGAGGATATTGATCACGAGGATATTACCCGAGACCAAACTAGC
ACTTTGAAAACCTGTCTGCCCCTTGAACTTCATAAAAATGAGAGCTGTCTGGCTACACGAGAGA
CGTCAAGTACGACTAGGGGCAGCTGTCTCCCGCCGCAAAAGACAAGCCTCATGATGACGCTCTG
TTTGGGTTCCATTTACGAGGACTTGAAAATGTATCAAACGGAGTTCCAGGCTATAAATGCGGCG
TTGCAGAACCATAACCATCAACAAATTATACTTGATAAAGGCATGTTGGTGGCGATTGATGAAC
TCATGCAGAGTCTCAATCACAACGGGGAAACGTTGAGACAGAAACCCCCAGTCGGTGAAGCGG
ACCCATATCGAGTAAAAATGAAGCTCTGCATTCTGCTTCACGCATTCAGCACTAGAGTTGTTACC
ATCAACCGGGTAATGGGATATCTCTCCAGTGCGtaG
IL21 (Human; codon optimized; bold denotes signal sequence)
(SEQ ID NO: 59)
ATGGAACGCATTGTGATCTGCCTGATGGTCATCTTCCTGGGCACCTTAGTGCACAAGTCGAG
CAGCCAGGGACAGGACAGGCACATGATTAGAATGCGCCAGCTCATCGATATCGTGGACCAGT
TGAAGAACTACGTGAACGACCTGGTGCCCGAGTTCCTGCCGGCCCCCGAAGATGTGGAAACC
AATTGCGAATGGTCGGCATTTTCCTGCTTTCAAAAGGCACAGCTCAAGTCCGCTAACACCGGG
AACAACGAACGGATCATCAACGTGTCCATCAAAAAGCTGAAGCGGAAGCCTCCCTCCACCAAC
GCCGGACGGAGGCAGAAGCATAGGCTGACTTGCCCGTCATGCGACTCCTACGAGAAGAAGC
CGCCGAAGGAGTTCCTGGAGCGGTTCAAGTCGCTCCTGCAAAAGATGATTCATCAGCACCTG
TCCTCCCGGACTCATGGGTCTGAGGATTCA
IL12p70_T2A_IL21 (Human; codon optimized; bold denotes signal
sequences) (SEQ ID NO: 60)
ATGTGCCATCAGCAACTCGTCATCTCCTGGTTCTCCCTTGTGTTCCTCGCTTCCCCTCTGGTC
GCCATTTGGGAACTGAAGAAGGACGTCTACGTGGTCGAGCTGGATTGGTACCCGGACGCCCC
TGGAGAAATGGTCGTGCTGACTTGCGATACGCCAGAAGAGGACGGCATAACCTGGACCCTGG
ATCAGAGCTCCGAGGTGCTCGGAAGCGGAAAGACCCTGACCATTCAAGTCAAGGAGTTCGGC
GACGCGGGCCAGTACACTTGCCACAAGGGTGGCGAAGTGCTGTCCCACTCCCTGCTGCTGCT
GCACAAGAAAGAGGATGGAATCTGGTCCACTGACATCCTCAAGGACCAAAAAGAACCGAAGA
ACAAGACCTTCCTCCGCTGCGAAGCCAAGAACTACAGCGGTCGGTTCACCTGTTGGTGGCTG
ACGACAATCTCCACCGACCTGACTTTCTCCGTGAAGTCGTCACGGGGATCAAGCGATCCTCAG
GGCGTGACCTGTGGAGCCGCCACTCTGTCCGCCGAGAGAGTCAGGGGAGACAACAAGGAAT
ATGAGTACTCCGTGGAATGCCAGGAGGACAGCGCCTGCCCTGCCGCGGAAGAGTCCCTGCC
TATCGAGGTCATGGTCGATGCCGTGCATAAGCTGAAATACGAGAACTACACTTCCTCCTTCTTT
ATCCGCGACATCATCAAGCCTGACCCCCCCAAGAACTTGCAGCTGAAGCCACTCAAGAACTCC
CGCCAAGTGGAAGTGTCTTGGGAATATCCAGACACTTGGAGCACCCCGCACTCATACTTCTCG
CTCACTTTCTGTGTGCAAGTGCAGGGAAAGTCCAAACGGGAGAAGAAAGACCGGGTGTTCAC
CGACAAAACCTCCGCCACTGTGATTTGTCGGAAGAACGCGTCAATCAGCGTCCGGGCGCAGG
ATAGATACTACTCGTCCTCCTGGAGCGAATGGGCCAGCGTGCCTTGTTCCGGTGGCGGATCA
GGCGGAGGTTCAGGAGGAGGCTCCGGAGGAGGTTCCCGGAACCTCCCTGTGGCAACCCCCG
ACCCTGGAATGTTCCCGTGCCTACACCACTCCCAAAACCTCCTGAGGGCTGTGTCGAACATGT
TGCAGAAGGCCCGCCAGACCCTTGAGTTCTACCCCTGCACCTCGGAAGAAATTGATCACGAG
GACATCACCAAGGACAAGACCTCGACCGTGGAAGCCTGCCTGCCGCTGGAACTGACCAAGAA
CGAATCGTGTCTGAACTCCCGCGAGACAAGCTTTATCACTAACGGCAGCTGCCTGGCGTCGA
GAAAGACCTCATTCATGATGGCGCTCTGTCTTTCCTCGATCTACGAAGATCTGAAGATGTATCA
GGTCGAGTTCAAGACCATGAACGCCAAGCTGCTCATGGACCCGAAGCGGCAGATCTTCCTGG
ACCAGAATATGCTCGCCGTGATTGATGAACTGATGCAGGCCCTGAATTTCAACTCCGAGACTG
TGCCTCAAAAGTCCAGCCTGGAAGAACCGGACTTCTACAAGACCAAGATCAAGCTGTGCATCC
TGTTGCACGCTTTCCGCATTCGAGCCGTGACCATTGACCGCGTGATGTCCTACCTGAACGCCA
GTAGACGGAAACGCGGAAGCGGAGAGGGCAGAGGCTCGCTGCTTACATGCGGGGACGTGGA
AGAGAACCCCGGTCCGATGGAACGCATTGTGATCTGCCTGATGGTCATCTTCCTGGGCACCT
TAGTGCACAAGTCGAGCAGCCAGGGACAGGACAGGCACATGATTAGAATGCGCCAGCTCAT
CGATATCGTGGACCAGTTGAAGAACTACGTGAACGACCTGGTGCCCGAGTTCCTGCCGGCCC
CCGAAGATGTGGAAACCAATTGCGAATGGTCGGCA1111CCTGCTTTCAAAAGGCACAGCTCA
AGTCCGCTAACACCGGGAACAACGAACGGATCATCAACGTGTCCATCAAAAAGCTGAAGCGG
AAGCCTCCCTCCACCAACGCCGGACGGAGGCAGAAGCATAGGCTGACTTGCCCGTCATGCGA
CTCCTACGAGAAGAAGCCGCCGAAGGAGTTCCTGGAGCGGTTCAAGTCGCTCCTGCAAAAGA
TGATTCATCAGCACCTGTCCTCCCGGACTCATGGGTCTGAGGATTCA
IL-12_2A_CCL21a (Human) (SEQ ID NO: 61)
ATGTGCCATCAGCAGCTTGTCATATCTTGGTTTTCACTTGTATTCCTGGCCAGCCCTTTGGTTGCG
ATCTGGGAGCTCAAGAAGGATGTGTACGTTGTAGAGCTGGACTGGTACCCCGATGCTCCCGGTG
AGATGGTCGTTTTGACATGTGACACTCCAGAAGAGGACGGTATTACGTGGACTCTGGACCAGTC
CTCCGAAGTTCTTGGTTCTGGTAAGACTCTGACTATCCAGGTGAAAGAATTTGGGGATGCGGGA
CAATACACATGCCACAAGGGAGGCGAGGTGTTGTCTCATAGTTTGCTGCTTCTCCACAAGAAAG
AGGATGGAATCTGGAGCACCGACATACTCAAGGATCAAAAGGAACCCAAAAATAAGACATTTC
TGCGATGTGAGGCTAAGAACTATAGTGGCCGCTTCACTTGTTGGTGGCTGACTACCATCAGCAC
AGATCTCACGTTTTCAGTAAAAAGTAGTAGAGGTTCAAGTGATCCTCAAGGGGTAACGTGCGGT
GCTGCAACACTGTCTGCTGAACGCGTAAGAGGAGATAATAAGGAGTACGAGTATTCCGTAGAAT
GCCAAGAGGACAGTGCTTGTCCTGCGGCCGAGGAGTCTCTCCCAATAGAAGTGATGGTGGACGC
GGTGCATAAACTGAAATATGAGAACTACACAAGCAGTTTTTTTATAAGAGATATCATCAAGCCC
GATCCGCCGAAGAATTTGCAACTTAAACCGCTTAAAAACTCACGCCAGGTTGAAGTATCCTGGG
AGTATCCGGATACATGGTCAACACCACACAGCTATTTTTCCCTTACCTTCTGTGTGCAGGTCCAA
GGGAAGAGCAAAAGGGAGAAGAAGGACAGGGTATTCACTGATAAAACTTCCGCGACGGTCATC
TGCCGAAAAAACGCTAGTATATCTGTACGGGCGCAGGATAGGTACTATAGTTCTTCTTGGTCTG
AGTGGGCCTCAGTTCCGTGCTCTGGGGGAGGAAGTGGAGGAGGGTCCGGCGGTGGAAGCGGGG
GAGGGAGTCGCAACTTGCCAGTGGCTACACCAGATCCAGGCATGTTTCCATGTCTGCATCATTC
CCAGAATCTCCTGAGAGCGGTGTCAAATATGCTCCAAAAAGCGAGACAAACACTGGAATTTTAC
CCGTGTACCAGTGAGGAGATTGATCACGAGGACATAACCAAGGACAAGACCTCAACTGTAGAA
GCGTGTTTGCCGCTGGAGTTGACTAAGAATGAGTCCTGCCTCAATTCCAGAGAAACTTCATTCAT
TACTAACGGCAGTTGTCTTGCATCCCGGAAAACGTCCTTTATGATGGCCCTTTGCCTTAGTTCAA
TTTACGAGGATCTTAAAATGTATCAAGTGGAGTTTAAAACCATGAATGCTAAACTTCTTATGGA
CCCCAAACGACAAATTTTTCTGGATCAGAATATGCTTGCCGTGATAGACGAACTCATGCAGGCG
CTTAATTTTAACTCCGAAACAGTTCCACAAAAATCTAGCCTTGAAGAACCTGATTTTTATAAAAC
GAAGATTAAACTGTGTATCCTGCTGCATGCCTTTCGCATCCGAGCTGTCACAATCGATAGGGTTA
TGTCCTACCTTAACGCGAGCCGGCGCAAGAGGGGTTCCGGAGAGGGAAGGGGTAGTCTGCTCA
CCTGCGGCGATGTTGAAGAAAATCCTGGTCCCATGGCGCAAAGTCTGGCTCTTTCACTCCTGATC
CTGGTCTTGGCCTTCGGGATTCCGAGGACCCAAGGAAGTGATGGTGGCGCCCAAGATTGTTGCC
TTAAATACAGCCAGCGGAAAATACCCGCGAAAGTGGTCAGGAGTTATAGAAAACAGGAGCCTT
CCCTGGGTTGTAGTATCCCCGCCATACTTTTCCTCCCGAGAAAACGGAGCCAGGCCGAACTGTG
CGCTGACCCTAAGGAACTTTGGGTGCAACAACTTATGCAACACCTGGATAAGACACCTTCTCCT
CAAAAGCCAGCTCAGGGCTGCCGAAAAGATAGAGGCGCCTCAAAAACCGGAAAAAAGGGCAA
AGGTTCTAAAGGATGTAAGCGGACTGAACGCTCTCAAACGCCTAAAGGGCCGtaG
IL-12_2A_CCL21a (Mouse) (SEQ ID NO: 62)
ATGTGTCCACAGAAGCTGACAATAAGTTGGTTTGCCATTGTCCTCCTGGTGAGCCCACTCATGGC
AATGTGGGAACTCGAAAAGGATGTCTACGTGGTAGAAGTAGATTGGACTCCAGACGCGCCAGG
GGAGACAGTGAATTTGACATGTGACACACCAGAAGAAGATGACATTACATGGACATCTGACCA
ACGCCATGGCGTAATAGGGAGTGGGAAAACACTCACGATCACAGTTAAAGAGTTCTTGGATGCT
GGTCAATATACTTGCCATAAAGGCGGCGAGACACTCAGCCACTCACATTTGCTTTTGCATAAAA
AAGAGAATGGCATTTGGAGCACTGAAATACTTAAGAACTTTAAGAACAAGACATTTCTCAAGTG
TGAGGCCCCTAATTACAGCGGCAGGTTCACGTGCTCATGGCTGGTCCAGCGCAACATGGACCTC
AAGTTTAACATAAAATCTTCTTCCTCTTCACCTGACTCCAGAGCTGTTACTTGCGGCATGGCTTC
TCTGAGCGCAGAAAAAGTAACGTTGGATCAAAGAGACTACGAAAAGTACTCTGTTTCTTGTCAA
GAGGATGTTACGTGCCCGACGGCCGAAGAAACGCTTCCAATTGAACTCGCGTTGGAAGCTCGCC
AACAAAACAAGTATGAAAACTACAGTACAAGCTTCTTTATACGGGATATAATTAAACCCGATCC
CCCCAAGAACTTGCAAATGAAACCACTTAAGAACAGCCAGGTGGAAGTTTCCTGGGAGTATCCA
GACTCATGGAGTACTCCTCACAGCTATTTTTCTCTGAAATTCTTTGTAAGGATACAACGGAAGAA
AGAGAAGATGAAAGAGACCGAGGAGGGTTGTAATCAGAAGGGAGCGTTTCTCGTGGAGAAAAC
GTCTACCGAAGTCCAATGTAAAGGTGGCAATGTGTGCGTCCAAGCTCAGGATAGATACTATAAT
TCAAGTTGCTCCAAGTGGGCCTGTGTTCCATGCCGCGTTCGGAGCGGGGGAGGTAGCGGAGGAG
GTAGTGGGGGTGGGTCAGGAGGAGGGAGTCGAGTTATCCCGGTGTCAGGCCCCGCACGCTGCTT
GAGCCAGAGTCGCAACCTCCTTAAGACAACAGATGACATGGTGAAAACAGCACGCGAAAAGCT
TAAACACTACTCTTGTACGGCGGAGGATATTGATCACGAGGATATTACCCGAGACCAAACTAGC
ACTTTGAAAACCTGTCTGCCCCTTGAACTTCATAAAAATGAGAGCTGTCTGGCTACACGAGAGA
CGTCAAGTACGACTAGGGGCAGCTGTCTCCCGCCGCAAAAGACAAGCCTCATGATGACGCTCTG
TTTGGGTTCCATTTACGAGGACTTGAAAATGTATCAAACGGAGTTCCAGGCTATAAATGCGGCG
TTGCAGAACCATAACCATCAACAAATTATACTTGATAAAGGCATGTTGGTGGCGATTGATGAAC
TCATGCAGAGTCTCAATCACAACGGGGAAACGTTGAGACAGAAACCCCCAGTCGGTGAAGCGG
ACCCATATCGAGTAAAAATGAAGCTCTGCATTCTGCTTCACGCATTCAGCACTAGAGTTGTTACC
ATCAACCGGGTAATGGGATATCTCTCCAGTGCGCGGCGCAAGAGGGGTTCCGGAGAGGGAAGG
GGTAGTCTGCTCACCTGCGGCGATGTTGAAGAAAATCCTGGTCCCATGGCGCAAATGATGACCC
TTTCCCTGCTGAGTCTTGTCCTCGCGCTCTGCATCCCGTGGACGCAGGGGTCTGATGGGGGGGGC
CAAGACTGTTGCCTGAAGTATTCACAAAAAAAGATACCGTACTCTATTGTCAGAGGGTACAGGA
AGCAAGAACCCTCCTTGGGTTGCCCTATACCAGCAATTCTTTTCTCCCCACGCAAGCATTCCAAA
CCAGAACTGTGTGCGAACCCCGAGGAGGGTTGGGTACAGAACTTGATGCGAAGGCTTGACCAG
CCCCCAGCCCCTGGCAAGCAGTCACCTGGGTGCAGAAAAAACAGAGGTACTTCAAAGAGCGGC
AAGAAAGGCAAAGGGAGTAAAGGATGTAAAAGAACGGAGCAGACCCAGCCTTCACGAGGCtaG
CCL21a_2A_IL-12 (Mouse) (SEQ ID NO: 63)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTT
GATGCGAAGGCTTGACCAGCCCCCAGCCCCTGGCAAGCAGTCACCTGGGTGCAGAAAAAACAG
AGGTACTTCAAAGAGCGGCAAGAAAGGCAAAGGGAGTAAAGGATGTAAAAGAACGGAGCAGA
CCCAGCCTTCACGAGGCCGGCGCAAGAGGGGTTCCGGAGAGGGAAGGGGTAGTCTGCTCACCT
GCGGCGATGTTGAAGAAAATCCTGGTCCCATGTGTCCACAGAAGCTGACAATAAGTTGGTTTGC
CATTGTCCTCCTGGTGAGCCCACTCATGGCAATGTGGGAACTCGAAAAGGATGTCTACGTGGTA
GAAGTAGATTGGACTCCAGACGCGCCAGGGGAGACAGTGAATTTGACATGTGACACACCAGAA
GAAGATGACATTACATGGACATCTGACCAACGCCATGGCGTAATAGGGAGTGGGAAAACACTC
ACGATCACAGTTAAAGAGTTCTTGGATGCTGGTCAATATACTTGCCATAAAGGCGGCGAGACAC
TCAGCCACTCACATTTGCTTTTGCATAAAAAAGAGAATGGCATTTGGAGCACTGAAATACTTAA
GAACTTTAAGAACAAGACATTTCTCAAGTGTGAGGCCCCTAATTACAGCGGCAGGTTCACGTGC
TCATGGCTGGTCCAGCGCAACATGGACCTCAAGTTTAACATAAAATCTTCTTCCTCTTCACCTGA
CTCCAGAGCTGTTACTTGCGGCATGGCTTCTCTGAGCGCAGAAAAAGTAACGTTGGATCAAAGA
GACTACGAAAAGTACTCTGTTTCTTGTCAAGAGGATGTTACGTGCCCGACGGCCGAAGAAACGC
TTCCAATTGAACTCGCGTTGGAAGCTCGCCAACAAAACAAGTATGAAAACTACAGTACAAGCTT
CTTTATACGGGATATAATTAAACCCGATCCCCCCAAGAACTTGCAAATGAAACCACTTAAGAAC
AGCCAGGTGGAAGTTTCCTGGGAGTATCCAGACTCATGGAGTACTCCTCACAGCTATTTTTCTCT
GAAATTCTTTGTAAGGATACAACGGAAGAAAGAGAAGATGAAAGAGACCGAGGAGGGTTGTAA
TCAGAAGGGAGCGTTTCTCGTGGAGAAAACGTCTACCGAAGTCCAATGTAAAGGTGGCAATGTG
TGCGTCCAAGCTCAGGATAGATACTATAATTCAAGTTGCTCCAAGTGGGCCTGTGTTCCATGCCG
CGTTCGGAGCGGGGGAGGTAGCGGAGGAGGTAGTGGGGGTGGGTCAGGAGGAGGGAGTCGAG
TTATCCCGGTGTCAGGCCCCGCACGCTGCTTGAGCCAGAGTCGCAACCTCCTTAAGACAACAGA
TGACATGGTGAAAACAGCACGCGAAAAGCTTAAACACTACTCTTGTACGGCGGAGGATATTGAT
CACGAGGATATTACCCGAGACCAAACTAGCACTTTGAAAACCTGTCTGCCCCTTGAACTTCATA
AAAATGAGAGCTGTCTGGCTACACGAGAGACGTCAAGTACGACTAGGGGCAGCTGTCTCCCGC
CGCAAAAGACAAGCCTCATGATGACGCTCTGTTTGGGTTCCATTTACGAGGACTTGAAAATGTA
TCAAACGGAGTTCCAGGCTATAAATGCGGCGTTGCAGAACCATAACCATCAACAAATTATACTT
GATAAAGGCATGTTGGTGGCGATTGATGAACTCATGCAGAGTCTCAATCACAACGGGGAAACGT
TGAGACAGAAACCCCCAGTCGGTGAAGCGGACCCATATCGAGTAAAAATGAAGCTCTGCATTCT
GCTTCACGCATTCAGCACTAGAGTTGTTACCATCAACCGGGTAATGGGATATCTCTCCAGTGCGt
aG
IL7 (Mouse) (SEQ ID NO: 64)
ATGTTTCATGTGTCCTTCAGGTACATATTTGGTATCCCACCACTTATATTGGTGCTCTTGCCTGTA
ACCAGCTCTGAATGTCATATAAAAGACAAGGAGGGCAAAGCATACGAGTCCGTATTGATGATCT
CAATCGATGAACTTGACAAGATGACAGGGACCGATTCTAATTGTCCAAATAACGAGCCAAACTT
CTTTCGGAAACACGTGTGTGATGATACAAAAGAAGCTGCTTTTCTTAACAGAGCTGCCAGAAAA
CTCAAGCAGTTCCTCAAGATGAATATATCCGAGGAATTTAACGTGCATCTCCTCACAGTATCTCA
GGGAACTCAAACCCTTGTAAACTGCACTTCTAAGGAGGAGAAGAATGTCAAAGAGCAGAAGAA
AAATGATGCATGTTTTTTGAAACGGCTGTTGAGGGAGATCAAAACATGCTGGAATAAAATCCTC
AAGGGCTCAATTtaG
IL15 (Human) (SEQ ID NO: 65)
ATGGAAACAGACACATTGCTGCTTTGGGTATTGTTGCTCTGGGTGCCTGGATCAACAGGAAACT
GGGTAAACGTAATTTCAGATCTGAAGAAGATCGAGGACCTTATTCAATCCATGCACATCGATGC
CACTCTCTACACCGAAAGCGACGTTCACCCATCTTGCAAGGTGACCGCTATGAAATGTGAATTG
TTGGAACTTCAGGTAATTTCTCTGGAGAGCGGCGATGCCTCAATACATGACACCGTTGAAAATC
TTATCATCCTTGCTAATGATTCACTCTCTAGTAATGGGAACGTAACAGAGAGCGGGTGTAAGGA
GTGTGAAGAACTGGAGGAGAAAAACATTAAGGAATTTTTGCAGTCATTCGTCCATATAGTGCAA
ATGTTCATAAACACTTCCAGAAGAAAGCGAGGCTCTGGGGAGGGGCGAGGCTCTCTGCTGACCT
GTGGGGATGTAGAAGAGAATCCAGGTCCCATGGACCGGCTGACCAGCTCATTCCTGCTTCTGAT
TGTGCCAGCCTACGTGCTCTCCATCACATGTCCTCCCCCAATGAGCGTCGAGCATGCTGACATCT
GGGTGAAGTCATACTCCTTGTACAGCAGAGAGAGATACATTTGTAATTCCGGATTCAAGCGCAA
GGCCGGCACCTCCTCTCTGACAGAGTGCGTCCTTAACAAAGCAACCAACGTAGCACATTGGACC
ACACCATCCTTGAAGTGCATACGAGAACCTAAATCTTGCGATAAGACTCATACTTGTCCACCTT
GTCCAGCCCCAGAACTGCTTGGCGGACCCTCAGTATTTTTGTTCCCACCAAAGCCAAAAGACAC
ACTCATGATATCCAGAACTCCTGAGGTGACCTGTGTCGTTGTAGACGTTTCCCACGAAGATCCTG
AAGTAAAATTCAACTGGTACGTGGATGGGGTCGAAGTCCATAACGCCAAGACTAAACCAAGGG
AGGAACAGTATAACTCTACTTACCGAGTAGTTTCTGTGTTGACCGTGCTGCACCAGGACTGGTTG
AACGGGAAGGAGTACAAATGCAAGGTGAGCAATAAAGCTCTGCCCGCACCAATCGAAAAGACA
ATATCTAAGGCCAAGGGGCAGCCACGAGAGCCCCAGGTATACACACTGCCACCCTCACGCGAT
GAATTGACTAAGAACCAGGTTTCCCTGACCTGTCTTGTAAAAGGTTTCTACCCTTCCGACATAGC
TGTTGAGTGGGAAAGTAACGGGCAGCCAGAGAACAATTACAAGACAACTCCACCCGTTCTTGAT
AGCGATGGATCATTTTTTCTGTATTCCAAACTCACTGTCGATAAAAGTCGCTGGCAGCAAGGCA
ATGTTTTTAGCTGCTCAGTCATGCACGAAGCACTGCATAATCACTACACACAAAAAAGTTTGTCC
CTTAGCCCTGGTAAGtaG
IL15 (Human) (SEQ ID NO: 66)
ATGTACTCAATGCAGTTGGCCTCCTGTGTAACATTGACCTTGGTCCTCTTGGTCAACAGCAATTG
GATCGATGTACGCTACGACTTGGAGAAGATTGAGTCCCTTATACAGAGTATACACATAGATACA
ACCTTGTATACTGACAGTGACTTCCATCCCAGCTGTAAAGTGACTGCAATGAACTGTTTTTTGTT
GGAGTTGCAAGTAATTCTGCATGAATACAGCAACATGACCCTCAATGAAACCGTTAGGAATGTC
CTTTATCTCGCAAATTCTACTCTGAGTAGCAATAAGAATGTTGCCGAAAGCGGCTGCAAGGAGT
GCGAAGAACTGGAGGAAAAAACTTTCACCGAGTTTCTCCAGAGTTTCATCAGAATTGTCCAAAT
GTTCATTAATACAAGTAGTGGTGGTGGGAGCGGGGGTGGAGGCAGTGGGGGAGGTGGGAGCGG
AGGTGGAGGGTCCGGAGGGGGGAGCCTTCAAGGCACTACTTGTCCTCCACCCGTATCCATCGAG
CACGCCGATATTCGAGTTAAAAATTATAGTGTTAATAGCAGAGAACGATACGTCTGCAACTCAG
GGTTTAAGAGAAAGGCCGGAACTTCAACTCTCATAGAATGCGTGATTAATAAGAATACTAACGT
CGCACATTGGACTACTCCCAGTCTCAAGTGCATACGCGATCCATCTCTCGCTCATTACTCACCAG
TACCTACAGTGGTTACTCCTAAGGTGACCTCTCAGCCCGAATCACCATCTCCCAGCGCAAAAGA
GCCTGAGGCCTTTTCTCCTAAATCAGACACTGCTATGACTACAGAAACAGCCATAATGCCAGGA
AGCCGGCTGACACCATCTCAAACTACCAGCGCAGGCACAACTGGGACTGGCTCCCACAAAAGC
TCACGCGCACCAAGTCTCGCCGCAACAATGACATTGGAGCCTACAGCCAGCACATCTCTTAGAA
TCACAGAAATTTCTCCCCACAGTAGCAAGATGACCAAGGTGGCAATTAGTACCAGCGTCCTTCT
TGTAGGAGCTGGAGTTGTGATGGCATTTTTGGCATGGTATATCAAAAGCAGGtaG
IL15 (Mouse) (SEQ ID NO: 67)
ATGAAGATCCTCAAGCCATACATGCGAAACACTAGTATTAGCTGTTACTTGTGTTTTCTGCTGAA
TAGTCATTTTTTGACTGAAGCAGGAATCCATGTATTTATACTCGGTTGTGTGTCTGTAGGTCTGC
CAAAGACTGAGGCTAATTGGATTGACGTGCGCTATGATCTTGAAAAAATAGAGTCCTTGATTCA
ATCAATACACATCGATACCACTCTCTACACCGACAGTGATTTCCATCCTTCCTGCAAGGTAACAG
CTATGAATTGCTTCCTCCTGGAGCTCCAAGTCATTCTCCATGAGTACTCCAACATGACTTTGAAC
GAAACTGTAAGAAACGTATTGTATCTGGCTAATAGCACCTTGTCTAGTAACAAAAATGTGGCAG
AGAGCGGCTGCAAAGAATGTGAAGAATTGGAAGAGAAAACATTTACAGAGTTCCTGCAATCCT
TTATTCGCATCGTCCAAATGTTTATCAATACCTCTtaG
IL15 (Mouse) (SEQ ID NO: 68)
ATGTATTCCATGCAACTTGCCAGTTGTGTAACCCTTACTCTCGTCCTGCTCGTTAATTCCGCTGGT
GCTAACTGGATAGATGTTCGATACGATCTGGAAAAGATTGAGTCCCTTATCCAATCCATTCATAT
AGATACCACCCTTTATACTGACAGCGACTTCCATCCTTCTTGCAAGGTGACCGCTATGAATTGTT
TCCTGCTGGAACTCCAAGTTATTCTGCATGAATACTCTAATATGACACTTAACGAGACCGTAAG
AAATGTTCTCTATCTCGCTAATAGTACTTTGAGCTCAAATAAGAACGTGGCCGAGTCTGGGTGTA
AGGAATGCGAAGAGCTGGAAGAAAAGACATTCACCGAGTTTCTCCAGTCTTTCATACGGATTGT
GCAGATGTTTATCAACACATCAGATTACAAAGACGACGATGATAAGtaG
IL18 (Mouse) (SEQ ID NO: 69)
ATGGCAGCCATGTCTGAGGACTCTTGTGTGAACTTTAAAGAAATGATGTTCATAGACAATACAC
TCTACTTTATACCTGAGGAGAATGGAGATTTGGAATCTGACAACTTTGGCAGGCTGCATTGCACT
ACCGCAGTTATCCGAAACATCAACGATCAGGTACTGTTTGTTGATAAAAGACAACCTGTATTCG
AGGACATGACCGACATAGATCAGTCTGCCTCAGAGCCCCAGACTAGGCTTATCATCTATATGTA
CAAGGACAGCGAAGTACGAGGCCTGGCTGTTACACTCTCAGTCAAAGACTCTAAGATGAGCAC
CCTGTCATGCAAGAACAAAATTATCAGTTTTGAGGAGATGGACCCACCTGAAAACATAGATGAC
ATTCAGTCAGACCTCATTTTTTTTCAAAAGCGGGTACCAGGACACAACAAAATGGAATTTGAAT
CATCACTCTACGAAGGACATTTCCTTGCATGCCAGAAAGAGGATGACGCATTCAAATTGATCCT
GAAAAAAAAGGACGAAAATGGTGATAAATCAGTCATGTTTACATTGACCAATCTTCACCAAAGT
taG
IL18 (Mouse) (SEQ ID NO: 70)
ATGGCTGCAATGTCTGAAGATAGCTGTGTCAACTTTAAGGAGATGATGTTCATTGATAATACTTT
GTACTTTATACCTGAAGAAAATGGAGACCTTGAGTCAGACAACTTCGGGAGACTGCACTGCACA
ACTGCCGTTATCCGAAACATAAATGATCAAGTATTGTTCGTGGACAAAAGACAACCAGTCTTTG
AGGATATGACAGACATCGACCAATCCGCATCTGAACCTCAGACTAGGCTGATCATCTATATGTA
CGCCGACTCCGAAGTAAGAGGCCTTGCTGTGACACTTAGTGTTAAGGATAGTAAGATGAGCACA
CTGTCCTGTAAGAATAAGATTATATCTTTTGAAGAGATGGACCCTCCCGAGAACATAGATGACA
TCCAGAGCGACTTGATCTTCTTTCAGAAGCGAGTGCCAGGCCATAACAAGATGGAATTTGAATC
ATCTCTTTATGAAGGCCATTTCCTCGCATGTCAAAAGGAGGACGATGCCTTCAAGCTCATTCTGA
AAAAAAAAGACGAGAACGGTGATAAGAGCGTGATGTTCACTCTGACAAATCTGCACCAGTCAta
G
IL18 (Human) (SEQ ID NO: 71)
ATGTATCGCATGCAACTCCTGTCCTGCATTGCTCTGAGCTTGGCTTTGGTAACCAACTCATACTT
CGGGAAACTGGAGAGTAAACTCTCCGTAATCAGGAATCTTAATGACCAAGTATTGTTTATTGAC
CAGGGCAACCGCCCGTTGTTCGAGGATATGACTGATTCTGACTGTCGGGATAACGCTCCGAGAA
CTATCTTTATCATTTCAATGTACAAGGACAGCCAACCGCGGGGTATGGCTGTGACAATCAGTGT
CAAATGTGAGAAGATTTCCACGCTGTCCTGCGAAAACAAGATAATTTCTTTCAAAGAAATGAAC
CCCCCTGACAATATAAAGGATACAAAGAGTGATATCATCTTCTTTCAGAGGTCCGTGCCCGGCC
ACGATAATAAGATGCAATTTGAAAGTTCATCTTATGAGGGGTACTTTTTGGCATGCGAGAAAGA
AAGGGATCTCTTCAAGTTGATCCTGAAGAAGGAGGACGAATTGGGCGACCGCTCCATCATGTTC
ACAGTCCAGAACGAGGACtaG
IL18 (Human) (SEQ ID NO: 72)
ATGTACCGCATGCAGCTCCTGAGTTGTATTGCCCTTTCCCTCGCTCTCGTTACCAATTCTTACTTC
GGTAAGCTTGCCTCTAAACTCTCTGTTATTAGGAACTTGAACGACCAAGTCCTTTTCATAGACCA
AGGGAACAGACCACTGTTTGAAGATATGACGGATAGCGATTGCCGAGATAATGCCCCTAGGAC
GATTTTTATCATTAGTATGTATGCGGACTCTCAACCGAGGGGGATGGCCGTTACTATAAGTGTGA
AATGCGAGAAAATATCAACGCTCAGTTGTGAGAACAAAATCATAAGTTTCAAGGAGATGAATC
CACCTGATAACATCAAAGACACTAAGTCTGATATTATATTTTTCCAACGAAGTGTTCCGGGACA
CGATAACAAAATGCAATTTGAGAGCTCCTCATACGAGGGCTACTTCCTCGCGTGTGAGAAAGAA
AGGGATTTGTTTAAGCTTATCCTCAAGAAAGAGGACGAGTTGGGGGATCGGAGCATAATGTTTA
CCGTACAGAATGAGGACtaG
IL21 (Mouse) (SEQ ID NO: 73)
ATGGAGCGGACACTCGTGTGTCTTGTCGTAATTTTTCTCGGGACAGTCGCACACAAGTCCTCACC
CCAGGGTCCTGATCGCCTTCTCATACGCCTCCGACATTTGATCGACATTGTAGAGCAGCTCAAA
ATTTACGAGAATGACCTCGATCCCGAGCTTTTGAGTGCTCCCCAAGACGTTAAGGGTCATTGCG
AGCACGCAGCTTTTGCTTGCTTCCAGAAGGCCAAGTTGAAACCAAGCAACCCTGGTAATAATAA
GACTTTCATCATCGACTTGGTCGCCCAACTCCGAAGGAGGCTGCCTGCCCGGCGCGGAGGAAAA
AAACAAAAGCATATTGCAAAGTGTCCTTCATGTGATTCATACGAAAAGCGGACTCCCAAAGAGT
TCTTGGAAAGGTTGAAATGGCTTCTTCAGAAGATGATTCATCAACATTTGTCAtaG
IFN-beta (Human) (SEQ ID NO: 74)
ATGACCAACAAATGCCTTTTGCAAATTGCCCTGCTTTTGTGTTTTAGCACTACCGCATTGAGCAT
GTCATATAACCTCCTCGGCTTCCTTCAGAGATCATCAAACTTTCAGTGTCAGAAACTGCTTTGGC
AACTTAATGGCAGGCTCGAATATTGTCTGAAAGATCGGATGAATTTCGACATTCCAGAAGAAAT
AAAACAGCTTCAACAATTCCAGAAAGAGGACGCCGCCCTGACTATTTACGAGATGCTCCAGAAT
ATCTTCGCCATTTTCCGGCAGGACAGCTCATCCACGGGGTGGAATGAGACTATTGTAGAAAATC
TTCTGGCTAATGTGTACCATCAAATTAATCACCTCAAAACGGTGCTTGAGGAAAAACTTGAAAA
GGAAGATTTCACACGGGGCAAGTTGATGTCCTCCCTGCACCTTAAACGATACTACGGCAGGATT
CTTCATTACTTGAAGGCTAAGGAGTATAGCCATTGCGCGTGGACAATTGTACGGGTAGAAATAC
TGCGAAACTTTTATTTCATCAACCGGCTCACTGGATACCTTAGAAATtaG
IFN-beta (Mouse) (SEQ ID NO: 75)
ATGAACAATCGGTGGATACTCCACGCCGCATTTCTCCTCTGCTTTAGCACGACGGCCCTGTCCAT
CAACTACAAACAGCTTCAGTTGCAGGAGCGGACTAACATAAGGAAGTGCCAGGAACTGCTGGA
ACAGCTTAATGGTAAAATTAATCTTACATACCGAGCTGACTTCAAAATTCCTATGGAAATGACC
GAGAAGATGCAGAAATCCTACACGGCATTCGCCATCCAGGAAATGCTCCAGAACGTATTTCTCG
TGTTCCGCAATAATTTCTCTTCTACGGGTTGGAACGAAACCATTGTTGTTAGACTGCTTGACGAA
CTGCATCAGCAAACCGTGTTCCTTAAAACCGTGCTTGAGGAGAAGCAGGAGGAGCGCCTGACTT
GGGAGATGTCTAGTACCGCACTTCACTTGAAATCCTACTACTGGCGCGTTCAGCGGTATCTGAA
GCTGATGAAGTATAACTCATACGCCTGGATGGTAGTGCGCGCAGAGATCTTCAGAAACTTTCTT
ATCATCCGGCGACTGACCCGAAACTTTCAGAATtaG
IFN-gamma (Human) (SEQ ID NO: 76)
ATGAAGTACACTAGCTATATATTGGCCTTCCAGCTTTGCATCGTATTGGGTAGCCTCGGATGCTA
TTGCCAAGACCCGTATGTCAAAGAAGCCGAAAATCTCAAAAAGTATTTCAATGCCGGACACTCA
GACGTCGCGGATAACGGTACACTGTTTCTTGGCATCCTGAAAAATTGGAAGGAAGAGAGTGACA
GAAAAATAATGCAGTCACAAATAGTGTCCTTTTACTTTAAGCTGTTCAAAAATTTCAAGGATGA
CCAAAGTATCCAGAAGAGTGTTGAAACTATCAAAGAGGACATGAATGTGAAATTCTTTAACAGT
AATAAGAAGAAGCGCGATGACTTCGAGAAACTCACTAATTACAGCGTAACGGATCTTAACGTCC
AACGCAAGGCAATCCACGAGCTTATACAGGTAATGGCTGAGCTTAGTCCCGCAGCCAAGACAG
GGAAGAGAAAAAGGTCTCAAATGCTTTTTCGGGGCCGGCGAGCTTCACAAtaG
IFN-gamma (Mouse) (SEQ ID NO: 77)
ATGAACGCTACGCATTGCATCCTCGCACTCCAATTGTTCCTCATGGCTGTGTCAGGGTGTTACTG
TCACGGTACTGTCATAGAAAGCCTCGAATCCCTGAATAACTATTTTAACAGTAGCGGTATAGAT
GTAGAAGAAAAGTCTCTCTTTCTTGACATCTGGAGGAATTGGCAAAAGGATGGAGACATGAAG
ATTCTCCAATCTCAGATTATATCATTTTACTTGAGGCTTTTTGAGGTTCTGAAGGATAACCAGGC
GATCAGCAATAATATCAGCGTAATTGAATCTCACCTTATTACAACATTTTTCTCAAATTCCAAGG
CAAAGAAAGATGCTTTCATGTCTATCGCGAAATTTGAGGTGAACAATCCTCAGGTACAAAGGCA
AGCCTTTAACGAGCTGATTAGAGTTGTACATCAGTTGTTGCCCGAAAGTAGTCTTAGAAAACGC
AAACGGAGCCGATGCtaG
IFN-alpha (Mouse) (SEQ ID NO: 78)
ATGGCAAGGTTGTGCGCTTTTCTCATGGTACTGGCTGTGCTCTCCTATTGGCCTACTTGTTCTCTG
GGATGCGACTTGCCACAGACCCACAATCTGCGGAATAAGAGGGCTCTGACTCTGCTGGTGCAAA
TGAGACGGCTCTCTCCACTTAGCTGTTTGAAAGATAGAAAGGATTTCGGGTTCCCCCAGGAGAA
GGTGGATGCCCAGCAGATCAAGAAGGCACAGGCTATCCCCGTCCTTTCCGAGCTGACCCAGCAA
ATTTTGAACATCTTTACAAGTAAGGATAGTTCAGCTGCATGGAATACCACACTTTTGGATTCTTT
TTGTAACGATCTGCATCAGCAGCTGAACGATCTCCAGGGATGCCTGATGCAGCAAGTCGGCGTG
CAAGAATTTCCACTCACCCAGGAGGACGCTCTGCTCGCAGTGCGAAAGTATTTTCACCGAATTA
CCGTGTACCTCCGGGAGAAAAAGCATTCACCCTGCGCTTGGGAAGTAGTCAGGGCCGAAGTATG
GAGAGCCCTTAGTAGCTCCGCTAATGTACTGGGCCGGTTGCGGGAAGAGAAAtaG
CCL21 (Human) (SEQ ID NO: 79)
ATGGCGCAAAGTCTGGCTCTTTCACTCCTGATCCTGGTCTTGGCCTTCGGGATTCCGAGGACCCA
AGGAAGTGATGGTGGCGCCCAAGATTGTTGCCTTAAATACAGCCAGCGGAAAATACCCGCGAA
AGTGGTCAGGAGTTATAGAAAACAGGAGCCTTCCCTGGGTTGTAGTATCCCCGCCATACTTTTCC
TCCCGAGAAAACGGAGCCAGGCCGAACTGTGCGCTGACCCTAAGGAACTTTGGGTGCAACAAC
TTATGCAACACCTGGATAAGACACCTTCTCCTCAAAAGCCAGCTCAGGGCTGCCGAAAAGATAG
AGGCGCCTCAAAAACCGGAAAAAAGGGCAAAGGTTCTAAAGGATGTAAGCGGACTGAACGCTC
TCAAACGCCTAAAGGGCCGtaG
CCL21a (Mouse) (SEQ ID NO: 80)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTT
GATGCGAAGGCTTGACCAGCCCCCAGCCCCTGGCAAGCAGTCACCTGGGTGCAGAAAAAACAG
AGGTACTTCAAAGAGCGGCAAGAAAGGCAAAGGGAGTAAAGGATGTAAAAGAACGGAGCAGA
CCCAGCCTTCACGAGGCtaG
Tail-less CCL21 (Human) (SEQ ID NO: 81)
ATGGCGCAAAGTCTGGCTCTTTCACTCCTGATCCTGGTCTTGGCCTTCGGGATTCCGAGGACCCA
AGGAAGTGATGGTGGCGCCCAAGATTGTTGCCTTAAATACAGCCAGCGGAAAATACCCGCGAA
AGTGGTCAGGAGTTATAGAAAACAGGAGCCTTCCCTGGGTTGTAGTATCCCCGCCATACTTTTCC
TCCCGAGAAAACGGAGCCAGGCCGAACTGTGCGCTGACCCTAAGGAACTTTGGGTGCAACAAC
TTATGCAACACCTGGATAAGACACCTTCTCCTCAAAAGCCAGCTCAGGGCtaG
Tail-less CCL21 (Mouse) (SEQ ID NO: 82)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTT
GATGCGAAGGCTTGACCAGCCCCCAGCCCCTGGCAAGCAGTCACCTGGGtaG
CCL19 (Mouse) (SEQ ID NO: 83)
ATGGCACCCCGCGTCACACCCTTGCTTGCTTTTTCTCTGCTTGTCCTCTGGACCTTCCCCGCTCCT
ACCCTTGGAGGAGCCAATGATGCCGAGGATTGCTGCCTGAGTGTTACACAAAGGCCAATACCAG
GGAATATAGTGAAGGCATTCCGGTATCTGCTCAATGAAGATGGGTGCAGAGTCCCCGCAGTTGT
CTTTACAACATTGCGAGGTTACCAGCTTTGTGCTCCCCCAGACCAGCCTTGGGTAGATCGCATTA
TTCGCCGGTTGAAGAAGAGCTCAGCAAAGAATAAGGGCAATTCCACACGGAGAAGCCCCGTCT
CCtaG
CCL19 (Mouse) (SEQ ID NO: 84)
ATGAAATCAGCAGTCCTTTTCTTGCTCGGGATTATTTTTCTGGAACAATGTGGAGTGAGGGGAAC
ACTCGTAATAAGAAACGCTCGGTGCTCATGCATATCAACATCACGGGGCACTATCCACTACAAA
TCCCTGAAGGATCTGAAGCAGTTCGCCCCAAGCCCTAACTGTAACAAGACCGAAATTATCGCAA
CTCTCAAAAATGGAGATCAGACTTGTCTTGACCCAGATTCAGCAAATGTCAAGAAGCTGATGAA
AGAGTGGGAAAAGAAGATTTCACAAAAAAAAAAGCAAAAACGCGGCAAGAAACATCAAAAGA
ACATGAAAAACAGGAAACCTAAGACTCCCCAGTCAAGGAGAAGATCCCGCAAGACAACCtaG
CXCL11 (Mouse) (SEQ ID NO: 85)
ATGAACAGAAAAGTTACCGCTATAGCACTTGCTGCCATAATATGGGCCACCGCAGCTCAAGGGT
TCCTGATGTTCAAGCAGGGCCGATGCCTCTGCATTGGCCCTGGAATGAAGGCCGTGAAAATGGC
CGAAATAGAAAAAGCTAGTGTCATATACCCCTCTAACGGTTGCGATAAAGTCGAGGTTATAGTC
ACAATGAAAGCTCATAAACGCCAACGCTGCCTCGACCCCCGGTCTAAGCAGGCTAGGCTCATAA
TGCAAGCAATCGAGAAGAAAAACTTTCTTAGACGGCAAAACATGtaG
CXCL10 (Mouse) (SEQ ID NO: 86)
ATGAACCCATCTGCCGCCGTTATTTTCTGTCTGATACTCCTTGGGCTGAGTGGCACACAAGGCAT
ACCCCTCGCCCGCACAGTCCGGTGTAATTGTATACATATTGACGACGGCCCTGTTAGAATGCGG
GCCATCGGTAAGCTGGAGATTATACCAGCAAGCCTTAGTTGTCCCAGGGTTGAAATCATAGCAA
CTATGAAAAAAAACGACGAACAAAGATGTTTGAATCCCGAGAGCAAGACAATCAAAAACCTTA
TGAAAGCATTTAGTCAAAAACGCTCTAAACGCGCTCCAtaG
CXCL10 (Human) (SEQ ID NO: 87)
ATGAATCAGACGGCAATCCTTATATGCTGCCTTATATTCCTTACTCTCTCAGGGATACAAGGGGT
ACCACTTTCTCGGACTGTTCGCTGCACTTGCATTTCAATATCTAACCAACCTGTAAATCCGCGGA
GCCTGGAAAAATTGGAGATTATACCTGCTTCTCAATTCTGCCCTCGGGTGGAAATCATCGCCACT
ATGAAGAAGAAGGGCGAGAAAAGGTGTCTGAATCCAGAGTCAAAGGCAATCAAAAACCTGCTG
AAAGCGGTGTCAAAGGAACGGTCCAAGAGATCACCCtaG
CXCL11-CXCL10 (Mouse) (SEQ ID NO: 88)
ATGAACAGGAAAGTAACAGCCATTGCATTGGCTGCCATCATCTGGGCCACCGCAGCACAGGGTT
TTCTGATGTTTAAGCAAGGGCGCTGTCTCTGTATAGGCCCAGGCATGAAGGCCGTGAAGATGGC
AGAGATTGAGAAGGCATCTGTGATTTATCCTTCTAACGGGTGCGATAAAGTCGAAGTTATTGTG
ACAATGAAGGCACACAAACGCCAACGGTGTTTGGACCCACGATCTAAACAGGCAAGATTGATT
ATGCAAGCCATCGAGAAAAAGAACTTTCTCCGAAGGCAAAATATGATCCCTTTGGCTCGGACAG
TGCGGTGTAACTGTATTCACATCGACGATGGGCCAGTACGGATGAGAGCAATAGGAAAGCTCG
AAATCATACCCGCCTCATTGTCTTGTCCCAGGGTGGAAATAATCGCCACTATGAAAAAGAACGA
TGAACAGAGGTGTCTCAACCCAGAGAGTAAGACTATCAAGAACCTTATGAAGGCATTCAGTCAG
AAGAGGTCAAAGCGAGCACCAtaG
XCL1 (Human) (SEQ ID NO: 89)
ATGAGACTTCTCATATTGGCGCTTCTCGGGATATGTTCTCTTACGGCATACATAGTTGAGGGGGT
GGGATCTGAGGTTAGCGATAAACGAACTTGTGTTAGTCTTACAACACAGAGGCTTCCAGTCTCC
AGGATAAAAACATATACGATAACTGAGGGATCTCTCAGAGCGGTCATCTTCATAACGAAGAGG
GGCCTGAAGGTCTGTGCTGACCCACAAGCGACTTGGGTAAGGGACGTTGTGCGGAGCATGGAC
AGGAAGAGCAATACTCGCAACAACATGATCCAAACCAAACCTACGGGCACCCAACAGTCAACC
AATACTGCGGTAACATTGACGGGGtaG
XCL1 (Mouse) (SEQ ID NO: 90)
ATGCGCCTCCTTCTGCTGACTTTTCTGGGTGTATGTTGCCTGACACCCTGGGTCGTAGAAGGAGT
AGGAACCGAGGTTCTGGAAGAGTCCTCATGTGTAAACTTGCAGACACAACGACTCCCCGTCCAA
AAAATCAAGACCTATATAATCTGGGAGGGGGCAATGCGGGCCGTCATTTTCGTGACTAAACGAG
GTCTCAAAATCTGCGCCGACCCCGAGGCTAAGTGGGTGAAGGCAGCCATTAAGACCGTGGATG
GGAGAGCCAGCACCAGAAAGAACATGGCCGAAACAGTACCTACTGGCGCACAGCGGTCAACCT
CAACTGCTATAACCTTGACAGGAtaG
m_sCD40L #1 (SEQ ID NO: 91)
ATGGAGACTGACACTCTGCTTCTGTGGGTGTTGCTGCTGTGGGTGCCTGGCAGTACAGGCGATA
TGCAACGAGGTGACGAGGACCCTCAAATCGCCGCCCATGTAGTCTCTGAAGCTAATAGCAACGC
TGCATCCGTCTTGCAGTGGGCAAAGAAAGGCTACTATACTATGAAGTCCAACTTGGTAATGCTT
GAAAACGGCAAGCAGTTGACTGTCAAGAGAGAGGGACTTTATTACGTCTATACCCAAGTCACAT
TCTGTAGCAATCGAGAACCCTCCTCACAGAGGCCTTTTATAGTGGGACTCTGGCTTAAACCAAG
TAGCGGCTCTGAGCGCATACTGTTGAAAGCCGCAAACACACACAGCTCTTCCCAACTCTGCGAG
CAGCAATCCGTGCATCTCGGTGGAGTATTTGAGCTTCAAGCCGGTGCCTCAGTGTTTGTGAACGT
CACTGAGGCCTCCCAGGTCATACATCGAGTTGGGTTCAGCTCCTTCGGCTTGCTCAAGCTCtaG
m_sCD40L #2 (SEQ ID NO: 92)
ATGGAAACTGATACATTGCTGCTCTGGGTTTTGCTGCTCTGGGTGCCTGGGAGTACAGGCGACA
TGAGGAGGCAGTTCGAGGATCTCGTTAAGGATATTACCCTTAATAAGGAGGAGAAGAAAGAAA
ACTCTTTTGAGATGCAACGAGGGGACGAAGATCCTCAGATCGCTGCTCACGTGGTCTCTGAAGC
TAACAGCAACGCCGCTTCTGTCCTCCAGTGGGCCAAGAAAGGTTATTACACCATGAAATCAAAC
CTTGTAATGCTTGAAAACGGGAAACAGCTTACAGTGAAGAGGGAAGGTCTTTACTACGTCTATA
CCCAGGTAACCTTCTGCTCAAACAGAGAACCATCAAGCCAGAGGCCATTCATAGTGGGGCTCTG
GCTCAAACCTTCCAGTGGCAGCGAGAGAATCTTGTTGAAAGCTGCTAATACACATAGTAGTAGC
CAGCTTTGCGAGCAACAGTCAGTCCACCTCGGGGGGGTGTTTGAGTTGCAAGCAGGGGCCTCAG
TATTCGTGAATGTCACTGAGGCTTCCCAGGTAATTCACAGGGTAGGCTTTAGTTCATTCGGTTTG
CTGAAGCTTtaG
m_sCD40L #3 (SEQ ID NO: 93)
ATGCGAAGAATGCAGCTTCTGCTCCTTATTGCTCTGAGTCTCGCCCTTGTCACCAACTCCGGGGA
CAGAATGAAACAAATCGAGGACAAAATTGAAGAAATACTGAGTAAAATATATCACATCGAAAA
CGAAATTGCACGCATTAAGAAATTGATTGGCGAACGCACCAGTGGCGGCTCTGGTGGCACCGG
AGGTTCAGGCGGGACCGGGGGCTCTGACAAAGTCGAAGAGGAGGTTAACCTTCATGAGGACTT
TGTGTTCATCAAGAAGCTGAAACGGTGCAATAAAGGAGAAGGTTCTTTGAGCCTCCTTAATTGC
GAAGAGATGCGACGACAGTTCGAGGATCTGGTTAAGGACATTACACTTAATAAGGAAGAGAAA
AAGGAGAACTCTTTCGAAATGCAGCGCGGCGATGAAGATCCCCAGATAGCCGCCCATGTCGTCT
CTGAGGCCAACTCTAACGCAGCATCCGTCCTCCAGTGGGCTAAGAAAGGATATTATACTATGAA
AAGCAATTTGGTCATGCTCGAAAACGGTAAACAGCTCACTGTTAAGAGAGAAGGCCTCTATTAC
GTATATACTCAAGTAACTTTCTGTTCTAATAGGGAACCCTCCTCTCAAAGACCTTTTATCGTAGG
ACTCTGGTTGAAACCAAGTAGCGGTAGTGAAAGGATTCTGCTCAAAGCAGCTAATACTCACTCC
AGCAGTCAACTGTGCGAACAACAAAGCGTTCACCTCGGGGGCGTCTTTGAACTTCAGGCAGGTG
CCAGTGTTTTCGTCAACGTAACAGAAGCATCCCAGGTAATTCATCGAGTAGGGTTTTCTAGCTTT
GGTTTGCTGAAGCTGtaG
anti-CD40_FGK4.5 (SEQ ID NO: 94)
ATGGAAACTGATCGCCTGTTGCTCTGGGTACTTCTTCTGTGGGTGCCTGGGTCCACTGGTGACAC
TGTACTTACACAATCACCCGCTTTGGCCGTTTCTCCTGGTGAACGGGTCACAATTAGTTGCCGAG
CTTCCGATTCTGTATCTACTCTTATGCATTGGTATCAACAAAAACCTGGTCAGCAGCCAAAATTG
CTCATTTATCTTGCTAGTCACTTGGAGTCCGGCGTACCTGCTCGATTCAGCGGTAGTGGGTCTGG
CACAGATTTCACTTTGACCATAGATCCCGTGGAGGCCGATGACACTGCAACCTACTATTGCCAG
CAATCCTGGAACGACCCTTGGACTTTCGGCGGCGGCACCAAGCTGGAACTCAAGCGAGCAGAT
GCTGCCCCAACCGTTAGTATATTCCCACCCTCAACCGAACAACTCGCCACAGGAGGCGCTAGTG
TCGTGTGTCTTATGAACAATTTCTATCCACGAGACATTAGCGTCAAGTGGAAAATTGATGGGAC
AGAAAGGCGAGATGGAGTTTTGGATTCAGTAACAGACCAGGATTCAAAGGATTCTACCTATAGC
ATGAGCTCCACCTTGAGCCTGACCAAAGCTGATTATGAATCTCATAACCTGTATACTTGTGAAGT
GGTGCATAAGACTTCTAGCTCACCAGTGGTTAAATCTTTTAACCGCAACGAATGTCGGCGCAAG
AGGGGTTCCGGAGAGGGAAGGGGTAGTCTGCTCACCTGCGGCGATGTTGAAGAAAATCCTGGT
CCCATGGACATTCGGCTCTCTTTGGTATTCCTGGTACTTTTTATAAAGGGGGTGCAATGTGAAGT
CCAGCTCGTGGAAAGCGGTGGGGGCCTGGTTCAGCCCGGTCGCAGCCTTAAACTTAGTTGCGCA
GCATCCGGATTTACATTTTCTGACTATAACATGGCCTGGGTTCGACAGGCACCCAAAAAAGGGC
TGGAGTGGGTCGCAACTATCATATACGATGGTTCCCGGACATACTATAGAGATTCAGTGAAGGG
GCGCTTTACAATAAGCAGGGACAATGCTAAGTCTACCTTGTATCTTCAGATGGACTCCCTGAGG
AGCGAAGATACAGCAACATATTATTGTGCTACAAACCGCTGGTTGCTGCTTCATTATTTCGACTA
CTGGGGTCAGGGCGTCATGGTAACTGTATCAAGCGCCGAGACCACAGCCCCTTCTGTATATCCA
TTGGCACCAGGTACTGCTCTGAAATCCAACTCAATGGTAACCCTTGGATGTCTGGTTAAGGGTTA
TTTTCCCGAGCCCGTCACAGTTACTTGGAACTCTGGGGCCCTTTCTAGCGGAGTCCATACCTTTC
CCGCCGTTTTGCAGAGTGGTCTGTACACCCTTACCTCAAGCGTCACAGTTCCATCTAGCACATGG
AGCTCCCAGGCAGTAACTTGTAATGTGGCCCATCCAGCCTCCTCAACTAAGGTAGATAAAAAGA
TCGTTCCCAGAGAATGCAATCCATGTGGATGCACCGGGTCTGAGGTCAGCAGTGTGTTCATTTTC
CCACCCAAGACTAAAGATGTATTGACTATTACTCTTACACCCAAAGTAACCTGCGTGGTGGTTG
ATATTAGTCAAAATGATCCCGAGGTACGGTTCTCTTGGTTTATCGACGACGTCGAAGTACATAC
AGCTCAGACACACGCTCCCGAGAAACAAAGCAATTCCACTCTTAGGAGCGTGTCCGAGTTGCCA
ATCGTACATAGGGATTGGCTTAATGGCAAGACCTTTAAGTGTAAGGTCAATTCAGGGGCATTCC
CCGCACCAATAGAGAAGAGTATAAGCAAACCCGAGGGGACACCCAGAGGTCCACAGGTCTATA
CAATGGCTCCCCCCAAGGAAGAGATGACCCAAAGTCAAGTCTCAATTACATGTATGGTGAAGG
GCTTTTATCCACCCGACATATACACTGAGTGGAAGATGAATGGACAGCCCCAAGAGAATTATAA
AAACACTCCCCCTACCATGGACACCGACGGGTCCTATTTTCTTTATAGTAAATTGAACGTGAAA
AAGGAGACCTGGCAACAAGGCAACACTTTCACCTGCTCCGTTCTTCACGAGGGCCTGCATAATC
ATCATACCGAAAAGTCTCTCAGTCATTCTCCAGGTAAGtaG
CD40L_2 (Human) (SEQ ID NO: 95)
ATGGAAACAGATACGTTGCTGTTGTGGGTACTTCTCCTTTGGGTCCCTGGCAGCACAGGGGACG
AGAATAGTTTCGAAATGCAGAAGGGCGACCAGAACCCACAGATCGCGGCTCACGTTATATCAG
AAGCAAGTAGTAAGACCACTTCCGTACTTCAGTGGGCTGAAAAAGGATATTACACCATGTCCAA
CAATCTCGTGACACTGGAGAACGGTAAACAACTTACGGTGAAACGACAGGGCCTCTATTACATC
TACGCTCAGGTGACATTCTGCTCAAATAGGGAGGCTTCTAGTCAAGCGCCCTTCATCGCCAGCC
TGTGCCTCAAATCTCCCGGCCGGTTCGAACGAATCCTGTTGCGAGCGGCCAATACCCATAGCTC
AGCTAAACCTTGCGGCCAGCAGAGTATTCATCTTGGTGGTGTGTTTGAACTTCAGCCGGGAGCA
TCTGTGTTCGTCAACGTAACGGACCCTAGCCAAGTGTCTCATGGGACAGGTTTTACATCCTTCGG
ACTCCTCAAGTTGtaG
Flt3L (Human) (SEQ ID NO: 96)
ATGACAGTTCTCGCGCCAGCTTGGAGTCCCACCACATACTTGCTTTTGCTTCTGCTTCTGTCCTCT
GGCCTGAGTGGGACCCAAGATTGTTCCTTTCAACATTCCCCAATTAGTTCTGATTTTGCAGTGAA
GATTAGAGAGCTCTCAGACTATCTGCTGCAAGATTATCCTGTCACAGTCGCTTCAAACCTGCAA
GACGAAGAGCTCTGCGGTGCCTTGTGGCGGTTGGTCTTGGCTCAAAGATGGATGGAGAGACTGA
AAACCGTAGCAGGCAGCAAGATGCAGGGTCTCCTGGAAAGGGTGAACACGGAAATCCATTTTG
TGACCAAGTGCGCGTTCCAGCCCCCACCGAGTTGTCTCCGGTTTGTTCAAACGAATATATCCCGG
TTGCTCCAGGAAACCTCAGAACAACTGGTGGCTTTGAAACCCTGGATCACAAGACAAAACTTTA
GTCGGTGCCTCGAACTCCAGTGCCAACCAGATTCTTCTACACTTCCCCCCCCGTGGTCCCCGCGC
CCGTTGGAAGCAACGGCCCCAtaG
TGFb TRAP (Human) (SEQ ID NO: 97)
ATGGCCTGGAGTCCTCTGTTTCTGACTCTTATAACTCACTGTGCCGGCAGTTGGGCTATACCCCC
TCATGTACAGAAGTCTGTAAACAACGACATGATTGTAACCGACAATAATGGCGCAGTGAAATTC
CCACAACTGTGTAAGTTCTGTGATGTACGGTTTAGTACATGCGACAATCAAAAAAGCTGTATGT
CTAACTGCTCTATTACATCCATATGTGAAAAACCTCAGGAGGTGTGTGTTGCCGTTTGGCGAAA
AAATGATGAGAATATCACACTGGAGACAGTATGTCATGACCCTAAACTGCCATACCATGATTTC
ATACTGGAGGACGCCGCCAGTCCTAAGTGCATTATGAAAGAGAAAAAGAAACCCGGTGAAACA
TTCTTTATGTGCTCTTGTAGCTCTGACGAGTGTAACGACAACATTATATTCAGCGAGGAGTACAA
TACAAGCAACCCCGATATACCACCTCACGTACAAAAAAGTGTCAACAACGATATGATTGTTACC
GACAATAACGGAGCTGTTAAGTTTCCTCAGTTGTGCAAGTTCTGCGATGTACGATTCTCTACCTG
CGACAACCAAAAGTCATGTATGTCTAACTGTTCCATAACCTCCATCTGCGAGAAGCCCCAGGAA
GTCTGCGTCGCCGTGTGGCGGAAAAACGACGAGAATATCACTCTTGAAACCGTTTGTCATGATC
CTAAACTGCCCTATCACGACTTTATTCTGGAAGATGCTGCTTCCCCTAAGTGTATCATGAAAGAA
AAGAAGAAACCTGGGGAGACATTCTTTATGTGTTCATGCTCCTCCGATGAGTGTAACGACAATA
TCATCTTCTCTGAGGAATACAACACTTCTAACCCTGATtaG
Fresolimumab (Human) (SEQ ID NO: 98)
ATGGCCTGGTCCCCTCTTTTTCTGACCCTCATCACACACTGTGCAGGCTCATGGGCTGAGACCGT
CTTGACCCAGTCCCCAGGAACTTTGTCTCTGTCTCCTGGTGAAAGAGCTACCCTTAGTTGTCGAG
CCTCTCAGTCCCTTGGTTCTAGCTATCTCGCTTGGTACCAGCAAAAGCCAGGCCAGGCCCCACG
ACTGCTGATCTACGGAGCATCTTCACGGGCTCCCGGCATTCCCGATCGATTTTCCGGATCTGGTA
GTGGTACAGATTTCACACTGACCATATCTCGCCTGGAGCCCGAGGACTTTGCTGTTTATTATTGT
CAGCAGTACGCCGATTCTCCTATCACTTTTGGACAGGGAACCCGCCTGGAGATTAAGCGCACAG
TAGCAGCTCCATCCGTCTTTATCTTTCCACCATCAGATGAACAGCTCAAGAGTGGGACCGCAAG
TGTAGTATGCCTGCTGAACAATTTTTACCCTAGAGAGGCCAAAGTGCAGTGGAAGGTGGATAAC
GCCCTCCAGAGTGGCAATAGTCAAGAAAGTGTTACTGAGCAAGATAGTAAGGACTCTACATACT
CTTTGAGTTCTACTTTGACCCTGTCAAAAGCAGATTATGAAAAACATAAGGTGTATGCATGTGA
AGTTACACACCAAGGGTTGTCCTCTCCAGTTACAAAATCTTTTAATAGAGGAGAGTGCCGCCGC
AAACGCGGTAGTGGAGAAGGTCGAGGCTCACTCTTGACCTGTGGCGACGTGGAAGAAAATCCC
GGTCCTATGGATTGGACTTGGAGGGTATTTTGTCTTTTGGCAGTAACACCTGGAGCTCACCCCCA
AGTACAGCTCGTCCAATCTGGTGCCGAGGTTAAAAAGCCTGGAAGTTCAGTGAAGGTCTCTTGC
AAGGCATCTGGATACACCTTTTCATCTAACGTCATATCCTGGGTACGGCAAGCCCCAGGACAGG
GACTTGAGTGGATGGGAGGGGTCATCCCCATCGTGGACATTGCTAATTACGCTCAGCGATTCAA
AGGGCGGGTTACTATAACTGCCGACGAGTCTACCTCAACTACCTACATGGAGTTGTCCTCTCTCC
GCTCCGAGGACACTGCTGTATATTACTGTGCCAGCACTCTCGGGTTGGTGTTGGATGCCATGGAC
TATTGGGGACAAGGAACCCTGGTGACAGTTAGCTCCGCAAGCACTAAAGGCCCTTCTGTTTTTC
CCTTGGCACCTTGTAGTAGGTCTACCTCTGAGTCTACAGCAGCACTTGGATGCTTGGTTAAGGAC
TATTTTCCCGAGCCAGTTACAGTCTCTTGGAACAGTGGTGCCCTCACAAGTGGGGTTCATACCTT
CCCCGCAGTCCTCCAGAGTAGTGGCCTTTACAGCCTCTCATCAGTTGTGACTGTTCCTAGTTCAT
CACTCGGTACTAAGACATATACATGTAACGTAGACCACAAGCCAAGCAACACAAAAGTAGACA
AACGAGTCGAATCTAAGTATGGACCCCCTTGTCCCTCCTGTCCTGCTCCCGAGTTCCTTGGGGGC
CCTTCCGTGTTCTTGTTTCCTCCCAAGCCCAAGGATACCCTCATGATCTCACGAACCCCAGAGGT
AACATGTGTGGTTGTTGACGTAAGTCAGGAAGATCCCGAAGTGCAATTTAATTGGTACGTGGAT
GGCGTCGAAGTCCATAACGCTAAAACAAAACCCCGAGAGGAACAATTCAATTCCACATATCGG
GTGGTGAGTGTATTGACCGTTCTTCACCAAGATTGGCTGAACGGCAAGGAGTATAAGTGTAAAG
TAAGCAACAAAGGTCTGCCAAGTAGCATAGAAAAAACAATATCTAAAGCTAAGGGCCAACCAA
GGGAACCACAAGTATATACATTGCCCCCCTCTCAGGAAGAGATGACAAAGAATCAAGTTAGCCT
GACCTGTTTGGTAAAGGGGTTCTATCCCTCAGATATAGCAGTCGAGTGGGAATCTAACGGCCAG
CCCGAGAATAATTATAAAACAACCCCCCCTGTGTTGGACTCAGACGGCAGCTTCTTTCTCTATTC
ACGGCTCACTGTTGATAAGTCCCGATGGCAGGAGGGGAATGTTTTCAGCTGTAGCGTGATGCAC
GAAGCTCTCCACAACCACTATACACAGAAAAGTTTGTCTTTGTCCCTTGGAAAAtaG
TGFb neutralizing peptide (Human) (SEQ ID NO: 99)
ATGAGTACATCCTTTCCAGAGCTGGATCTGGAGAATTTTGAGTATGACGACAGTGCCGAAGCCT
GCTACCTCGGGGACATAGTCGCATTCGGGACAATCTTTTTGTCTGTATTTTACGCCCTGGTGTTT
ACATTTGGCCTGGTTGGAAATCTGTTGGTCGTACTCGCTCTCACCAATTCCCGAAAACCCAAAA
GTATAACAGACATATACCTGTTGAATCTGGCACTGAGTGACCTTTTGTTCGTCGCCACCCTTCCT
TTTTGGACACACTACCTTATCAGTCACGAGGGGCTTCATAATGCTATGTGCAAGCTCACTACTGC
CTTCTTCTTTATCGGATTCTTCGGGGGTATCTTTTTTATCACAGTTATTAGCATTGACCGATACCT
TGCCATAGTGCTCGCAGCCAACTCAATGAACAACCGCACCGTGCAGCATGGAGTGACTATTTCC
TTGGGTGTGTGGGCCGCTGCTATACTTGTCGCCAGCCCTCAATTCATGTTTACCAAAAGGAAAG
ACAATGAGTGCCTCGGAGATTACCCTGAGGTGTTGCAAGAAATGTGGCCTGTACTTCGAAATAG
CGAAGTGAATATACTCGGCTTTGCTCTTCCTCTGCTCATCATGTCATTCTGTTATTTTCGAATAAT
CCAAACATTGTTCAGCTGTAAGAACCGAAAGAAAGCCCGCGCCGTACGCCTGATTCTGCTCGTT
GTGTTCGCCTTTTTTCTGTTTTGGACTCCTTACAACATAATGATATTCCTGGAGACTCTCAAATTC
TATAACTTTTTTCCCTCCTGTGATATGAAAAGGGACCTTAGATTGGCTCTCAGTGTCACTGAAAC
AGTAGCCTTTAGCCATTGTTGTCTCAACCCTTTCATATATGCATTTGCAGGGGAAAAGTTCCGGC
GGTATCTCGGACATTTGTATCGGAAGTGCTTGGCCGTGTTGTGTGGTCATCCTGTCCATACCGGA
TTCTCTCCTGAGAGTCAACGGAGCCGCCAAGATTCAATCCTGTCCAGTTTCACTCACTATACTTC
AGAGGGGGATGGCAGCCTTCTGCTC
Kynureinase #1 (SEQ ID NO: 100)
ATGGAGACCGACACTTTGTTGCTGTGGGTACTTTTGTTGTGGGTCCCAGGATCTACCGGGGATAT
GGAACCCTCTCCTCTTGAACTGCCAGTAGACGCCGTGCGCCGCATTGCAGCCGAGTTGAATTGC
GATCCAACAGATGAACGCGTTGCCCTGAGGCTCGACGAAGAGGATAAATTGTCACATTTCAGGA
ACTGCTTTTACATTCCAAAGATGAGGGATCTTCCATCCATAGATCTTAGCCTCGTGTCCGAGGAT
GACGATGCCATATATTTTCTTGGGAACAGTCTTGGGTTGCAGCCAAAAATGGTACGGACATATC
TCGAAGAGGAGCTGGACAAATGGGCTAAAATGGGTGCTTACGGCCACGACGTGGGAAAACGCC
CCTGGATAGTTGGCGACGAATCTATCGTGAGTCTTATGAAAGATATAGTTGGAGCACATGAGAA
AGAAATTGCACTGATGAATGCCCTTACTATCAATCTGCATCTCCTCTTGCTTTCATTCTTTAAGCC
CACTCCTAAACGCCACAAAATACTTTTGGAAGCAAAAGCCTTTCCAAGCGACCACTACGCTATT
GAGTCACAAATACAACTCCATGGACTTGATGTGGAAAAGTCTATGCGGATGGTAAAACCACGCG
AAGGCGAGGAGACCCTTCGAATGGAGGACATACTTGAGGTCATCGAAGAAGAAGGAGATAGTA
TAGCAGTTATCCTTTTCAGCGGGCTGCACTTCTACACAGGTCAACTCTTTAACATTCCAGCTATT
ACTAAGGCAGGCCACGCTAAAGGATGCTTCGTGGGCTTTGACCTTGCACACGCAGTAGGAAACG
TAGAGCTCCGCTTGCACGATTGGGGCGTTGATTTCGCCTGCTGGTGTTCATATAAGTATCTTAAC
TCAGGAGCTGGTGGGTTGGCAGGCGCATTCGTACACGAGAAACACGCTCATACCGTAAAGCCTG
CACTGGTAGGGTGGTTCGGACACGATCTCTCTACCCGCTTCAATATGGATAATAAACTCCAGCTT
ATACCTGGCGCCAATGGATTCAGGATCTCAAATCCTCCTATTTTGCTCGTTTGCAGTTTGCACGC
ATCTCTTGAGGTGTTCCAGCAGGCTACCATGACTGCACTCCGCCGGAAGTCAATCCTTTTGACCG
GATACTTGGAGTATATGCTGAAACATTATCACTCAAAAGATAACACTGAGAATAAGGGCCCCAT
AGTAAACATTATCACTCCATCTCGGGCTGAAGAGCGCGGCTGCCAACTCACATTGACTTTTTCCA
TTCCCAAGAAGTCAGTGTTCAAAGAGTTGGAGAAACGGGGGGTTGTATGTGATAAGCGGGAGC
CAGATGGAATCCGCGTTGCCCCAGTCCCCCTCTATAATTCTTTTCACGATGTATACAAGTTTATT
AGACTGCTGACAAGTATCTTGGACTCATCTGAGCGATCTtaG
Kynureinase #2 (SEQ ID NO: 101)
ATGGAACCCTCTCCTCTTGAACTGCCAGTAGACGCCGTGCGCCGCATTGCAGCCGAGTTGAATT
GCGATCCAACAGATGAACGCGTTGCCCTGAGGCTCGACGAAGAGGATAAATTGTCACATTTCAG
GAACTGCTTTTACATTCCAAAGATGAGGGATCTTCCATCCATAGATCTTAGCCTCGTGTCCGAGG
ATGACGATGCCATATATTTTCTTGGGAACAGTCTTGGGTTGCAGCCAAAAATGGTACGGACATA
TCTCGAAGAGGAGCTGGACAAATGGGCTAAAATGGGTGCTTACGGCCACGACGTGGGAAAACG
CCCCTGGATAGTTGGCGACGAATCTATCGTGAGTCTTATGAAAGATATAGTTGGAGCACATGAG
AAAGAAATTGCACTGATGAATGCCCTTACTATCAATCTGCATCTCCTCTTGCTTTCATTCTTTAA
GCCCACTCCTAAACGCCACAAAATACTTTTGGAAGCAAAAGCCTTTCCAAGCGACCACTACGCT
ATTGAGTCACAAATACAACTCCATGGACTTGATGTGGAAAAGTCTATGCGGATGGTAAAACCAC
GCGAAGGCGAGGAGACCCTTCGAATGGAGGACATACTTGAGGTCATCGAAGAAGAAGGAGATA
GTATAGCAGTTATCCTTTTCAGCGGGCTGCACTTCTACACAGGTCAACTCTTTAACATTCCAGCT
ATTACTAAGGCAGGCCACGCTAAAGGATGCTTCGTGGGCTTTGACCTTGCACACGCAGTAGGAA
ACGTAGAGCTCCGCTTGCACGATTGGGGCGTTGATTTCGCCTGCTGGTGTTCATATAAGTATCTT
AACTCAGGAGCTGGTGGGTTGGCAGGCGCATTCGTACACGAGAAACACGCTCATACCGTAAAG
CCTGCACTGGTAGGGTGGTTCGGACACGATCTCTCTACCCGCTTCAATATGGATAATAAACTCC
AGCTTATACCTGGCGCCAATGGATTCAGGATCTCAAATCCTCCTATTTTGCTCGTTTGCAGTTTG
CACGCATCTCTTGAGGTGTTCCAGCAGGCTACCATGACTGCACTCCGCCGGAAGTCAATCCTTTT
GACCGGATACTTGGAGTATATGCTGAAACATTATCACTCAAAAGATAACACTGAGAATAAGGGC
CCCATAGTAAACATTATCACTCCATCTCGGGCTGAAGAGCGCGGCTGCCAACTCACATTGACTT
TTTCCATTCCCAAGAAGTCAGTGTTCAAAGAGTTGGAGAAACGGGGGGTTGTATGTGATAAGCG
GGAGCCAGATGGAATCCGCGTTGCCCCAGTCCCCCTCTATAATTCTTTTCACGATGTATACAAGT
TTATTAGACTGCTGACAAGTATCTTGGACTCATCTGAGCGATCTtaG
VEGF (SEQ ID NO: 102)
ATGAATTTCTTGCTGAGCTGGGTGCATTGGACACTCGCATTGTTGCTGTACTTGCACCATGCCAA
GTGGTCCCAGGCTGCACCCACTACTGAGGGCGAGCAAAAGTCTCATGAGGTGATTAAATTTATG
GACGTTTACCAACGATCATACTGTCGGCCAATCGAAACCCTCGTAGATATATTCCAGGAGTACC
CAGACGAGATCGAATACATTTTCAAGCCCTCATGTGTCCCATTGATGCGATGTGCTGGGTGCTGT
AACGACGAAGCACTTGAATGTGTCCCCACCTCCGAGAGTAACATCACAATGCAAATAATGAGA
ATCAAGCCCCACCAATCCCAACATATCGGTGAAATGTCATTCCTTCAGCATTCCCGCTGCGAGT
GCCGGCCTAAGAAGGACCGCACCAAACCAGAGAACCATTGTGAACCCTGTTCTGAGAGACGGA
AGCACTTGTTCGTACAGGACCCTCAAACATGCAAGTGCAGCTGTAAGAATACCGACTCACGGTG
TAAAGCTAGGCAACTGGAGCTTAATGAAAGGACCTGCCGATGCGATAAACCCAGGAGGtaa
GM-CSF (SEQ ID NO: 103)
ATGTGGTTGCAGAATTTGCTCTTCCTGGGGATTGTGGTCTACAGCCTCTCCGCACCTACCCGCTC
TCCTATCACAGTTACAAGACCCTGGAAACATGTGGAGGCCATTAAAGAAGCATTGAATTTGTTG
GACGATATGCCCGTCACCCTGAATGAAGAAGTAGAAGTTGTTTCTAATGAGTTCAGCTTTAAAA
AATTGACCTGTGTGCAGACACGGCTTAAAATTTTTGAACAGGGACTTAGAGGAAACTTTACTAA
GCTGAAGGGGGCACTTAACATGACAGCTTCTTATTATCAGACCTATTGTCCTCCAACACCTGAA
ACCGACTGTGAAACACAGGTAACCACTTACGCCGATTTTATTGATTCTTTGAAAACATTCCTCAC
CGATATACCATTTGAGTGTAAGAAGCCAGGCCAAAAGtaG
Anti-PDI (SEQ ID NO: 104)
ATGGAAACTGACACACTTCTTCTGTGGGTCTTGCTCCTGTGGGTCCCAGGCTCTACTGGTGACAG
TCCTGATAGGCCATGGAACCCACCTACCTTTAGTCCAGCCTTGCTCGTCGTAACCGAAGGGGAC
AACGCTACATTCACCTGCTCTTTTAGCAATACTTCTGAGAGTTTTCATGTAGTCTGGCATCGGGA
GAGTCCATCCGGACAAACAGATACTTTGGCCGCTTTTCCAGAGGATAGGTCTCAACCTGGGCAA
GACGCAAGGTTTCGAGTCACACAGCTTCCTAACGGGAGAGATTTTCACATGTCTGTAGTTCGGG
CACGCCGAAATGATTCTGGCACATATGTTTGCGGTGTGATCTCACTTGCTCCAAAGATTCAAATA
AAGGAGAGCCTTCGCGCCGAGTTGCGGGTGACTGAGCGGGAGCCCAAGTCCTGCGACAAAACC
CATACTTGTCCACCCTGTGGCGGCGGGTCATCCGGTGGCGGGTCTGGGGGGCAACCAAGAGAGC
CACAGGTATATACTCTTCCCCCCAGCAGAGAAGAAATGACAAAAAACCAAGTGTCCCTGACATG
TCTGGTTAAAGGATTTTATCCCAGTGACATTGCTGTAGAATGGGAATCCAATGGTCAACCCGAG
AATAACTACAAAACCACTCCTCCAGTATTGGACAGTGACGGTTCCTTCTTCCTCTATTCCAAACT
TACAGTGGATAAATCCCGCTGGCAGCAAGGGAATGTATTCAGCTGTAGTGTCATGCACGAAGCT
CTTCATAACCATTATACACAGAAATCTCTTTCCCTGAGCCCAGGTAAAtaG
Adenosine Deaminase (ADA) #1 (Mouse) (SEQ ID NO: 105)
ATGGAGACTGATACACTTTTGCTCTGGGTTTTGCTCTTGTGGGTACCAGGGTCTACTGGAGATGC
ACAAACTCCTGCATTCAACAAGCCTAAGGTAGAGCTTCATGTCCATTTGGACGGAGCCATAAAA
CCTGAAACCATACTCTATTTCGGCAAGAAACGGGGTATAGCACTTCCCGCTGATACCGTGGAAG
AGTTGAGAAATATCATTGGCATGGACAAACCTCTTAGCCTGCCTGGCTTTCTTGCAAAGTTCGAC
TACTATATGCCAGTTATAGCAGGGTGTAGAGAAGCAATAAAGCGAATCGCCTATGAGTTCGTTG
AGATGAAGGCTAAAGAAGGAGTTGTTTACGTGGAAGTCCGGTACTCACCTCATCTGCTTGCTAA
TAGCAAGGTGGACCCAATGCCATGGAATCAAACTGAAGGTGATGTAACCCCTGACGATGTGGTC
GATTTGGTCAATCAAGGTCTCCAAGAAGGCGAGCAGGCTTTCGGCATTAAGGTAAGAAGTATAT
TGTGCTGTATGCGACATCAACCTTCATGGTCCCTGGAGGTCCTCGAATTGTGCAAAAAGTACAA
TCAAAAAACAGTGGTCGCAATGGATCTCGCTGGAGATGAGACCATAGAAGGTTCCTCTCTTTTC
CCCGGTCATGTCGAAGCATATGAAGGGGCTGTCAAAAATGGTATCCACCGCACCGTCCACGCAG
GGGAAGTAGGGTCCCCAGAAGTAGTCAGGGAAGCCGTTGACATTTTGAAAACAGAAAGAGTCG
GGCATGGCTACCATACAATAGAGGACGAAGCCTTGTACAATCGACTTTTGAAAGAAAATATGCA
CTTCGAGGTCTGTCCCTGGAGTTCATATCTCACCGGAGCATGGGACCCCAAAACAACCCACGCC
GTCGTACGCTTCAAGAATGATAAGGCAAACTACAGTTTGAATACAGATGATCCACTGATATTCA
AGTCAACACTTGACACTGACTACCAGATGACAAAAAAAGATATGGGTTTCACCGAAGAAGAGT
TCAAGAGATTGAACATTAACGCAGCAAAAAGCTCCTTCCTGCCAGAGGAAGAGAAAAAAGAAT
TGCTTGAAAGGTTGTATCGAGAATACCAA
Adenosine Deaminase (ADA) #2 (Mouse) (SEQ ID NO: 106)
ATGGCACAAACTCCAGCTTTTAATAAGCCCAAAGTGGAACTTCATGTTCATCTGGATGGGGCAA
TTAAGCCCGAAACTATATTGTACTTTGGCAAAAAGAGGGGTATTGCCCTGCCAGCAGATACCGT
TGAGGAGCTTCGCAACATCATTGGGATGGACAAGCCCCTCTCTCTGCCAGGTTTTCTCGCTAAAT
TCGATTATTATATGCCTGTTATTGCTGGTTGCCGGGAGGCCATCAAGAGGATAGCCTACGAGTTT
GTTGAGATGAAGGCCAAAGAGGGCGTGGTGTACGTAGAGGTCAGATACAGCCCTCACCTGCTTG
CCAACAGCAAGGTGGACCCAATGCCCTGGAACCAAACCGAGGGGGATGTCACTCCCGACGACG
TTGTAGACCTCGTAAATCAGGGCCTTCAAGAGGGCGAGCAGGCATTTGGCATAAAAGTCCGGTC
TATACTCTGCTGTATGAGGCACCAACCCTCCTGGTCTTTGGAGGTACTTGAGTTGTGTAAGAAAT
ACAATCAAAAGACTGTAGTCGCCATGGATCTTGCAGGCGATGAAACCATCGAGGGTAGCTCCTT
GTTCCCTGGACATGTTGAAGCCTACGAGGGGGCCGTAAAAAATGGGATACACAGGACTGTCCA
CGCTGGTGAAGTCGGAAGCCCAGAGGTGGTAAGGGAGGCAGTTGACATACTCAAGACAGAGCG
GGTTGGACACGGATACCACACAATTGAGGACGAGGCCCTGTATAACCGCCTCCTCAAAGAGAA
CATGCATTTTGAGGTGTGTCCTTGGTCCAGCTACCTGACTGGTGCTTGGGACCCTAAAACAACTC
ACGCCGTGGTCCGGTTCAAGAACGATAAAGCCAATTACTCTTTGAATACCGACGACCCCCTCAT
ATTCAAATCAACATTGGATACCGACTACCAAATGACCAAAAAGGATATGGGGTTTACTGAAGAG
GAGTTCAAGAGGCTCAACATAAATGCCGCTAAATCCTCCTTTCTCCCCGAGGAAGAAAAAAAAG
AACTCCTTGAGCGGCTGTATAGGGAGTATCAA
4-1BBL #1 (Mouse) (SEQ ID NO: 107)
ATGGAAACAGATACACTCTTGCTCTGGGTACTGCTTCTGTGGGTCCCCGGCTCTACTGGGGATGA
AGATGATGTAACTACTACAGAAGAACTCGCTCCCGCTCTTGTCCCCCCACCCAAGGGTACCTGC
GCCGGTTGGATGGCTGGCATCCCAGGACATCCAGGTCACAACGGTACCCCCGGAAGAGATGGT
CGGGATGGAACTCCCGGCGAGAAGGGCGAAAAAGGGGATGCAGGGCTTCTGGGACCTAAAGGT
GAAACAGGGGACGTTGGAATGACTGGTGCAGAAGGGCCTCGCGGCTTTCCTGGCACCCCTGGG
AGGAAAGGAGAGCCCGGAGAGCTCCAGAGAACTGAACCTCGGCCTGCACTCACTATAACTACT
TCCCCTAATCTTGGGACCCGCGAGAACAACGCCGATCAGGTTACACCTGTAAGCCATATCGGGT
GCCCCAATACTACCCAGCAAGGGAGTCCCGTGTTCGCAAAGCTTTTGGCTAAAAACCAAGCATC
CCTGTGTAACACTACTCTTAATTGGCATTCACAAGACGGTGCTGGTAGCTCTTATCTTTCTCAGG
GGCTGCGGTACGAAGAAGATAAGAAGGAATTGGTTGTGGATTCTCCAGGACTCTATTATGTCTT
TCTCGAATTGAAGCTCAGTCCCACCTTCACAAACACTGGACACAAAGTCCAGGGCTGGGTAAGT
CTGGTACTCCAAGCAAAGCCCCAGGTTGACGATTTCGACAATTTGGCACTCACCGTAGAGCTTT
TCCCATGCTCCATGGAAAATAAACTTGTTGATCGGTCATGGTCACAGCTCTTGCTGCTTAAGGCA
GGGCATCGCCTCTCAGTGGGTCTGAGAGCTTATTTGCATGGTGCACAAGATGCTTACAGGGATT
GGGAATTGTCCTACCCAAACACTACAAGTTTCGGGTTGTTCCTTGTCAAACCTGATAACCCATGG
GAGtaG
4-1BBL #2 (Mouse) (SEQ ID NO: 108)
ATGGAAACTGATACACTCCTCCTGTGGGTCCTTCTTTTGTGGGTGCCCGGATCAACCGGCGATGG
CTGGATGGCAGGCATCCCAGGACACCCAGGACACAACGGTACTCCAGGTCGAGACGGTCGGGA
TGGGACTCCTGGGGAGAAAGGCGAGAAAGGGGACGCTGGTTTGCTCGGTCCTAAGGGGGAAAC
CGGGGATGTAGGAATGACAGGGGCTGAAGGGCCTCGGGGATTTCCTGGGACACCAGGCAGGAA
GGGTGAACCAGGGGAGGCCCTCCAGCGCACCGAGCCACGGCCAGCTCTGACCATAACAACAAG
TCCAAACCTGGGCACACGCGAAAACAATGCTGACCAGGTGACTCCTGTAAGTCACATCGGATGC
CCTAACACTACACAACAGGGCTCTCCTGTATTTGCAAAGCTTCTCGCAAAAAATCAAGCATCAC
TTTGTAATACAACCCTGAACTGGCATTCTCAGGACGGAGCAGGGTCCTCTTATTTGTCTCAAGGG
CTCCGCTACGAAGAAGATAAAAAGGAATTGGTTGTTGACAGTCCAGGTTTGTATTATGTGTTTTT
GGAACTTAAGCTGTCACCAACCTTCACTAACACCGGCCACAAGGTCCAAGGCTGGGTTAGTCTT
GTTTTGCAAGCCAAACCTCAAGTGGATGATTTTGACAATCTGGCTTTGACTGTTGAGCTTTTTCC
ATGCAGTATGGAGAATAAACTGGTTGATCGGTCATGGTCACAGCTCCTTCTGCTCAAGGCCGGA
CATAGGCTGAGTGTGGGACTTCGGGCCTACTTGCACGGCGCCCAGGACGCATACCGAGACTGGG
AACTCAGCTACCCTAACACAACTTCTTTTGGGTTGTTCCTTGTCAAACCCGATAATCCTTGGGAAt
aG
HPGE2 #1 (Mouse) (SEQ ID NO: 109)
ATGGAGACTGATACTTTGCTCCTGTGGGTTCTTCTCCTGTGGGTTCCTGGTTCCACAGGGGATAT
GCATGTCAATGGCAAGGTAGCACTCGTGACTGGGGCTGCACAGGGTATCGGGAAAGCTTTTGCC
GAGGCCCTGTTGCTGCATGGCGCCAAGGTCGCTTTGGTAGATTGGAACTTGGAGGCTGGAGTTA
AATGCAAAGCTGCACTCGACGAACAATTTGAGCCTCAAAAAACCCTCTTTGTGCAGTGTGACGT
TGCTGACCAAAAGCAACTCAGGGACACATTCAGGAAGGTCGTAGACCATTTCGGACGCCTCGAT
ATACTCGTTAATAATGCCGGGGTAAACAACGAAAAGAACTGGGAACAAACATTGCAAATCAAC
CTGGTAAGTGTCATTAGCGGAACTTATCTGGGTCTTGATTATATGAGCAAGCAGAACGGGGGCG
AGGGCGGGATCATTATCAACATGTCAAGTCTTGCCGGATTGATGCCAGTTGCTCAGCAGCCTGT
TTACTGTGCCAGCAAGCACGGTATTATTGGGTTTACCCGGAGTGCCGCCATGGCCGCAAATCTT
ATGAAGAGTGGGGTAAGACTGAATGTTATCTGCCCAGGTTTCGTAGATACCCCAATCCTGGAGA
GCATCGAGAAGGAGGAAAATATGGGACAATACATTGAATATAAAGATCAAATCAAGGCTATGA
TGAAGTTCTACGGGGTTCTGCATCCATCCACAATTGCCAACGGGCTCATTAATCTGATTGAGGAC
GACGCCTTGAACGGAGCTATAATGAAAATCACAGCTTCCAAAGGCATTCACTTCCAAGATTATG
ATATATCACCCTTGCTTGTCAAGGCTCCTCTGACAAGT
HPGE2 #2 (Mouse) (SEQ ID NO: 110)
ATGCATGTCAATGGCAAGGTAGCACTCGTGACTGGGGCTGCACAGGGTATCGGGAAAGCTTTTG
CCGAGGCCCTGTTGCTGCATGGCGCCAAGGTCGCTTTGGTAGATTGGAACTTGGAGGCTGGAGT
TAAATGCAAAGCTGCACTCGACGAACAATTTGAGCCTCAAAAAACCCTCTTTGTGCAGTGTGAC
GTTGCTGACCAAAAGCAACTCAGGGACACATTCAGGAAGGTCGTAGACCATTTCGGACGCCTCG
ATATACTCGTTAATAATGCCGGGGTAAACAACGAAAAGAACTGGGAACAAACATTGCAAATCA
ACCTGGTAAGTGTCATTAGCGGAACTTATCTGGGTCTTGATTATATGAGCAAGCAGAACGGGGG
CGAGGGCGGGATCATTATCAACATGTCAAGTCTTGCCGGATTGATGCCAGTTGCTCAGCAGCCT
GTTTACTGTGCCAGCAAGCACGGTATTATTGGGTTTACCCGGAGTGCCGCCATGGCCGCAAATC
TTATGAAGAGTGGGGTAAGACTGAATGTTATCTGCCCAGGTTTCGTAGATACCCCAATCCTGGA
GAGCATCGAGAAGGAGGAAAATATGGGACAATACATTGAATATAAAGATCAAATCAAGGCTAT
GATGAAGTTCTACGGGGTTCTGCATCCATCCACAATTGCCAACGGGCTCATTAATCTGATTGAG
GACGACGCCTTGAACGGAGCTATAATGAAAATCACAGCTTCCAAAGGCATTCACTTCCAAGATT
ATGATATATCACCCTTGCTTGTCAAGGCTCCTCTGACAAGT

Methods of Engineering Cells

Also provided herein are compositions and methods for engineering cells to produce one or more effector molecules.

In general, cells are engineered to produce effector molecules through introduction (i.e., delivery) of polynucleotides encoding the one or more effector molecules into the cell's cytosol and/or nucleus. For example, the polynucleotides encoding the one or more effector molecules can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more effector molecules. The transgenes encoding the one or more effector molecules can be configured to express the one or more effector molecules.

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,535,362; 6,083,716, 7,371,570, 7,348,178; 7,323,177; 7,319,033; 7,318,919; and 7,306,793 and international Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat & amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

### Genomic Editing Systems

A genomic editing systems can be used to engineer a host genome to encode an engineered nucleic acid, such as an engineered nucleic acid encoding one or more of the effector molecules described herein. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid encoding one or more of the effector molecules described herein, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid encoding one or more of the effector molecules described herein. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fok1. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

Delivery Vehicles

Also provided herein are compositions for delivering a cargo/payload (a “delivery vehicle”).

The cargo can comprise nucleic acids (e.g., any of the engineered nucleic acids described herein), as described above. The cargo can comprise proteins, carbohydrates, lipids, small molecules, and/or combinations thereof. The cargo can be any of the effector molecules described herein. The cargo can be a combination of the effector molecules described herein, e.g., two or more of the of the effector molecules described herein.

The delivery vehicle can comprise any composition suitable for delivering a cargo. The delivery vehicle can comprise any composition suitable for delivering a protein (e.g., any of the effector molecules described herein). The delivery vehicle can be any of the lipid structure delivery systems described herein. For example, a delivery vehicle can be a lipid-based structure including, but not limited to, a lipid-based nanoparticle, a liposome, a micelle, an exosome, a vesicle, an extracellular vesicle, a cell, or a tissue. The delivery vehicle can be any of the nanoparticles described herein, such as nanoparticles comprising lipids (as previously described), inorganic nanomaterials, and other polymeric materials.

The delivery vehicle can be capable of delivering the cargo to a cell, such as delivering any of the effector molecules described herein to a cell. The delivery vehicle can be capable of delivering the cargo to a cell, such as delivering any of the effector molecules described herein to a cell. The delivery vehicle can be configured to target a specific cell, such as configured with a re-directing antibody to target a specific cell. The delivery vehicle can be capable of delivering the cargo to a cell in vivo.

The delivery vehicle can be capable of delivering the cargo to a tissue or tissue environment (e.g., a tumor microenvironment), such as delivering any of the effector molecules described herein to a tissue or tissue environment in vivo. Delivering a cargo can include secreting the cargo, such as secreting any of the effector molecules described herein. Accordingly, the delivery vehicle can be capable of secreting the cargo, such as secreting any of the effector molecules described herein. The delivery vehicle can be capable of secreting the cargo to a tissue or tissue environment (e.g., a tumor microenvironment), such as secreting any of the effector molecules described herein a tissue or tissue environment. The delivery vehicle can be configured to target a specific a tissue or tissue environment (e.g., a tumor microenvironment), such as configured with a re-directing antibody to target a specific a tissue or tissue environment.

Methods of Treatment

Further provided herein are methods that include delivering, or administering, to a subject (e.g., a human subject) engineered cells as provided herein to produce in vivo at least one effector molecule produced by the engineered cells. Further provided herein are methods that include delivering, or administering, to a subject (e.g., a human subject) engineered cells as provided herein to produce in vivo at least two effector molecule produced by the engineered cells.

Further provided herein are methods that include delivering, or administering, to a subject (e.g., a human subject) any of the delivery vehicles described herein, such as any of the delivery vehicles described herein comprising any of the effector molecules described herein. Further provided herein are methods that include delivering, or administering, to a subject (e.g., a human subject) any of the delivery vehicles described herein, such as any of the delivery vehicles described herein comprising two or more effector molecules.

In some embodiments, the engineered cells or delivery vehicles are administered via intravenous, intraperitoneal, intratracheal, subcutaneous, intratumoral, oral, anal, intranasal (e.g., packed in a delivery particle), or arterial (e.g., internal carotid artery) routes. Thus, the engineered cells or delivery vehicles may be administered systemically or locally (e.g., to a TME or via intratumoral administration). An engineered cell can be isolated from a subject, such as a subject known or suspected to have cancer. An engineered cell can be allogenic with reference to the subject being administered a treatment. Allogenic modified cells can be HLA-matched to the subject being administered a treatment. Delivery vehicles can be any of the lipid structure delivery systems described herein. Delivery vehicles can be any of the nanoparticles described herein.

Engineered cells or delivery vehicles can be administered alone or in combination with other treatments, either simultaneously or sequentially dependent upon the condition to be treated. For example, engineered cells or delivery vehicles can be administered in combination with a checkpoint inhibitor therapy. Exemplary checkpoint inhibitors include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-AZAR antibodies, anti-phosphatidylserine antibodies, anti-CD27 antibodies, anti-TNFa antibodies, anti-TREM1 antibodies, and anti-TREM2 antibodies. Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MEDI4736/Imfinzi®—Medimmune/AstraZeneca), atezolizumab (anti-PD-L1; Tecentriq®—Roche/Genentech), BMS-936559 (anti-PD-L1—BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca). In other examples, engineered cells or delivery vehicles can be administered in combination with TGFbeta inhibitors, VEGF inhibitors, or HPGE2. In another example, engineered cells or delivery vehicles can be administered in combination with an anti-CD40 antibody.

Some methods comprise selecting a subject (or patient population) having a tumor (or cancer) and treating that subject with engineered cells or delivery vehicles that modulate tumor-mediated immunosuppressive mechanisms.

The engineered cells or delivery vehicles of the present disclosure may be used, in some instances, to treat cancer, such as ovarian cancer. Other cancers are described herein. For example, the engineered cells may be used to treat bladder tumors, brain tumors, breast tumors, cervical tumors, colorectal tumors, esophageal tumors, gliomas, kidney tumors, liver tumors, lung tumors, melanomas, ovarian tumors, pancreatic tumors, prostate tumors, skin tumors, thyroid tumors, and/or uterine tumors. The engineered cells or delivery vehicles of the present disclosure can be used to treat cancers with tumors located in the peritoneal space of a subject.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells.

### In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the effector molecules described herein. The methods provided herein also include delivering a composition in vivo capable of producing two or more of the effector molecules described herein. Compositions capable of in vivo production of effector molecules include, but are not limited to, any of the engineered nucleic acids described herein. Compositions capable of in vivo production of effector molecules can be a naked mRNA or a naked plasmid.

### Additional Embodiments

Provided below are enumerated embodiments describing specific non-limiting embodiments of the present invention:

Embodiment 1. A tumor cell engineered to produce two or more effector molecules.
Embodiment 2. The engineered cell of embodiment 1, wherein the tumor cell is selected from the group consisting of a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, an ovarian tumor cell, a pancreatic tumor cell, a prostate tumor cell, a skin tumor cell, a thyroid tumor cell, and a uterine tumor cell.
Embodiment 3. The engineered cell of embodiment 1 or embodiment 2, wherein the cell was engineered via transduction with an oncolytic virus.
Embodiment 4. The engineered cell of embodiment 3, wherein the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.
Embodiment 5. The engineered cell of embodiment 3 or embodiment 4, wherein the oncolytic virus is a recombinant oncolytic virus comprising one more transgenes encoding one or more of the two or more effector molecules.
Embodiment 6. An erythrocyte engineered to produce two or more effector molecules.
Embodiment 7. A platelet cell engineered to produce two or more effector molecules.
Embodiment 8. A bacterial cell engineered to produce two or more mammalian effector molecules.
Embodiment 9. The engineered cell of embodiment 8, wherein the bacterial cell is selected from the group consisting of Clostridium beijerinckii, Clostridium sporogenes, Clostridium novyi, Escherichia coli, Pseudomonas aeruginosa, Listeria monocytogenes, Salmonella typhimurium, and Salmonella choleraesuis.
Embodiment 10. The engineered cell of any one of embodiments 1-9, wherein each of the two or more effector molecules comprises a secretion signal.
Embodiment 11. The engineered cell of embodiment 10, wherein each of the two or more effector molecules is secreted from the cell.
Embodiment 12. The engineered cell of any one of embodiments 1 and 6-11, wherein the cell was engineered via transfection with an isolated nucleic acid.
Embodiment 13. The engineered cell of embodiment 12, wherein the isolated nucleic acid is a cDNA comprising a polynucleotide sequence encoding one or more of the two or more effector molecules.
Embodiment 14. The engineered cell of embodiment 12, wherein the isolated nucleic acid is an mRNA comprising a polynucleotide sequence encoding one or more of the two or more effector molecules.
Embodiment 15. The engineered cell of embodiment 12, wherein the isolated nucleic acid is a naked plasmid comprising a polynucleotide sequence encoding one or more of the two or more effector molecules.
Embodiment 16. The engineered cell of any one of embodiments 1-15, wherein the two or more effector molecules are encoded by a polynucleotide sequence.
Embodiment 17. The engineered cell of embodiment 16, wherein the polynucleotide sequence comprises a promoter.
Embodiment 18. The engineered cell of embodiment 17, wherein the promoter comprises an exogenous promoter polynucleotide sequence.
Embodiment 19. The engineered cell of embodiment 17, wherein the promoter comprises an endogenous promoter.
Embodiment 20. The engineered cell of any one of embodiments 16-19, wherein the polynucleotide sequence further comprises a linker polynucleotide sequence.
Embodiment 21. The engineered cell of embodiment 20, wherein the promoter is operably linked to the polynucleotide sequence such that the two or more effector molecules are capable of being transcribed as a single polynucleotide comprising the formula (L-E)x,
wherein
L comprises a linker polynucleotide sequence,
E comprises a polynucleotide encoding one of the two or more effector molecules,

### X=2 to 20, and

wherein for the first iteration of the (L-E) unit L is absent.
Embodiment 22. The engineered cell of embodiment 20 or embodiment 21, wherein the linker polynucleotide sequence is operably associated with the translation of the two or more effector molecules as separate polypeptides.
Embodiment 23. The engineered cell of any one of embodiments 20-22, wherein the linker polynucleotide sequence encodes a 2A ribosome skipping tag.
Embodiment 24. The engineered cell of embodiment 23, wherein the 2A ribosome skipping tag is selected from the group consisting of P2A, T2A, E2A, and F2A.
Embodiment 25. The engineered cell of embodiment 20-24, wherein the linker polynucleotide sequence encodes a cleavable polypeptide.
Embodiment 26. The engineered cell of embodiment 25, wherein the cleavable polypeptide comprises a furin polypeptide sequence.
Embodiment 27. The engineered cell of any one of embodiments 20-22, wherein the linker polynucleotide sequence encodes an Internal Ribosome Entry Site (IRES).
Embodiment 28. The engineered cell of any one of embodiments 20-27, wherein the linker polynucleotide sequence encodes an additional promoter.
Embodiment 29. The engineered cell of embodiment 28, wherein the promoter is operably linked to the polynucleotide sequence such that a first polynucleotide encoding one of the two or more effector molecules is capable of being transcribed,
wherein the additional promoter is operably linked to the polynucleotide sequence such that a second polynucleotide encoding a second of the two or more effector molecules is capable of being transcribed, and wherein the first and the second polynucleotides are separate polynucleotides.
Embodiment 30. The engineered cell of embodiment 28 or embodiment 29, wherein the promoter and the additional promoter are identical.
Embodiment 31. The engineered cell of embodiment 28 or embodiment 29, wherein the promoter and the additional promoter are different.
Embodiment 32. The engineered cell of any one of embodiments 1-7 and 10-31, wherein the engineered cell is a human cell.
Embodiment 33. The engineered cell of embodiment 32, wherein the human cell is an isolated cell from a subject.
Embodiment 34. The engineered cell of any one of embodiments 1-33, wherein the engineered cell is a cultured cell.
Embodiment 35. The engineered cell of any one of embodiments 17-34, wherein the promoter and/or the additional promoter comprises a constitutive promoter.
Embodiment 36. The engineered cell of embodiment 35, wherein the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 37. The engineered cell of any one of embodiments 17-36, wherein the promoter and/or the additional promoter comprises an inducible promoter.
Embodiment 38. The engineered cell of embodiment 37, wherein the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.
Embodiment 39. The engineered cell of any one of embodiments 10-38, wherein one secretion signal peptide comprises a native secretion signal peptide native to a corresponding effector molecule of the two or more effector molecules.
Embodiment 40. The engineered cell of any one of embodiments 10-39, wherein one secretion signal peptide comprises a non-native secretion signal peptide non-native to at least one of the two or more effector molecules.
Embodiment 41. The engineered cell of embodiment 40, wherein the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12.
Embodiment 42. The engineered cell of any one of embodiments 10-41, wherein each secretion signal peptide is identical.
Embodiment 43. The engineered cell of any one of embodiments 1-42, wherein a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.
Embodiment 44. The engineered cell of any one of embodiments 1-43, wherein a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme.
Embodiment 45. The engineered cell of embodiment 44, wherein the therapeutic class of the first effector molecule and the second effector molecule are different.
Embodiment 46. The engineered cell of any one of embodiments 43-45, wherein the cytokine is selected from the group consisting of IL12, IL7, IL21, IL18, IL15, Type I interferons, and Interferon-gamma.
Embodiment 47. The engineered cell of embodiment 46, wherein the IL12 cytokine is an IL12p70 fusion protein.
Embodiment 48. The engineered cell of any one of embodiments 43-47, wherein the chemokine is selected from the group consisting of CCL21a, CXCL10, CXCL11, CXCL13, CXCL10-11 fusion, CCL19, CXCL9, and XCL1.
Embodiment 49. The engineered cell of any one of embodiments 43-48, wherein the growth factor is selected from the group consisting of FLT3L and GM-CSF.
Embodiment 50. The engineered cell of any one of embodiments 43-49, wherein the co-activation molecule is selected from the group consisting of 4-1BBL and CD40L.
Embodiment 51. The engineered cell of any one of embodiments 43-50, wherein the tumor microenvironment modifier is selected from the group consisting of adenosine deaminase, TGFbeta inhibitors, immune checkpoint inhibitors, VEGF inhibitors, and HPGE2.
Embodiment 52. The engineered cell of embodiment 51, wherein the TGFbeta inhibitors are selected from the group consisting of an anti-TGFbeta peptide, an anti-TGFbeta antibody, a TGFb-TRAP, and combinations thereof.
Embodiment 53. The engineered cell of embodiment 51, wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 54. The engineered cell of embodiment 51, wherein the VEGF inhibitors comprise anti-VEGF antibodies, anti-VEGF peptides, or combinations thereof.
Embodiment 55. The engineered cell of any one of embodiments 1-54, wherein at least one of the two or more effector molecules is a human-derived effector molecule.
Embodiment 56. The engineered cell of any one of embodiments 1-55, wherein one effector molecule comprises IL12.
Embodiment 57. The engineered cell of embodiment 56, wherein a second effector molecule comprises CCL21a, IL7, IL15, or IL21.
Embodiment 58. The engineered cell of any one of embodiments 21-57, wherein the polynucleotide sequence comprises:
a) an SFFV promoter; and
b) a polynucleotide described in a formula, oriented from 5′ to 3′, comprising

S1-E1-L-S2-E2

wherein
S1 comprises a polynucleotide sequence encoding a first secretion signal peptide, wherein the first secretion signal peptide is a human IL12 secretion signal peptide;
E1 comprises a polynucleotide sequence encoding a first effector molecule, wherein the first effector molecule is a human IL12p70 fusion protein;
L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;
S2 comprises a polynucleotide sequence encoding a second secretion signal peptide, wherein the second secretion signal peptide is a human IL21 secretion signal peptide;
E2 comprises a polynucleotide sequence encoding a second effector molecule, wherein the second effector molecule is human IL21; and
wherein the SFFV promoter is operably linked to the polynucleotide, the first secretion signal peptide is operably linked to the first effector molecule, and the second secretion signal peptide is operably linked to the second effector molecule.
Embodiment 59. The engineered cell of embodiment 58, wherein the polynucleotide sequence comprises the polynucleotide sequence of SEQ ID NO: 144.
Embodiment 60. The engineered cell of any one of embodiments 16-59, wherein the polynucleotide sequence is integrated into the genome of the engineered cell.
Embodiment 61. The engineered cell of any one of embodiments 16-60, wherein the polynucleotide sequence comprises one or more viral vector polynucleotide sequences.
Embodiment 62. The engineered cell of embodiment 61, wherein the one or more viral vector polynucleotide sequences comprise lentiviral, retroviral, retrotransposon, adenoviral, or adeno-associated viral polynucleotide sequences.
Embodiment 63. A population of cells comprising one or more engineered cells of any one of embodiments 1-62.
Embodiment 64. A composition comprising the engineered cell of any one of embodiments 1-62 or the population of cells of embodiment 63, and a pharmaceutically acceptable carrier.
Embodiment 65. A method of treating a subject having cancer, the method comprising administering a therapeutically effective dose of any of the engineered cell of any one of embodiments 1-62, the population of cells of embodiment 63, or the composition of embodiment 64.
Embodiment 66. A method of reducing tumor volume in a subject, the method comprising administering to a subject having a tumor a composition comprising any of the engineered cell of any one of embodiments 1-62 or the population of cells of embodiment 63, and a pharmaceutically acceptable carrier.
Embodiment 67. The method of embodiment 65 or embodiment 66, wherein the administering comprises systemic administration.
Embodiment 68. The method of embodiment 65 or embodiment 66, wherein the administering comprises intratumoral administration or intraperitoneal administration.
Embodiment 69. The method of any one of embodiments 65-68, wherein the engineered cell is derived from the subject.
Embodiment 70. The method of any one of embodiments 65-68, wherein the engineered cell is allogeneic with reference to the subject.
Embodiment 71. The method of any one of embodiments 65-70, wherein the method further comprises administering a checkpoint inhibitor.
Embodiment 72. The method of embodiment 71, wherein the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 73. The method of any one of embodiments 65-72, wherein the method further comprises administering an anti-CD40 antibody.
Embodiment 74. The method of any one of embodiments 65-73, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.
Embodiment 75. The method of any one of embodiments 65-73, wherein the tumor is a tumor located in a peritoneal space.
Embodiment 76. A lipid structure delivery system comprising a lipid-based structure comprising two or more effector molecules.
Embodiment 77. The lipid-based structure of embodiment 76, wherein the two or more effector molecules are encoded by a polynucleotide sequence.
Embodiment 78. A lipid-based structure comprising an engineered nucleic acid, wherein the engineered nucleic acid comprises a polynucleotide sequence encoding two or more effector molecules.
Embodiment 79. The lipid-based structure of embodiment 78, wherein the engineered nucleic acid is a cDNA.
Embodiment 80. The lipid-based structure of embodiment 78, wherein the engineered nucleic acid is an mRNA.
Embodiment 81. The lipid-based structure of embodiment 78, wherein the engineered nucleic acid is a naked plasmid.
Embodiment 82. The lipid-based structure of any one of embodiments 77-81, wherein the polynucleotide sequence comprises a promoter.
Embodiment 83. The lipid-based structure of embodiment 82, wherein the promoter comprises an exogenous promoter polynucleotide sequence.
Embodiment 84. The lipid-based structure of embodiment 82, wherein the promoter comprises an endogenous promoter.
Embodiment 85. The lipid-based structure of any one of embodiments 77-84, wherein the polynucleotide sequence further comprises a linker polynucleotide sequence.
Embodiment 86. The lipid-based structure of embodiment 85, wherein the promoter is operably linked to the polynucleotide sequence such that the two or more effector molecules are capable of being transcribed as a single polynucleotide comprising the formula (L-E)x,
wherein
L comprises a linker polynucleotide sequence,
E comprises a polynucleotide encoding one of the two or more effector molecules,

### X=2 to 20, and

wherein for the first iteration of the (L-E) unit L is absent.
Embodiment 87. The lipid-based structure of embodiment 85 or embodiment 86, wherein the linker polynucleotide sequence is operably associated with the translation of the two or more effector molecules as separate polypeptides.
Embodiment 88. The lipid-based structure of any one of embodiments 85-87, wherein the linker polynucleotide sequence encodes a 2A ribosome skipping tag.
Embodiment 89. The lipid-based structure of embodiment 88, wherein the 2A ribosome skipping tag is selected from the group consisting of P2A, T2A, E2A, and F2A.
Embodiment 90. The lipid-based structure of embodiment 85-89, wherein the linker polynucleotide sequence encodes a cleavable polypeptide.
Embodiment 91. The lipid-based structure of embodiment 90, wherein the cleavable polypeptide comprises a furin polypeptide sequence.
Embodiment 92. The lipid-based structure of any one of embodiments 85-87, wherein the linker polynucleotide sequence encodes an Internal Ribosome Entry Site (IRES).
Embodiment 93. The lipid-based structure of any one of embodiments 85-92, wherein the linker polynucleotide sequence encodes an additional promoter.
Embodiment 94. The lipid-based structure of embodiment 93, wherein the promoter is operably linked to the polynucleotide sequence such that a first polynucleotide encoding one of the two or more effector molecules is capable of being transcribed,
wherein the additional promoter is operably linked to the polynucleotide sequence such that a second polynucleotide encoding a second of the two or more effector molecules is capable of being transcribed, and
wherein the first and the second polynucleotides are separate polynucleotides.
Embodiment 95. The lipid-based structure of embodiment 93 or embodiment 94, wherein the promoter and the additional promoter are identical.
Embodiment 96. The lipid-based structure of embodiment 93 or embodiment 94, wherein the promoter and the additional promoter are different.
Embodiment 97. The lipid-based structure of any one of embodiments 82-96, wherein the promoter and/or the additional promoter comprises a constitutive promoter.
Embodiment 98. The lipid-based structure of embodiment 97, wherein the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 99. The lipid-based structure of any one of embodiments 82-98, wherein the promoter and/or the additional promoter comprises an inducible promoter.
Embodiment 100. The lipid-based structure of embodiment 99, wherein the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.
Embodiment 101. The lipid-based structure of any one of embodiments 76-100, wherein each of the two or more effector molecules comprises a secretion signal.
Embodiment 102. The lipid-based structure of embodiment 101, wherein one secretion signal peptide comprises a native secretion signal peptide native to a corresponding effector molecule of the two or more effector molecules.
Embodiment 103. The lipid-based structure of embodiment 101 or embodiment 102, wherein one secretion signal peptide comprises a non-native secretion signal peptide non-native to at least one of the two or more effector molecules.
Embodiment 104. The lipid-based structure of embodiment 103, wherein the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12.
Embodiment 105. The lipid-based structure of any one of embodiments 101-104, wherein each secretion signal peptide is identical.
Embodiment 106. The lipid-based structure of any one of embodiments 76-105, wherein a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.
Embodiment 107. The lipid-based structure of any one of embodiments 76-106, wherein a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme.
Embodiment 108. The lipid-based structure of embodiment 107, wherein the therapeutic class of the first effector molecule and the second effector molecule are different.
Embodiment 109. The lipid-based structure of any one of embodiments 106-108, wherein the cytokine is selected from the group consisting of IL12, IL7, IL21, IL18, IL15, Type I interferons, and Interferon-gamma.
Embodiment 110. The lipid-based structure of embodiment 109, wherein the IL12 cytokine is an IL12p70 fusion protein.
Embodiment 111. The lipid-based structure of any one of embodiments 106-110, wherein the chemokine is selected from the group consisting of CCL21a, CXCL10, CXCL11, CXCL13, CXCL10-11 fusion, CCL19, CXCL9, and XCL1.
Embodiment 112. The lipid-based structure of any one of embodiments 106-111, wherein the growth factor is selected from the group consisting of FLT3L and GM-CSF.
Embodiment 113. The lipid-based structure of any one of embodiments 106-112, wherein the co-activation molecule is selected from the group consisting of 4-1BBL and CD40L.
Embodiment 114. The lipid-based structure of any one of embodiments 106-113, wherein the tumor microenvironment modifier is selected from the group consisting of adenosine deaminase, TGFbeta inhibitors, immune checkpoint inhibitors, VEGF inhibitors, and HPGE2.
Embodiment 115. The lipid-based structure of embodiment 114, wherein the TGFbeta inhibitors are selected from the group consisting of an anti-TGFbeta peptide, an anti-TGFbeta antibody, a TGFb-TRAP, and combinations thereof.
Embodiment 116. The lipid-based structure of embodiment 114, wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 117. The lipid-based structure of embodiment 114, wherein the VEGF inhibitors comprise anti-VEGF antibodies, anti-VEGF peptides, or combinations thereof.
Embodiment 118. The lipid-based structure of any one of embodiments 76-117, wherein at least one of the two or more effector molecules is a human-derived effector molecule.
Embodiment 119. The lipid-based structure of any one of embodiments 76-118, wherein one effector molecule comprises IL12.
Embodiment 120. The lipid-based structure of embodiment 119, wherein a second effector molecule comprises CCL21a, IL7, IL15, or IL21.
Embodiment 121. The lipid-based structure of any one of embodiments 86-120, wherein the polynucleotide sequence comprises:
a) an SFFV promoter; and
b) a polynucleotide described in a formula, oriented from 5′ to 3′, comprising

S1-E1-L-S2-E2

wherein
S1 comprises a polynucleotide sequence encoding a first secretion signal peptide, wherein the first secretion signal peptide is a human IL12 secretion signal peptide;
E1 comprises a polynucleotide sequence encoding a first effector molecule, wherein the first effector molecule is a human IL12p70 fusion protein;
L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;
S2 comprises a polynucleotide sequence encoding a second secretion signal peptide, wherein the second secretion signal peptide is a human IL21 secretion signal peptide;
E2 comprises a polynucleotide sequence encoding a second effector molecule, wherein the second effector molecule is human IL21; and
wherein the SFFV promoter is operably linked to the polynucleotide, the first secretion signal peptide is operably linked to the first effector molecule, and the second secretion signal peptide is operably linked to the second effector molecule.
Embodiment 122. The lipid-based structure of embodiment 121, wherein the polynucleotide sequence comprises the polynucleotide sequence of SEQ ID NO: 144.
Embodiment 123. The lipid-based structure of any one of embodiments 76-122, wherein the lipid-based structure comprises a extracellular vesicle.
Embodiment 124. The lipid-based structure of embodiment 123, wherein the extracellular vesicle is selected from the group consisting of a nanovesicle and an exosome.
Embodiment 125. The lipid-based structure of any one of embodiments 76-122, wherein the lipid-based structure comprises a lipid nanoparticle or a micelle.
Embodiment 126. The lipid-based structure of any one of embodiments 76-122, wherein the lipid-based structure comprises a liposome.
Embodiment 127. A composition comprising the lipid-based structure of any one of embodiments 76-126 and a pharmaceutically acceptable carrier.
Embodiment 128. A method of treating a subject having cancer, the method comprising administering a therapeutically effective dose of any of the lipid-based structures of any one of embodiments 76-126 or the composition of embodiment 127.
Embodiment 129. A method of reducing tumor volume in a subject, the method comprising administering to a subject having a tumor a composition comprising any of the lipid-based structures of any one of embodiments 77-126 and a pharmaceutically acceptable carrier.
Embodiment 130. The method of embodiment 128 or embodiment 129, wherein the administering comprises systemic administration.
Embodiment 131. The method of embodiment 128 or embodiment 129, wherein the administering comprises intratumoral administration or intraperitoneal administration.
Embodiment 132. The method of any one of embodiments 128-131, wherein the lipid-based structure is capable of engineering a cell in the in the subject to produce two or more effector molecules that modulate tumor-mediated immunosuppressive mechanisms.
Embodiment 133. The method of embodiment 132, wherein the cell is a tumor cell, an immune cell, an erythrocyte, or a platelet cell.
Embodiment 134. The method of any one of embodiments 128-133, wherein the method further comprises administering a checkpoint inhibitor.
Embodiment 135. The method of embodiment 134, wherein the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 136. The method of any one of embodiments 128-135, wherein the method further comprises administering an anti-CD40 antibody.
Embodiment 137. The method of any one of embodiments 128-136, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.
Embodiment 138. The method of any one of embodiments 128-136, wherein the tumor is a tumor located in a peritoneal space.
Embodiment 139. A nanoparticle comprising two or more effector molecules.
Embodiment 140. The nanoparticle of embodiment 139, wherein the two or more effector molecules are encoded by a polynucleotide sequence.
Embodiment 141. A nanoparticle comprising an engineered nucleic acid, wherein the engineered nucleic acid comprises a polynucleotide sequence encoding two or more effector molecules.
Embodiment 142. The nanoparticle of embodiment 141, wherein the engineered nucleic acid is a cDNA.
Embodiment 143. The nanoparticle of embodiment 141, wherein the engineered nucleic acid is an mRNA.
Embodiment 144. The nanoparticle of embodiment 141, wherein the engineered nucleic acid is a naked plasmid.
Embodiment 145. The nanoparticle of any one of embodiments 140-144, wherein the polynucleotide sequence comprises a promoter.
Embodiment 146. The nanoparticle of embodiment 145, wherein the promoter comprises an exogenous promoter polynucleotide sequence.
Embodiment 147. The nanoparticle of embodiment 145, wherein the promoter comprises an endogenous promoter.
Embodiment 148. The nanoparticle of any one of embodiments 140-147, wherein the polynucleotide sequence further comprises a linker polynucleotide sequence.
Embodiment 149. The nanoparticle of embodiment 148, wherein the promoter is operably linked to the polynucleotide sequence such that the two or more effector molecules are capable of being transcribed as a single polynucleotide comprising the formula (L-E)x,
wherein
L comprises a linker polynucleotide sequence,
E comprises a polynucleotide encoding one of the two or more effector molecules,

### X=2 to 20, and

wherein for the first iteration of the (L-E) unit L is absent.
Embodiment 150. The nanoparticle of embodiment 148 or embodiment 149, wherein the linker polynucleotide sequence is operably associated with the translation of the two or more effector molecules as separate polypeptides.
Embodiment 151. The nanoparticle of any one of embodiments 148-150, wherein the linker polynucleotide sequence encodes a 2A ribosome skipping tag.
Embodiment 152. The nanoparticle of embodiment 151, wherein the 2A ribosome skipping tag is selected from the group consisting of P2A, T2A, E2A, and F2A.
Embodiment 153. The nanoparticle of embodiment 148-152, wherein the linker polynucleotide sequence encodes a cleavable polypeptide.
Embodiment 154. The nanoparticle of embodiment 153, wherein the cleavable polypeptide comprises a furin polypeptide sequence.
Embodiment 155. The nanoparticle of any one of embodiments 148-150, wherein the linker polynucleotide sequence encodes an Internal Ribosome Entry Site (IRES).
Embodiment 156. The nanoparticle of any one of embodiments 148-155, wherein the linker polynucleotide sequence encodes an additional promoter.
Embodiment 157. The nanoparticle of embodiment 156, wherein the promoter is operably linked to the polynucleotide sequence such that a first polynucleotide encoding one of the two or more effector molecules is capable of being transcribed,
wherein the additional promoter is operably linked to the polynucleotide sequence such that a second polynucleotide encoding a second of the two or more effector molecules is capable of being transcribed, and wherein the first and the second polynucleotides are separate polynucleotides.
Embodiment 158. The nanoparticle of embodiment 156 or embodiment 157, wherein the promoter and the additional promoter are identical.
Embodiment 159. The nanoparticle of embodiment 156 or embodiment 157, wherein the promoter and the additional promoter are different.
Embodiment 160. The nanoparticle of any one of embodiments 156-159, wherein the promoter and/or the additional promoter comprises a constitutive promoter.
Embodiment 161. The nanoparticle of embodiment 160, wherein the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 162. The nanoparticle of any one of embodiments 156-161, wherein the promoter and/or the additional promoter comprises an inducible promoter.
Embodiment 163. The nanoparticle of embodiment 162, wherein the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.
Embodiment 164. The nanoparticle of any one of embodiments 139-163, wherein each of the two or more effector molecules comprises a secretion signal.
Embodiment 165. The nanoparticle of embodiment 164, wherein one secretion signal peptide comprises a native secretion signal peptide native to at least one of the two or more effector molecules.
Embodiment 166. The nanoparticle of embodiment 164 or embodiment 165, wherein one secretion signal peptide comprises a non-native secretion signal peptide non-native to at least one of the two or more effector molecules.
Embodiment 167. The nanoparticle of embodiment 166, wherein the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12.
Embodiment 168. The nanoparticle of any one of embodiments 164-167, wherein each secretion signal peptide is identical.
Embodiment 169. The nanoparticle of any one of embodiments 139-168, wherein a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.
Embodiment 170. The nanoparticle of any one of embodiments 139-169, wherein a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme.
Embodiment 171. The nanoparticle of embodiment 170, wherein the therapeutic class of the first effector molecule and the second effector molecule are different.
Embodiment 172. The nanoparticle of any one of embodiments 169-171, wherein the cytokine is selected from the group consisting of IL12, IL7, IL21, IL18, IL15, Type I interferons, and Interferon-gamma.
Embodiment 173. The nanoparticle of embodiment 172, wherein the IL12 cytokine is an IL12p70 fusion protein.
Embodiment 174. The nanoparticle of any one of embodiments 169-173, wherein the chemokine is selected from the group consisting of CCL21a, CXCL10, CXCL11, CXCL13, CXCL10-11 fusion, CCL19, CXCL9, and XCL1.
Embodiment 175. The nanoparticle of any one of embodiments 169-174, wherein the growth factor is selected from the group consisting of FLT3L and GM-CSF.
Embodiment 176. The nanoparticle of any one of embodiments 169-175, wherein the co-activation molecule is selected from the group consisting of 4-1BBL and CD40L.
Embodiment 177. The nanoparticle of any one of embodiments 169-176, wherein the tumor microenvironment modifier is selected from the group consisting of adenosine deaminase, TGFbeta inhibitors, immune checkpoint inhibitors, VEGF inhibitors, and HPGE2.
Embodiment 178. The nanoparticle of embodiment 177, wherein the TGFbeta inhibitors are selected from the group consisting of an anti-TGFbeta peptide, an anti-TGFbeta antibody, a TGFb-TRAP, and combinations thereof.
Embodiment 179. The nanoparticle of embodiment 177, wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 180. The nanoparticle of embodiment 177, wherein the VEGF inhibitors comprise anti-VEGF antibodies, anti-VEGF peptides, or combinations thereof.
Embodiment 181. The nanoparticle of any one of embodiments 139-180, wherein at least one of the two or more effector molecules is a human-derived effector molecule.
Embodiment 182. The nanoparticle of any one of embodiments 139-181, wherein one effector molecule comprises IL12.
Embodiment 183. The nanoparticle of embodiment 182, wherein a second effector molecule comprises CCL21a, IL7, IL15, or IL21.
Embodiment 184. The nanoparticle of any one of embodiments 149-183, wherein the polynucleotide sequence comprises:
a) an SFFV promoter; and
b) a polynucleotide described in a formula, oriented from 5′ to 3′, comprising

S1-E1-L-S2-E2

wherein
S1 comprises a polynucleotide sequence encoding a first secretion signal peptide, wherein the first secretion signal peptide is a human IL12 secretion signal peptide;
E1 comprises a polynucleotide sequence encoding a first effector molecule, wherein the first effector molecule is a human IL12p70 fusion protein;
L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;
S2 comprises a polynucleotide sequence encoding a second secretion signal peptide, wherein the second secretion signal peptide is a human IL21 secretion signal peptide;
E2 comprises a polynucleotide sequence encoding a second effector molecule, wherein the second effector molecule is human IL21; and wherein the SFFV promoter is operably linked to the polynucleotide, the first secretion signal peptide is operably linked to the first effector molecule, and the second secretion signal peptide is operably linked to the second effector molecule.
Embodiment 185. The nanoparticle of embodiment 184, wherein the polynucleotide sequence comprises the polynucleotide sequence of SEQ ID NO: 144.
Embodiment 186. The nanoparticle of any one of embodiments 139-185, wherein the nanoparticle comprises an inorganic material.
Embodiment 187. A composition comprising the nanoparticle of any one of embodiments 139-186 and a pharmaceutically acceptable carrier.
Embodiment 188. A method of treating a subject having cancer, the method comprising administering a therapeutically effective dose of any of the nanoparticles of any one of embodiments 139-186 or the composition of embodiment 187.
Embodiment 189. A method of reducing tumor volume in a subject, the method comprising administering to a subject having a tumor a composition comprising any of the nanoparticles of any one of embodiments 139-186 and a pharmaceutically acceptable carrier.
Embodiment 190. The method of embodiment 188 or embodiment 189, wherein the administering comprises systemic administration.
Embodiment 191. The method of embodiment 188 or embodiment 189, wherein the administering comprises intratumoral administration or intraperitoneal administration.
Embodiment 192. The method of any one of embodiments 188-191, wherein the nanoparticle is capable of engineering a cell in the subject to produce two or more effector molecules that modulate tumor-mediated immunosuppressive mechanisms.
Embodiment 193. The method of embodiment 192, wherein the cell is a tumor cell, an immune cell, an erythrocyte, or a platelet cell.
Embodiment 194. The method of any one of embodiments 188-193, wherein the method further comprises administering a checkpoint inhibitor.
Embodiment 195. The method of embodiment 194, wherein the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 196. The method of any one of embodiments 188-195, wherein the method further comprises administering an anti-CD40 antibody.
Embodiment 197. The method of any one of embodiments 188-196, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.
Embodiment 198. The method of any one of embodiments 188-196, wherein the tumor is a tumor located in a peritoneal space.
Embodiment 199. A virus engineered to comprise a heterologous nucleic acid, wherein the heterologous nucleic acid comprises a polynucleotide sequence encoding two or more effector molecules.
Embodiment 200. The engineered virus of embodiment 199, wherein the virus is selected from the group consisting of a lentivirus, a retrovirus, an oncolytic virus, an adenovirus, an adeno-associated virus (AAV), and a virus-like particle (VLP).
Embodiment 201. An oncolytic virus engineered to comprise a heterologous nucleic acid, wherein the heterologous nucleic acid comprises a polynucleotide sequence encoding two or more effector molecules.
Embodiment 202. The engineered virus of any one of embodiments 199-201, wherein the engineered nucleic acid comprises DNA.
Embodiment 203. The engineered virus of any one of embodiments 199-201, wherein the engineered nucleic acid comprises RNA.
Embodiment 204. The engineered virus of any one of embodiments 199-203, wherein the polynucleotide sequence comprises a promoter.
Embodiment 205. The engineered virus of embodiment 204, wherein the promoter comprises an exogenous promoter polynucleotide sequence.
Embodiment 206. The engineered virus of embodiment 204, wherein the promoter comprises an endogenous promoter.
Embodiment 207. The engineered virus of any one of embodiments 199-206, wherein the polynucleotide sequence further comprises a linker polynucleotide sequence.
Embodiment 208. The engineered virus of embodiment 207, wherein the promoter is operably linked to the polynucleotide sequence such that the two or more effector molecules are capable of being transcribed as a single polynucleotide comprising the formula (L-E)x,
wherein
L comprises a linker polynucleotide sequence,
E comprises a polynucleotide encoding one of the two or more effector molecules,

### X=2 to 20, and

wherein for the first iteration of the (L-E) unit L is absent.
Embodiment 209. The engineered virus of embodiment 207 or embodiment 208, wherein the linker polynucleotide sequence is operably associated with the translation of the two or more effector molecules as separate polypeptides.
Embodiment 210. The engineered virus of any one of embodiments 207-209, wherein the linker polynucleotide sequence encodes a 2A ribosome skipping tag.
Embodiment 211. The engineered virus of embodiment 210, wherein the 2A ribosome skipping tag is selected from the group consisting of P2A, T2A, E2A, and F2A.
Embodiment 212. The engineered virus of embodiment 207-211, wherein the linker polynucleotide sequence encodes a cleavable polypeptide.
Embodiment 213. The engineered virus of embodiment 212, wherein the cleavable polypeptide comprises a furin polypeptide sequence.
Embodiment 214. The engineered virus of any one of embodiments 207-209, wherein the linker polynucleotide sequence encodes an Internal Ribosome Entry Site (IRES).
Embodiment 215. The engineered virus of any one of embodiments 207-214, wherein the linker polynucleotide sequence encodes an additional promoter.
Embodiment 216. The engineered virus of embodiment 215, wherein the promoter is operably linked to the polynucleotide sequence such that a first polynucleotide encoding one of the two or more effector molecules is capable of being transcribed,
wherein the additional promoter is operably linked to the polynucleotide sequence such that a second polynucleotide encoding a second of the two or more effector molecules is capable of being transcribed, and wherein the first and the second polynucleotides are separate polynucleotides.
Embodiment 217. The engineered virus of embodiment 215 or embodiment 216, wherein the promoter and the additional promoter are identical.
Embodiment 218. The engineered virus of embodiment 215 or embodiment 216, wherein the promoter and the additional promoter are different.
Embodiment 219. The engineered virus of any one of embodiments 202-218, wherein the promoter and/or the additional promoter comprises a constitutive promoter.
Embodiment 220. The engineered virus of embodiment 219, wherein the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 221. The engineered virus of any one of embodiments 202-220, wherein the promoter and/or the additional promoter comprises an inducible promoter.
Embodiment 222. The engineered virus of embodiment 221, wherein the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.
Embodiment 223. The engineered virus of any one of embodiments 199-222, wherein each of the two or more effector molecules comprises a secretion signal.
Embodiment 224. The engineered virus of embodiment 223, wherein one secretion signal peptide comprises a native secretion signal peptide native to a corresponding effector molecule of the two or more effector molecules.
Embodiment 225. The engineered virus of embodiment 223 or embodiment 224, wherein one secretion signal peptide comprises a non-native secretion signal peptide non-native to at least one of the two or more effector molecules.
Embodiment 226. The engineered virus of embodiment 225, wherein the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12.
Embodiment 227. The engineered virus of any one of embodiments 223-226, wherein each secretion signal peptide is identical.
Embodiment 228. The engineered virus of any one of embodiments 199-227, wherein a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.
Embodiment 229. The engineered virus of any one of embodiments 199-228, wherein a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme.
Embodiment 230. The engineered virus of embodiment 229, wherein the therapeutic class of the first effector molecule and the second effector molecule are different.
Embodiment 231. The engineered virus of any one of embodiments 228-230, wherein the cytokine is selected from the group consisting of IL12, IL7, IL21, IL18, IL15, Type I interferons, and Interferon-gamma.
Embodiment 232. The engineered virus of embodiment 231, wherein the IL12 cytokine is an IL12p70 fusion protein.
Embodiment 233. The engineered virus of any one of embodiments 228-232, wherein the chemokine is selected from the group consisting of CCL21a, CXCL10, CXCL11, CXCL13, CXCL10-11 fusion, CCL19, CXCL9, and XCL1.
Embodiment 234. The engineered virus of any one of embodiments 228-233, wherein the growth factor is selected from the group consisting of FLT3L and GM-CSF.
Embodiment 235. The engineered virus of any one of embodiments 228-234, wherein the co-activation molecule is selected from the group consisting of 4-1BBL and CD40L.
Embodiment 236. The engineered virus of any one of embodiments 228-235, wherein the tumor microenvironment modifier is selected from the group consisting of adenosine deaminase, TGFbeta inhibitors, immune checkpoint inhibitors, VEGF inhibitors, and HPGE2.
Embodiment 237. The engineered virus of embodiment 236, wherein the TGFbeta inhibitors are selected from the group consisting of an anti-TGFbeta peptide, an anti-TGFbeta antibody, a TGFb-TRAP, and combinations thereof.
Embodiment 238. The engineered virus of embodiment 236, wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 239. The engineered virus of embodiment 236, wherein the VEGF inhibitors comprise anti-VEGF antibodies, anti-VEGF peptides, or combinations thereof.
Embodiment 240. The engineered virus of any one of embodiments 199-239, wherein at least one of the two or more effector molecules is a human-derived effector molecule.
Embodiment 241. The engineered virus of any one of embodiments 199-240, wherein one effector molecule comprises IL12.
Embodiment 242. The engineered virus of embodiment 241, wherein a second effector molecule comprises CCL21a, IL7, IL15, or IL21.
Embodiment 243. The engineered virus of any one of embodiments 208-242, wherein the polynucleotide sequence comprises:
a) an SFFV promoter; and
b) a polynucleotide described in a formula, oriented from 5′ to 3′, comprising

S1-E1-L-S2-E2

wherein
S1 comprises a polynucleotide sequence encoding a first secretion signal peptide, wherein the first secretion signal peptide is a human IL12 secretion signal peptide;
E1 comprises a polynucleotide sequence encoding a first effector molecule, wherein the first effector molecule is a human IL12p70 fusion protein;
L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;
S2 comprises a polynucleotide sequence encoding a second secretion signal peptide, wherein the second secretion signal peptide is a human IL21 secretion signal peptide;
E2 comprises a polynucleotide sequence encoding a second effector molecule, wherein the second effector molecule is human IL21; and
wherein the SFFV promoter is operably linked to the polynucleotide, the first secretion signal peptide is operably linked to the first effector molecule, and the second secretion signal peptide is operably linked to the second effector molecule.
Embodiment 244. The engineered virus of embodiment 243, wherein the polynucleotide sequence comprises the polynucleotide sequence of SEQ ID NO: 144.
Embodiment 245. The engineered virus of any one of embodiments 200-244, wherein the two or more effector molecules are capable of being transcribed and/or translated in a tumor cell.
Embodiment 246. The engineered virus of embodiment 245, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.
Embodiment 247. The engineered virus of any one of embodiments 200-246, wherein the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.
Embodiment 248. A pharmaceutical composition comprising the engineered virus of any one of embodiments 199-247 and a pharmaceutically acceptable carrier.
Embodiment 249. A method of treating a subject having cancer, the method comprising administering a therapeutically effective dose of any of the engineered viruses of any one of embodiments 199-247 or the composition of embodiment 248.
Embodiment 250. A method of reducing tumor volume in a subject, the method comprising administering to a subject having a tumor a composition comprising any of the engineered viruses of any one of embodiments 199-247 and a pharmaceutically acceptable carrier.
Embodiment 251. The method of embodiment 249 or embodiment 250, wherein the administering comprises systemic administration.
Embodiment 252. The method of embodiment 249 or embodiment 250, wherein the administering comprises intratumoral administration or intraperitoneal administration.
Embodiment 253. The method of any one of embodiments 249-252, wherein the engineered virus infects a cell in the subject and produces two or more effector molecules that modulate tumor-mediated immunosuppressive mechanisms.
Embodiment 254. The method of embodiment 253, wherein the cell is a tumor cell.
Embodiment 255. The method of any one of embodiments 249-254, wherein the method further comprises administering a checkpoint inhibitor.
Embodiment 256. The method of embodiment 255, wherein the checkpoint inhibitor is selected from the group consisting of an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody.
Embodiment 257. The method of any one of embodiments 249-256, wherein the method further comprises administering an anti-CD40 antibody.
Embodiment 258. The method of any one of embodiments 249-257, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.
Embodiment 259. The method of any one of embodiments 249-257, wherein the tumor is a tumor located in a peritoneal space.
Embodiment 260. A method of treating a subject having cancer, the method comprising administering a therapeutically effective dose of a composition, wherein the composition comprises two or more effector molecules.
Embodiment 261. A method of reducing tumor volume in a subject, the method comprising administering to a subject having a tumor a therapeutically effective dose of a composition, wherein the composition comprises two or more effector molecules.
Embodiment 262. A method of treating a subject having cancer, the method comprising administering a therapeutically effective dose of a composition, wherein the composition comprises an engineered nucleic acid, and wherein the engineered nucleic acid comprises a polynucleotide sequence encoding two or more effector molecules.
Embodiment 263. A method of reducing tumor volume in a subject, the method comprising administering to a subject having a tumor a therapeutically effective dose of a composition, wherein the composition comprises an engineered nucleic acid, and wherein the engineered nucleic acid comprises a polynucleotide sequence encoding two or more effector molecules.
Embodiment 264. The method of any one of embodiments 260-263, wherein the administering comprises one or more intraperitoneal injections.
Embodiment 265. The method of any one of embodiments 260-263, wherein the administering comprises one or more intratumoral injections.
Embodiment 266. The method of any one of embodiments 260-263, wherein the administering comprises systemic administration.
Embodiment 267. The method of any one of embodiments 262-266, wherein the engineered nucleic acid is an mRNA.
Embodiment 268. The method of any one of embodiments 262-266, wherein the engineered nucleic acid is a cDNA.
Embodiment 269. The method of any one of embodiments 262-266, wherein the composition comprises naked mRNA.
Embodiment 270. The method of any one of embodiments 262-266, wherein the composition comprises a naked plasmid.
Embodiment 271. The method of any one of embodiments 262-268, wherein the composition comprises a delivery system selected from the group consisting of a viral system, a transposon system, and a nuclease genomic editing system.
Embodiment 272. The method of embodiment 271, wherein the viral system is selected from the group consisting of a lentivirus, a retrovirus, a retrotransposon, an oncolytic virus, an adenovirus, an adeno-associated virus (AAV), and a virus-like particle (VLP).
Embodiment 273. The method of embodiment 272, wherein the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof
Embodiment 274. The method of embodiment 271, wherein the nuclease genomic editing system is selected from the group consisting of a zinc-finger system, a TALEN system, and a CRISPR system.
Embodiment 275. The method of any one of embodiments 260-268, wherein the composition comprises an erythrocyte or a platelet cell.
Embodiment 276. The method of any one of embodiments 260-268, wherein the composition comprises a lipid structure delivery system comprising a lipid-based structure.
Embodiment 277. The method of embodiment 276, wherein the lipid-based structure is selected from the group consisting of an extracellular vesicle, a lipid nanoparticle, a micelle, nanovesicle, an exosome, and a liposome.
Embodiment 278. The method of any one of embodiments 260-268, wherein the composition comprises a nanoparticle.
Embodiment 279. The method of embodiment 278, wherein the nanoparticle comprises an inorganic material.
Embodiment 280. The method of embodiment 278 or embodiment 279, wherein the nanoparticle encapsulates the engineered nucleic acid or encapsulates the two or more effector molecules.
Embodiment 281. The method of any one of embodiments 262-280, wherein the polynucleotide sequence comprises a promoter.
Embodiment 282. The method of embodiment 281, wherein the promoter comprises an exogenous promoter polynucleotide sequence.
Embodiment 283. The method of embodiment 281, wherein the promoter comprises an endogenous promoter.
Embodiment 284. The method of any one of embodiments 260-283, wherein the polynucleotide sequence further comprises a linker polynucleotide sequence.
Embodiment 285. The method of embodiment 284, wherein the promoter is operably linked to the polynucleotide sequence such that the two or more effector molecules are capable of being transcribed as a single polynucleotide comprising the formula (L-E)x, wherein
L comprises a linker polynucleotide sequence,
E comprises a polynucleotide encoding one of the two or more effector molecules,

### X=2 to 20, and

wherein for the first iteration of the (L-E) unit L is absent.
Embodiment 286. The method of embodiment 284 or embodiment 285 wherein the linker polynucleotide sequence is operably associated with the translation of the two or more effector molecules as separate polypeptides.
Embodiment 287. The method of any one of embodiments 284-286, wherein the linker polynucleotide sequence encodes a 2A ribosome skipping tag.
Embodiment 288. The method of embodiment 287, wherein the 2A ribosome skipping tag is selected from the group consisting of P2A, T2A, E2A, and F2A.
Embodiment 289. The method of embodiment 284-288, wherein the linker polynucleotide sequence encodes a cleavable polypeptide.
Embodiment 290. The method of embodiment 289, wherein the cleavable polypeptide comprises a furin polypeptide sequence.
Embodiment 291. The method of any one of embodiments 284-286, wherein the linker polynucleotide sequence encodes an Internal Ribosome Entry Site (IRES).
Embodiment 292. The method of any one of embodiments 284-291, wherein the linker polynucleotide sequence encodes an additional promoter.
Embodiment 293. The method of embodiment 292, wherein the promoter is operably linked to the polynucleotide sequence such that a first polynucleotide encoding one of the two or more effector molecules is capable of being transcribed,
wherein the additional promoter is operably linked to the polynucleotide sequence such that a second polynucleotide encoding a second of the two or more effector molecules is capable of being transcribed, and
wherein the first and the second polynucleotides are separate polynucleotides.
Embodiment 294. The method of embodiment 292 or embodiment 293, wherein the promoter and the additional promoter are identical.
Embodiment 295. The method of embodiment 292 or embodiment 293, wherein the promoter and the additional promoter are different.
Embodiment 296. The method of any one of embodiments 281-295, wherein the promoter and/or the additional promoter comprises a constitutive promoter.
Embodiment 297. The method of embodiment 296, wherein the constitutive promoter is selected from the group consisting of CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
Embodiment 298. The method of any one of embodiments 281-297, wherein the promoter and/or the additional promoter comprises an inducible promoter.
Embodiment 299. The method of embodiment 298, wherein the inducible promoter is selected from the group consisting of minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.
Embodiment 300. The method of any one of embodiments 260-299, wherein each of the two or more effector molecules comprises a secretion signal.
Embodiment 301. The method of embodiment 300, wherein one secretion signal peptide comprises a native secretion signal peptide native to a corresponding effector molecule of the two or more effector molecules.
Embodiment 302. The method of embodiment 300 or embodiment 301, wherein one secretion signal peptide comprises a non-native secretion signal peptide non-native to at least one of the two or more effector molecules.
Embodiment 303. The method of embodiment 302, wherein the non-native secretion signal peptide is selected from the group consisting of IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, IL21, CCL2, TIMP2, VEGFB, osteoprotegerin, serpin E1, GROalpha, and CXCL12.
Embodiment 304. The method of any one of embodiments 300-303, wherein each secretion signal peptide is identical.
Embodiment 305. The method of any one of embodiments 260-304, wherein a first effector molecule is selected from a therapeutic class, wherein the therapeutic class is selected from the group consisting of a cytokine, a chemokine, a growth factor, a co-activation molecule, a tumor microenvironment modifier a, a receptor, a ligand, an antibody, a polynucleotide, a peptide, and an enzyme.
Embodiment 306. The method of any one of embodiments 260-305, wherein a second effector molecule is selected from a therapeutic class, wherein the therapeutic class is selected from the group consisting of a cytokine, a chemokine, a growth factor, a co-activation molecule, a tumor microenvironment modifier, a receptor, a ligand, an antibody, a polynucleotide, a peptide, and an enzyme.
Embodiment 307. The method of embodiment 306, wherein the therapeutic class of the first effector molecule and the second effector molecule are different.
Embodiment 308. The method of any one of embodiments 305-307, wherein the cytokine is selected from the group consisting of IL12, IL7, IL21, IL18, IL15, Type I interferons, and Interferon-gamma.
Embodiment 309. The method of embodiment 308, wherein the IL12 cytokine is an IL12p70 fusion protein.
Embodiment 310. The method of any one of embodiments 305-309, wherein the chemokine is selected from the group consisting of CCL21a, CXCL10, CXCL11, CXCL13, CXCL10-11 fusion, CCL19, CXCL9, and XCL1.
Embodiment 311. The method of any one of embodiments 305-310, wherein the growth factor is selected from the group consisting of FLT3L and GM-CSF.
Embodiment 312. The method of any one of embodiments 305-311, wherein the co-activation molecule is selected from the group consisting of 4-1BBL and CD40L.
Embodiment 313. The method of any one of embodiments 305-312, wherein the tumor microenvironment modifier is selected from the group consisting of adenosine deaminase, TGFbeta inhibitors, immune checkpoint inhibitors, VEGF inhibitors, and HPGE2.
Embodiment 314. The method of embodiment 313, wherein the TGFbeta inhibitors are selected from the group consisting of an anti-TGFbeta peptide, an anti-TGFbeta antibody, a TGFb-TRAP, and combinations thereof.
Embodiment 315. The method of embodiment 313, wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.
Embodiment 316. The method of embodiment 313, wherein the VEGF inhibitors comprise anti-VEGF antibodies, anti-VEGF peptides, or combinations thereof.
Embodiment 317. The method of any one of embodiments 260-316, wherein at least one of the two or more effector molecules is a human-derived effector molecule.
Embodiment 318. The method of any one of embodiments 260-317, wherein one effector molecule comprises IL12.
Embodiment 319. The method of embodiment 318, wherein a second effector molecule comprises CCL21a, IL7, IL15, or IL21.
Embodiment 320. The method of any one of embodiments 285-319, wherein the polynucleotide sequence comprises:
a) an SFFV promoter; and
b) an expression cassette described in a formula, oriented from 5′ to 3′, comprising

S1-E1-L-S2-E2

wherein
S1 comprises a polynucleotide sequence encoding a first secretion signal peptide, wherein the first secretion signal peptide is a human IL12 secretion signal peptide;
E1 comprises a polynucleotide sequence encoding a first effector molecule, wherein the first effector molecule is a human IL12p70 fusion protein;
L comprises a linker polynucleotide sequence, wherein the linker polynucleotide sequence encodes a Furin recognition polypeptide sequence, a Gly-Ser-Gly polypeptide sequence, and a T2A ribosome skipping tag in a Furin:Gly-Ser-Gly:T2A orientation from N-terminus to C-terminus;
S2 comprises a polynucleotide sequence encoding a second secretion signal peptide, wherein the second secretion signal peptide is a human IL21 secretion signal peptide;
E2 comprises a polynucleotide sequence encoding a second effector molecule, wherein the second effector molecule is human IL21; and
wherein the SFFV promoter is operably linked to the expression cassette, the first secretion signal peptide is operably linked to the first effector molecule, and the second secretion signal peptide is operably linked to the second effector molecule.
Embodiment 321. The method of embodiment 320, wherein the polynucleotide sequence comprises the polynucleotide sequence of SEQ ID NO: 144.
Embodiment 322. The method of any one of embodiments 263-321, wherein the tumor is selected from the group consisting of a bladder tumor, a brain tumor, a breast tumor, a cervical tumor, a colorectal tumor, an esophageal tumor, a glioma, a kidney tumor, a liver tumor, a lung tumor, a melanoma, an ovarian tumor, a pancreatic tumor, a prostate tumor, a skin tumor, a thyroid tumor, and a uterine tumor.

### EXAMPLES

Below are examples of specific embodiments for carrying out the present invention. The examples are offered for illustrative purposes only, and are not intended to limit the scope of the present invention in any way. For example, the experiments described and performed below demonstrate the general utility of engineering cells to secrete payloads (e.g., effector molecules) and delivering those cells to induce an immunogenic response against tumors. Specifically, the examples described below demonstrate the ability to induce an immunogenic response against tumors through delivery of engineered mesenchymal stem cells (MSCs) secreting a variety of the effector molecules described herein. The use of MSCs is not intended to limit the scope of the present invention directed to engineering the cells described herein.

Efforts have been made to ensure accuracy with respect to numbers used (e.g., amounts, temperatures, etc.), but some experimental error and deviation should, of course, be allowed for.

### Example 1

This Example describes the in vitro characterization of MSCs with individual and combination immunotherapy payloads. Direct anti-cancer effects of immunotherapy-expressing MSCs on cancer cells are first measured. Next, the effects of immunotherapy-expressing MSCs on co-cultures with primary immune cells (focusing on T cells) and cancer cells are measured. The immuno-stimulatory properties of immunotherapy-expressing MSCs are rank-ordered based on inflammatory biomarker panels in both mouse and human cell systems. Immunotherapy-expressing MSCs that significantly enhance cancer cell killing either on their own or together with T cells are identified, and the top candidates to advance to in vivo testing are selected.

Methods: Immunotherapy-expressing MSCs are engineered to express the effector molecules listed in Table 1 are evaluated for their functional effects using in vitro models relevant to cancer therapy. Human ovarian cancer cells (e.g., OVCAR8 and SKOV3) and human immune cells isolated from circulating PBMCs are used to test the hMSCs expressing hITs. Mouse ovarian cancer cells (e.g., ID8) and mouse immune cells are used to test the mMSCs expressing mITs.

Checkpoint inhibitors. Cell-binding assays are used to verify the activity of the expressed antibodies. The targets of the antibodies, CTLA4 and PD1, both negatively regulate T cells, but they are upregulated at different stages of T-cell activation (Boutros C, et al. (2016) Nat Rev Clin Oncol 13(8):473-486; Valsecchi M E (2015) New Engl J Med 373(13):1270-1270). CTLA4 is briefly upregulated in the priming phase, whereas PD1 is consistently expressed in the effector phase of T cell activation (Pardoll D M (2012) Nat Rev Cancer 12(4):252-264; Legat A, et al. (2013) Front Immunol 4:455). Anti-CTLA4 antibody binds to CTLA4 on the T-cell surface, blocking CTLA4 from shutting down T-cell activation in the early stage, and the human anti-PD1 antibody binds to PD1, preventing tumor cells from inhibiting T-cell activity.

T cells are isolated from PBMC by negative selection using EASYSEP™ magnetic bead (STEMCELL Technologies). The isolated T cells are activated by Human T-Activator CD3/28 Dynabeads (Thermo Fisher) and expression of CTLA-4 and PD-1 is monitored over 5 days to select for optimal timing of expression for each surface marker. On the appropriate days, conditioned media from the MSCs expressing antibodies for CTLA-4 or PD-1, or control conditioned media from non-expressing MSCs, are applied to the activated T cells to validate direct cell-surface-receptor binding of these antibodies. Fluorochrome-labeled secondary detection antibodies together with flow cytometry should confirm binding.

Chemokines. CCL21 chemokine functionality is confirmed using cell migration assays and isolated naive T cells, which express chemokine receptor CCR7 that is responsive to CCL21 chemotaxis. Specifically, CCL21-expressing or control MSCs are added to one compartment of a trans-well and then cell migration is assessed by isolated naive T cells from the other compartment, followed by enumeration of numbers of migrated T cells (Justus C R, et al. (2014) J Vis Exp (88)).

Cytokines. The activity of IL2, IL12, and IL15 is measured. ELISA assays specific to IL2, IL12, and IL15 are used to detect levels of these cytokines in MSC supernatants. Functional bioactivity assays employ the CTLL-2 cell line to assess of IL2 or IL15-mediated proliferation, or the NKG cell line to assess IL12-mediated IFN-gamma production by MSC supernatants. Multiplexed cytokine profiling assays using LUMINEX® technology may also be used to assess cytokine expression and effects on immune cells.

STING pathway. STING pathway activation is measured with the constitutive STING mutant payload. Using LUMINEX® beads, the secretion of Type I interferons (e.g. IFN-alpha2 and IFN-beta) with expression of the STING mutant are profiled in MSCs.

Direct effects of immunotherapy-expressing MSCs on ovarian cancer cells. Any direct effects of MSCs on ovarian cancer cell growth and viability are tested in vitro. For example, mMSC or hMSC candidates are co-cultured with the mouse ovarian cancer cell line (ID8) or human ovarian cancer cell lines (OVCAR8 and SKOV3) and cancer cell cytotoxicity is measured by the well-characterized lactate dehydrogenase (LDH) assay. After 24 hours of co-culture, the supernatants are collected and measured for LDH levels correlated to cellular death via an enzymatic reaction that is subsequently quantified by specific absorbance on a plate reader. Additionally, cancer cell numbers are assessed by counting live versus dead cells by Trypan Blue exclusion and live versus apoptotic/dead cells by flow cytometric measurement using Annexin-V and propidium iodide staining.

Effects of immunotherapy-expressing MSCs on T cell and ovarian cancer cell co-culture systems. Tests determine whether immunotherapy-expressing MSCs can stimulate immune cells, such as T cells, to have improved anti-cancer activity against ovarian cancer cells in vitro. Specifically, mMSC-mIT candidates are co-cultured with mouse splenocytes and the ID8 cancer cell line, or hMSC-hIT candidates are co-cultured with human PBMCs and the OVCAR8 or SKOV3 cell lines. The co-culture assays entail using PBMCs/splenocytes with the ovarian cancer cells, with or without the MSCs, and stimulation with anti-CD3/28 beads. To assess cancer cell death, 16 hour killing assays are performed using techniques such as LDH cytotoxicity measurements, combining dye-labeled ovarian cancer cells with non-labeled effector PBMCs/splenocytes at fixed ratios and assaying killing by flow cytometry (Jedema I, et al. (2004) Blood 103(7):2677-2682), and apoptosis readouts by flow cytometry using Annexin-V with propidium iodide. T cell activation/proliferation is specifically assay by CFSE cell division at 3-5 days and cytokine production of IFN-gamma at 1-3 days.

An alternative strategy to generate T cells expressing CTLA-4 and PD1 is to activate with phytohaemagglutinin (PHA) to express the cell surface receptors PD1 and CTLA4. On Day 3, —99% of the activated T cells should express PD1 while ˜15% of them should express CTLA4 (Pardoll D M (2012) Nat Rev Cancer 12(4):252-264; Legat A, et al. (2013) Front Immunol 4:455). On Day 10, the activated T cells should be in the effector phase, when CTLA4 expression is downregulated but PD1 expression is maintained. Direct cell-surface-receptor binding of these antibodies is evaluated. On Day 3 and Day 10 post-induction, MSCs with the respective checkpoint inhibitor antibody expression constructs are applied to the T cell cultures. Labeled detection antibodies are used together with flow cytometry to confirm binding. Commercial antibodies are used as controls.

### Example 2

This Example describes the in vivo characterization of MSCs expressing immunotherapy payloads in a syngeneic ovarian cancer model. The anti-tumor efficacy of immunotherapy-expressing MSCs is characterized using syngeneic mouse models of ovarian cancer (mMSC-mIT with mouse immune system). Tumor homing of engineered MSCs and expression of individual and combinatorial immunotherapies in a syngeneic ovarian mouse model are measured. Ovarian tumor burden and mouse survival with engineered MSC treatments are also measured. This Example should demonstrate selective homing of engineered MSCs to the TME and localized production of immunotherapy factors in ovarian tumors versus other body sites. This Example should also demonstrate significant reductions in tumor burden and extension of mouse survival with immunotherapy-expressing engineered MSCs.

Methods: The mouse ID8 cell line originated from spontaneous transformation of mouse ovarian epithelial surface cells (MOSE), is used to create a syngeneic ovarian tumor model (Roby K F, et al. (2000) Carcinogenesis 21(4):585-591). Derivatives of the ID8 cell line are also used (e.g., ID8-VEGF (ID8-Defb29/Vegf-a), ID8-P53DN, ID8-P53KO-PTEN KO, ID8-P53KO—BRCA2 KO, ID8-P53KO-BRCA1 KO, ID8-PD53KO-Nf1KO). The ID8 cell line is infected with a lentivirus expressing Renilla luciferase (rLuc) to allow for in vivo bioluminescence imaging that is orthogonal to MSCs expressing Firefly luciferase (ffLuc). Successful rLuc expression is confirmed in ID8 in vitro prior to establishing the syngeneic ovarian cancer model in mice. For the syngeneic model, 5×10⁵ ID8 cells are injected into the peritoneal cavity of C57BL/6 mice between 6 to 8 weeks old (36, 54). MSCs are engineered as in Example 1, along with an ffLuc-expressing plasmid.

mMSC-mIT candidates are introduced into the syngeneic mouse model starting on day 25 (after tumor cell injection) at a dose of 10⁶ MSC per animal once per week for 5 weeks (Dembinski J L, et al. (2013) Cytotherapy 15(1):20-32). The ovarian tumor load and mMSC-mIT candidates are visualized over time through rLuc and ffLuc bioluminescence imaging, respectively, as well as histological analyses following terminal time points. Mice are euthanized when they develop signs of distress, such as body-weight loss, ruffled fur, poor body posture, distended abdomen, and jaundice. Survival curves for the mice are measured. Distal metastasis of tumor cells is quantified by bioluminescence imaging (BLI) and by necropsy at time of euthanasia. Immune system profiling and activity is measured at different time points as biomarkers of response to the therapy.

To assess for variability in the expected anti-tumor effects of the MSCs, the dose of ID8 cells used to establish the model is varied (e.g., increase the number of cells to 5×10⁶), the dose of MSCs used is changed, and the time when MSCs are delivered after tumor establishment is modulated.

Even though mMSCs have been shown to home to ovarian tumors in mouse models, it is possible that some payloads disrupt this homing activity. In these instances, expression of these payloads may be engineered to be inducible. This can be achieved, for example, with a phloretin-inducible system (Gitzinger M, et al. (2009) Proc Natl Acad Sci USA 106(26):10638-10643). Alternatively, the Dimerizer system may be used to link a synthetic zinc-finger DNA-binding domain with a transactivator domain using a small molecule (Clackson T, et al. (1998) Proc Natl Acad Sci USA 95(18):10437-10442). Alternatively or additionally, inducible payload expression constructs that are triggered in the tumor microenvironment based on signals such as low O₂ may be constructed.

Lentiviral ffLuc constructs may also be used to infect MSCs.

### Example 3

This Example describes the in vivo characterization of the efficacy of MSCs expressing immunotherapy payloads in xenograft models of human ovarian cancer in mice with human immune cells. The activity of engineered MSCs in human ovarian cancer models in immunodeficient mice that are engrafted with human immune cells via CD34+ cell transplants (hMSC-hIT with humanized immune system) is tested. Homing of engineered MSCs and expression of individual and combinatorial immunotherapies in human xenograft ovarian tumors in mice with human immune cells are measured. Ovarian tumor burden and mouse survival with engineered MSC treatments are also tested. This Example should demonstrate elevated homing of engineered MSCs and localized production of immunotherapy factors into human xenograft ovarian tumors versus other body sites in mice. This Example should also demonstrate significant reductions in tumor burden and extension of mouse survival with immunotherapy-expressing engineered MSCs correlating with changes in the immune system composition.

Methods. To enable translation of engineered MSCs into human clinical trials, hMSC-hIT constructs are tested in humanized mouse models of human cancers. The effects of the immunotherapy-expressing hMSCs in mice are modeled by using xenografts of human ovarian cancer cell lines in immuno-deficient mice (NSG) engrafted with CD34⁺ hematopoietic stem cells (HSCs).

For human ovarian cancer cells, OVCAR8 and SKOV3 cell lines are used. Similar assays as described in Example 3 are used to investigate tumor load and mouse survival over time.

Two alternative approaches may also be used. (1) Human T cells can be infused into the mice. (2) Human PBMCs can be infused into the mice.

Expression Vector: pL + MCS
(SEQ ID NO: 111)
ACGCGTGTAGTCTTATGCAATACTCTTGTAGTCTTGCAACATGGTAACGATGAGTTAGCAACATGC
CTTACAAGGAGAGAAAAAGCACCGTGCATGCCGATTGGTGGAAGTAAGGTGGTACGATCGTGCCT
TATTAGGAAGGCAACAGACGGGTCTGACATGGATTGGACGAACCACTGAATTGCCGCATTGCAGA
GATATTGTATTTAAGTGCCTAGCTCGATACAATAAACGGGTCTCTCTGGTTAGACCAGATCTGAGC
CTGGGAGCTCTCTGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAGACCCTTTTAGTCA
GTGTGGAAAATCTCTAGCAGTGGCGCCCGAACAGGGACCTGAAAGCGAAAGGGAAACCAGAGCT
CTCTCGACGCAGGACTCGGCTTGCTGAAGCGCGCACGGCAAGAGGCGAGGGGCGGCGACTGGTGA
GTACGCCAAAAATTTTGACTAGCGGAGGCTAGAAGGAGAGAGATGGGTGCGAGAGCGTCAGTATT
AAGCGGGGGAGAATTAGATCGCGATGGGAAAAAATTCGGTTAAGGCCAGGGGGAAAGAAAAAAT
ATAAATTAAAACATATAGTATGGGCAAGCAGGGAGCTAGAACGATTCGCAGTTAATCCTGGCCTG
TTAGAAACATCAGAAGGCTGTAGACAAATACTGGGACAGCTACAACCATCCCTTCAGACAGGATC
AGAAGAACTTAGATCATTATATAATACAGTAGCAACCCTCTATTGTGTGCATCAAAGGATAGAGAT
AAAAGACACCAAGGAAGCTTTAGACAAGATAGAGGAAGAGCAAAACAAAAGTAAGACCACCGCA
CAGCAAGCGGCCACTGATCTTCAGACCTGGAGGAGGAGATATGAGGGACAATTGGAGAAGTGAAT
TATATAAATATAAAGTAGTAAAAATTGAACCATTAGGAGTAGCACCCACCAAGGCAAAGAGAAGA
GTGGTGCAGAGAGAAAAAAGAGCAGTGGGAATAGGAGCTTTGTTCCTTGGGTTCTTGGGAGCAGC
AGGAAGCACTATGGGCGCAGCCTCAATGACGCTGACGGTACAGGCCAGACAATTATTGTCTGGTA
TAGTGCAGCAGCAGAACAATTTGCTGAGGGCTATTGAGGCGCAACAGCATCTGTTGCAACTCACA
GTCTGGGGCATCAAGCAGCTCCAGGCAAGAATCCTGGCTGTGGAAAGATACCTAAAGGATCAACA
GCTCCTGGGGATTTGGGGTTGCTCTGGAAAACTCATTTGCACCACTGCTGTGCCTTGGAATGCTAG
TTGGAGTAATAAATCTCTGGAACAGATTGGAATCACACGACCTGGATGGAGTGGGACAGAGAAAT
TAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGG
CTGTGGTATATAAAATTATTCATAATGATAGTAGGAGGCTTGGTAGGTTTAAGAATAGTTTTTGCT
GTACTTTCTATAGTGAATAGAGTTAGGCAGGGATATTCACCATTATCGTTTCAGACCCACCTCCCA
ACCCCGAGGGGACCCGACAGGCCCGAAGGAATAGAAGAAGAAGGTGGAGAGAGAGACAGAGAC
AGATCCATTCGATTAGTGAACGGATCTCGACGGTATCGGTTAACTTTTAAAAGAAAAGGGGGGAT
TGGGGGGTACAGTGCAGGGGAAAGAATAGTAGACATAATAGCAACAGACATACAAACTAAAGAA
TTACAAAAACAAATTACAAAAATCAAAATTTTATCTCGACATGGTGGCGACCGGTAGCGCTAGCG
GATCGATAAGCTTGATATCGCCTGCAGCCGAATTCCTTGACTTGGGATCCGCGTCAAGTGGAGCAA
GGCAGGTGGACAGTCCTGCAGGCATGCGTGACTGACTGAGGCCGCGACTCTAGTTTAAACTGCGT
GACTGACTCTAGAAGATCCGGCAGTGCGGCCGCGTCGACAATCAACCTCTGGATTACAAAATTTGT
GAAAGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCCTGTGT
TGCCACCTGGATTCTGCGCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTGGTACCTTTAAGACCAATGACTTACAAGGCAGCTGTA
GATCTTAGCCACTTTTTAAAAGAAAAGGGGGGACTGGAAGGGCTAATTCACTCCCAACGAAAATA
AGATCTGCTTTTTGCTTGTACTGGGTCTCTCTGGTTAGACCAGATCTGAGCCTGGGAGCTCTCTGGC
TAACTAGGGAACCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTGGAAAATCTCTA
GCAGTAGTAGTTCATGTCATCTTATTATTCAGTATTTATAACTTGCAAAGAAATGAATATCAGAGA
GTGAGAGGAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCA
TGTCTGGCTCTAGCTATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAG
GAGGCTTTTTTGGAGGCCTAGACTTTTGCAGAGACGGCCCAAATTCGTAATCATGGTCATAGCTGT
TTCCTGTGTGAAATTGTTATCCGCTCACAATTCCACACAACATACGAGCCGGAAGCATAAAGTGTA
AAGCCTGGGGTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGAAAG
AACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGGACAGTATTTGGTATCTGCGCTC
TGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGA
TCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGGCGAAAACTCT
CAAGGATCTTACCGCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAATGCCGCAAAAAAG
GGAATAAGGGCGACACGGAAATGTTGAATACTCATACTCTTCCTTTTTCAATATTATTGAAGCATT
TATCAGGGTTATTGTCTCATGAGCGGATACATATTTGAATGTATTTAGAAAAATAAACAAATAGGG
GTTCCGCGCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGGTGAAAAC
CTCTGACACATGCAGCTCCCGGAGACGGTCACAGCTTGTCTGTAAGCGGATGCCGGGAGCAGACA
AGCCCGTCAGGGCGCGTCAGCGGGTGTTGGCGGGTGTCGGGGCTGGCTTAACTATGCGGCATCAG
AGCAGATTGTACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATCGGTGCGGGC
CTCTTCGCTATTACGCCAGCTGGCGAAAGGGGGATGTGCTGCAAGGCGATTAAGTTGGGTAACGC
CAGGGTTTTCCCAGTCACGACGTTGTAAAACGACGGCCAGTGCCAAGCTG

### Example 4. 4T1 Triple Negative Breast Carcinoma

In the following experiments, MSCs were engineered to express one of the following effector molecules, then administered, alone or in combinations, to an orthotopic breast cancer mouse model: IFNβ, IFNγ, IL12, IL15, IL36γ, IL7, TRAIL, cGAS, CCL21a, OX40L, CD40L, or HACv-PD1. In some examples, a checkpoint inhibitor (anti-CD40, anti-PD1, or anti-CTLA-4 antibody) was injected in combination with administration with the engineered MSCs.

MSC Homing

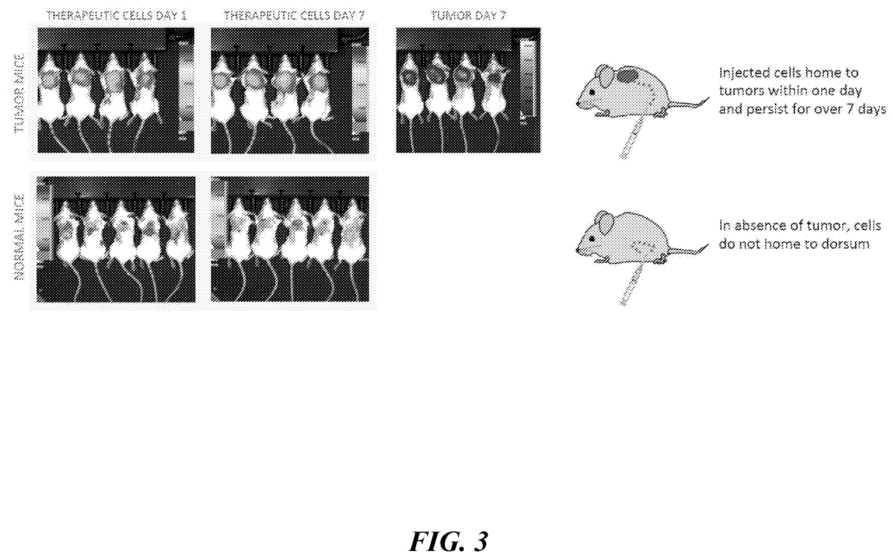

The following experiments demonstrate that murine MSCs home to tumors in an orthotopic mouse model of breast cancer. Luciferase-expressing 4T1 breast tumor cells (5×10⁵) were orthotopically implanted into the dorsal fat pad of female BALB/cJ mice. After 5 days, mice were intraperitoneally injected with 1 million fluorescently-labeled (with XenoLight DiR (Caliper Life Sciences)) murine BM-derived MSCs (BM-MSCs, therapeutic cells). At days 1 and 7 after MSC injection, fluorescence analysis was used to determine MSC localization using the Ami HT live animal imager (Spectral Instruments). On day 7, tumor localization and size was determined through the 4T1 cell's luciferase bioluminescence reporter using the Ami HT imager. As shown in FIG. 3, the injected MSCs co-localized to the site of the tumor, indicating that these cells do in fact specifically home in vivo to sites of 4T1 breast tumors. The injected MSCs home to tumors within one day and persist for over 7 days. In contrast, injected MSCs do not home to the dorsum in the absence of tumor in normal mice. These results suggest that MSCs can be used as a delivery vehicle for anti-cancer molecules, proteins or compounds.

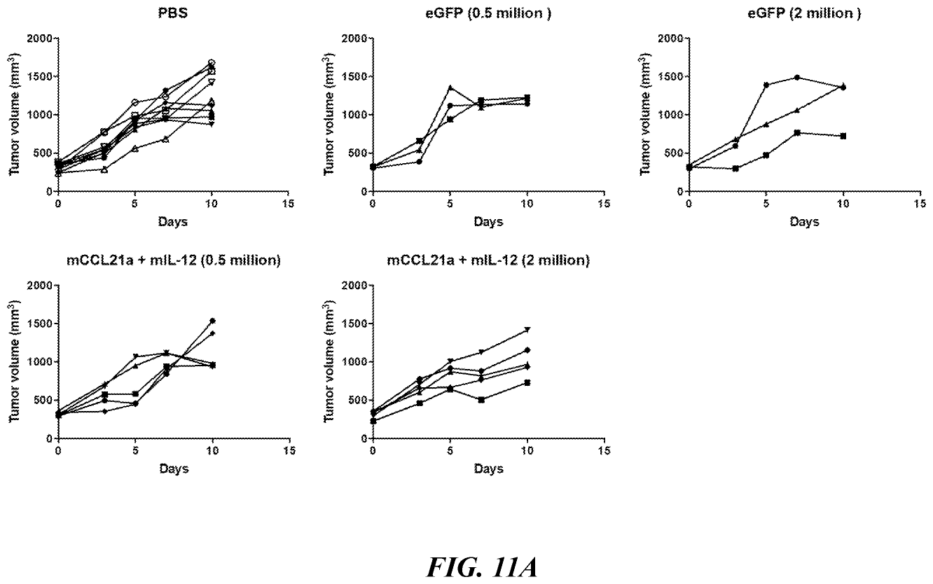

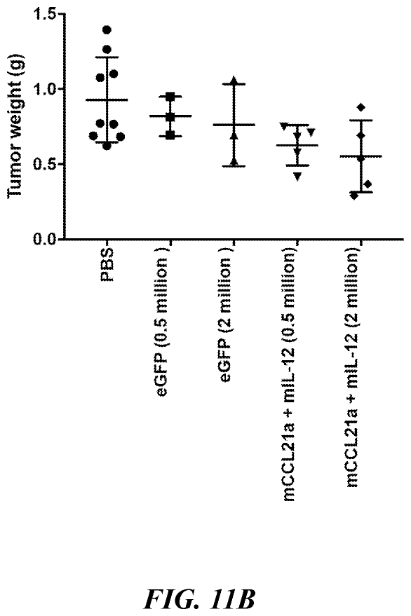

To determine whether engineered human MSCs can home toward mouse tumors, different lines of engineered human MSC expressing either GFP, IL2 or CCL21a were injected into BALB/c mice with 4T1 tumors. Efficacy was determined by tumor volume from caliper measurement every other day. FIGS. 11A-11B show that human MSCs do not home to mouse 4T1 tumors.

In Vivo Efficacy

The following experiments demonstrate the in vivo efficacy of MSCs expressing immunotherapy effectors (payloads) in the orthotopic model of breast cancer. 4T1-Neo-Fluc mouse breast tumor cells (Imanis Life Sciences, 5×10⁵ cells) were implanted orthotopically into the dorsal fat pad of female BALB/cJ mice (The Jackson Laboratory). Mice were then randomized into the treatment groups 5 days after tumor implantation. Mice received intraperitoneal injection of either control MSC growth media or engineered MSCs (2×10⁶ cells) expressing different immunotherapy effectors (payloads) once a week for two weeks. Each immunotherapy was expressed by a different MSC, and MSCs were combined (1:1 ratio) for combinatorial treatment. Tumor growth was monitored by caliper measurements every other day, and mouse weights were recorded twice weekly. Mice were euthanized 14 days after first MSC treatment and tissues were collected for further analysis.

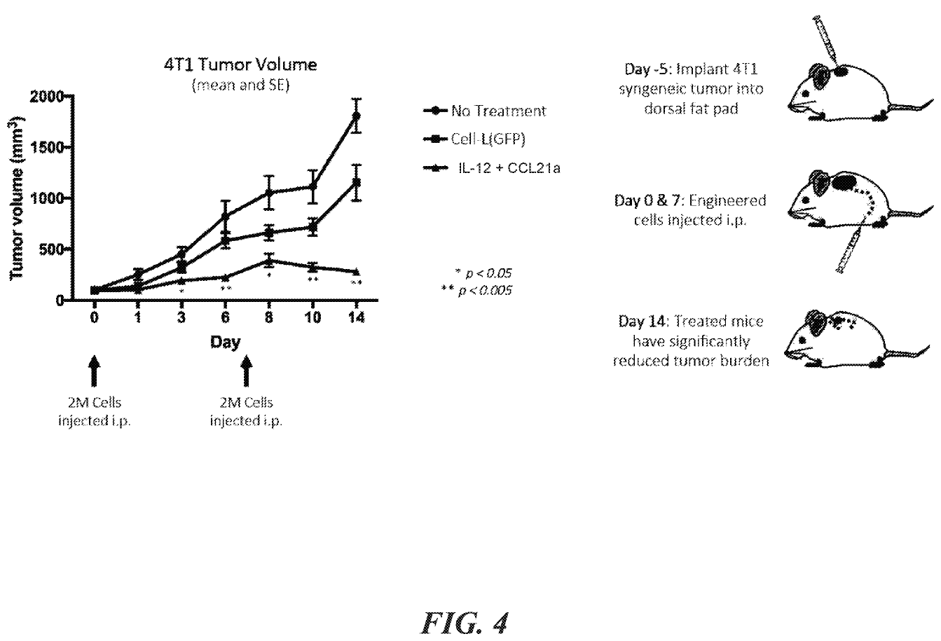

FIG. 4 shows that tumor growth was delayed in mice treated with engineered MSCs expressed combinatorial genes IL-12 and CCL21a compared to controls treated with media.

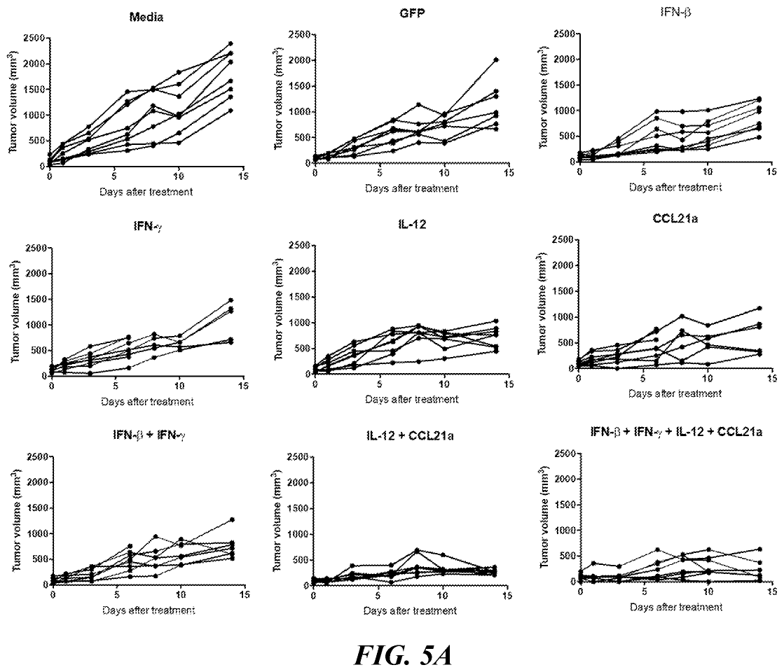

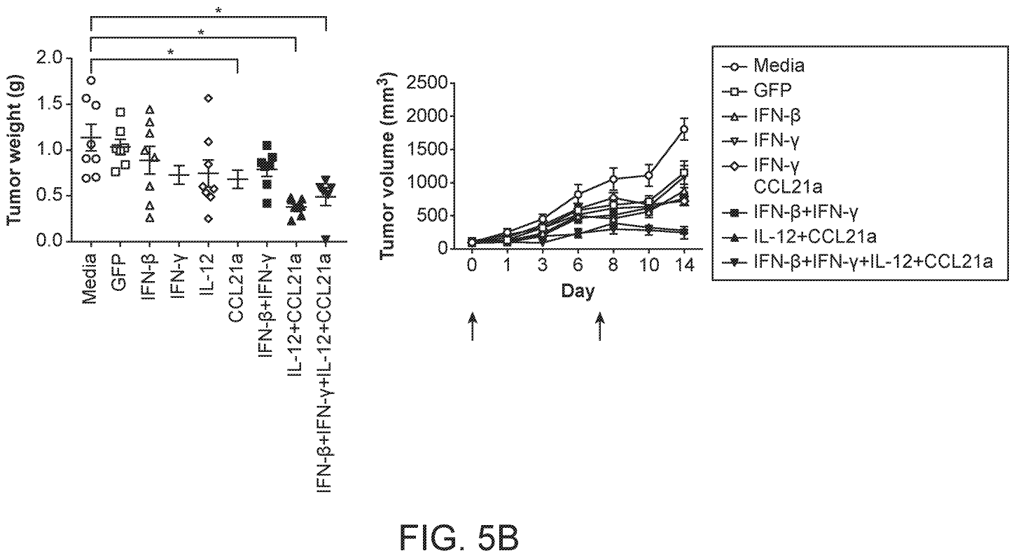

FIGS. 5A-5C show that engineered MSCs that express single immunotherapy effectors (e.g., IFN-β, IFN-γ, IL-12 or CCL21a) inhibited growth of syngeneic 4T1 mouse tumors compared to media-treated mice. Surprisingly, a synergistic effect on tumor growth was observed when the immunotherapy effectors were combined, particularly the combination of IL-12 and CCL21a, and the combination of IFN-β, IFN-γ, IL-12 and CCL21a (FIGS. 5A-5C).

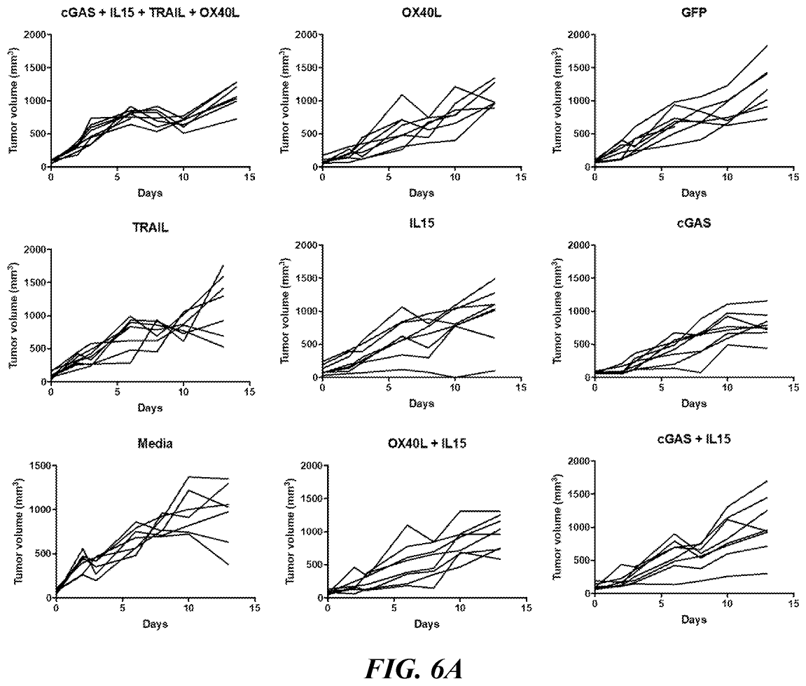

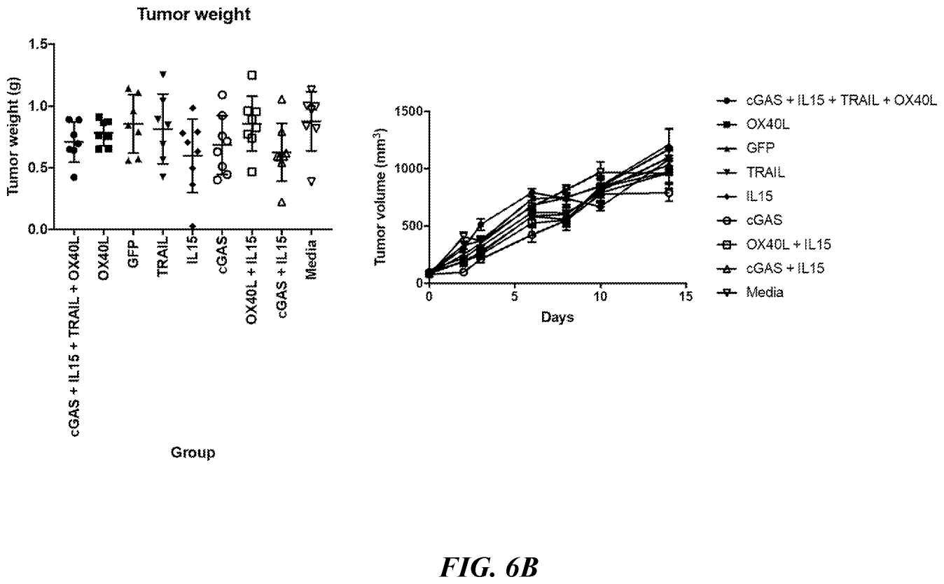

FIGS. 6A-6B show that engineered MSCs expressing OX40L, TRAIL, IL15, cGAS, or combinations thereof do not inhibit tumor growth.

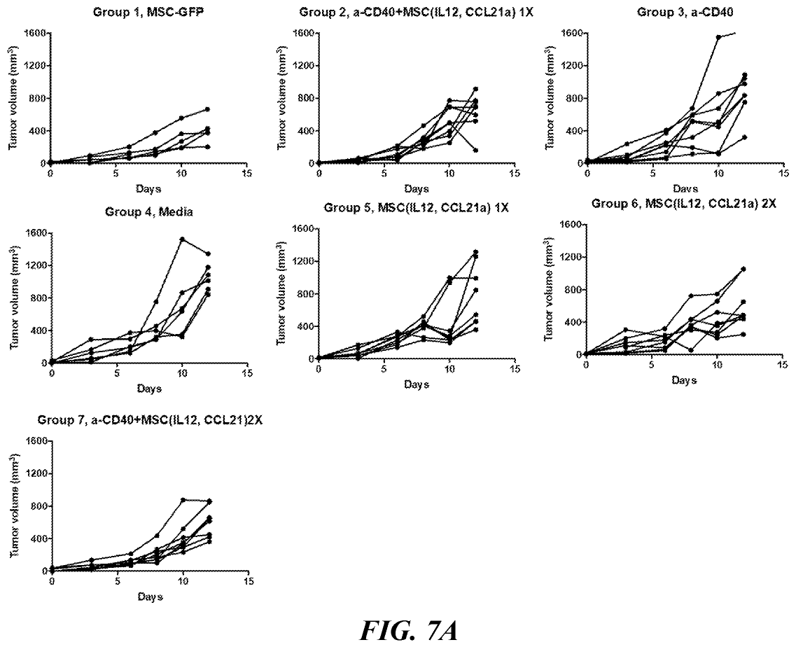

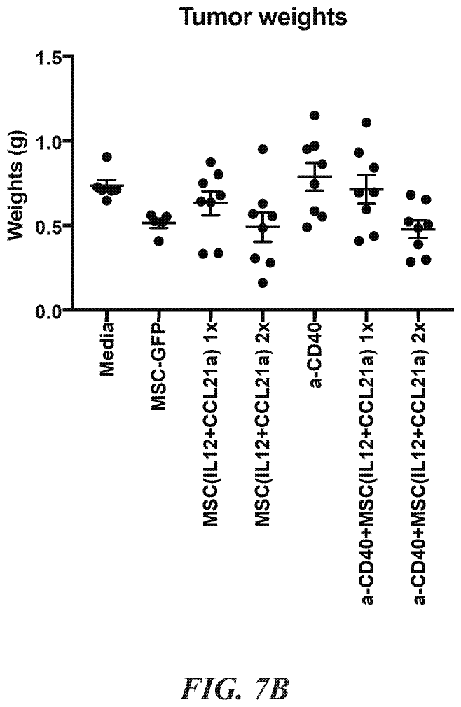

FIGS. 7A-7B show that engineered MSCs expressing IL-12 and CCL21a inhibit tumor growth; however the addition of anti-CD40 antibody does not reduce tumor growth.

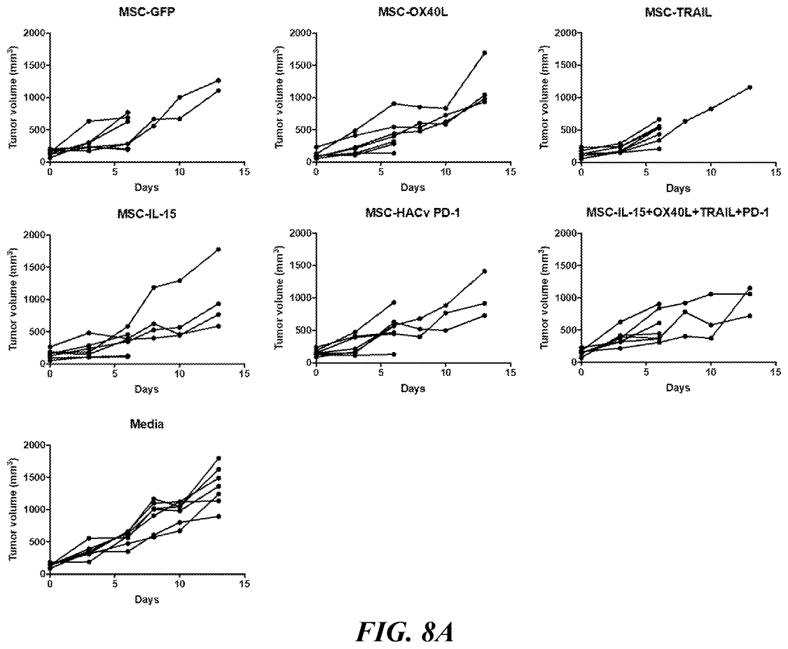

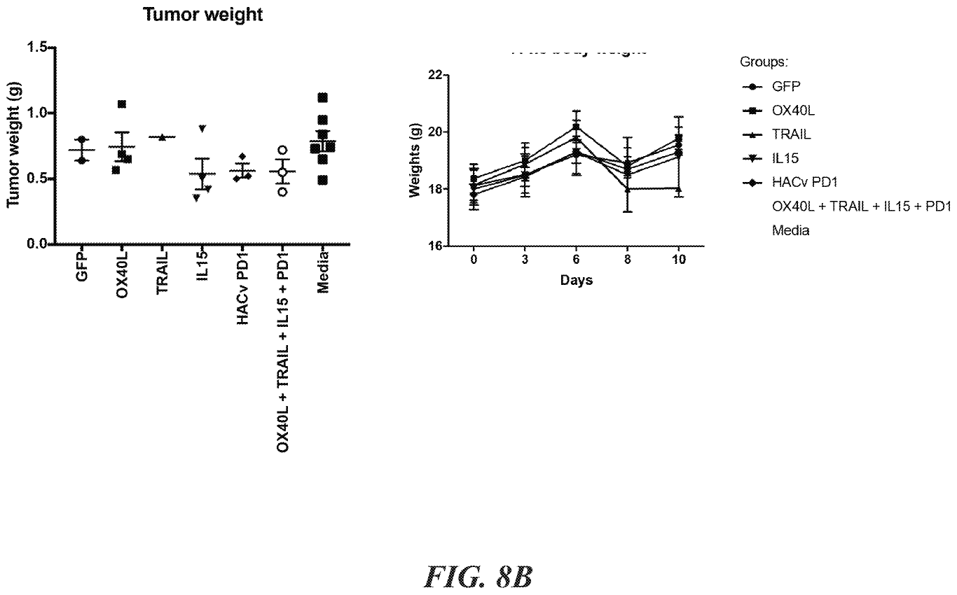

FIGS. 8A-8B show that engineered MSCs expressing OX40L, TRAIL, IL15, HACvPD-1, or combinations thereof do not inhibit tumor growth significantly in a subcutaneous breast cancer model.

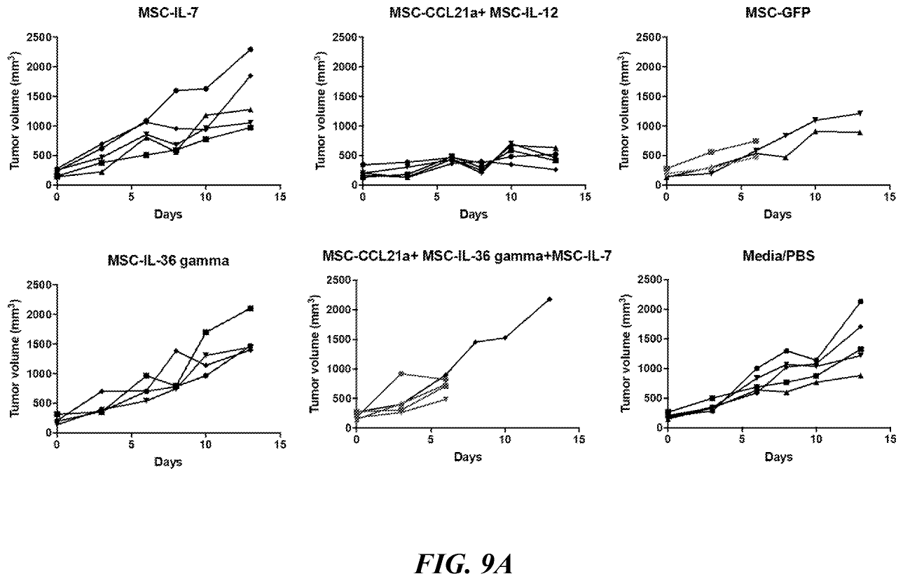

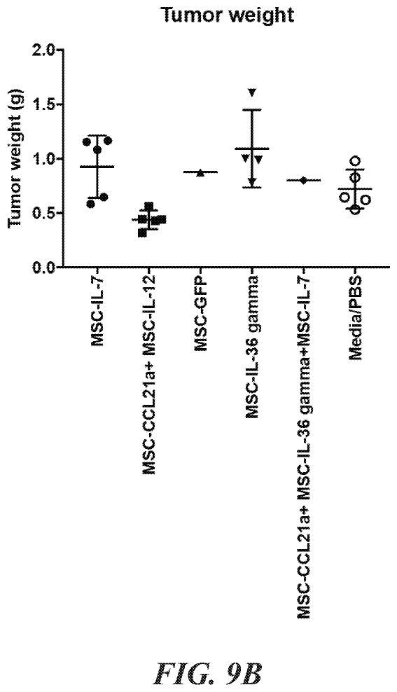

FIGS. 9A-9B show that engineered MSCs expressing IL-12 and CCL21a inhibit tumor growth; however the combination of MSCs expressing CCL21a, IL-36 gamma and IL-7 does not reduce tumor growth. Some of the effector combinations tested, however, may cause toxicity.

Dose Escalation

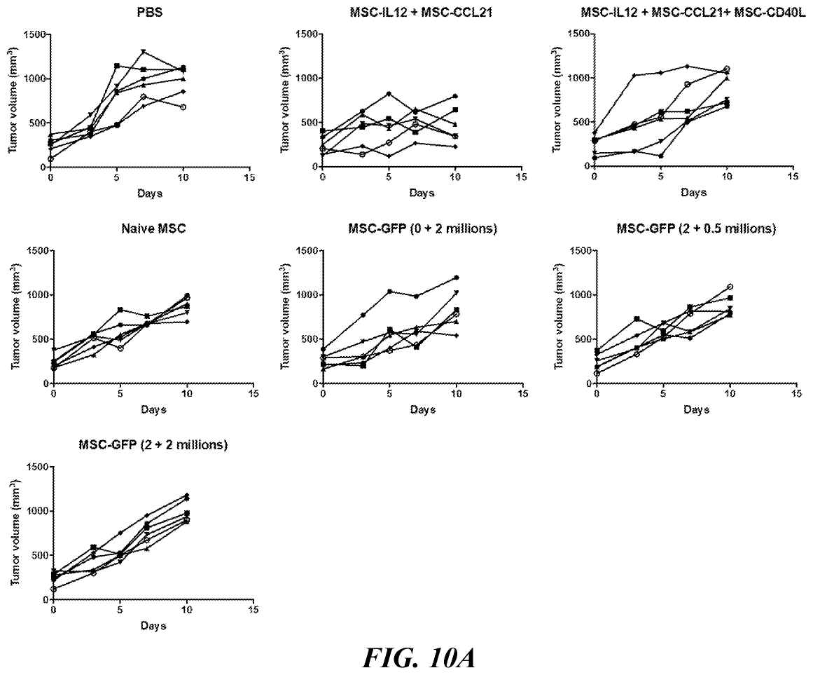

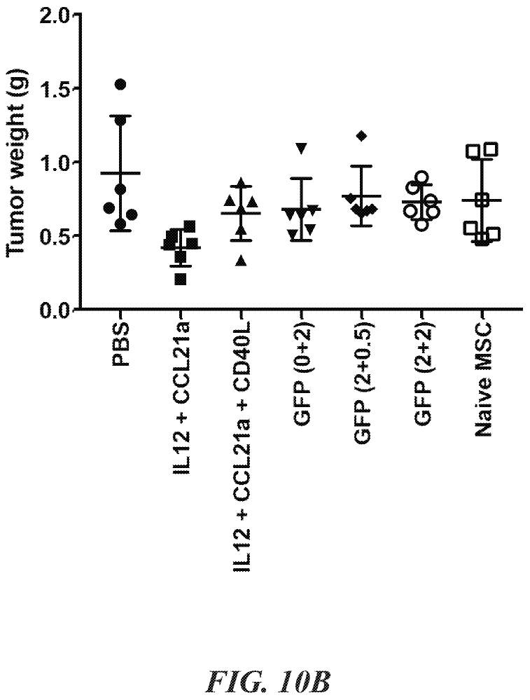

A dose escalation study was performed. This experiment determined that engineered MSC cell expression GFP does not elicit toxicity (FIGS. 10A-10B).

Effect on Large Tumors

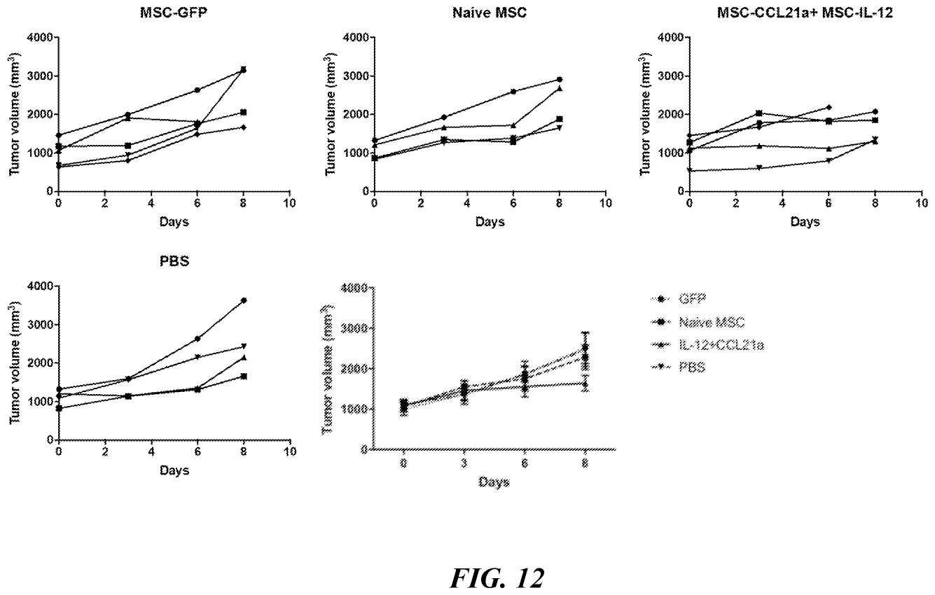

This experiment tested whether engineered mouse MSCs expressing IL12 and CCL21a can reduce tumor burden from larger tumor (>800 mm³). Larger tumor are more difficult to treat than small tumor, and this experiment demonstrates this effector combination can reduce tumor expansion (FIGS. 12A-12B).

Checkpoint Inhibitors

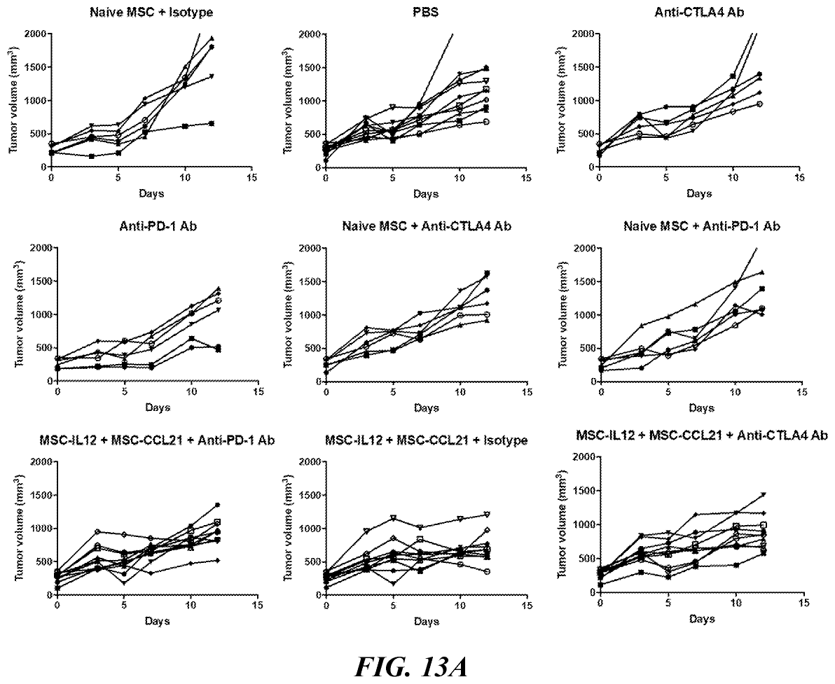

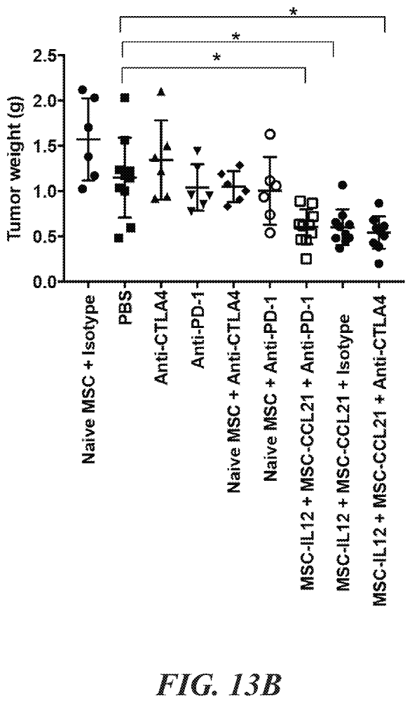

FIG. 13A shows that engineered MSCs expressing IL-12 and CCL21 are sufficient to inhibit tumor growth, although the addition of a checkpoint inhibitor (anti-PD-1 antibody or anti-CTLA-4 antibody) by injection did not increase efficacy in a subcutaneous tumor model.

### Example 5. CT26 Colorectal Carcinoma

In the following experiments, MSCs were engineered to express one of the following effector molecules, then administered, alone or in combinations, to a colorectal carcinoma mouse model: IFNβ, IL12, IL15, IL36γ, IL7, CCL21a, HACv-PD1, or 41BB. In some examples, a checkpoint inhibitor (anti-CD40 or anti-CTLA-4 antibody) was injected in combination with administration with the engineered MSCs.

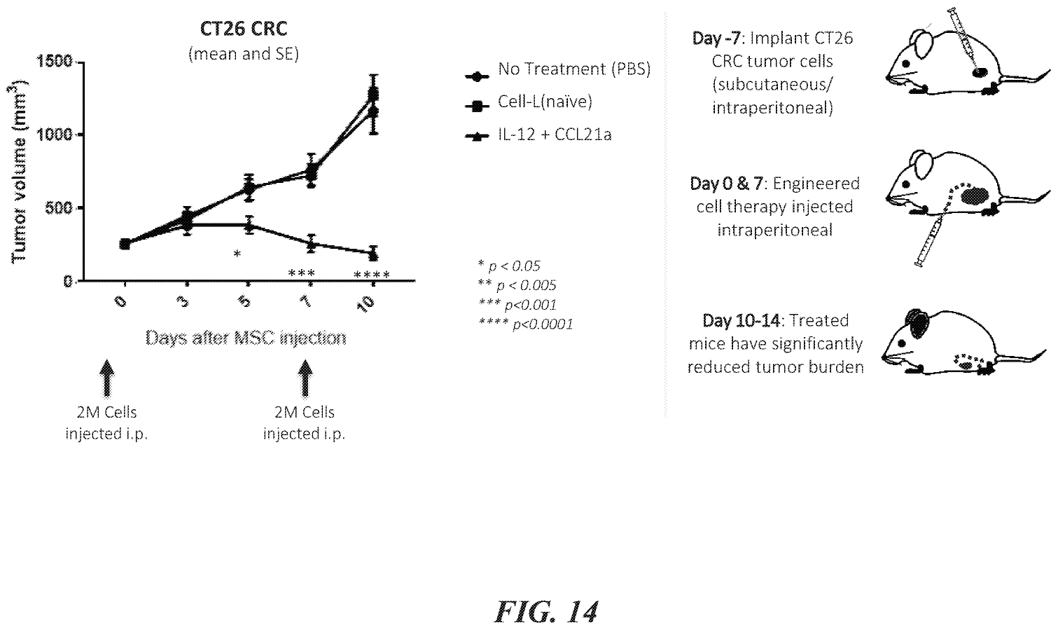

FIG. 14 shows that engineered MSCs expressing IL-12 and CCL21a induced significant tumor growth delay.

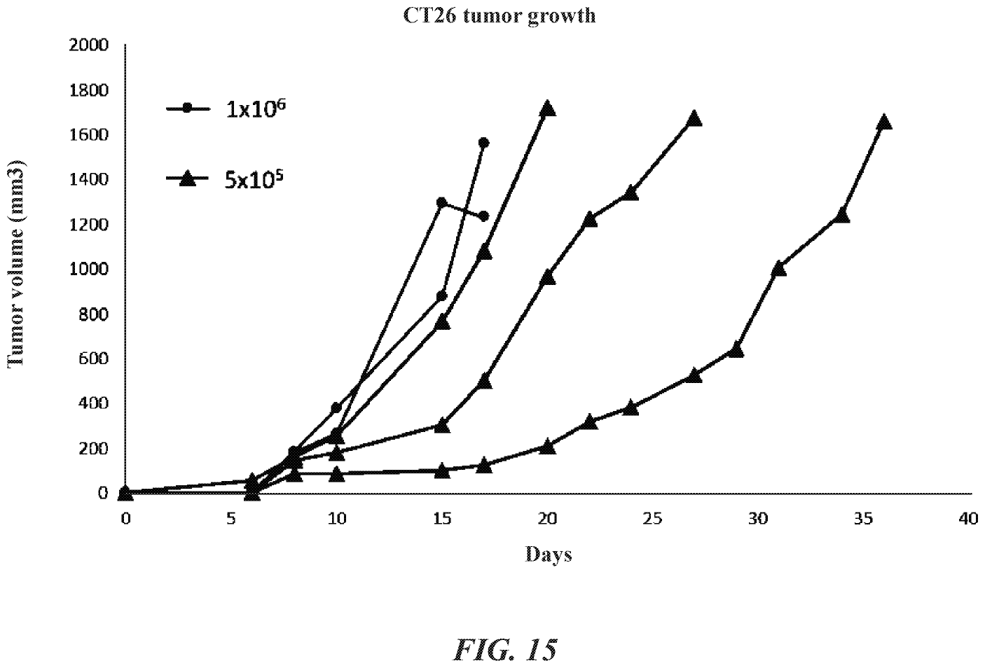

FIG. 15 shows tumor growth kinetics in the CT26 mouse model to determine optimal time for dosing the engineered MSC cells.

In Vivo Efficacy

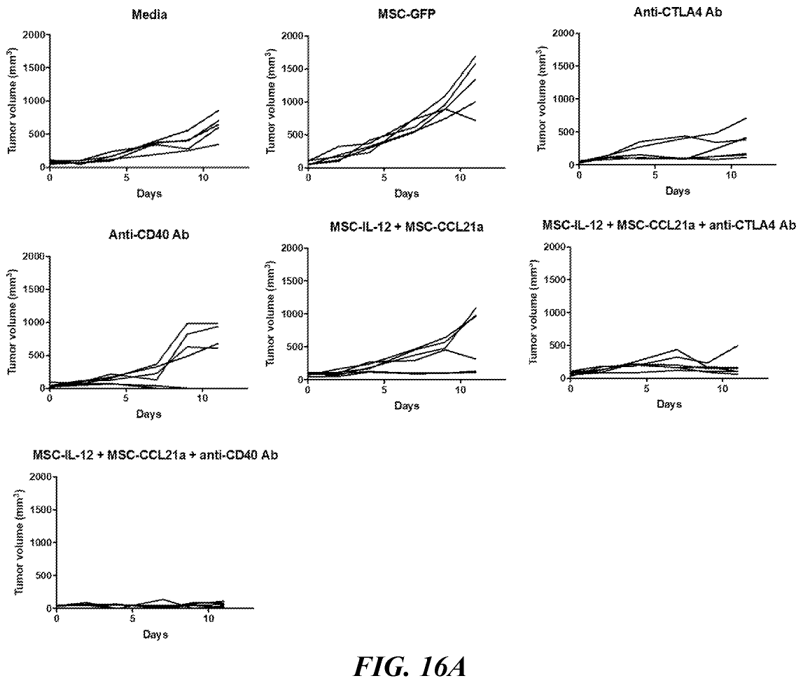

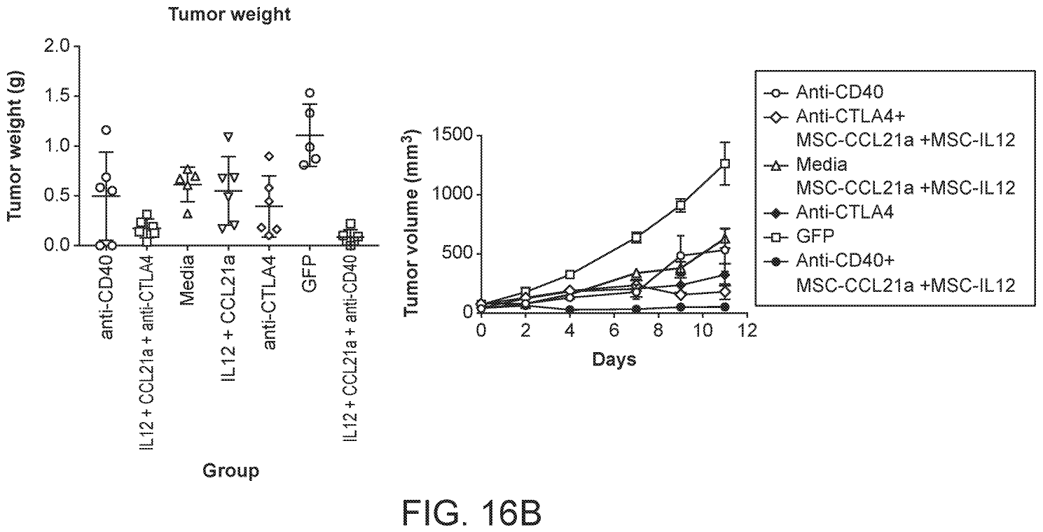

The following experiments demonstrate the in vivo efficacy of MSCs expressing immunotherapy effectors (payloads) in the subcutaneous mouse model of colon (colorectal) cancer. CT26-Neo-Fluc mouse colon cancer cells (Imanis Life Sciences, 5×10⁵) were injected subcutaneously into the flanks of female BALB/cJ mice (The Jackson Laboratory). Seven days after tumor implantation, mice were then randomized into the following treatment groups: control MSC growth media, engineered MSCs (MSC-12+CCL21a), anti-CD40 antibody, anti-CTLA4 antibody (Bio X cell), MSC-12+CCL21a in combination with anti-CD40 antibody or MSC-12+CCL21a in combination with anti-CTLA4 antibody. Engineered MSCs (2×10⁶ cells) were injected intraperitoneally (ip) once a week for two weeks (Day 0 and 7). Anti-CD40 antibodies were injected ip (100 μg) on Days 0 and 3. Anti-CTLA4 antibodies were injected ip (100 μg) on Days 0, 3 and 7. Tumor growth was monitored by caliper measurements every other day, and mouse weights were recorded twice weekly. Mice were euthanized 11 days after first MSC treatment and tumors were collected and weighed. The tumor weight of individual mice in each treatment group was measured and the results are shown in the bottom left of FIG. 16B (left graph). The average tumor volume of each treatment group was monitored over time (FIG. 16B, right graph). Treatment Groups 2 (IL-12+CCL21a+anti-CTLA4 antibody), 4 (IL-12+CCL21a) and 7 (IL-12+CCL21a+anti-CD40 antibody) inhibited the average growth of CT26 colon tumors compared to GFP-treated mice (FIG. 16B, right graph). Similar results were observed when the tumor volume of individual mice in each treatment group was measured over time (FIG. 16A). Therefore, combinatorial treatment with MSCs expressing immunotherapies inhibited the growth of colon cancer cells in vivo.

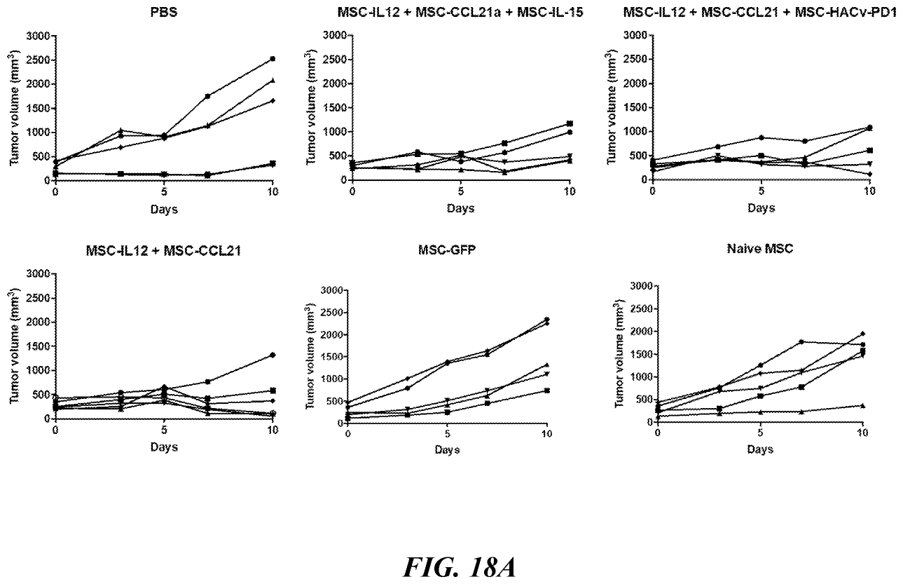

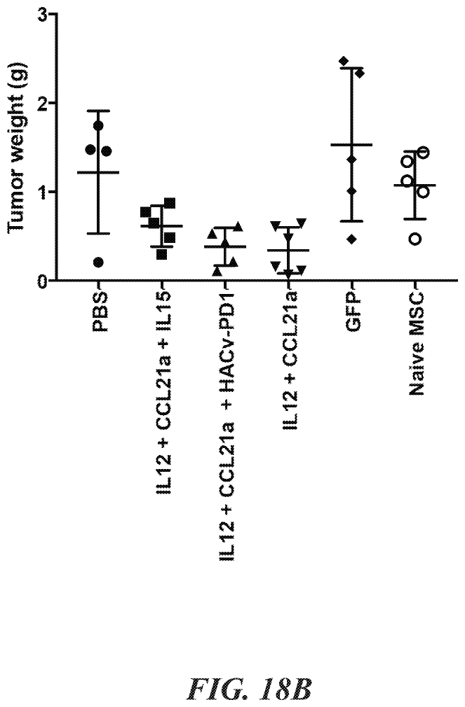

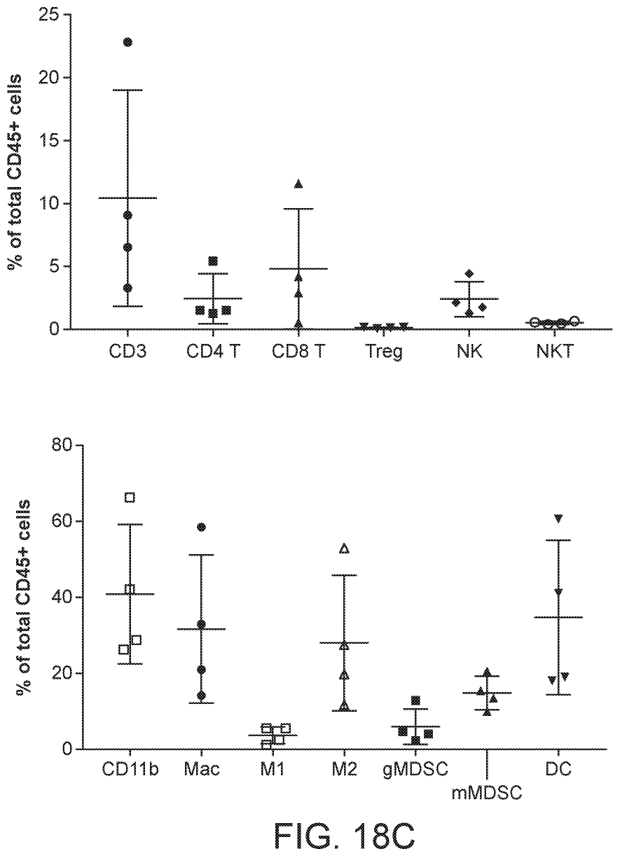

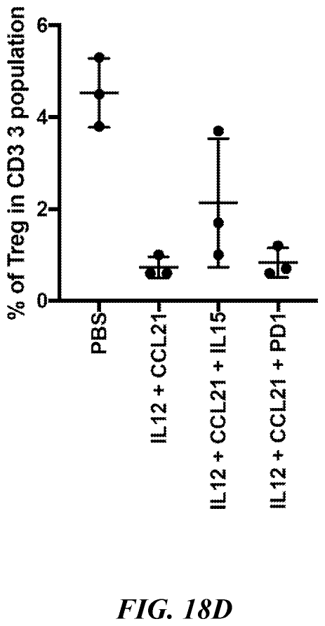

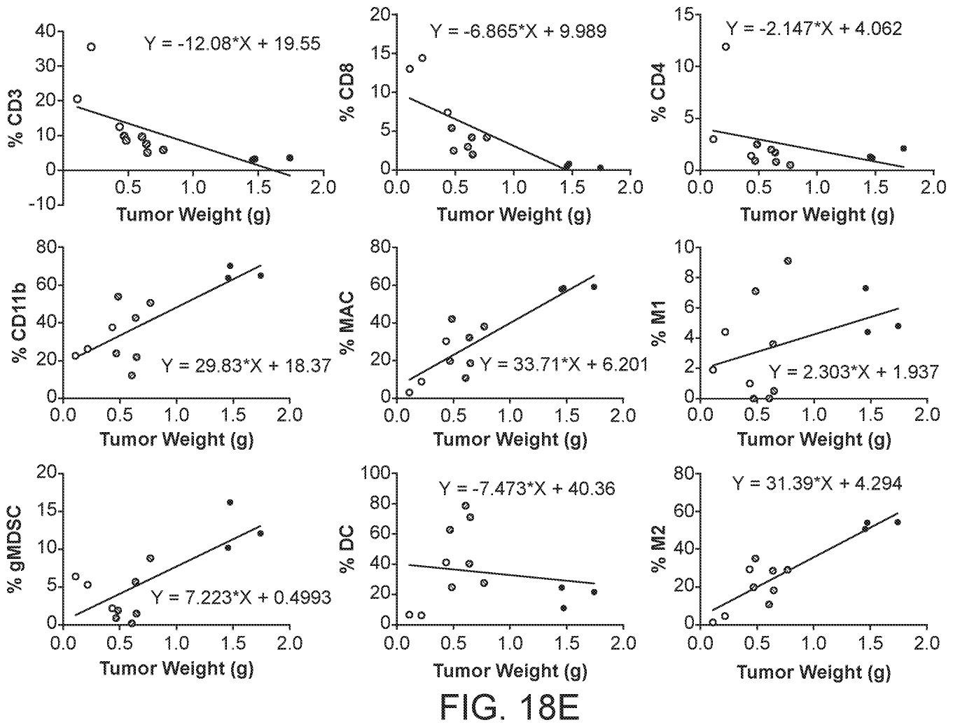

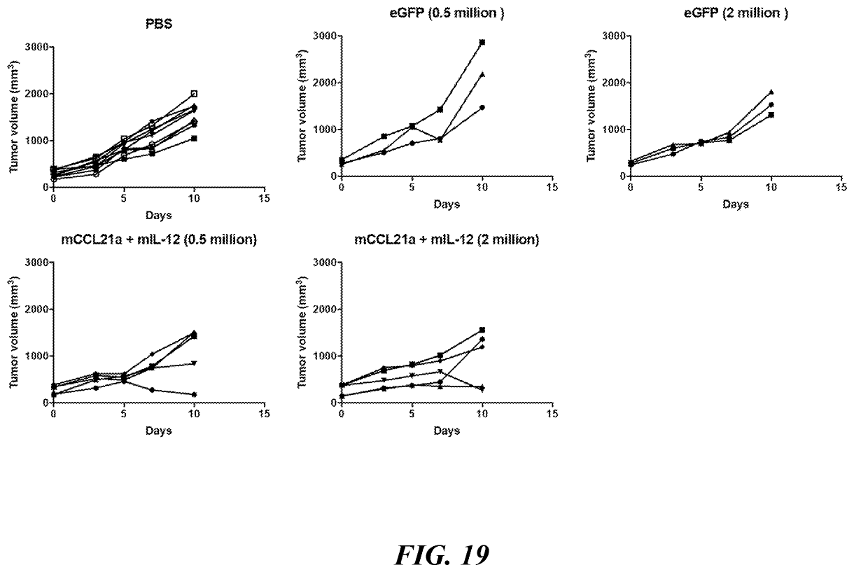

FIG. 18A shows that engineered MSCs expressing IL-12, CCL21a, and either IL15 or HACvPD-1 inhibit tumor growth significantly in a mouse model of colorectal cancer. FIG. 18B shows the tumor weight for individual mice in each treatment. FIG. 18C is a representative graph of the infiltrating immune population within the tumor microenvironment. FIG. 18D shows the percentage of regulatory T cells (Treg) in the total CD3 population. There was a significant decrease in the numbers of Tregs in the tumor microenvironment treated with engineered MSC-IL2 and CCL21a. FIG. 18E correlates the percentage of immune infiltration with tumor weight. Samples with increase in lymphocytes (CD3+) were found to correlate with low tumor weight, while samples with high myeloid (CD11b+) infiltration were correlated with higher tumor burden.

Long-Term Survival

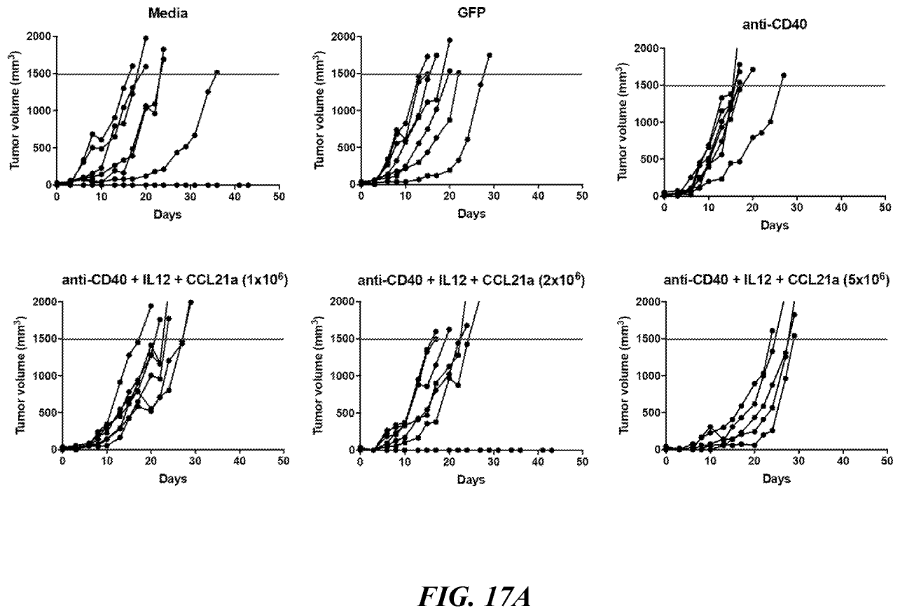

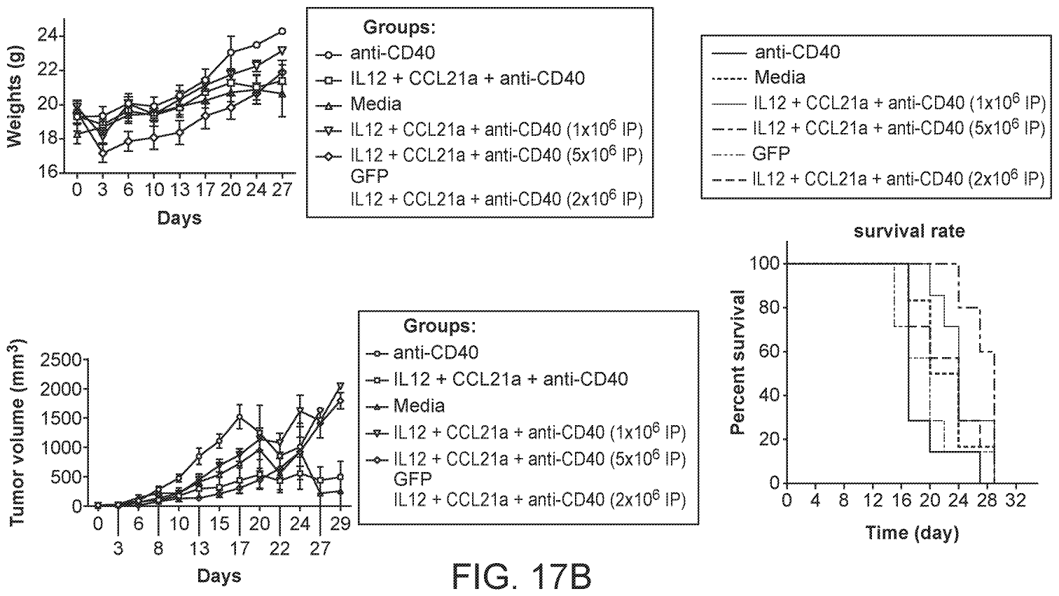

Mice were dosed twice with different concentration of engineered MSC-IL12 and CCL21a therapy in combination with injected anti-CD40 antibody. After the second dose, tumor volume was monitored twice a week until tumor burden is greater than 1500 mm³ and the mice were sacrificed. FIG. 17A shows the tumor volume of the individual group. FIG. 17B, left graph, tracks the mice weight and tumor volume from individual group over time. FIG. 17B, right graph, shows the survival plot of the different groups.

MSC Efficacy

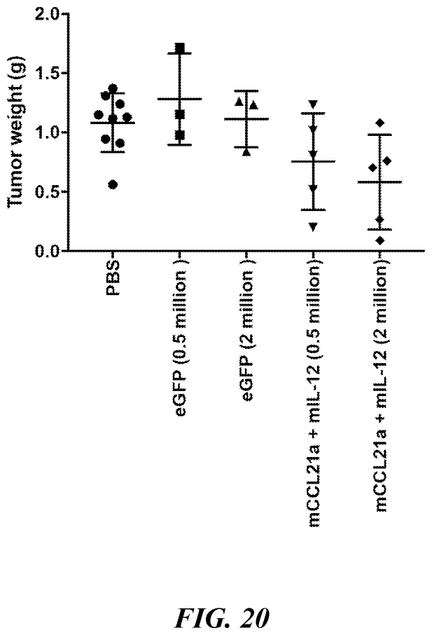

FIG. 20A shows the tumor volume for individual mice in each treatment. FIG. 20B shows the tumor weight for individual mice in each treatment. Efficacy was determined by tumor volume from caliper measurement every other day.

Tumor Growth Kinetics

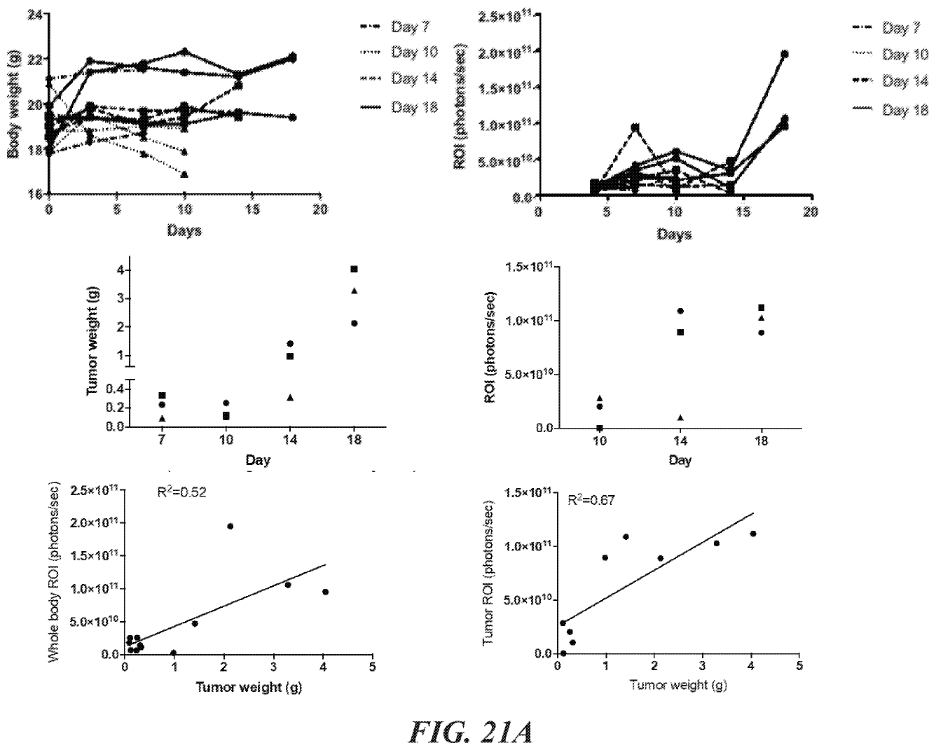

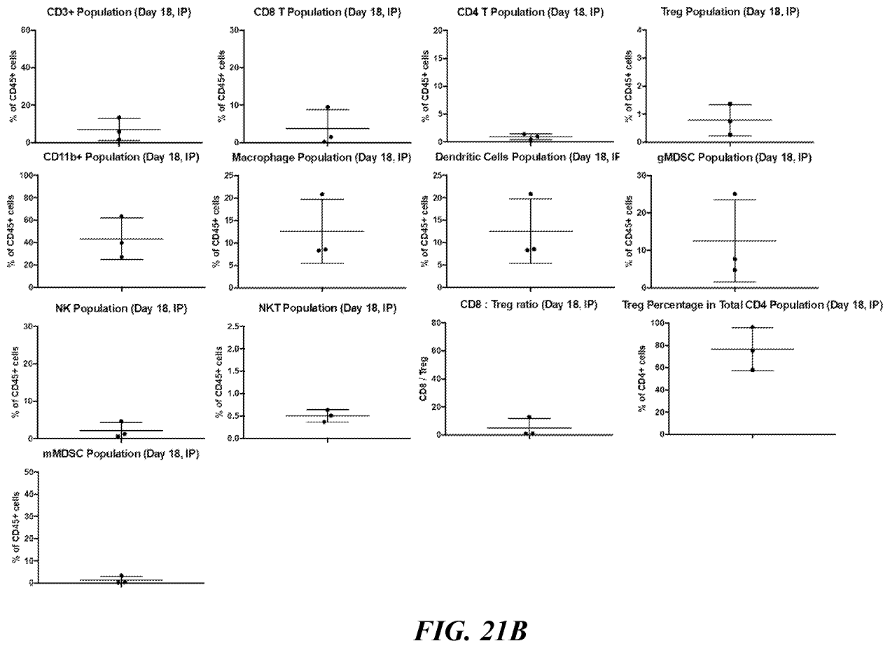

FIGS. 21A-21B show the kinetics of CT26-LUC (luciferase) tumor growth in the intraperitoneal space. A CT26 cell line was injected at day 0 and three (3) mice were harvested at day 7, day 10, day 14, and day 18 to determine the kinetics of tumor growth. The first row of FIG. 21A measures the mice body weight and ROI with an IVIS imager to monitor tumor burden. The second row monitors the tumor weight and the ROI of the tumor of individual mice in each group. The third row correlates the tumor weight with either whole body ROI or tumor ROI. FIG. 21B shows the immune profile of three (3) mice in the day 18 group to better understand the tumor microenvironment.

Tumor Infiltrate Statistics/Immune Percentage/Tumor Weight

Subcutaneous Mouse Model

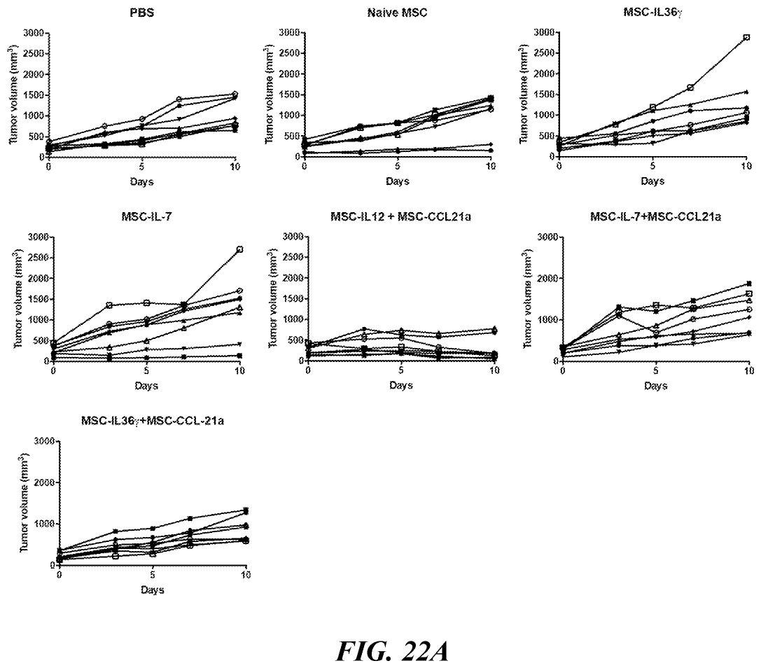

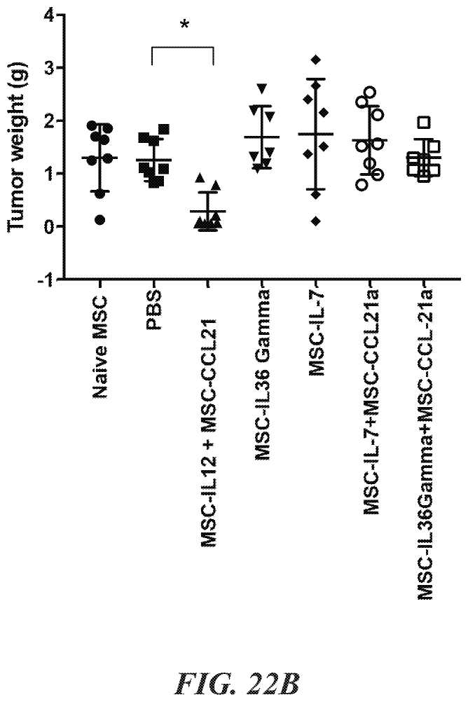

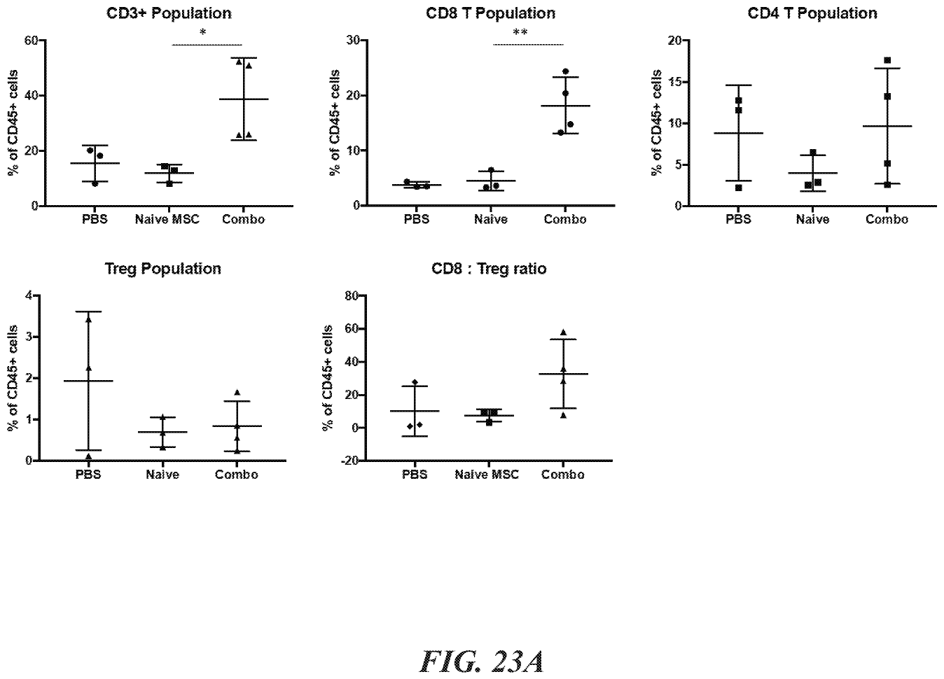

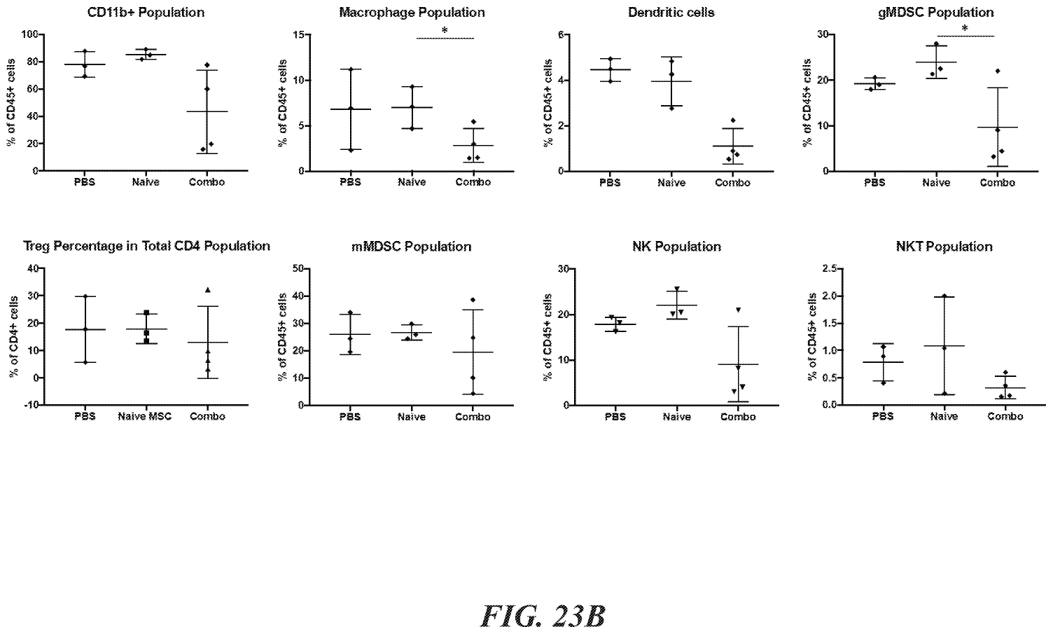

FIG. 22A includes data indicating that engineered MSCs expressing IL-12 and CCL21a inhibit tumor growth in an subcutaneous mouse model of colorectal cancer; however the combination of MSCs expressing CCL21a and IL-36 gamma or IL-7 does not reduce tumor growth. FIGS. 23A-23B include the tumor immune infiltrate statistics. Three mice were selected from PBS, Naïve MSC, and MSC-IL12+MSC-CCL21a (combo) group to run flow cytometry to immune profile tumor microenvironment. FIG. 23A shows a significant increase in infiltrating CD3 and CD8 cytotoxic T population in the combo group compared to the group dosed with naïve MSC. FIG. 23B shows a significant reduction in granulocytic myeloid-derived suppressor cells (gMDSCs) and macrophage population in the combo group compared to group treated with Naïve MSC.

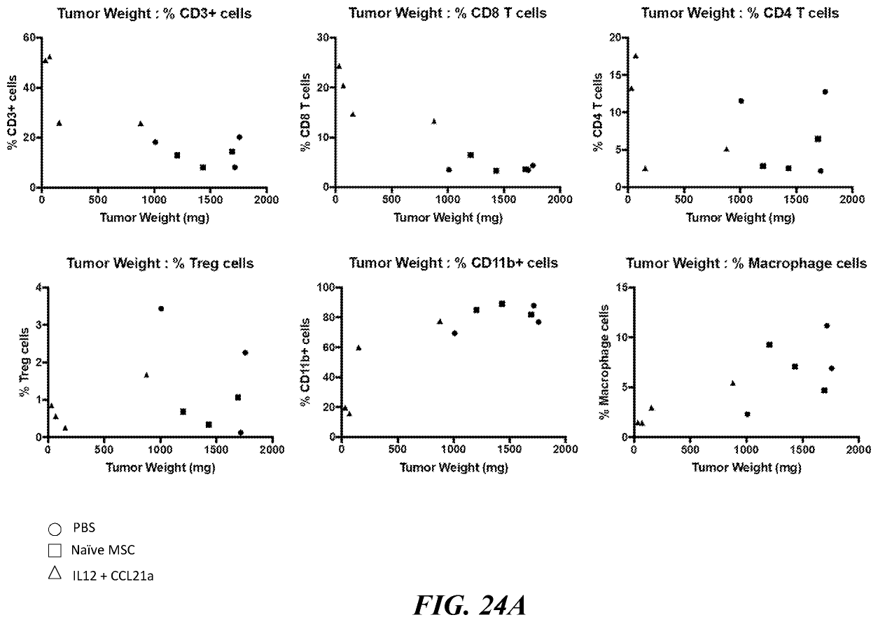

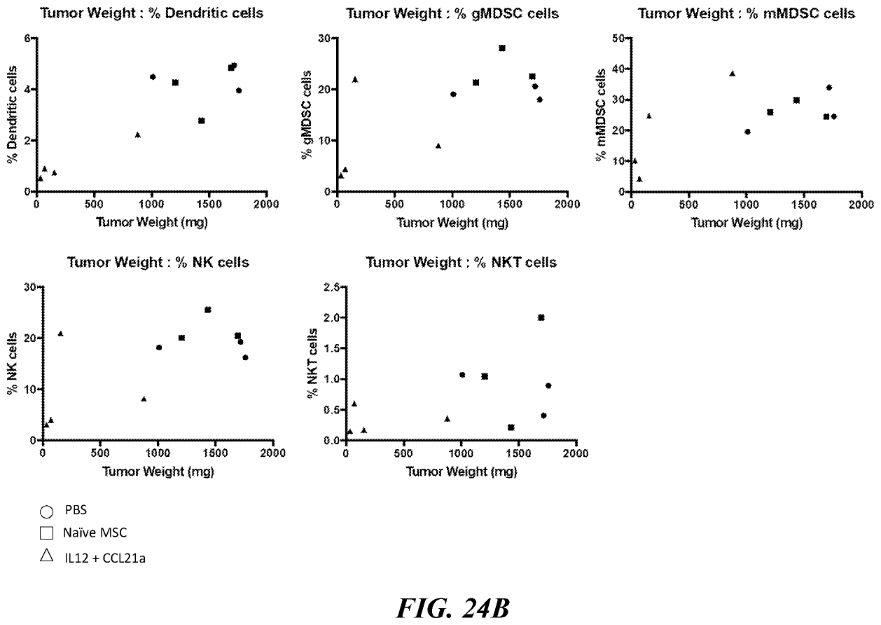

FIGS. 24A-24B include data relating to immune percentage and tumor weight, showing that samples with more CD3+ and CD8+ T cells (top left and center graph) correlate strongly with a decrease in tumor weight. These figures also show that samples with fewer CD11b myeloid cells, including macrophage, dendritic cells, and MDSC, display lower tumor burden (lower center and right graph of FIG. 24A and upper row of FIG. 24B).

Orthotopic Mouse Model

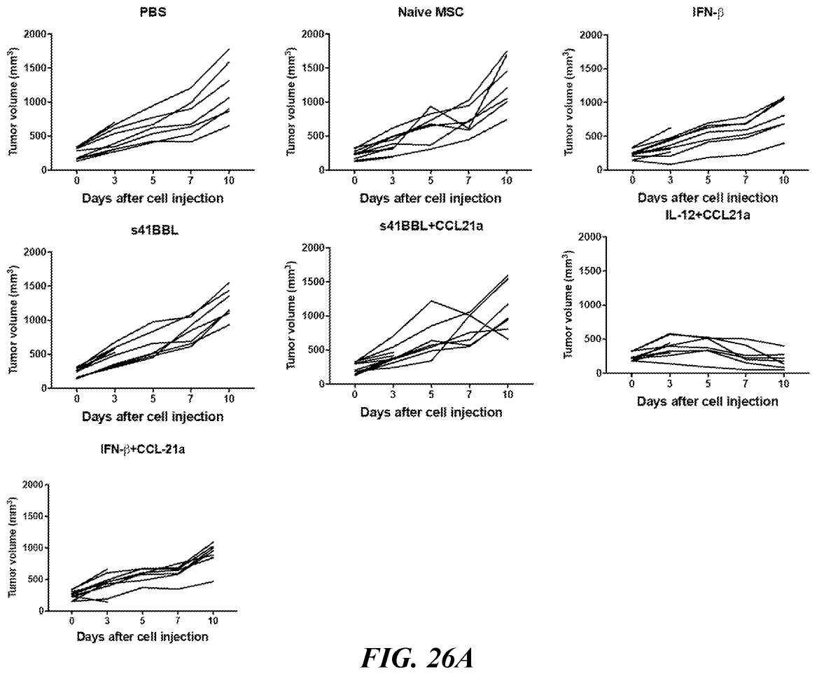

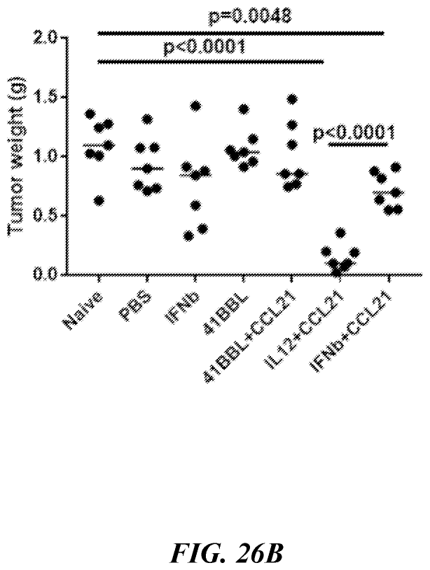

FIG. 26A shows that engineered MSCs expressing IL-12 and CCL21a, or CCL21a and IFN-β, inhibit tumor growth in an orthotopic mouse model of colorectal cancer; however the combination of MSCs expressing CCL21a and s41BBL does not reduce tumor growth. Each effector was expressed by a different MSC, and the MSCs were combined (at a 1:1 ratio) for combinatorial treatment. Each chart shows the effect of engineered MSCs expressing the indicated immunotherapies alone or in combination on the growth of 4T1 breast tumors in mice (n=6-8). Each line of FIG. 26A represents an individual mouse. FIG. 26B shows the tumor weight for individual mice in each treatment. MSC-IL12+MSC-CCL21a shows best efficacy compared to mice injected with naïve MSC. Treatment efficacy was also observed in the group treated with MSC-IFNb+MSC-CCL21a.

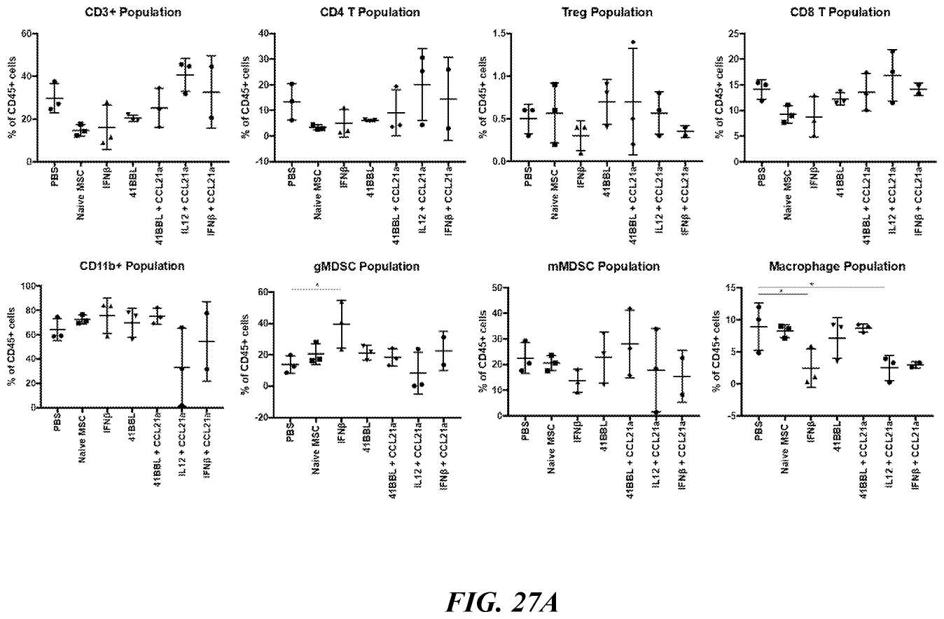

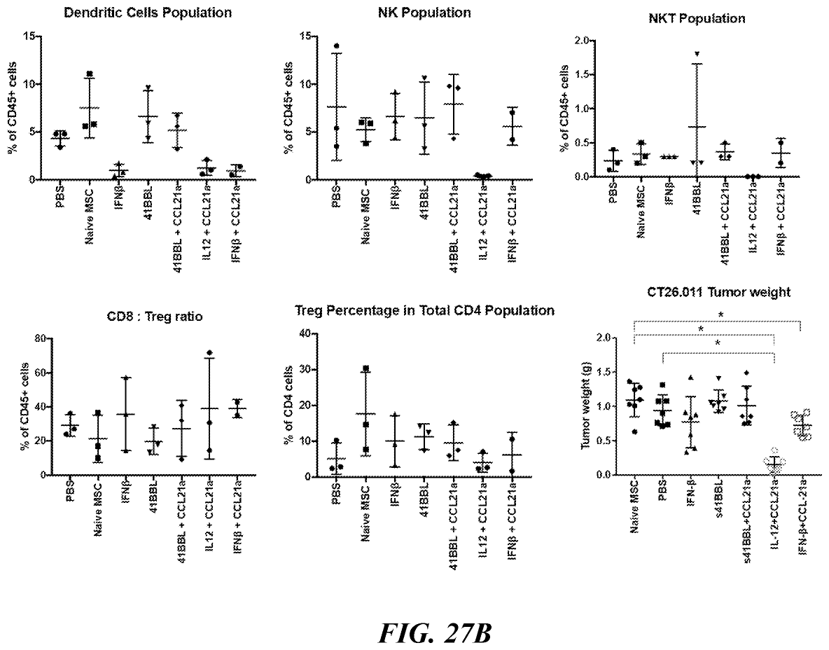

FIGS. 27A-27B are graphs that show immune profiles of each group treated with indicated engineered MSC. A consistent decrease in macrophage population was observed after treating with MSC-IL12+MSC-CCL21a (FIG. 27A). A general trend of increased infiltration in CD3+ population and decreased infiltration in CD11b+ population was also observed when compared to group treated with MSC-IL12+MSC-CCL21a against naïve MSC (FIG. 27A and FIG. 27B).

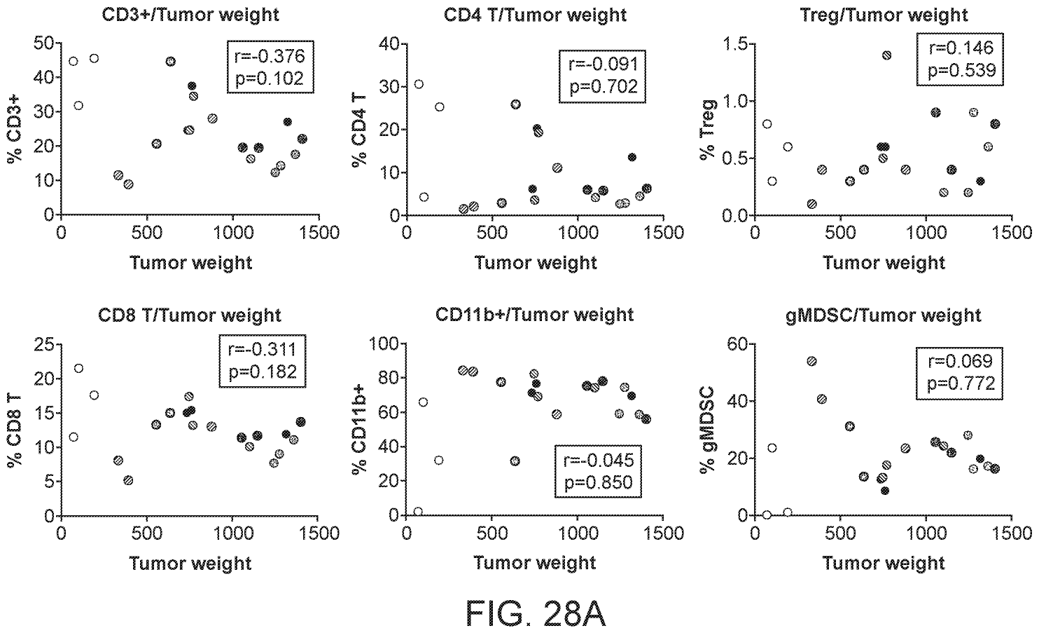

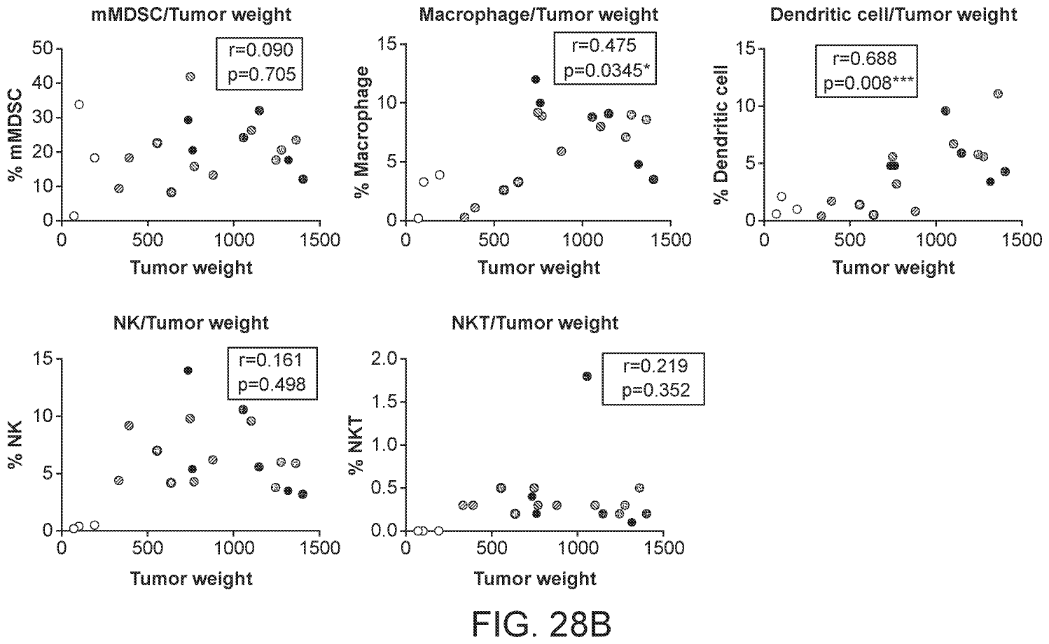

FIG. 28A-28B show the correlation of immune infiltration with tumor weight. Samples with low macrophage and dendritic cells have lower tumor burden (FIG. 28B, top center and top right). FIG. 28C shows the average tumor weight from each group. Statistical significance was observed with both MSC-IL12+MSC-CCL21a, or MSC-IFNb+MSC-CCL21a compared with naïve MSC.

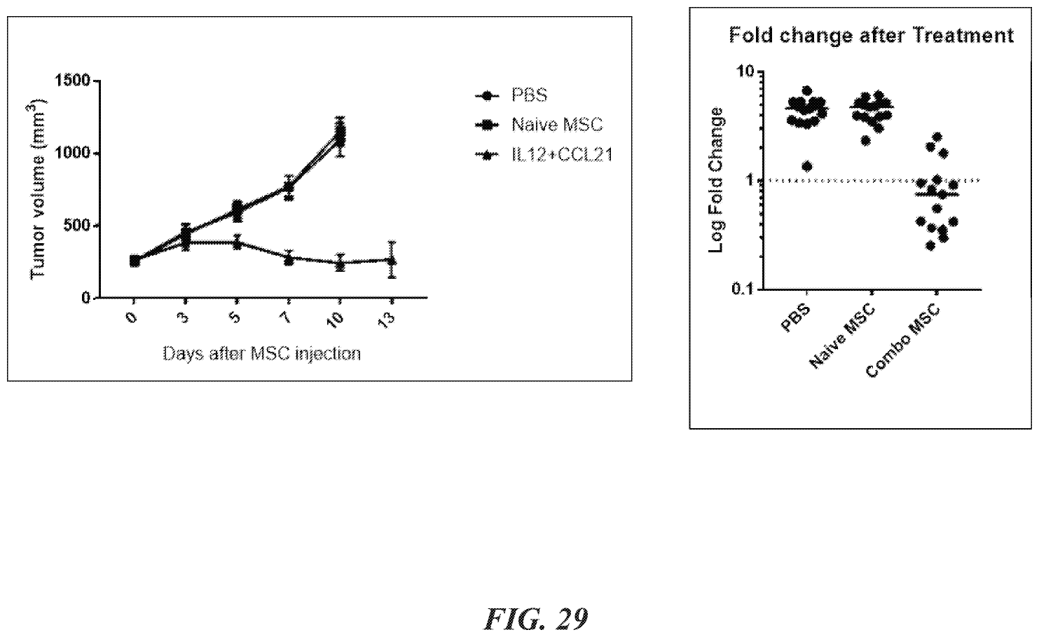

FIG. 29 shows graphs combining the in vivo data from the colorectal cancer models above (FIG. 22A and FIG. 26A). The combined CT26 data from FIG. 22A and FIG. 26A capture three groups: tumor only (PBS), treated with naïve MSC, and treated with MSC-IL12+MSC-CCL21a.

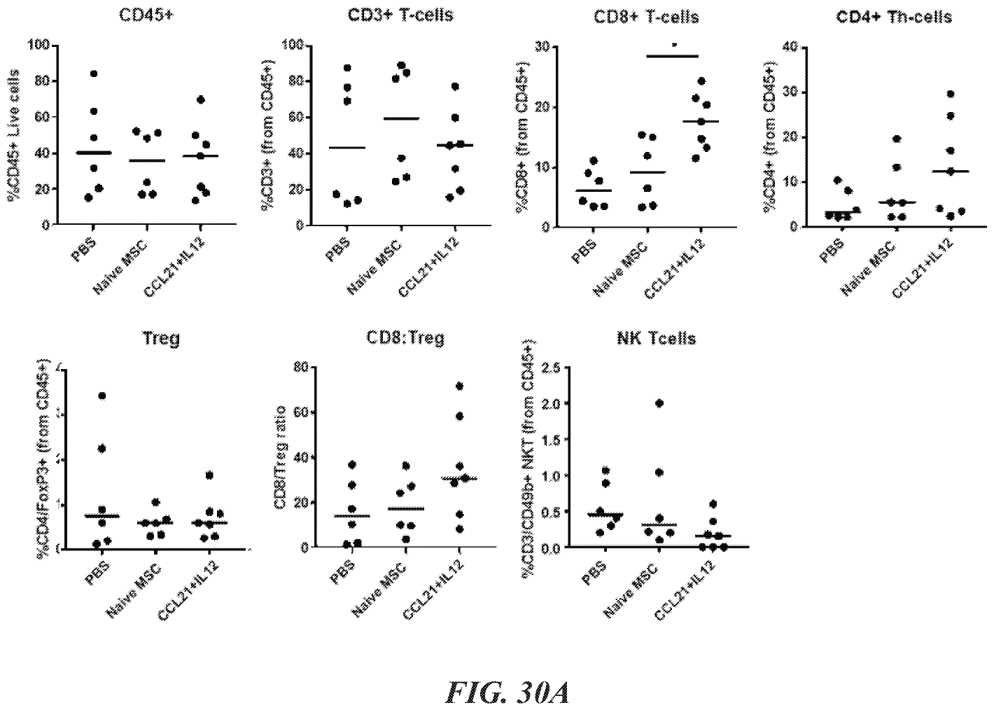

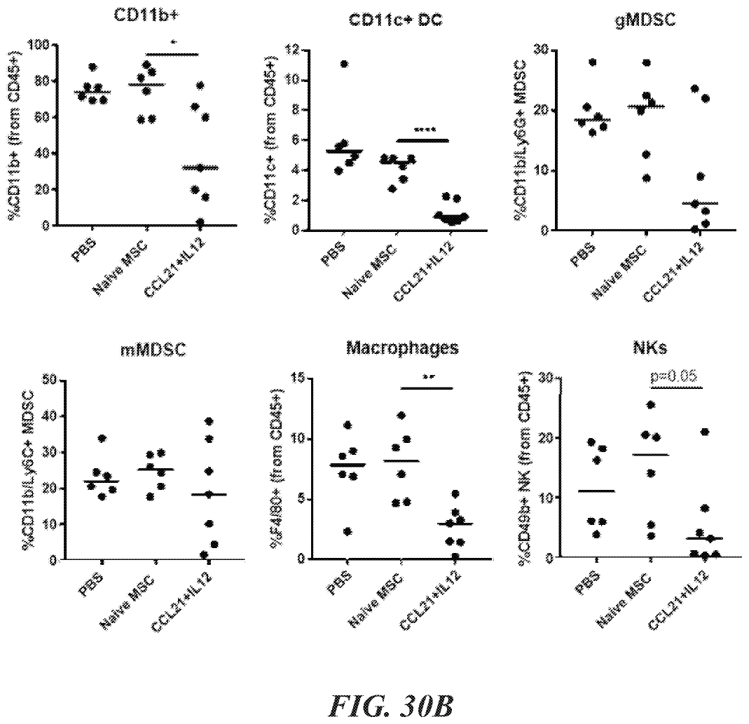

FIGS. 30A-30C also show combined data from FIG. 22A and FIG. 26A. The graphs show the average number of immune infiltration from the flow cytometry experiment data. Statistical significance was observed in CD8+T from FIG. 30A, demonstrating the ability of MSC-IL12+MSC-CCL21a to repolarize tumor microenvironment and allow more cytotoxic T cell infiltration. Furthermore, there was a reduction in CD11b+ myeloid population infiltration in the groups that were treated by MSC-IL12+MSC-CCL21a (FIG. 30B). The data collected using dendritic cells and the macrophage population was statistical significance.

IL12 and CCL21a Therapy in Intraperitoneal and Subcutaneous Mouse Models of Colorectal Cancer

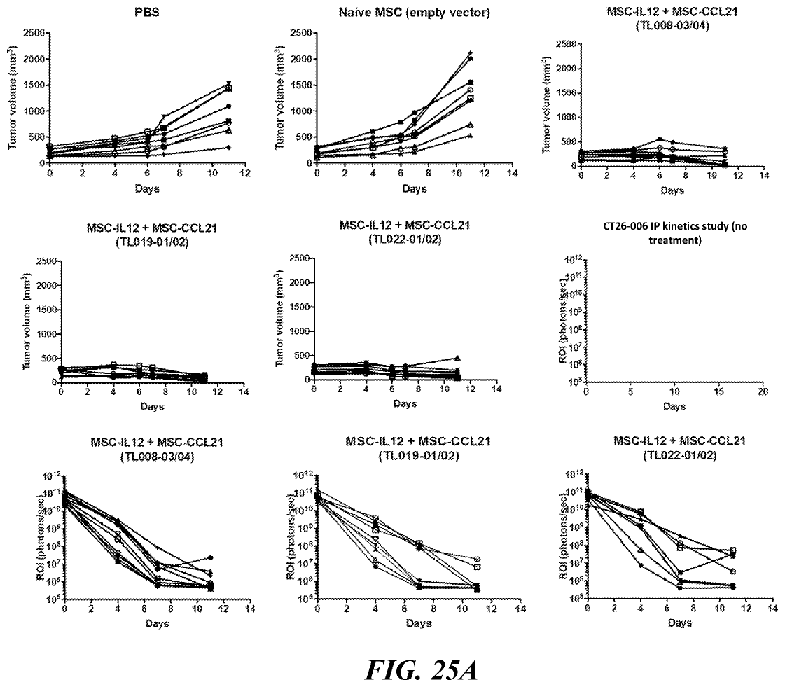

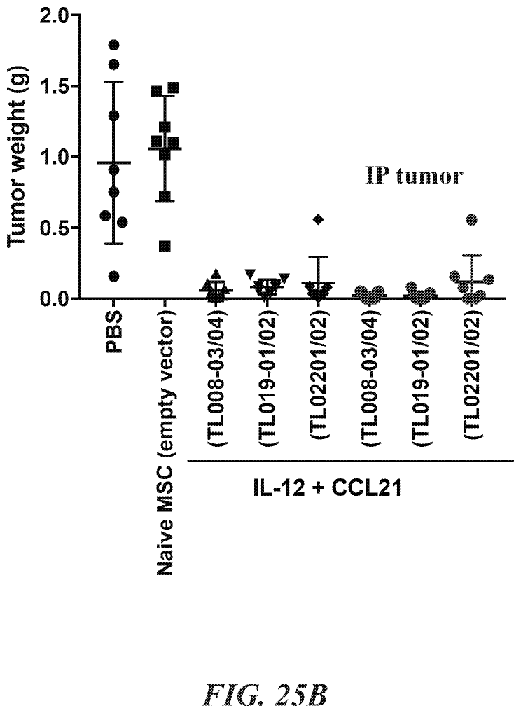

FIGS. 25A-25B include data from MSC-IL-12+CCL21a therapy in intraperitoneal and subcutaneous colorectal cancer mouse models. Three different lots of a lentiviral transduced line was tested for MSC-IL12 and CCL21a (TLOO8-3/4, TL019-01/02, and TL022-01/02; each TL number represents one lot). FIG. 25A shows that all three lots of MSC-IL12+MSC-CCL21a can reduce tumor burden in both subcutaneous and intraperitoneal model (first 5 graphs are from the SC model and last 3 are from the IP model). Tumors from all mice were collected on day 11. FIG. 25B shows the average tumor weight from each group.

### Example 6. MSC Combination Cytokine Therapy Methods

The following methods were used in experiments, as indicated.

### Methods:

MSC Culturing

Bone-marrow derived C57BL/6 and Balb/C murine MSCs (mMSCs) were purchased from Cyagen (Cat. No. MUBMX-01001 and MUCMX-01001, respectively). mMSC culturing media was composed of: MEM Corning Cat #10-022-CV (500m1)+MSC FBS Gibco Cat #12662-029 (final conc 10%)+L-Glut (200 mM) Stem cell 07100 (Final conc 2 mM)+PenStrep 100X VWR Cat #97063-708 (Final conc 1×)+murine FGF Peprotech Cat #450-33-100uG (Final cone-1:10,000 dilution). TrypLE Express was purchased (ThermoFisher—#12604021). PBS did not contain magnesium, calcium, or phenol red.

mMSCs were passaged according to the protocol below:
1. mMSCs should be passaged at 70-90% confluency.
2. Aspirate media from dish/flask.
3. Rinse plate with PBS (e.g. 2 mL for 10 cm dish, 3m1 for 15 cm dish).
4. Add TrypLE Express (e.g. 2 mL for 10 cm dish, 3m1 for 15 cm dish)
5. Incubate for 3-4 minutes at 37 degrees.
6. Knock plate on side to dislodge cells. Confirm by microscopy that most cells
have been dislodged.
7. Wash cells off plate using media (e.g. 8 mL for 10 cm dish).
8. Place cells in 15 conical and centrifuge 400×g for 5 min.
9. Aspirate media.
10. Resuspend cells in appropriate media and plate cells into fresh plates/flasks.
Note: 70% confluent cells can be
split 1:3. 90% confluent cells can be split 1:4. Alternatively, cells can be plated at 3000-5000 cells/cm2.

Bone-marrow derived human MSCs were purchased (RoosterBank-hBM-1M-XF, RoosterBio). Various hMSC culturing media were purchased: Xeno-free hMSC media—(RoosterBio—#KT-016); +FBS (serum-containing) hMSC media (Lonza—MSCGM media—#PT-3001). TrypLE Express was purchased (ThermoFisher—#12604021). PBS did not contain magnesium, calcium, or phenol red.

hMSCs were passaged according to the exemplary protocol below:
1. hMSCs should be passaged at 70-90% confluency.
2. Aspirate media from dish/flask.
3. Rinse plate with PBS (e.g. 2 mL for 10 cm dish).
4. Add TrypLE Express (e.g. 2 mL for 10 cm dish)
5. Incubate for 3-4 minutes at 37 degrees or 5 minutes RT.
6. Knock plate on side to dislodge cells. Confirm by microscopy that most cells have been dislodged.
7. Wash cells off plate using Lonza MSCGM media (e.g. 8 mL for 10 cm dish).
8. Place cells in 15 conical and centrifuge 400×g for 5 min.
9. Aspirate media.
10. Resuspend cells in Rooster xeno-free media and plate cells into fresh plates/flasks. Note: 70% confluent cells can be split 1:3. 90% confluent cells can be split 1:4. Alternatively, cells can be plated at 3000-5000 cells/cm2.

hMSCs were thawed according to the exemplary protocol below:
1. Pre-warm hMSC media to 37°.
2. Remove hMSC aliquot from liquid nitrogen.
3. Thaw by holding the tube 1/2 submerged in 37° bath for 60-90 seconds, until 2/3 of the frozen sample has thawed.
4. Wipe the tube with 70% ethanol to sterilize tube.
5. Add 0.5 mL media to the cryotube, gently pipette 2-3 times, and then transfer cells into 9 mL media (10 mL total) in 15 mL conical tube.
6. Centrifuge 400×g for 5 min.
7. Aspirate media, and then gently resuspend pellet in appropriate volume of Rooster xeno-free media. Plate cells at a concentration of 3000-5000 cells/cm2.

Lentiviral Production

Lentivirus was produced using: Lenti-X 293T packaging cell line (Clontech, Cat #632180); LX293T Complete growth medium, without antibiotics; DMEM, hi-glucose; 1 mM Sodium Pyruvate; 10% FBS, heat-inactivated; Opti-Mem I Reduced Serum Media (Gibco/Thermo Fisher; Cat #31985); FuGene HD (Promega, Cat #E2311); Envelope, Packaging, and Transfer Vector plasmids; VSV-G-pseudotyped envelope vector (pMD2.G); Packaging vector that contains Gag, Pol, Rev, and Tat that can be used with 2nd and 3rd generation transfer vectors (psMAX2). 293T(FT) cells from 90% confluent 10 cm dishes were lifted and dispensed at 1:3 dilution late in the afternoon the day before transfection and incubated cells as normal overnight at 37° C., 5% CO2 (cells should be 60-85% confluent the next day at time of transfection).

A transfection reaction was prepped for each 10 cm dish according to the protocol below:
1. Prep transfection reaction for each 10 cm dish in a separate 1.7 mL tube.
2. Add 900 uL Opti-Mem I at RT.
3. Add 9 ug vector backbone (containing gene of interest) per reaction.
4. Add Bug packaging vector per reaction.
5. Add 1 ug envelope vector per reaction (pMD2.G).
6. Mix thoroughly by quickly vortexing for 3 seconds.
7. Add 55 uL Fugene HD per reaction.
8. Mix by quickly pipetting up and down 20-30 times.
9. Let sit at RT for 10 min (allowing DNA complexes to form).
10. Slowly add mixture in dropwise manner around the dish, then mix by gently rocking back-forth and up-down for 5-10 seconds (do not swirl).
11. Place dish into virus incubator.

Viral supernatants were harvested on days 2 and 3 using a serological pipette. Cellular debris was removed using a Millipore steriflip 0.45 um filters. A Lenti-X Concentrator (Cat. Nos. 631231 & 631232) was used according to the protocol: 1) Combine 1 volume of Lenti-X Concentrator with 3 volumes of clarified supernatant. Mix by gentle inversion; 2) Incubate mixture on ice or at 4° C. for 30 minutes to overnight; (3) Centrifuge sample at 1,500×g for 45 minutes at 4° C.; (4) Carefully remove and discard supernatant, taking care not to disturb the pellet; (5) Gently resuspend the pellet in 1/10 to 1/100th of the original volume using sterile PBS+0.1% BSA.

Vectors

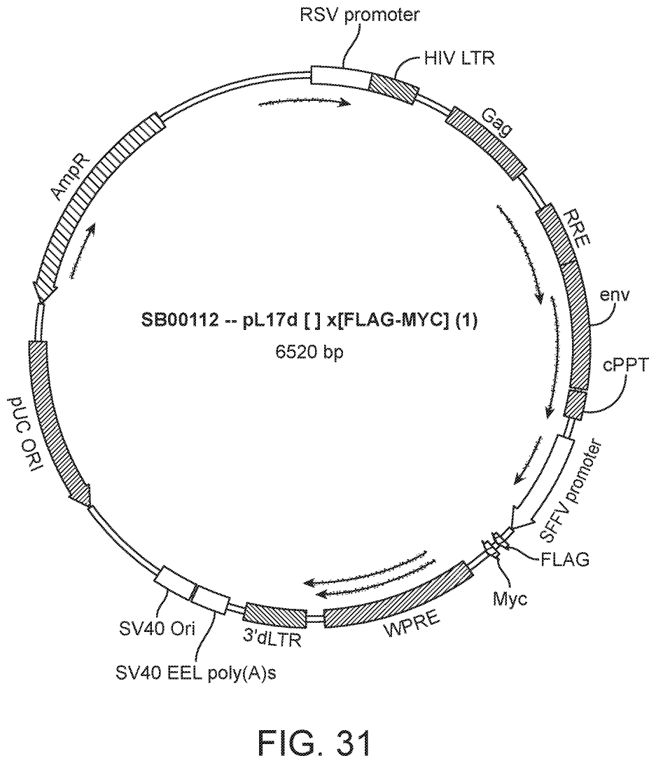

Cytokine expression cassettes were cloned into a pL17D, the vector map of which is shown in FIG. 31 with salient features annotated; e.g., a SFFV promoter; a FLAG and MYC epitope tag; LTRs, etc.

Lentiviral Transduction

Murine MSCs were seeded in 6-well plates and infected when cells were 50% confluent. Virus was added at the appropriate MOI and incubated for 3 hours to transduce cells. Following infection, fresh media was added to the cells.

Human MSCs were transduced following the exemplary protocol below:
1. 200,000 human MSCs were plated in each well of 6-well plate, in 2 mL xeno-free human MSC media.
2. After 2 hours, the media was removed and replaced with 1 mL of PBS.
3. Appropriate amount of virus was added to each well, as indicate by MOI below, and cells were incubated with virus for 3 hours with occasional rocking, at 37 degrees and 5% CO2.
4. Virus was removed after 3 hours, plates were washed with media, and then the MSCs were cultured normally (as noted above) until cells reached senescence. Cells were counted at each passage, so that total cell numbers could be determined.

### Example 7: MSC Combination Cytokine Therapy (CT26)

In the following example, balb/c mMSCs were engineered to express various cytokines using the lentiviral transduction method described in Example 6.

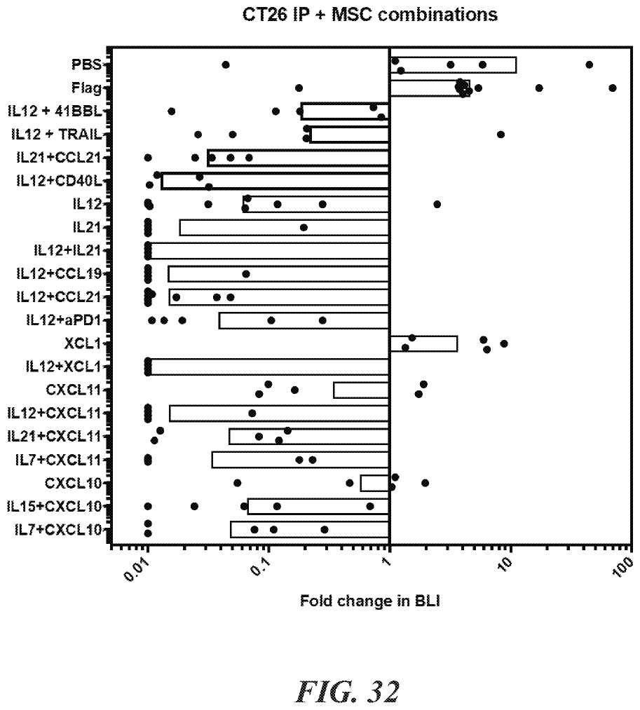

CT26 tumor cells (5×10⁴ cells in 100 μl) engineered to constitutively express luciferase enzyme (Cat no: CL043, Lot no: CL-IM147 Imanis Life Sciences) were injected into the peritoneal space of immunocompetent balb/c (age 6-8 weeks). One week after tumor implantation, tumor burden was measured by luciferase imaging (BLI) using an AMI imager. Mice were randomized into treatment groups and treated with intraperitoneally delivered mMSCs (1×10⁶) expressing effector molecules as single agent or as a combination of mMSCs to deliver a combination of agents. MSC-Flag-Myc and PBS were used as a negative control. Tumor burden was assessed at day 12 and 17. Bioluminescent signal (photons/second) was normalized for each individual mouse relative to the initial signal (pre-treatment). Reduction of BLI signal by more than 100 fold (0.01) was equivalent to a complete cure (no tumor was evident at the time of necropsy). As shown in FIG. 32, MSCs engineered to express different effector molecules either alone or in combination demonstrated efficacy in reducing CT26 tumor burden in an IP tumor model as assessed by BLI levels.

### Example 8: MSC Combination Cytokine Therapy (B16F10)

In the following example, C57BL/6 mMSCs were engineered to express various cytokines using the lentiviral transduction method described in Example 6.

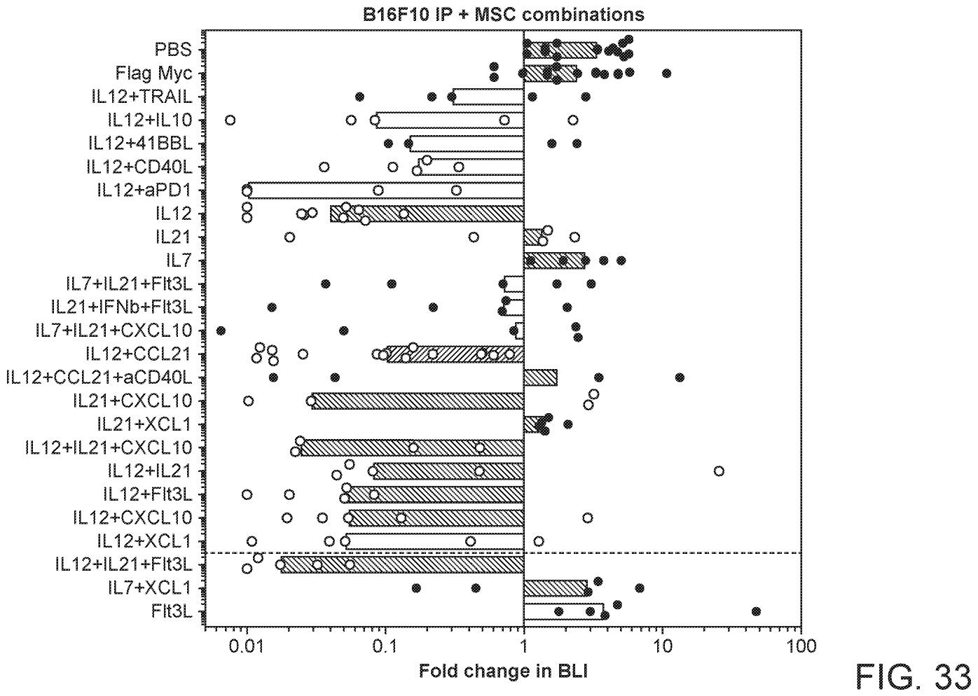

B16F10 tumor cells (5×10⁴ cells in 100 μl) engineered to constitutively express luciferase enzyme (B16F10-Fluc-Puro Cat #:CL052, lot #: CL-IM150 Imanis Life Sciences) were injected into the peritoneal space of immunocompetent C57BL/6 (age 6-8 weeks). One week after tumor implantation, tumor burden was measured by luciferase imaging (BLI) using an AMI imager. Mice were randomized into treatment groups and treated with intraperitoneally delivered mMSCs (1×10⁶) expressing effector molecules as single agent or as a combination of mMSCs to deliver a combination of agents. MSC-Flag-Myc and PBS were used as a negative control. Tumor burden was assessed at day 12 and 17. Bioluminescent signal (photons/second) was normalized for each individual mouse relative to the initial signal (pre-treatment). Reduction of BLI signal by more than 100 fold (0.01) was equivalent to a complete cure (no tumor was evident at the time of necropsy). As shown in FIG. 33, MSCs engineered to express different effector molecules either alone or in combination demonstrated efficacy in reducing B16F10 tumor burden in an IP tumor model as assessed by BLI levels.

### Example 9: Engineered Human MSC Cytokine Production

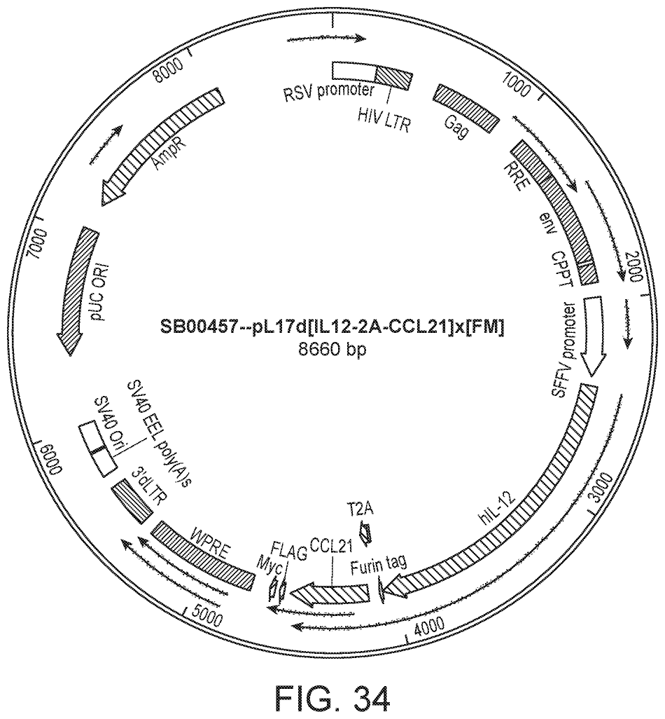

In the following example, bone-marrow derived hMSCs (derived from 3 human volunteer healthy donors) were engineered to express human IL12 (p70) and human CCL21a from a single lentiviral expression vector using the lentiviral transduction method described in Example 6. The lentiviral expression vector (schematic vector map of which is shown in FIG. 34) used a 2A ribosome skipping elements to express both cytokines from a single transcript.

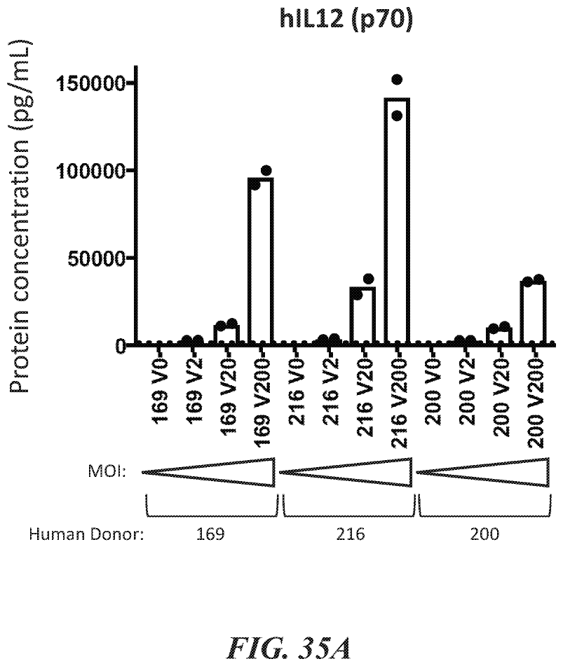

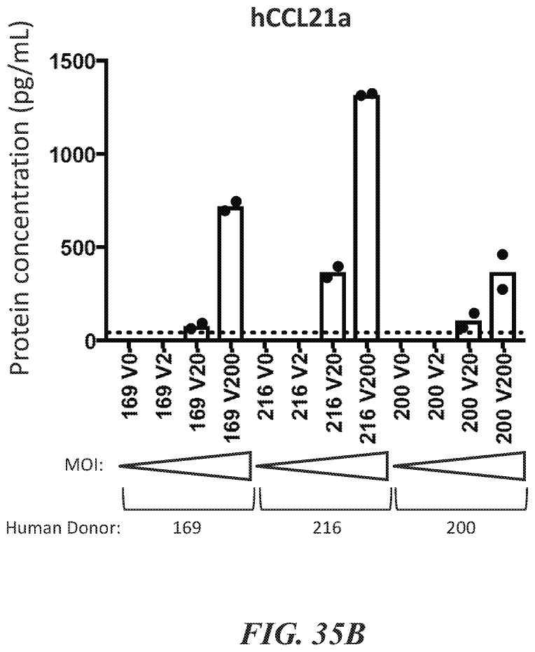

As shown in FIG. 35, engineered hMSCs were able to produce both hIL12 (FIG. 35A) and hCCL21a (FIG. 35B), as assessed by cytokine ELISA. Notably, protein secretion was correlated with the amount of viral particles (MOI) used during the transduction of MSCs.

### Example 10: Engineered Human MSC Functional Assessment

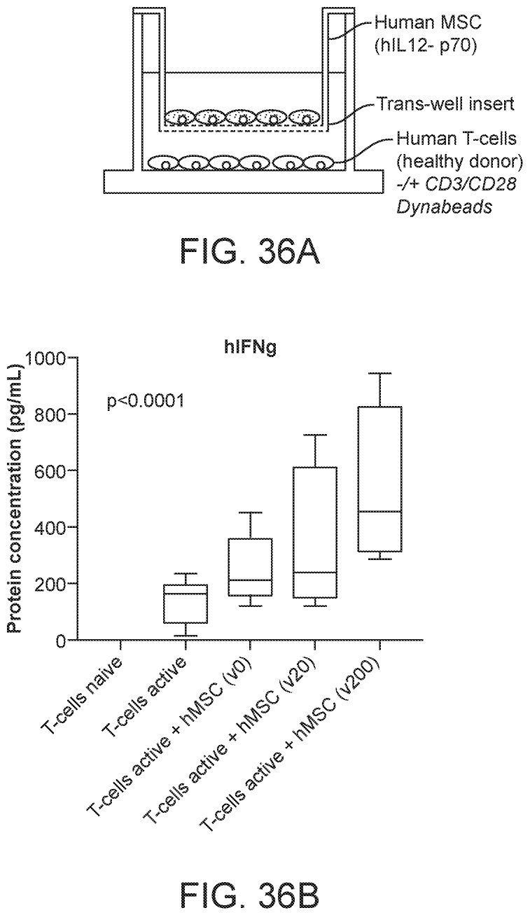

In the following example, bone-marrow derived hMSCs were engineered to express human IL12 (p70) using the lentiviral transduction method described in Example 6. Engineered hMSCs were co-cultured into 0.4 μm transwell inserts with human T-cells isolated from healthy blood donors (a schematic representation of the transwell assay is shown in FIG. 36A). To assess IL12 induced Th1 polarization on activated naïve T-cells, IFNγ production by T-cells was measured by ELISA on the supernatant collected from the lower compartment (T-cells). As shown in FIG. 36B, IFNγ production was increased in a MOI dose-dependent manner by co-culturing CD3 T-cells with hMSCs expressing IL12p70.

### Example 11: MSCs Home to Tumors in an IP Model

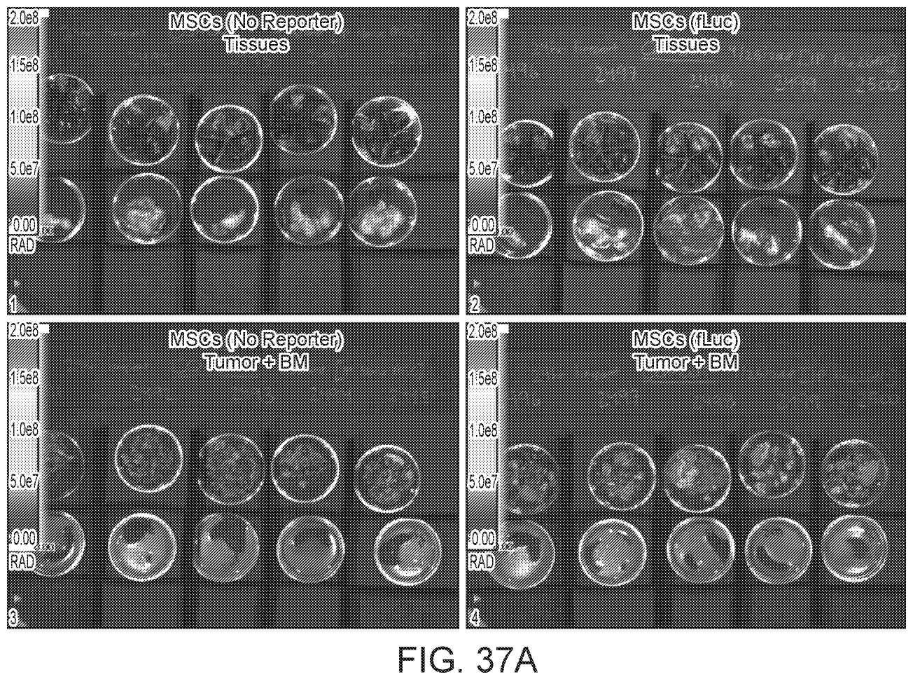

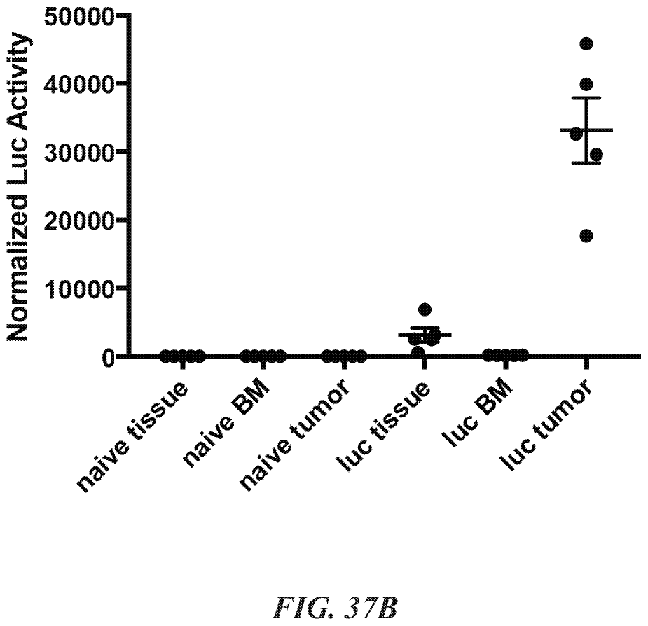

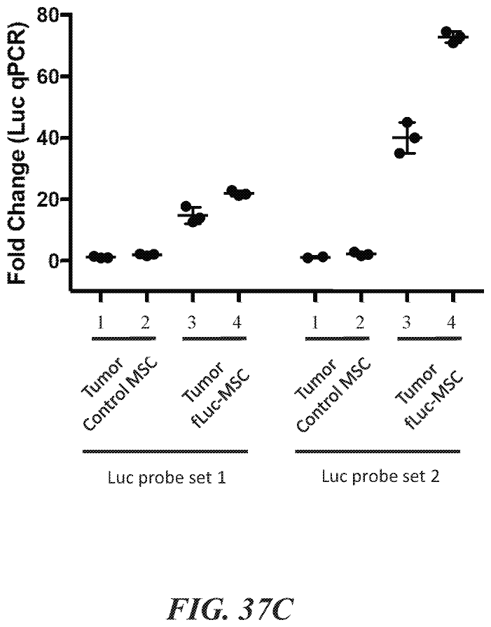

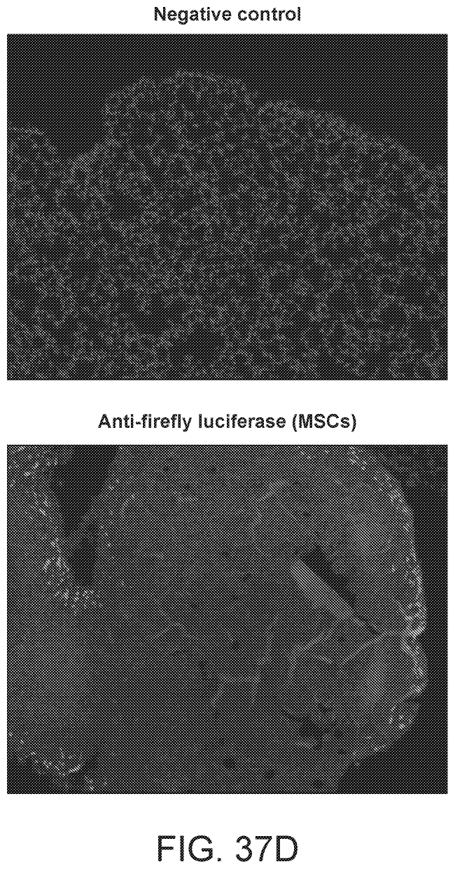

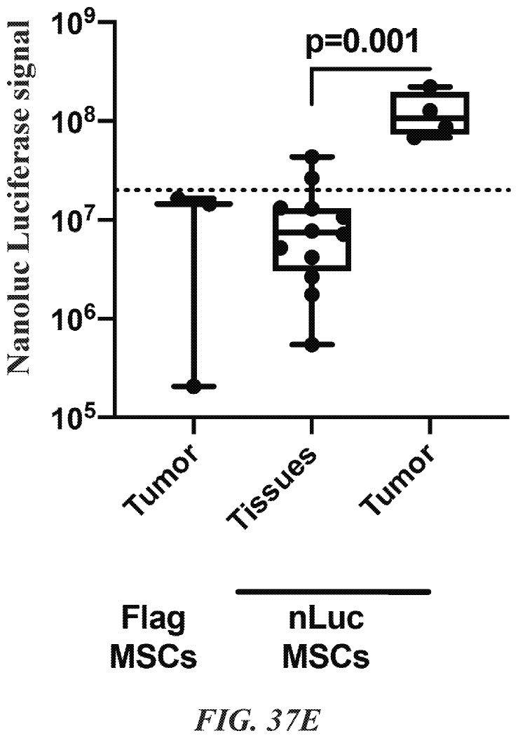

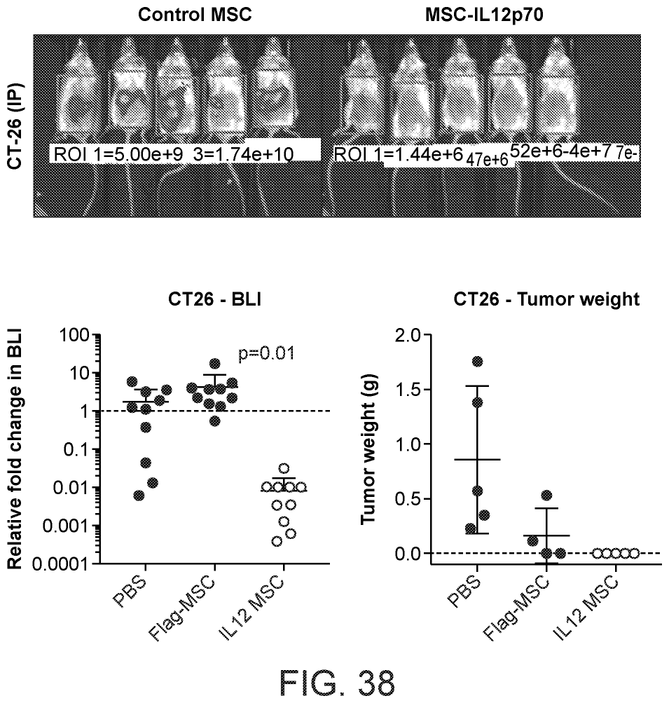

In the following example, balb/c MSCs (2×10⁶ cells) expressing fLUC were injected IP into CT-26 IP tumor-bearing mice. Mice were euthanized and tissues were collected 24 hours after injection. As shown in FIG. 37, fLUC-MSCs were significantly enriched in the tumors as detected by bioluminescence imaging (images shown in FIG. 37A, quantification of images in FIG. 37B), quantitative real time PCR (FIG. 37C), and fluorescence microscopy against firefly luciferase (FIG. 37D).

Additionally, C57Bl/6 mice were implanted with 5×10⁴B16F10-fLUC cells IP. 7 days after tumor implantation, 1×10⁶ C57Bl/6 murine BM-MSCs engineered to express Nanoluc-EGFP were injected IP. Mice were euthanized at 24 hours post injection of MSCs and peritoneal organs (stomach, kidney, liver, colon, spleen, pancreas, omentum/tumor, ovaries and Fallopian tubes) were imaged ex-vivo for nanoluc signaling (NanoGlo Substrate Kit, Vendor: Promega, Catalog No.: N1110). As shown in FIG. 37E, murine MSC nanoluc signal was preferentially enriched in the tumor compared to the other organs in the peritoneal cavity in a B16F10 tumor model.

### Example 12: IL12 Producing MSCs Reduce CT26 Tumor Burden in an IP Model

In the following example, balb/c mMSCs were engineered to express murine IL12p70 using the lentiviral transduction method described in Example 6.

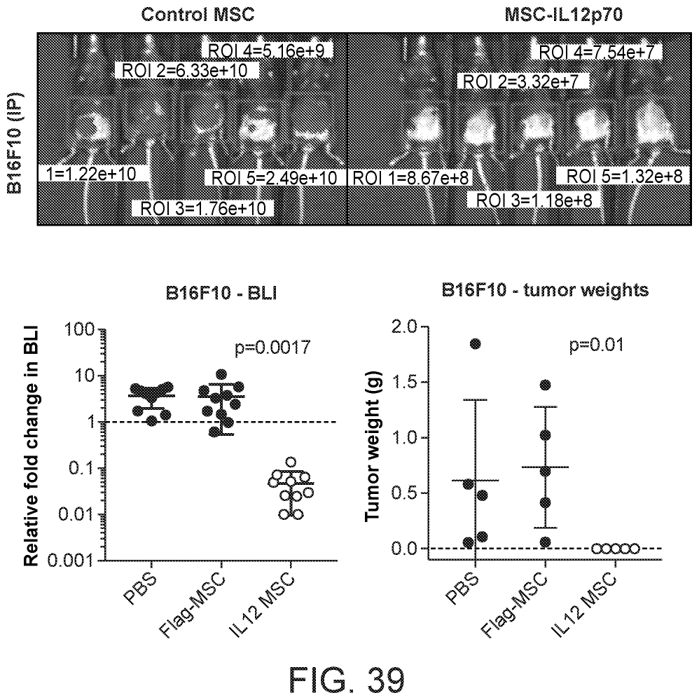

CT26 tumor cells (5×10⁴ cells in 100 μl) engineered to constitutively express luciferase enzyme (Cat no: CL043, Lot no: CL-IM147 Imanis Life Sciences) were injected into the peritoneal space of immunocompetent balb/c (age 6-8 weeks). One week after tumor implantation, tumor burden was measured by luciferase imaging (BLI) using an AMI imager. Mice were randomized into treatment groups and treated with intraperitoneally delivered mMSCs (1×10⁶ cells) expressing IL12p70. MSC-Flag-Myc and PBS were used as a negative control. As shown in FIG. 38, IL12p70 expressing MSCs led to reduction in tumor burden as assessed by BLI (top panels and bottom left panel) and a complete elimination of detectable intraperitoneal tumors by tumor weight (bottom right panel) in a CT26 model.

### Example 13: IL12 Producing MSCs Reduce B16F10 Tumor Burden in an IP Model

In the following example, C57BL/6 mMSCs were engineered to express murine IL12p70 using the lentiviral transduction method described in Example 6.

B16F10 tumor cells (5×10⁴ cells in 100 μl) engineered to constitutively express luciferase enzyme (B16F10-Fluc-Puro Cat #:CL052, lot #: CL-IM150 Imanis Life Sciences) were injected into the peritoneal space of immunocompetent C57BL/6 (age 6-8 weeks). One week after tumor implantation, tumor burden was measured by luciferase imaging (BLI) using an AMI imager. Mice were randomized into treatment groups and treated with intraperitoneally delivered mMSCs 1×10⁶ expressing IL12p70. MSC-Flag-Myc and PBS were used as a negative control. As shown in FIG. 39, IL12p70 expressing MSCs led to reduction in tumor burden as assessed by BLI (top panels and bottom left panel) and a complete elimination of detectable intraperitoneal tumors by tumor weight (bottom right panel) in a B16F10 model.

### Example 14: MSCs Producing IL12 and CCL21a Reduce Tumor Burden and Prolong Survival in a CT26 IP Tumor Model

In the following example, balb/c mMSCs were engineered to express murine IL12 (p70) and murine CCL21a from a single lentiviral expression vector. The lentiviral expression vector used a 2A ribosome skipping elements to express both cytokines from a single transcript using the lentiviral transduction method described in Example 6.

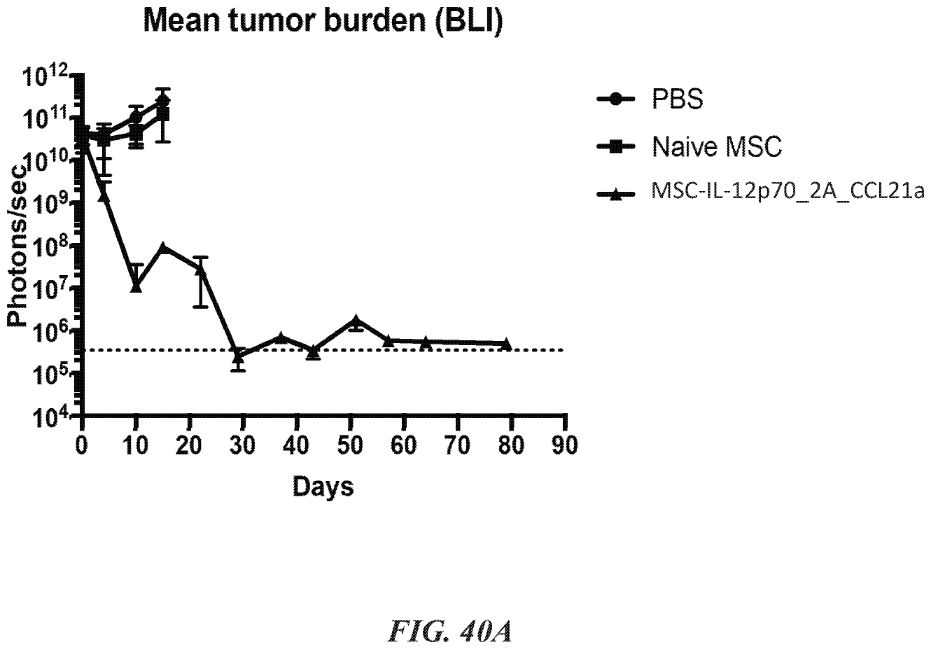

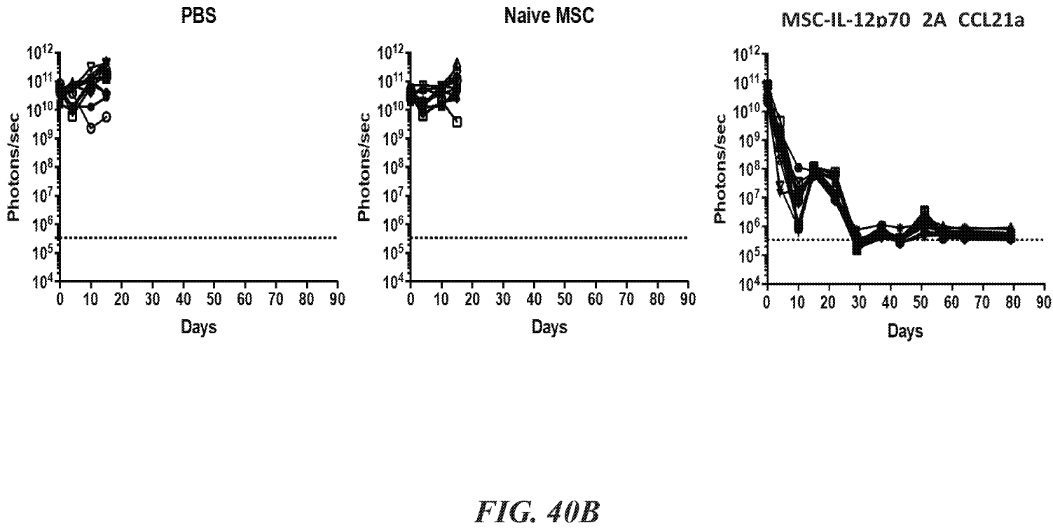

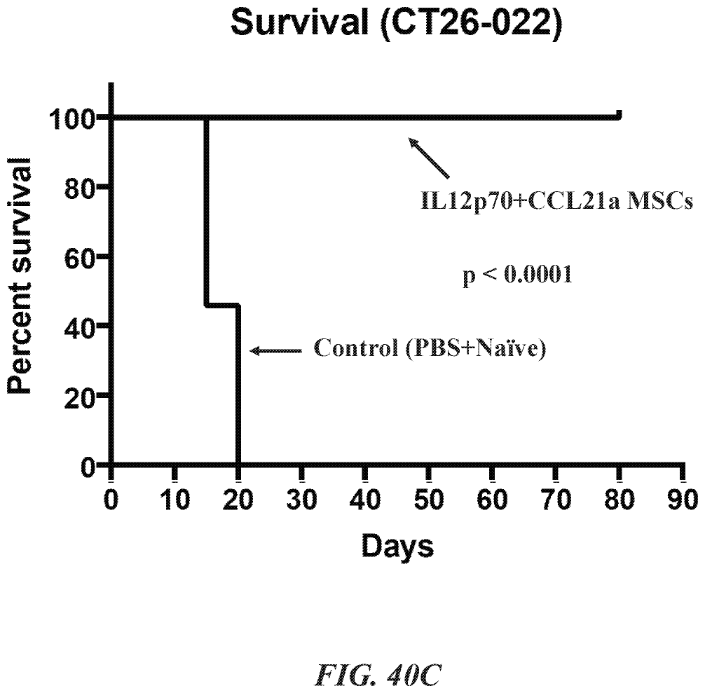

CT26 tumor cells (1×10⁶ cells) engineered to constitutively express luciferase enzyme (Cat no: CL043, Lot no: CL-IM147 Imanis Life Sciences) were injected into the peritoneal space of immunocompetent balb/c mice (age 6-8 weeks). One week after tumor implantation, tumor burden was measured by luciferase imaging (BLI) using an AMI imager. Mice were randomized into treatment groups and treated with intraperitoneally delivered mMSCs 1×10⁶ expressing IL12p70 and CCL21a by the same MSC (“MSC-IL-12p70_2A_CCL21a”). MSC-Flag-Myc and PBS were used as a negative control. As shown in FIG. 40, IL12p70/CCL21a expressing MSCs led to reduction in tumor burden as assessed by BLI (top panels and bottom left panel) and a complete elimination of detectable intraperitoneal tumors by tumor weight (bottom right panel) in a CT26 model. FIG. 40A demonstrates the mean tumor burden as assessed by BLI for PBS treated (circle), MSC-Flag-Myc (“Naïve MSC” square), and IL12p70/CCL21a expressing MSCs (triangle). FIG. 40B demonstrates the tumor burden in individual mice as assessed by BLI for PBS treated, MSC-Flag-Myc (“Naïve MSC”), and IL12p70/CCL21a expressing MSCs (left, middle, and right panels, respectively). Notably, as shown in FIG. 40C, treatment with IL12p70/CCL21a expressing MSCs led to prolonged survival (100% survival greater than 90 days), while control treated mice all died or were euthanized by Day 20.

### Example 15: MSCs Producing IL12 and IL21 Reduce Tumor Burden and Prolong Survival in a B16F10 IP Tumor Model

In the following example, C57BL/6 mMSCs were engineered to express murine IL12 (p70) or murine IL21 (i.e., each MSC engineered to express only a single agent) using the lentiviral transduction method described in Example 6.

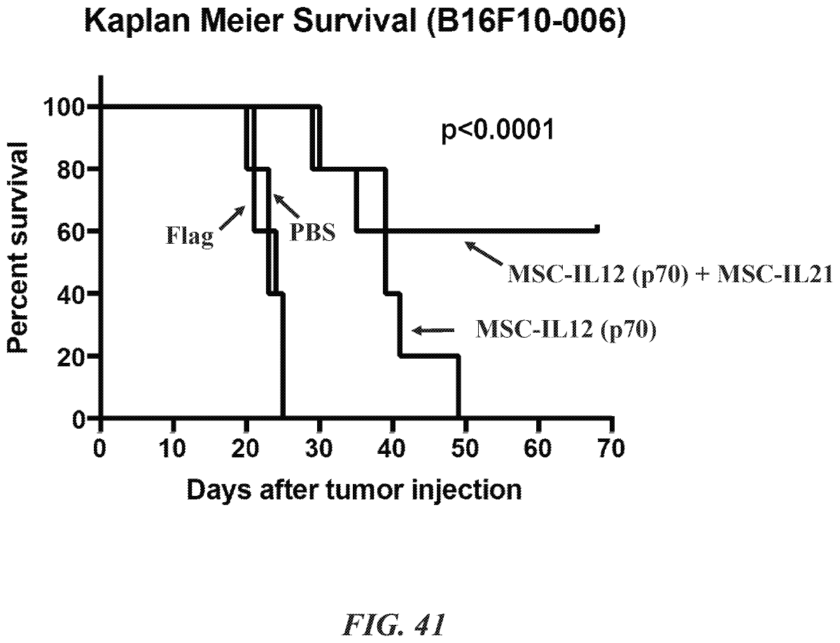

B16F10 tumor cells (5×10⁴ cells in 100 μl) engineered to constitutively express luciferase enzyme (B16F10-Fluc-Puro Cat #:CL052, lot #: CL-IM150 Imanis Life Sciences) were injected into the peritoneal space of immunocompetent C57BL/6 (age 6-8 weeks). One week after tumor implantation, tumor burden was measured by luciferase imaging (BLI) using an AMI imager. Mice were randomized into treatment groups and treated with intraperitoneally delivered mMSCs (1×10⁶ cells) expressing IL12p70 in combination with mMSCs (1×10⁶ cells) expressing IL21, or mMSCs (1×10⁶ cells) expressing IL12p70 alone. MSC-Flag-Myc and PBS were used as a negative control. As shown in FIG. 41, treatment with IL12p70 expressing MSCs led to prolonged survival relative to control treated mice but all mice still all died or were euthanized by Day 50. In contrast, treatment with IL12p70 expressing MSCs in combination with IL21 expressing MSCs led to prolonged survival relative to treatment with IL12p70 expressing MSCs (60% survival past 60 days). Thus, IL21 expression by MSCs enhanced the efficacy of IL12p70 expressing MSCs.

### Example 16: Allogeneic MSCs Producing IL12 and CCL21a Reduce Tumor Burden and Prolong Survival in a CT26 IP Tumor Model

In the following example, balb/c mMSCs (syngeneic) and C57BL/6 mMSCs (allogeneic) were engineered to express murine IL12 (p70) and murine CCL21a from a single lentiviral expression vector. The lentiviral expression vector used a 2A ribosome skipping elements to express both cytokines from a single transcript using the lentiviral transduction method described in Example 6.

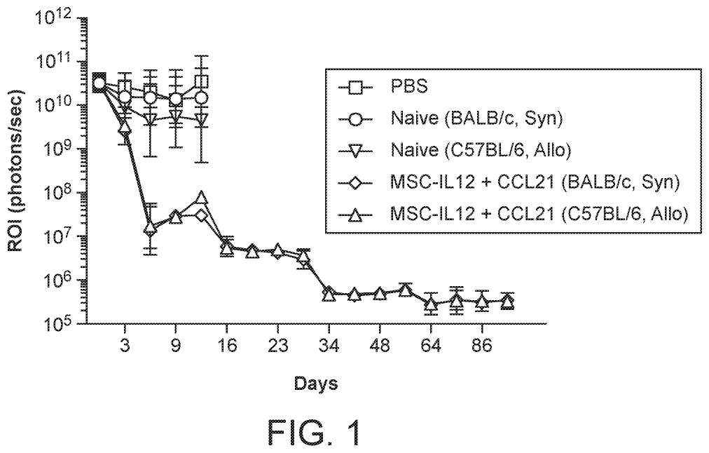

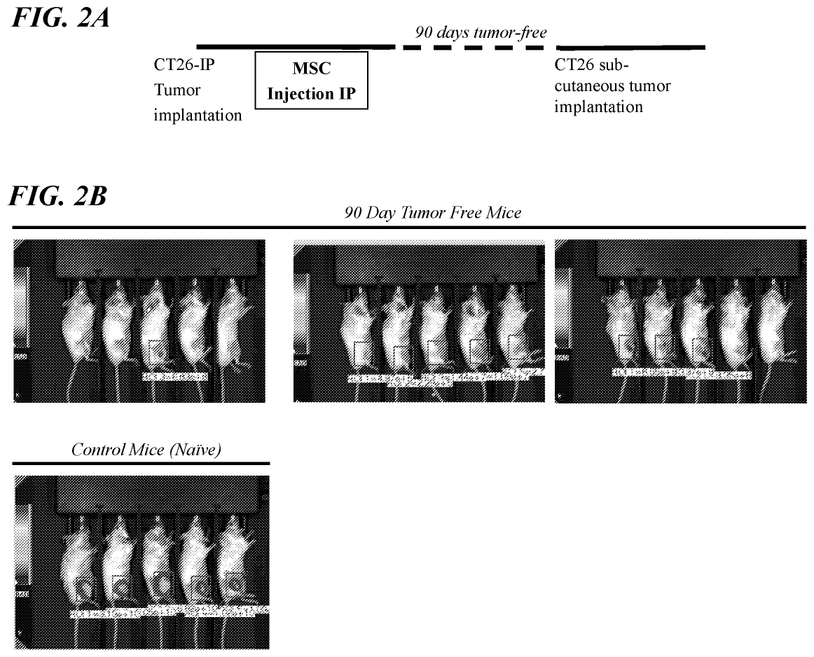

CT26 tumor cells (1×10⁶ cells) engineered to constitutively express luciferase enzyme (Cat no: CL043, Lot no: CL-IM147 Imanis Life Sciences) were injected into the peritoneal space of immunocompetent balb/c mice (age 6-8 weeks). One week after tumor implantation, tumor burden was measured by luciferase imaging (BLI) using an AMI imager. Mice were randomized into treatment groups and treated with intraperitoneally delivered mMSCs (1×10⁶ cells) expressing IL12p70 and CCL21a by the same MSC (“MSC-IL12+CCL21”). Both balb/c control mMSCs (syngeneic) and C57BL/6 control mMSCs (allogeneic) were engineered to express MSC-Flag-Myc (“Naïve”). PBS was also used as a negative control. As shown in FIG. 1, both syngeneic and allogeneic MSCs expressing IL12p70/CCL21a led to reduction in tumor burden as assessed by BLI in a CT26 model, while control treatments did not. Additionally, mice that were previously treated with mMSCs expressing IL12p70 and CCL21a in both syngeneic and allogeneic models and were determined to be tumor free for 90 days were subsequently challenged with CT26 tumor cells (0.5×10⁶ cells in 100 μl PBS) implanted subcutaneously in the thigh, as schematized in FIG. 2A. As shown in FIG. 2B, tumor free mice rejected the tumor implant in contrast to naïve control mice where the tumor became established. Thus, treatment with MSCs expressing IL12p70/CCL21a led to prolonged tumor burden reduction as well as immunological memory.

### Example 17: MSCs Producing IL12 and CCL21a Demonstrate Enhanced Growth Relative to Unmodifed Cells

In the following example, human MSCs from 3 different donors were engineered at different multiplicity of infections (MOIs) to express and secrete human IL-12 and human CCL21a from a single lentiviral expression vector. The lentiviral expression vector used a 2A ribosome skipping elements to express both cytokines from a single transcript using the lentiviral transduction method described in Example 6.

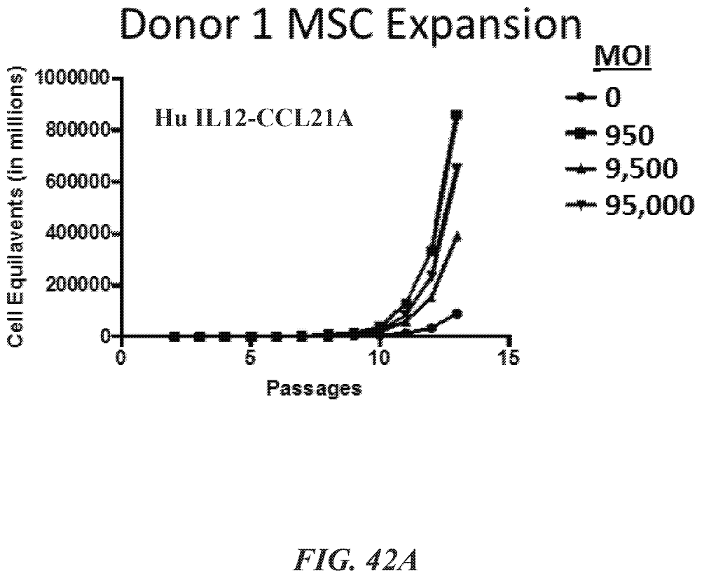

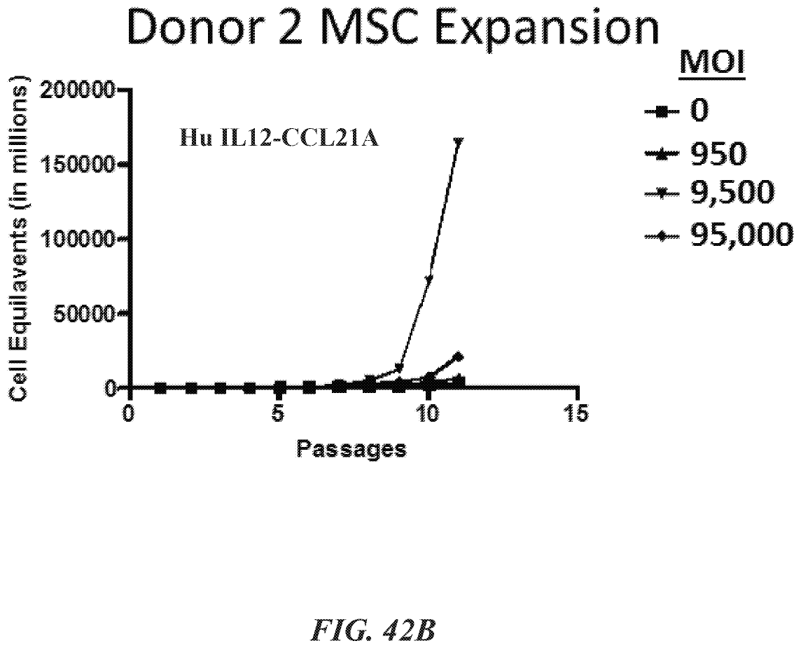

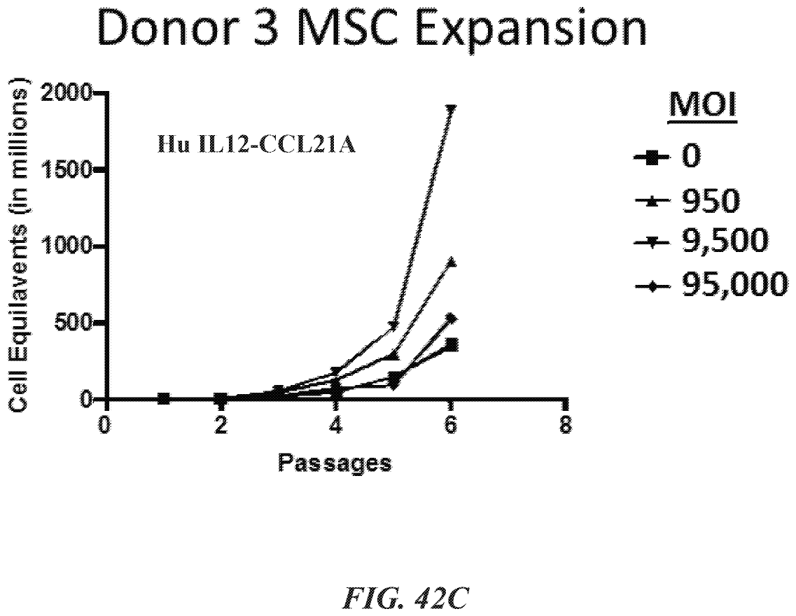

As shown in FIG. 42, the genetically engineered MSCs (M01=95000, 9500, or 950) exhibited enhanced cell expansion and growth compared to the non-genetically engineered human MSCs (MOI=0) in the three donors tested (FIG. 42A, Donor 1; FIG. 42B, Donor 2; FIG. 42C, Donor 3). Human MSCs genetically engineered with lentivirus to express GFP did not show a similar enhanced cell expansion or growth phenotype (data not shown).

### Example 18: Selection of Promoter for Sustained Protein Expression in Human Bone-Marrow MSCs (BM-MSCs)

In the following example, various promoters were tested for driving expression of a reporter EGFP construct in human MSCs. Promoters tested were CMV, SFFV, EF1a, EF1a-LTR, EFS, MND, PGK, UbC (see Table 4). Cells were transduced using equivalent MOI (multiplicity of infection) using the lentiviral transduction method described in Example. EGFP percentage and Median Fluorescence Intensity (MFI) were quantified over serial passages using flow cytometry.

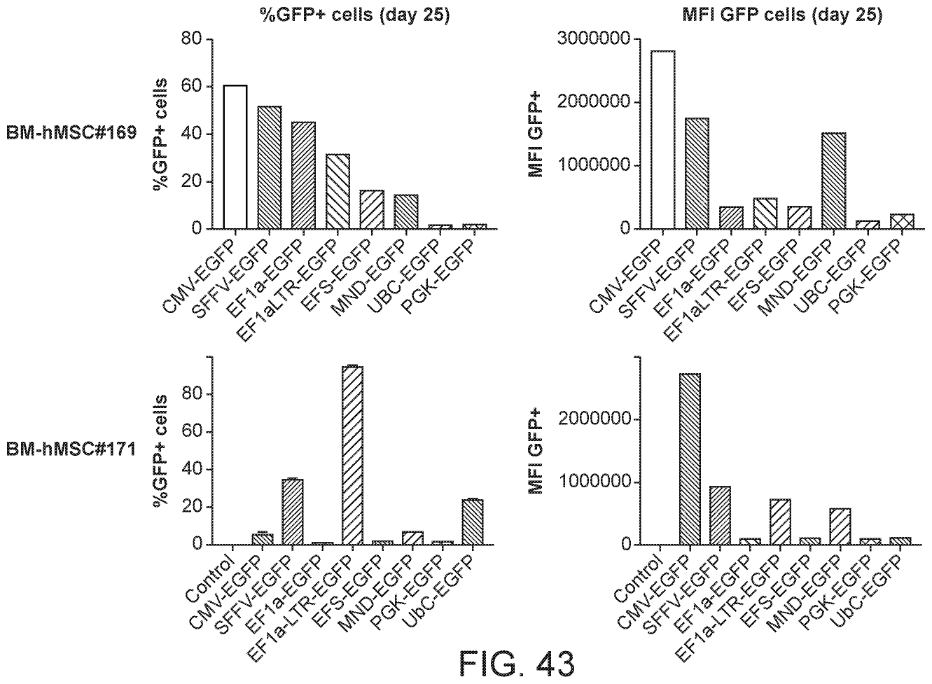

As shown in FIG. 43, two independent human BM-MSC cell lines from 2 different donors (top and bottom row, respectively were engineered and percent GFP (left panels) and MFI (right panels) of engineered cells was assessed at day 25 post transduction. The SFFV promoter demonstrated GFP expression in both cell lines by both GFP percentage and MFI.

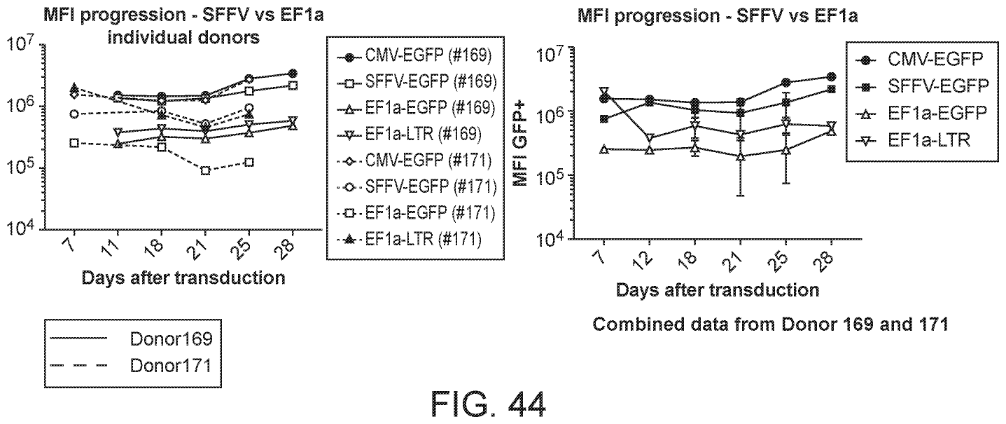

As shown in FIG. 44, EGFP MFI was tracked over time (day 7 to day 28 post-transduction) for either the two independent human BM-MSC cell lines individually (left panel) or with data from the two independent human BM-MSC cell lines combined (right panel). Protein expression was stable over time during more than 28 days. Additionally, in comparison to EF1a promoters, SFFV promoter consistently drove almost ten-fold more protein expression as quantified by MFI.

### Example 19: Engineering Human MSCs to Produce IL12 and IL21

In the following example, human bone-marrow MSCs were stably transduced to express IL12p70 and IL21 from various constructs using the lentiviral transduction method described in Example 6. Cells were expanded for 3 to 4 passages post-transduction and 0.2×10⁶ cells were seeded in 6-well plates in 4 mL of media. Conditioned media was collected after 24 hours and ELISAs were performed to determine the IL-12 and IL-21 concentrations produced.

Various constructs were tested with different combinations and/or arrangements of promoter-signal sequence 1-cytokine 1-2A linker-signal sequence 2-cytokine 2. The combinations tested are described below in Table 7. Specific details of construct SB00880 are presented below in Table 8.

TABLE 7
IL-12 and IL-21 Expression Constructs

| Construct | | | | Codon |
|---|---|---|---|---|
| Name | | | Back- | Opti- |
| (SB #) | Promoter | Insert | bone | mization |
| SB00743 | SFFV_1 | IL12ss-IL12 | pL23d | None |
| SB00763 | EFa1 | IL12ss-IL12-fT2A*- | pL40g | None |
| | (pEF6) | IL21ss-IL21 | | |
| SB00765 | EFa1 | IL12ss-IL12-fT2A-IL12ss-IL21 | pL40g | None |
| | (pEF6) | | | |
| SB00766 | EFa1 | IL12ss-IL12-fT2A-IL8ss-IL21 | pL40g | None |
| | (pEF6) | | | |
| SB00767 | EFa1 | IL12ss-IL12-IT2A-IL21 | pL40g | None |
| | (pEF6) | | | |
| SB00768 | EFa1 | IL21ss-IL21-fT2A-IL12ss-IL12 | pL40g | None |
| | (pEF6) | | | |
| SB00769 | EFa1 | IL12ss-IL21-IT2A-IL12ss-IL12 | pL40g | None |
| | (pEF6) | | | |
| SB00770 | EFa1 | IL6ss-IL21-fT2A-IL12ss-IL12 | pL40g | None |
| | (pEF6) | | | |
| SB00771 | EFa1 | IL8ss-IL21-fT2A-IL12ss-IL12 | pL40g | None |
| | (pEF6) | | | |
| SB00772 | EFa1 | IL21SS-IL21-IT2A-IL12 | pL40g | None |
| | (pEF6) | | | |
| SB00773 | EFa1 | IL12ss-IL21-IT2A-IL12 | pL40g | None |
| | (pEF6) | | | |
| SB00774 | EFa1 | IL6ss-IL21-IT2A-IL12 | pL40g | None |
| | (pEF6) | | | |
| SB00775 | EFa1 | IL8ss-IL21-IT2A-IL12 | pL40g | None |
| | (pEF6) | | | |
| SB00772 | EFa1 | IL21SS-IL21-IT2A-IL12 | pL40g | None |
| | (pEF6) | | | |
| SB00620 | SFFV_1 | IL2ss-IL21 | pL17d | None |
| SB00838 | SFFV_1 | IL12ss-IL12-IT2A- | pL41g | None |
| | | IL21SS-IL21 | | |
| SB00839 | SFFV_1 | IL12ss-IL12-IT2A-IL8ss-IL21 | pL41g | None |
| SB00840 | SFFV_1 | IL12ss-IL12-fT2A-IL21 | pL41g | None |
| SB00841 | SFFV_1 | IL21ss-IL21-fT2A-IL12ss-IL12 | pL41g | None |
| SB00843 | SFFV_1 | IL21ss-IL21-fT2A-IL12 | pL41g | None |
| SB00844 | SFFV_1 | IL8ss-IL21-IT2A-IL12 | pL41g | None |
| SB00868 | SFFV_1 | IL12ss-IL12 | pL41g | Yes |
| SB00870 | EFa1 | IL12ss-IL12-IT2A- | pL40g | Yes |
| | (pEF6) | IL21SS-IL21 | | |
| SB00872 | EFa1 | IL12ss-IL12-IT2A-IL21 | pL40g | Yes |
| | (pEF6) | | | |
| SB00869 | EFa1 | IL21SS-IL21-IT2A- | pL40g | Yes |
| | (pEF6) | IL12ss-IL12 | | |
| SB00871 | EFa1 | IL21SS-IL21-IT2A-IL12 | pL40g | Yes |
| | (pEF6) | | | |
| SB00879 | SFFV_1 | IL21ss-IL21-fT2A- | pL41g | Yes |
| | | IL12ss-IL12 | | |
| SB00880 | SFFV_1 | IL12ss-IL12-IT2A- | pL41g | Yes |
| | | IL21SS-IL21 | | |
| SB00881 | SFFV_1 | IL21ss-IL21-fT2A-IL12 | pL41g | Yes |
| SB00882 | SFFV_1 | IL12ss-IL12-fT2A-IL21 | pL41g | Yes |
| SB00862 | SFFV_1 | IL21SS-IL21 | pL23d | None |
| SB00863 | SFFV_1 | IL2ss-IL21 | pL41g | Yes |
| SB00968 | SFFV_1 | IL2ss-IL21-fT2A-IL12ss-IL12 | pL41g | Yes |
| SB00969 | SFFV_1 | IL8ss-IL21-fT2A-IL12ss-IL12 | pL41g | Yes |
| SB00970 | SFFV_1 | IL12ss-IL12-IT2A-IL2ss-IL21 | pL41g | Yes |
| SB00971 | SFFV_1 | IL12ss-IL12-IT2A-IL8ss-IL21 | pL41g | Yes |
| SB00862 + | SFFV_1 | Co-transduction IL12 + IL21 | pL41g | None |
| SB00743 | | | | |
| SB00868 + | SFFV_1 | Co-transduction IL12 + IL21 | pL41g | Yes |
| 863 | | (IL2ssIL21) | | |

* fT2A refers to Furin-T2A

TABLE 8
SB00880 Expression Construct Sequences

SFFV promoter (SEQ ID NO: 17)
GTAACGCCATTTTGCAAGGCATGGAAAAATACCAAACCAAGAATAGAGAAGTTCAGATCAAGG
GCGGGTACATGAAAATAGCTAACGTTGGGCCAAACAGGATATCTGCGGTGAGCAGTTTCGGCCC
CGGCCCGGGGCCAAGAACAGATGGTCACCGCAGTTTCGGCCCCGGCCCGAGGCCAAGAACAGA
TGGTCCCCAGATATGGCCCAACCCTCAGCAGTTTCTTAAGACCCATCAGATGTTTCCAGGCTCCC
CCAAGGACCTGAAATGACCCTGCGCCTTATTTGAATTAACCAATCAGCCTGCTTCTCGCTTCTGT
TCGCGCGCTTCTGCTTCCCGAGCTCTATAAAAGAGCTCACAACCCCTCACTCGGCGCGCCAGTCC
TCCGACAGACTGAGTCGCCCGGG
Human IL-12 signal sequence; codon optimized (nucleic acid)
(SEQ ID NO: 32)
ATGTGCCATCAGCAACTCGTCATCTCCTGGTTCTCCCTTGTGTTCCTCGCTTCCCCTCTGGTCGCC
Human IL-12 signal sequence (amino acid) (SEQ ID NO: 112)
MCHQQLVISWFSLVFLASPLVA
Human IL-12 protein without signal sequence; codon optimized
(nucleic acid) (SEQ ID NO: 136)
ATTTGGGAACTGAAGAAGGACGTCTACGTGGTCGAGCTGGATTGGTACCCGGACGCCCCTGGAG
AAATGGTCGTGCTGACTTGCGATACGCCAGAAGAGGACGGCATAACCTGGACCCTGGATCAGA
GCTCCGAGGTGCTCGGAAGCGGAAAGACCCTGACCATTCAAGTCAAGGAGTTCGGCGACGCGG
GCCAGTACACTTGCCACAAGGGTGGCGAAGTGCTGTCCCACTCCCTGCTGCTGCTGCACAAGAA
AGAGGATGGAATCTGGTCCACTGACATCCTCAAGGACCAAAAAGAACCGAAGAACAAGACCTT
CCTCCGCTGCGAAGCCAAGAACTACAGCGGTCGGTTCACCTGTTGGTGGCTGACGACAATCTCC
ACCGACCTGACTTTCTCCGTGAAGTCGTCACGGGGATCAAGCGATCCTCAGGGCGTGACCTGTG
GAGCCGCCACTCTGTCCGCCGAGAGAGTCAGGGGAGACAACAAGGAATATGAGTACTCCGTGG
AATGCCAGGAGGACAGCGCCTGCCCTGCCGCGGAAGAGTCCCTGCCTATCGAGGTCATGGTCGA
TGCCGTGCATAAGCTGAAATACGAGAACTACACTTCCTCCTTCTTTATCCGCGACATCATCAAGC
CTGACCCCCCCAAGAACTTGCAGCTGAAGCCACTCAAGAACTCCCGCCAAGTGGAAGTGTCTTG
GGAATATCCAGACACTTGGAGCACCCCGCACTCATACTTCTCGCTCACTTTCTGTGTGCAAGTGC
AGGGAAAGTCCAAACGGGAGAAGAAAGACCGGGTGTTCACCGACAAAACCTCCGCCACTGTGA
TTTGTCGGAAGAACGCGTCAATCAGCGTCCGGGCGCAGGATAGATACTACTCGTCCTCCTGGAG
CGAATGGGCCAGCGTGCCTTGTTCCGGTGGCGGATCAGGCGGAGGTTCAGGAGGAGGCTCCGG
AGGAGGTTCCCGGAACCTCCCTGTGGCAACCCCCGACCCTGGAATGTTCCCGTGCCTACACCAC
TCCCAAAACCTCCTGAGGGCTGTGTCGAACATGTTGCAGAAGGCCCGCCAGACCCTTGAGTTCT
ACCCCTGCACCTCGGAAGAAATTGATCACGAGGACATCACCAAGGACAAGACCTCGACCGTGG
AAGCCTGCCTGCCGCTGGAACTGACCAAGAACGAATCGTGTCTGAACTCCCGCGAGACAAGCTT
TATCACTAACGGCAGCTGCCTGGCGTCGAGAAAGACCTCATTCATGATGGCGCTCTGTCTTTCCT
CGATCTACGAAGATCTGAAGATGTATCAGGTCGAGTTCAAGACCATGAACGCCAAGCTGCTCAT
GGACCCGAAGCGGCAGATCTTCCTGGACCAGAATATGCTCGCCGTGATTGATGAACTGATGCAG
GCCCTGAATTTCAACTCCGAGACTGTGCCTCAAAAGTCCAGCCTGGAAGAACCGGACTTCTACA
AGACCAAGATCAAGCTGTGCATCCTGTTGCACGCTTTCCGCATTCGAGCCGTGACCATTGACCG
CGTGATGTCCTACCTGAACGCCAGT
Human IL-12 protein without signal sequence (amino acid)
(SEQ ID NO: 137); p35 subunit in bold; p40 subunit in italics
IWELKKDVYVVELDWYPDAPGEMVVLTCDTPEEDGITWTLDQSSEVLGSGKTLTIQVKEFGDAGQY
TCHKGGEVLSHSLLLLHKKEDGIWSTDILKDQKEPKNKTFLRCEAKNYSGRFTCWWLTTISTDLTFS
VKSSRGSSDPQGVTCGAATLSAERVRGDNKEYEYSVECQEDSACPAAEESLPIEVMVDAVHKLKYE
NYTSSFFIRDIIKPDPPKNLQLKPLKNSRQVEVSWEYPDTWSTPHSYFSLTFCVQVQGKSKREKKDRV
FTDKTSATVICRKNASISVRAQDRYYSSSWSEWASVPCSGGGSGGGSGGGSGGGS

[symbol]

[symbol]

[symbol]

[symbol]

[symbol]

[symbol]

[symbol]

[symbol]

[symbol]

[symbol]

[symbol]

[symbol]

Human IL-12 protein with signal sequence (amino acid)
(SEQ ID NO: 138); p35 subunit in bold; p40 subunit in italics
MCHQQLVISWFSLVFLASPLVAIWELKKDVYVVELDWYPDAPGEMVVLTCDTPEEDGITWTLDQSS
EVLGSGKTLTIQVKEFGDAGQYTCHKGGEVLSHSLLLLHKKEDGIWSTDILKDQKEPKNKTFLRCEA
KNYSGRFTCWWLTTISTDLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSRQVEVSWEYPDTWSTPHSY
FSLTFCVQVQGKSKREKKDRVFTDKTSATVICRKNASISVRAQDRYYSSSWSEWASVPCSGGGSGG
GSGGGSGGGSRNLPVA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Furin-T2A Linker (nucleic acid) (SEQ ID NO: 139)
AGACGGAAACGCGGAAGCGGAGAGGGCAGAGGCTCGCTGCTTACATGCGGGGACGTGGAAGA
GAACCCCGGTCCG
Furin-T2A Linker (amino acid) (SEQ ID NO: 140)
RRKRGSGEGRGSLLTCGDVEENPGP
Human IL-21 signal sequence; codon optimized (nucleic acid)
(SEQ ID NO: 55)
ATGGAACGCATTGTGATCTGCCTGATGGTCATCTTCCTGGGCACCTTAGTGCACAAGTCGAGCA
GC
Human IL-21 signal sequence (amino acid) (SEQ ID NO: 135)
MERIVICLMVIFLGTLVHKSSS
Human IL-21 protein without signal sequence; codon optimized
(nucleic acid) (SEQ ID NO: 141)
CAGGGACAGGACAGGCACATGATTAGAATGCGCCAGCTCATCGATATCGTGGACCAGTTGAAG
AACTACGTGAACGACCTGGTGCCCGAGTTCCTGCCGGCCCCCGAAGATGTGGAAACCAATTGCG
AATGGTCGGCATTTTCCTGCTTTCAAAAGGCACAGCTCAAGTCCGCTAACACCGGGAACAACGA
ACGGATCATCAACGTGTCCATCAAAAAGCTGAAGCGGAAGCCTCCCTCCACCAACGCCGGACG
GAGGCAGAAGCATAGGCTGACTTGCCCGTCATGCGACTCCTACGAGAAGAAGCCGCCGAAGGA
GTTCCTGGAGCGGTTCAAGTCGCTCCTGCAAAAGATGATTCATCAGCACCTGTCCTCCCGGACTC
ATGGGTCTGAGGATTCA
Human IL-21 protein without signal sequence (amino acid)
(SEQ ID NO: 142)
QGQDRHMIRMRQLIDIVDQLKNYVNDLVPEFLPAPEDVETNCEWSAFSCFQKAQLKSANTGNNERII
NVSIKKLKRKPPSTNAGRRQKHRLTCPSCDSYEKKPPKEFLERFKSLLQKMIHQHLSSRTHGSEDS
Human IL-21 protein with signal sequence (amino acid)
(SEQ ID NO: 143)
MERIVICLMVIFLGTLVHKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPSCDSYEKKPPKEFLERFKS
LLQKMIHQHLSSRTHGSEDS
SB00880 Cassette (SFFV-IL12ss-IL12-fT2A-IL21ss-IL21)
(SEQ ID NO: 144)
GTAACGCCATTTTGCAAGGCATGGAAAAATACCAAACCAAGAATAGAGAAGTTCAGATCAAGG
GCGGGTACATGAAAATAGCTAACGTTGGGCCAAACAGGATATCTGCGGTGAGCAGTTTCGGCCC
CGGCCCGGGGCCAAGAACAGATGGTCACCGCAGTTTCGGCCCCGGCCCGAGGCCAAGAACAGA
TGGTCCCCAGATATGGCCCAACCCTCAGCAGTTTCTTAAGACCCATCAGATGTTTCCAGGCTCCC
CCAAGGACCTGAAATGACCCTGCGCCTTATTTGAATTAACCAATCAGCCTGCTTCTCGCTTCTGT
TCGCGCGCTTCTGCTTCCCGAGCTCTATAAAAGAGCTCACAACCCCTCACTCGGCGCGCCAGTCC
TCCGACAGACTGAGTCGCCCGGGGGATCCGCGGAATTCGCCGCCACCATGTGCCATCAGCAACT
CGTCATCTCCTGGTTCTCCCTTGTGTTCCTCGCTTCCCCTCTGGTCGCCATTTGGGAACTGAAGAA
GGACGTCTACGTGGTCGAGCTGGATTGGTACCCGGACGCCCCTGGAGAAATGGTCGTGCTGACT
TGCGATACGCCAGAAGAGGACGGCATAACCTGGACCCTGGATCAGAGCTCCGAGGTGCTCGGA
AGCGGAAAGACCCTGACCATTCAAGTCAAGGAGTTCGGCGACGCGGGCCAGTACACTTGCCAC
AAGGGTGGCGAAGTGCTGTCCCACTCCCTGCTGCTGCTGCACAAGAAAGAGGATGGAATCTGGT
CCACTGACATCCTCAAGGACCAAAAAGAACCGAAGAACAAGACCTTCCTCCGCTGCGAAGCCA
AGAACTACAGCGGTCGGTTCACCTGTTGGTGGCTGACGACAATCTCCACCGACCTGACTTTCTCC
GTGAAGTCGTCACGGGGATCAAGCGATCCTCAGGGCGTGACCTGTGGAGCCGCCACTCTGTCCG
CCGAGAGAGTCAGGGGAGACAACAAGGAATATGAGTACTCCGTGGAATGCCAGGAGGACAGC
GCCTGCCCTGCCGCGGAAGAGTCCCTGCCTATCGAGGTCATGGTCGATGCCGTGCATAAGCTGA
AATACGAGAACTACACTTCCTCCTTCTTTATCCGCGACATCATCAAGCCTGACCCCCCCAAGAAC
TTGCAGCTGAAGCCACTCAAGAACTCCCGCCAAGTGGAAGTGTCTTGGGAATATCCAGACACTT
GGAGCACCCCGCACTCATACTTCTCGCTCACTTTCTGTGTGCAAGTGCAGGGAAAGTCCAAACG
GGAGAAGAAAGACCGGGTGTTCACCGACAAAACCTCCGCCACTGTGATTTGTCGGAAGAACGC
GTCAATCAGCGTCCGGGCGCAGGATAGATACTACTCGTCCTCCTGGAGCGAATGGGCCAGCGTG
CCTTGTTCCGGTGGCGGATCAGGCGGAGGTTCAGGAGGAGGCTCCGGAGGAGGTTCCCGGAAC
CTCCCTGTGGCAACCCCCGACCCTGGAATGTTCCCGTGCCTACACCACTCCCAAAACCTCCTGAG
GGCTGTGTCGAACATGTTGCAGAAGGCCCGCCAGACCCTTGAGTTCTACCCCTGCACCTCGGAA
GAAATTGATCACGAGGACATCACCAAGGACAAGACCTCGACCGTGGAAGCCTGCCTGCCGCTG
GAACTGACCAAGAACGAATCGTGTCTGAACTCCCGCGAGACAAGCTTTATCACTAACGGCAGCT
GCCTGGCGTCGAGAAAGACCTCATTCATGATGGCGCTCTGTCTTTCCTCGATCTACGAAGATCTG
AAGATGTATCAGGTCGAGTTCAAGACCATGAACGCCAAGCTGCTCATGGACCCGAAGCGGCAG
ATCTTCCTGGACCAGAATATGCTCGCCGTGATTGATGAACTGATGCAGGCCCTGAATTTCAACTC
CGAGACTGTGCCTCAAAAGTCCAGCCTGGAAGAACCGGACTTCTACAAGACCAAGATCAAGCT
GTGCATCCTGTTGCACGCTTTCCGCATTCGAGCCGTGACCATTGACCGCGTGATGTCCTACCTGA
ACGCCAGTAGACGGAAACGCGGAAGCGGAGAGGGCAGAGGCTCGCTGCTTACATGCGGGGACG
TGGAAGAGAACCCCGGTCCGATGGAACGCATTGTGATCTGCCTGATGGTCATCTTCCTGGGCAC
CTTAGTGCACAAGTCGAGCAGCCAGGGACAGGACAGGCACATGATTAGAATGCGCCAGCTCAT
CGATATCGTGGACCAGTTGAAGAACTACGTGAACGACCTGGTGCCCGAGTTCCTGCCGGCCCCC
GAAGATGTGGAAACCAATTGCGAATGGTCGGCATTTTCCTGCTTTCAAAAGGCACAGCTCAAGT
CCGCTAACACCGGGAACAACGAACGGATCATCAACGTGTCCATCAAAAAGCTGAAGCGGAAGC
CTCCCTCCACCAACGCCGGACGGAGGCAGAAGCATAGGCTGACTTGCCCGTCATGCGACTCCTA
CGAGAAGAAGCCGCCGAAGGAGTTCCTGGAGCGGTTCAAGTCGCTCCTGCAAAAGATGATTCA
TCAGCACCTGTCCTCCCGGACTCATGGGTCTGAGGATTCATGA
SB00880 Full Vector (PL41g + SB00880 Cassette) (SEQ ID NO: 145)
TGACTCCTGCGCAGTCCAAAAAAAAAGGCTCCAAAAGGAGCCTTTAATTGTATCGGTGGGCCCT
TAGAAAAACTCATCGAGCATCAAATGAAACTGCAATTTATTCATATCAGGATTATCAATACCAT
ATTTTTGAAAAAGCCGTTTCTGTAATGAAGGAGAAAACTCACCGAGGCAGTTCCATAGGATGGC
AAGATCCTGGTATCGGTCTGCGATTCCGACTCGTCCAACATCAATACAACCTATTAATTTCCCCT
CGTCAAAAATAAGGTTATCAAGTGAGAAATCACCATGAGTGACGACTGAATCCGGTGAGAATG
GCAAAAGCTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAATACGCGAT
CGCTGTTAAAAGGACAATTACAAACAGGAATCGAATGCAACCGGCGCAGGAACACTGCCAGCG
CATCAACAATATTTTCACCTGAATCAGGATATTCTTCTAATACCTGGAATGCTGTTTTCCCGGGG
ATCGCAGTGGTGAGTAACCATGCATCATCAGGAGTACGGATAAAATGCTTGATGGTCGGAAGA
GGCATAAATTCCGTCAGCCAGTTTAGTCTGACCATCTCATCTGTAACATCATTGGCAACGCTACC
TTTGCCATGTTTCAGAAACAACTCTGGCGCATCGGGCTTCCCATACAATCGATAGATTGTCGCAC
CTGATTGCCCGACATTATCGCGAGCCCATTTATACCCATATAAATCAGCATCCATGTTGGAATTT
AATCGCGGCCTCGAGCAAGACGTTTCCCGTTGAATATGGCTCATAACACCCCTTGTATTACTGTT
TATGTAAGCAGACAGTTTTATTGTTCATGATGATATATTTTTATCTTGTGCAATGTAACATCAGA
GATTTTGAGACACAACGTGGTTTAAACAAATAGTCAAAAGCCTCCGGCGACTAGTCGGGGTCAT
TAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAG
GGACTTTCCATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGG
GGATTTCCAAGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCGTGTACGGTG
GGAGGTCTATATAAGCAGAGCTGGTTTAGTGAACCGGGTCTCTCTGGTTAGACCAGATTTGAGC
CTGGGAGCTCTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCAGACCCTTTTA
GTCAGTGTGGAAAATCTCTAGCAGTGGCGCCCGAACAGGGACCTGAAAGCGAAAGGGAAACCA
GAGGAGCTCTCTCGACGCAGGACTCGGCTTGCTGAAGCGCGCACGGCAAGAGGCGAGGGGCGG
CGACTGCAGAGTACGCCAAAATTTTGACTAGCGGAGGCTAGAAGGAGAGAGATGGGTGCGAGA
GCGTCAGTATTAAGCGGGGGAAAATAGCGGCCGCCACAATTTTAAAAGAAAAGGGGGGATTGG
GGGGTACAGTGCAGGGGAAAGAATAGTAGACATAATAGCAACAGACATACAAACTAAAGAATT
ACAAAAACAAATTACAAAAATTCAAATTTTCGGGGGATCCGTAACGCCATTTTGCAAGGCATGG
AAAAATACCAAACCAAGAATAGAGAAGTTCAGATCAAGGGCGGGTACATGAAAATAGCTAACG
TTGGGCCAAACAGGATATCTGCGGTGAGCAGTTTCGGCCCCGGCCCGGGGCCAAGAACAGATG
GTCACCGCAGTTTCGGCCCCGGCCCGAGGCCAAGAACAGATGGTCCCCAGATATGGCCCAACCC
TCAGCAGTTTCTTAAGACCCATCAGATGTTTCCAGGCTCCCCCAAGGACCTGAAATGACCCTGC
GCCTTATTTGAATTAACCAATCAGCCTGCTTCTCGCTTCTGTTCGCGCGCTTCTGCTTCCCGAGCT
CTATAAAAGAGCTCACAACCCCTCACTCGGCGCGCCAGTCCTCCGACAGACTGAGTCGCCCGGG
GGATCCGCGGAATTCGCCGCCACCATGTGCCATCAGCAACTCGTCATCTCCTGGTTCTCCCTTGT
GTTCCTCGCTTCCCCTCTGGTCGCCATTTGGGAACTGAAGAAGGACGTCTACGTGGTCGAGCTG
GATTGGTACCCGGACGCCCCTGGAGAAATGGTCGTGCTGACTTGCGATACGCCAGAAGAGGAC
GGCATAACCTGGACCCTGGATCAGAGCTCCGAGGTGCTCGGAAGCGGAAAGACCCTGACCATT
CAAGTCAAGGAGTTCGGCGACGCGGGCCAGTACACTTGCCACAAGGGTGGCGAAGTGCTGTCC
CACTCCCTGCTGCTGCTGCACAAGAAAGAGGATGGAATCTGGTCCACTGACATCCTCAAGGACC
AAAAAGAACCGAAGAACAAGACCTTCCTCCGCTGCGAAGCCAAGAACTACAGCGGTCGGTTCA
CCTGTTGGTGGCTGACGACAATCTCCACCGACCTGACTTTCTCCGTGAAGTCGTCACGGGGATC
AAGCGATCCTCAGGGCGTGACCTGTGGAGCCGCCACTCTGTCCGCCGAGAGAGTCAGGGGAGA
CAACAAGGAATATGAGTACTCCGTGGAATGCCAGGAGGACAGCGCCTGCCCTGCCGCGGAAGA
GTCCCTGCCTATCGAGGTCATGGTCGATGCCGTGCATAAGCTGAAATACGAGAACTACACTTCC
TCCTTCTTTATCCGCGACATCATCAAGCCTGACCCCCCCAAGAACTTGCAGCTGAAGCCACTCAA
GAACTCCCGCCAAGTGGAAGTGTCTTGGGAATATCCAGACACTTGGAGCACCCCGCACTCATAC
TTCTCGCTCACTTTCTGTGTGCAAGTGCAGGGAAAGTCCAAACGGGAGAAGAAAGACCGGGTGT
TCACCGACAAAACCTCCGCCACTGTGATTTGTCGGAAGAACGCGTCAATCAGCGTCCGGGCGCA
GGATAGATACTACTCGTCCTCCTGGAGCGAATGGGCCAGCGTGCCTTGTTCCGGTGGCGGATCA
GGCGGAGGTTCAGGAGGAGGCTCCGGAGGAGGTTCCCGGAACCTCCCTGTGGCAACCCCCGAC
CCTGGAATGTTCCCGTGCCTACACCACTCCCAAAACCTCCTGAGGGCTGTGTCGAACATGTTGC
AGAAGGCCCGCCAGACCCTTGAGTTCTACCCCTGCACCTCGGAAGAAATTGATCACGAGGACAT
CACCAAGGACAAGACCTCGACCGTGGAAGCCTGCCTGCCGCTGGAACTGACCAAGAACGAATC
GTGTCTGAACTCCCGCGAGACAAGCTTTATCACTAACGGCAGCTGCCTGGCGTCGAGAAAGACC
TCATTCATGATGGCGCTCTGTCTTTCCTCGATCTACGAAGATCTGAAGATGTATCAGGTCGAGTT
CAAGACCATGAACGCCAAGCTGCTCATGGACCCGAAGCGGCAGATCTTCCTGGACCAGAATAT
GCTCGCCGTGATTGATGAACTGATGCAGGCCCTGAATTTCAACTCCGAGACTGTGCCTCAAAAG
TCCAGCCTGGAAGAACCGGACTTCTACAAGACCAAGATCAAGCTGTGCATCCTGTTGCACGCTT
TCCGCATTCGAGCCGTGACCATTGACCGCGTGATGTCCTACCTGAACGCCAGTAGACGGAAACG
CGGAAGCGGAGAGGGCAGAGGCTCGCTGCTTACATGCGGGGACGTGGAAGAGAACCCCGGTCC
GATGGAACGCATTGTGATCTGCCTGATGGTCATCTTCCTGGGCACCTTAGTGCACAAGTCGAGC
AGCCAGGGACAGGACAGGCACATGATTAGAATGCGCCAGCTCATCGATATCGTGGACCAGTTG
AAGAACTACGTGAACGACCTGGTGCCCGAGTTCCTGCCGGCCCCCGAAGATGTGGAAACCAATT
GCGAATGGTCGGCATTTTCCTGCTTTCAAAAGGCACAGCTCAAGTCCGCTAACACCGGGAACAA
CGAACGGATCATCAACGTGTCCATCAAAAAGCTGAAGCGGAAGCCTCCCTCCACCAACGCCGG
ACGGAGGCAGAAGCATAGGCTGACTTGCCCGTCATGCGACTCCTACGAGAAGAAGCCGCCGAA
GGAGTTCCTGGAGCGGTTCAAGTCGCTCCTGCAAAAGATGATTCATCAGCACCTGTCCTCCCGG
ACTCATGGGTCTGAGGATTCATGAGGTTAGTCGACAATCAACCTCTGGATTACAAAATTTGTGA
AAGATTGACTGGTATTCTTAACTATGTTGCTCCTTTTACGCTATGTGGATACGCTGCTTTAATGCC
TTTGTATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTTGCTGA
CGCAACCCCCACTGGTTGGGGCATTGCCACCACCTGTCAGCTCCTTTCCGGGACTTTCGCTTTCC
CCCTCCCTATTGCCACGGCGGAACTCATCGCCGCCTGCCTTGCCCGCTGCTGGACAGGGGCTCG
GCTGTTGGGCACTGACAATTCCGTGGTGTTGTCGGGGAAATCATCGTCCTTTCCTTGGCTGCTCG
CCTGTGTTGCCACCTGGATTCTGCGCGGGACGTCCTTCTGCTACGTCCCTTCGGCCCTCAATCCA
GCGGACCTTCCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGCTTAGTACTGGTACCTTTAAGACCAATG
ACTTACAAGGCAGCTGTAGATCTTAGCCACTTTTTAAAAGAAAAGGGGGGACTGGAAGGGCTA
ATTCACTCCCAACGAAGACAAGATTCCGGAATTTATTTGTGAAATTTGTGATGCTATTGCTTTAT
TTGTAAACCGGTGCAGCTGCTTTTTGCCTGTACTGGGTCTCTCTGGTTAGACCAGATCTGAGCCT
GGGAGCTCTCTGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAGACCCTTTTAGT
CAGTGTGGAAAATCTCTAGCATCTAGAGTATGCAAAGCATGCATCTCAATTAGTCAGCAACCAG
GTGTGGAAAGTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTTCCGCCCATTC
TCCGCCCCATGGCTGACTAATTTTTTTTATTTATGCAGAGGCCGAGGCCGCCTCGGCCTCTGAGC
TATTCCAGAAGTAGTGAGGAGGCTTTTTTGGAGGCCTAGGCTAGAGATCATAATCAGCCATACC
ACATTTGTAGAGGTTTTACTTGCTTTAAAAAACCTCCCACACCTCCCCCTGAACCTGAAACATAA
AATGAATGCAATTGTTGTTGTTAACTTGTTTATTGCAGCTTATAATGGTTACAAATAAAGCAATA
GCATCACAAATTTCACAAATAAAGCATTTTTTTCACTGCATTCTAGTTGTGGTTTGTCCAAACTC
ATCAATGTATCTTATCATGTCTGCTAGCCGGGCTTTTTTTTCTTAGGCCTTCTTCCGCTTCCTCGC
TCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGA
AGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAAA
GTATATATGAGTAAACTTGGTCTGACAGTTACCAATGCTTAATCAGTGAGGCACCTATCTCAGC
GATCTGTCTATTTCGTTCATCCATAGTTGCC

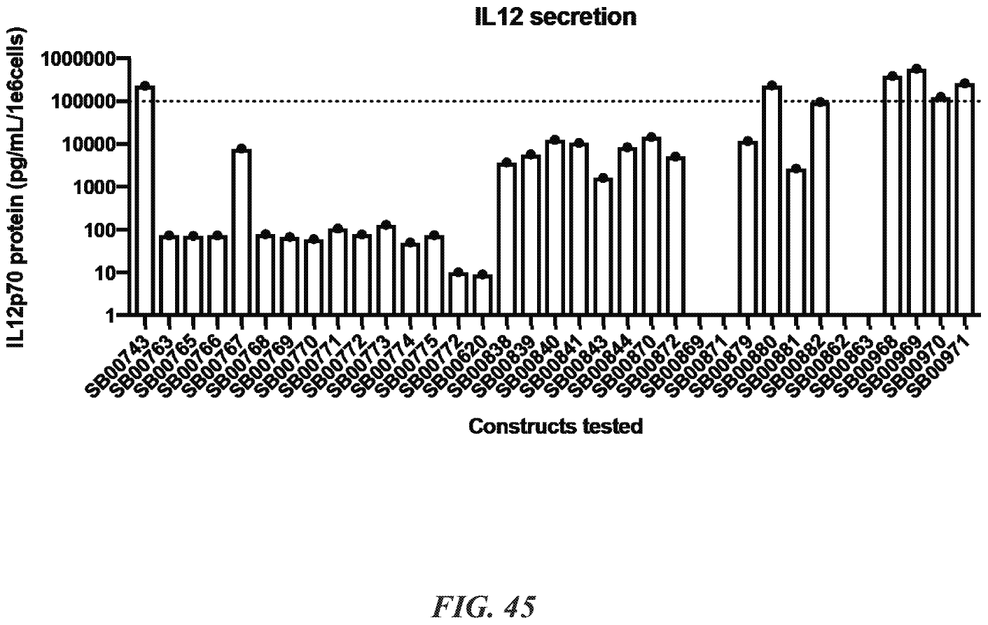

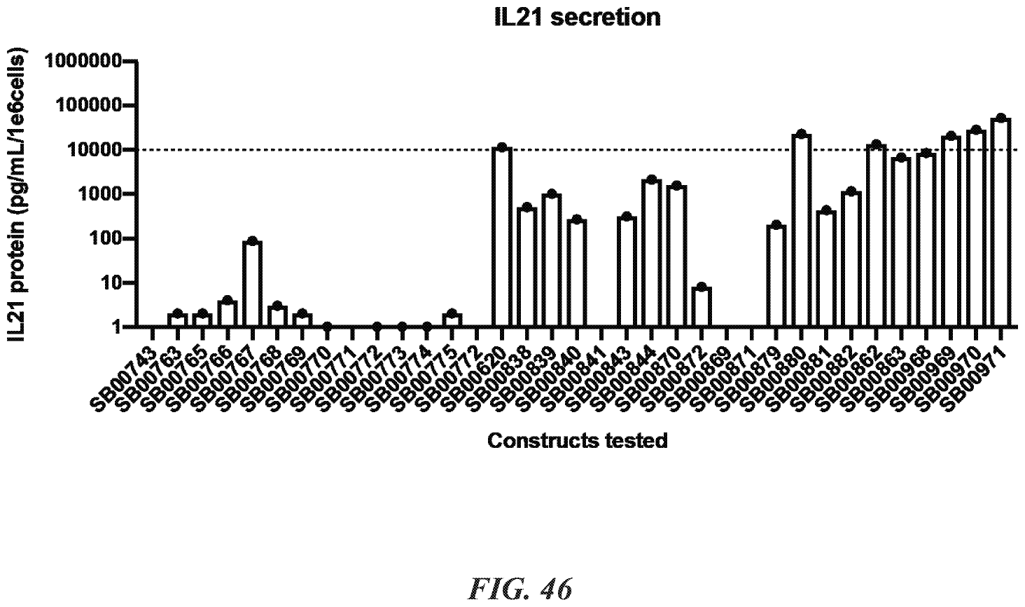

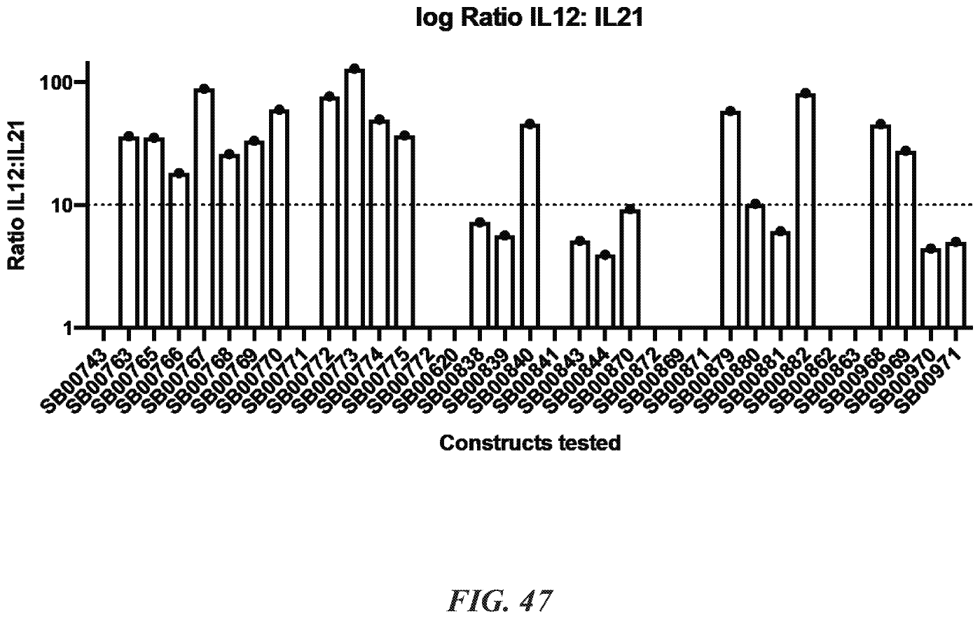

Secretion of IL-12p70 and IL-21 by engineered MSCs are shown in FIG. 45 and FIG. 46, respectively, as assessed by ELISA. SB00880 demonstrated expression of both cytokines by engineered MSCs at higher levels than the majority of constructs tested. Additionally, the ratio of IL-12 to IL-21 was determined, as assessed by ELISA and shown in FIG. 47. MSCs engineered using SB00880 demonstrated a 10 fold higher ratio of IL-12p70 relative to IL-21. Notably, a ratio of 10:1 has demonstrated pre-clinical efficacy (data not shown).

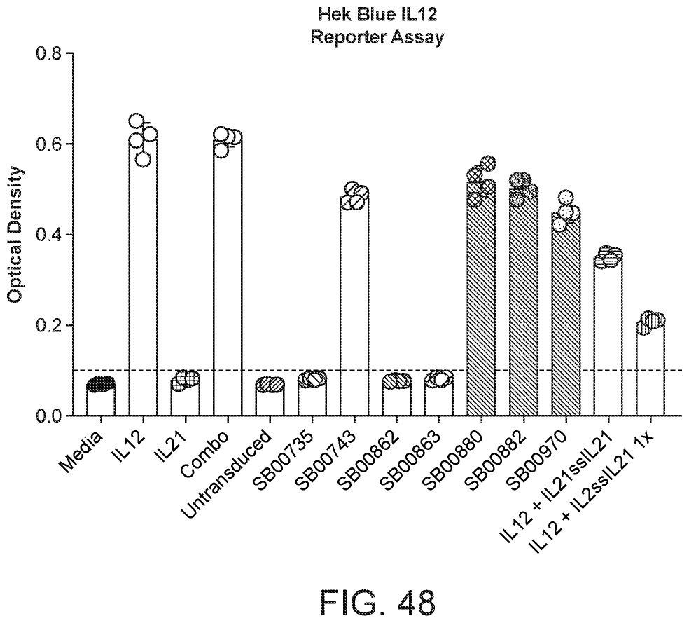

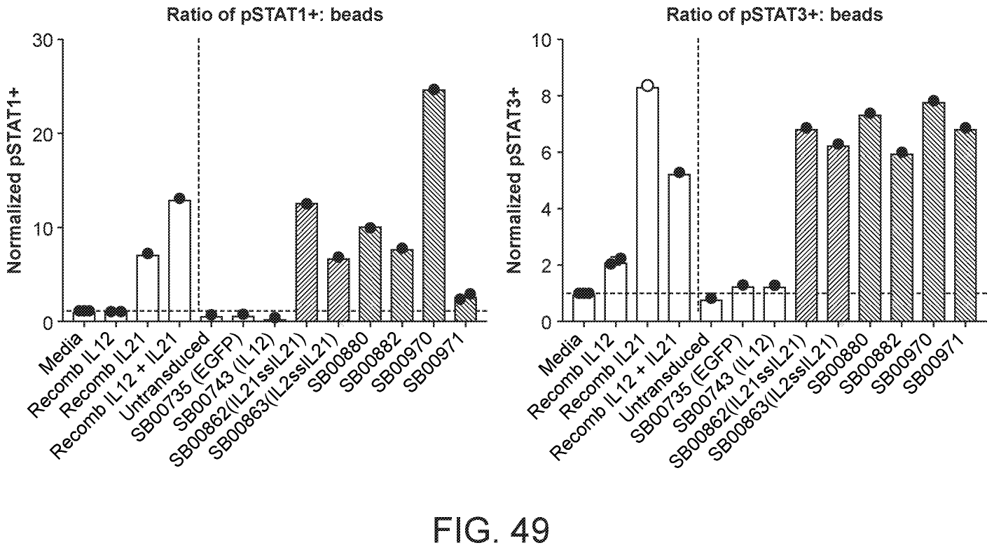

Functional assays demonstrating expression of IL-12p70 by engineered MSCs were performed. HEK-293T cells with a STAT4-SEAP reporter, which reports IL12p70 binding to its receptor and signaling through the JAK-STAT4 pathway, were used to determine potency and activity of IL12p70 produced by engineered hMSCs. Engineered MSCs were cultured for 24 hours and media was collected and incubated with HEK-293T STAT4-SEAP reporter cells. SEAP production was determined with spectrophotometer. As shown in FIG. 48, all constructs that encode IL-12 demonstrated reporter activity indicating functional IL12p70 signaling.

Functional assays demonstrating expression of IL-21 by engineered MSCs were performed. NK-92 human natural killer cells were used to determine function of IL-21 produced by engineered hMSCs. Engineered hMSCs were cultured for 24 hours and conditioned media was collected and used to treat NK-92 cells that were deprived from IL-2. Intracellular phospho-flow was performed to quantify phospho-STAT1 and phospho-STAT3 activation as a readout for IL-21 activity. As shown in FIG. 49, all constructs that encode IL-21 demonstrated STAT1 (left panel) and STAT3 (right panel) phosphorylation indicating functional IL-21 signaling.

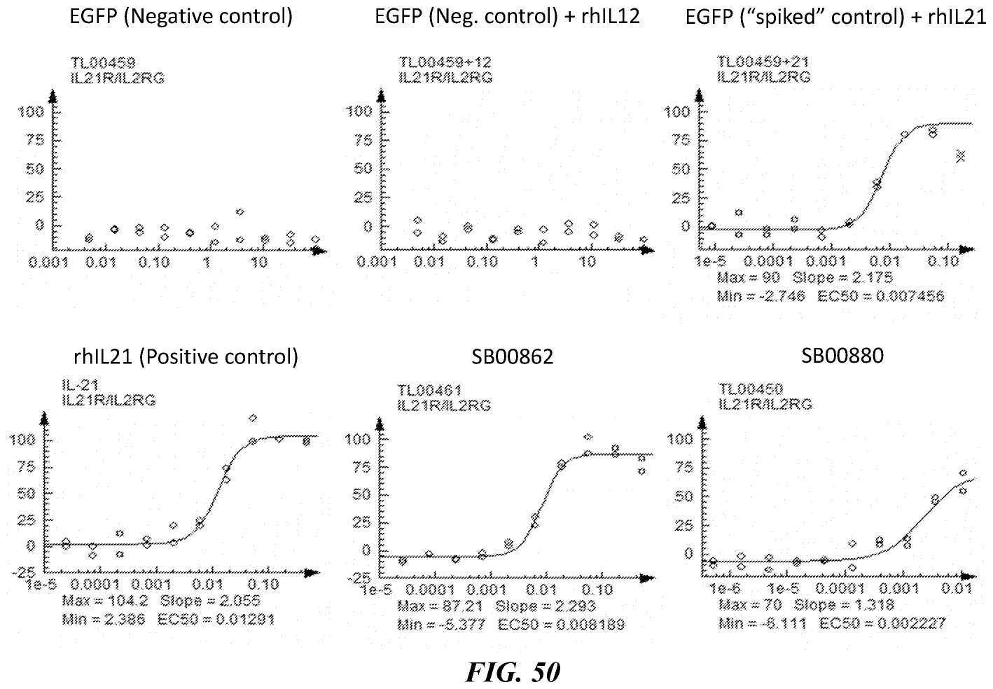

Functional assays for IL-21 was also performed using a IL21R-U2OS IL21R/IL2RG dimerization reporter (PathHunter® U2OS IL21R/IL2RG Dimerization Cell Line, DiscoverX Cat. No: 93-1035C3). Reporter cells were incubated with conditioned media from engineered human MSCs or the appropriate positive (recombinant cytokine) or negative controls. As shown in FIG. 50, all constructs that encode IL-21 demonstrated dimerization.

### Example 20: Optimization of Signal Peptide Sequences

In the following example, effector molecules are modified to replace their native signal peptide sequence with an exogenous signal peptide sequence (see Table 5 for exemplary signal peptide sequences that are tested). Modified effector molecules are tested for functional improvements such as improved expression and maintained secretion, such as in particular environments (e.g., tumor microenvironments). Functional performance for the modified effector molecules is also tested in tumor models (e.g., improved ability to clear tumors, improved ability to clear tumors in different environments, or improved ability to clear different types of tumors).

### Example 21: Enrichment of Engineered MSCs

In the following example, MSCs are engineered to express effector molecules within a population of cells that include non-engineered cells, such as non-engineered MSCs. The engineered MSCs are enriched within the population by contacting the engineered MSCs with a growth factor (such as the effector molecules described in Table 1) such that those engineered MSCs that are enriched are a sub-population of engineered MSCs that express a receptor or receptor ligand for the growth factor. The sub-population of engineered MSCs of interest are contacted with the growth factor in various manners:
1. In an autocrine manner by genetically engineering the MSCs themselves to express the factors.
2. In a paracrine manner by genetically engineering feeder or support cells to express the factors and supply those factors to the MSCs, or by using conditioned media containing the factors from the feeder or support cells (such as 293 Ts) engineered to express these factors.
3. In an endocrine manner, by injecting recombinant protein or nucleic acid versions of these factors into patients following MSC transplantation.
4. Via addition of soluble recombinant protein versions of these factors to the MSC culture conditions.
5. Via coating of the tissue culture plate/flask surfaces used for MSC propagation with recombinant versions of these factors.

### REFERENCES

1. Kidd S, et al. (2009) Stem Cells 27(10):2614-2623.
2. Dembinski J L, et al. (2013) Cytotherapy 15(1):20-32.
3. Siegel R L, et al. (2016) CA Cancer J Clin 66(1):7-30.
4. Dizon D M J (2010) Gynecol Oncol 116(3).
5. Woo S R, et al. (2015) Trends Immunol 36(4):250-256.
6. Hamanishi J, et al. (2016) Int Immunol 28(7):339-348.
7. Li S, et al. (2012) Oncolytic Virother 1:1-21.
8. Koneru M, et al. (2015) J Transl Med 13:102.
9. Cruz C R, et al. (2010) Cytotherapy 12(6):743-749.
10. Li Y Q, et al. (2013) PLoS One 8(10):e76379.
11. Wiedemann G M, et al. (2016) Oncoimmunology 5(9):e1175794.
12. Squillaro T, et al. (2016) Cell Transplant 25(5):829-848.
13. Studeny M, et al. (2004) J Natl Cancer Inst 96(21):1593-1603.
14. Ling X, et al. (2010) Cancer Microenviron 3(1):83-95.
15. Schukur L, et al. (2015) Sci Transl Med 7(318):318ra201.
16. Howlader N N A, Krapcho M, Garshell J, Miller D, Altekruse S F, Kosary C L, Yu M, Ruhl J, Tatalovich Z, Mariotto A, Lewis D R, Chen H S, Feuer E J and Cronin K A. (2015).
17. Lengyel E (2010) Am J Pathol 177(3):1053-1064.
18. McGuire W P, et al. (1996) The New England journal of medicine 334(1):1-6.
19. McGuire W P, et al. (1989) Annals of internal medicine 111(4):273-279.
20. Adams S F & Benencia F (2015) Future Oncology 11(9):1293-1296.
21. Maude S L, et al. (2014) N Engl J Med 371(16):1507-1517.
22. Bargou R, et al. (2008) Science 321(5891):974-977.
23. Kershaw M H, et al. (2014) Clin Trans Immunol 3:e16.
24. Gilham D E, et al. (2012) Trends Mol Med 18(7):377-384.
25. Klinger M, et al. (2012) Blood 119(26):6226-6233.
26. Fu J, et al. (2015) Sci Transl Med 7(283):283ra252.
27. Moynihan K D, et al. (2016) Nat Med 22(12):1402-1410.
28. Mohammadi M, et al. (2016) Cancer Gene Ther 23(9):285-286.
29. Wang D, et al. (2013) Cell Transplant 22(12):2267-2277.
30. Nowakowski A, et al. (2016) Stem Cells Int 2016:4956063.
31. Sun Z, et al. (2014) J Hematol Oncol 7:14.
32. Ando M, et al. (2015) Stem Cell Reports 5(4):597-608.
33. Zhao Q, et al. (2015) Proc Natl Acad Sci USA 112(2):530-535.
34. Xie C, et al. (2013) Br J Cancer 109(5):1198-1205.
35. Parker B S, et al. (2016) Nat Rev Cancer 16(3):131-144.
36. Roby K F, et al. (2000) Carcinogenesis 21(4):585-591.
37. Sharma A D, et al. (2015) J Vis Exp (95):e52242.
38. Waterman R S, et al. (2012) PLoS One 7(9):e45590.
39. Dubinett S M, et al. (2010) Cancer J 16(4):325-335.
40. Tang E D & Wang C Y (2015) PLoS One 10(3):e0120090.
41. Cieri N, et al. (2013) Blood 121(4):573-584.
42. Fitzgerald K A, et al. (2003) Nat Immunol 4(5):491-496.
43. Wong A S, et al. (2016) Proc Natl Acad Sci USA 113(9):2544-2549.
44. Wong A S, et al. (2015) Nat Biotechnol 33(9):952-961.
45. Nissim L, et al. (2014) Mol Cell 54(4):698-710.
46. Deng P, et al. (2016) Neural Regen Res 11(5):702-705.
47. Beegle J R, et al. (2016) Mol Ther Methods Clin Dev 3:16053.
48. Boutros C, et al. (2016) Nat Rev Clin Oncol 13(8):473-486.
49. Valsecchi M E (2015) New Engl J Med 373(13):1270-1270.
50. Pardoll D M (2012) Nat Rev Cancer 12(4):252-264.
51. Legat A, et al. (2013) Front Immunol 4:455.
52. Justus C R, et al. (2014) J Vis Exp (88).
53. Jedema I, et al. (2004) Blood 103(7):2677-2682.
54. Peng D, et al. (2015) Nature 527(7577):249-253.
55. Gitzinger M, et al. (2009) Proc Natl Acad Sci USA 106(26):10638-10643.
56. Clackson T, et al. (1998) Proc Natl Acad Sci USA 95(18):10437-10442.
57. Siuti P, et al. (2013) Nature Biotechnology 31(5):448-452.
58. Farzadfard F & Lu T K (2014) Science 346(6211):1256272.
59. Perli S D, et al. (2016) Science 353(6304).
60. Roquet N, et al. (2016) Science 353(6297):aad8559.
61. Wong A S L, et al. (2016) Proceedings of the National Academy of Sciences.
62. Gardner T S, et al. (2000) Nature 403(6767):339-342.
63. Deans T L, et al. (2007) Cell 130(2):363-372.
64. Warren L, et al. (2010) Cell Stem Cell 7(5):618-630.
65. Yang B X, et al. (2015) Cell 163(1):230-245.
66. Kumar R M, et al. (2014) Nature 516(7529):56-61.
67. Zhang J, et al. (2016) Cell Stem Cell 19(1):66-80.
68. Cahan P, et al. (2014) Cell 158(4):903-915.
69. Doulatov S, et al. (2013) Cell Stem Cell 13(4):459-470.
70. Kim K, et al. (2011) Nat Biotechnol 29(12):1117-1119.
71. Chavez A, et al. (2016) Nat Methods 13(7):563-567.
72. Slomovic S & Collins J J (2015) Nat Methods 12(11):1085-1090.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A virus engineered to comprise a heterologous nucleic acid, wherein the heterologous nucleic acid comprises a polynucleotide sequence encoding two or more effector molecules, optionally wherein the virus is selected from the group consisting of a lentivirus, a retrovirus, an oncolytic virus, an adenovirus, an adeno-associated virus (AAV), and a virus-like particle (VLP),
optionally wherein the heterologous nucleic acid comprises DNA or RNA,
optionally wherein the polynucleotide sequence comprises a promoter.

2. A tumor cell engineered to produce two or more effector molecules,
optionally wherein the tumor cell is selected from the group consisting of: a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, an ovarian tumor cell, a pancreatic tumor cell, a prostate tumor cell, a skin tumor cell, a thyroid tumor cell, and a uterine tumor cell,
optionally wherein the cell was engineered:
(a) via transduction with an oncolytic virus, optionally wherein the oncolytic virus is selected from the group consisting of: an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof, or
(b) via transfection with an isolated nucleic acid, optionally wherein the isolated nucleic acid is a cDNA, an mRNA, or a naked plasmid comprising a polynucleotide sequence encoding one or more of the two or more effector molecules,
optionally wherein the two or more effector molecules are encoded by a polynucleotide sequence,
optionally wherein the polynucleotide sequence comprises a promoter,
optionally wherein the polynucleotide sequence further comprises a linker polynucleotide sequence.

3. An erythrocyte or platelet cell engineered to produce two or more effector molecules.

4. A bacterial cell engineered to produce two or more mammalian effector molecules,
optionally wherein the bacterial cell is selected from the group consisting of Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

5. A lipid structure delivery system comprising a lipid-based structure comprising two or more effector molecules, optionally wherein the two or more effector molecules are encoded by a polynucleotide sequence, or the lipid-based structure comprises an engineered nucleic acid comprising a polynucleotide sequence encoding two or more effector molecules,
optionally wherein the engineered nucleic acid is a cDNA, an mRNA, or a naked plasmid,
optionally wherein the polynucleotide sequence comprises a promoter.

6. A nanoparticle comprising two or more effector molecules, optionally wherein the two or more effector molecules are encoded by a polynucleotide sequence, or wherein the nanoparticle comprises an engineered nucleic acid, wherein the engineered nucleic acid comprises a polynucleotide sequence encoding two or more effector molecules, optionally wherein the engineered nucleic acid is a cDNA, an mRNA, or a naked plasmid,
optionally wherein the polynucleotide sequence comprises a promoter.

7. The virus, engineered cell, lipid structure, or nanoparticle of any one of claims 1-6, wherein each of the two or more effector molecules comprises a secretion signal, and wherein each of the two or more effector molecules is secreted from the engineered cell of any one of claims 2-4 or a cell contacted by the virus of claim 1, lipid structure of claim 5, or nanoparticle of claim 6.

8. The virus, engineered cell, lipid structure, or nanoparticle of any one of claims 1-7, wherein the promoter is present and operably linked to the polynucleotide sequence such that the two or more effector molecules are capable of being transcribed as a single polynucleotide comprising the formula (L-E)x,
wherein
L comprises a linker polynucleotide sequence,
E comprises a polynucleotide encoding one of the two or more effector molecules,
X=2 to 20, and
wherein for the first iteration of the (L-E) unit L is absent,
optionally wherein the linker polynucleotide sequence is operably associated with the translation of the two or more effector molecules as separate polypeptides.

9. The virus, engineered cell, lipid structure, or nanoparticle of any one of claims 1-8, wherein a first effector molecule is selected from a therapeutic class, wherein the therapeutic class is selected from the group consisting of: a cytokine, a chemokine, a growth factor, a co-activation molecule, a tumor microenvironment modifier a, a receptor, a ligand, an antibody, a polynucleotide, a peptide, and an enzyme, and wherein a second effector molecule is independently selected from a therapeutic class, wherein the therapeutic class is selected from the group consisting of: a cytokine, a chemokine, a growth factor, a co-activation molecule, a tumor microenvironment modifier, a receptor, a ligand, an antibody, a polynucleotide, a peptide, and an enzyme,
optionally wherein the therapeutic class of the first effector molecule and the second effector molecule are different,
optionally wherein the cytokine is selected from the group consisting of IL12, IL7, IL21, IL18, IL15, Type I interferons, and Interferon-gamma,
optionally wherein the chemokine is selected from the group consisting of CCL21a, CXCL10, CXCL11, CXCL13, CXCL10-11 fusion, CCL19, CXCL9, and XCL1,
optionally wherein the growth factor is selected from the group consisting of FLT3L and GM-CSF,
optionally wherein the co-activation molecule is selected from the group consisting of 4-1BBL and CD40L,
optionally wherein the tumor microenvironment modifier is selected from the group consisting of adenosine deaminase, TGFbeta inhibitors, immune checkpoint inhibitors, VEGF inhibitors, and HPGE2,
optionally wherein the immune checkpoint inhibitors are selected from the group consisting of anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GALS antibodies, anti-A2AR antibodies, anti-phosphatidylserine antibodies, anti-CD27 antibodies, anti-TNFa antibodies, anti-TREM1 antibodies, and anti-TREM2 antibodies.

10. A pharmaceutical composition comprising the virus, engineered cell, lipid structure, or nanoparticle of any one of claims 1-9, and a pharmaceutically acceptable carrier, pharmaceutically acceptable excipient, or any combination thereof.

11. A method of treating a subject having cancer, the method comprising administering a therapeutically effective dose of the virus, engineered cell, lipid structure, or nanoparticle of any one of claims 1-9, or the pharmaceutical composition of claim 10.

12. A method of reducing tumor volume in a subject, the method comprising administering to a subject having a tumor a composition comprising the virus, engineered cell, lipid structure, or nanoparticle of any one of claims 1-9, or the pharmaceutical composition of claim 10.

13. A method of reducing tumor volume in a subject, the method comprising administering to a subject having a tumor a therapeutically effective dose of a composition, wherein the composition comprises two or more effector molecules or wherein the composition comprises an engineered nucleic acid comprising a polynucleotide sequence encoding two or more effector molecules.

14. The method of any one of claims 11-13, wherein the administering comprises one or more intraperitoneal injections or one or more intratumoral injections, optionally wherein the administering comprises systemic administration.

15. The method of claim 13 or claim 14, wherein the composition comprises a delivery system selected from the group consisting of: a viral system, a transposon system, and a nuclease genomic editing system,
optionally wherein the viral system is selected from the group consisting of a lentivirus, a retrovirus, a retrotransposon, an oncolytic virus, an adenovirus, an adeno-associated virus (AAV), and a virus-like particle (VLP),
optionally wherein the oncolytic virus is selected from the group consisting of an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof, optionally wherein the nuclease genomic editing system is selected from the group consisting of a zinc-finger system, a TALEN system, and a CRISPR system.

16. The method of any one of claims 13-15, wherein the composition comprises:
(a) an erythrocyte or a platelet cell;
(b) a lipid structure delivery system comprising a lipid-based structure, optionally wherein the lipid-based structure is selected from the group consisting of: an extracellular vesicle, a lipid nanoparticle, a micelle, nanovesicle, an exosome, and a liposome; or
(c) a nanoparticle, optionally wherein the nanoparticle comprises an inorganic material, optionally wherein the nanoparticle encapsulates the engineered nucleic acid or encapsulates the two or more effector molecules.


SEQUENCE LISTING (145 sequences)

SEQ ID NO: 1 - DNA, 31 bp, Artificial Sequence
  Description of Artificial Sequence Synthetic oligonucleotide
agagggtatataatggaagctcgacttccag

SEQ ID NO: 2 - DNA, 52 bp, Artificial Sequence
  Description of Artificial Sequence Synthetic oligonucleotide
gggaatttccggggactttccgggaatttccggggactttccgggaatttcc

SEQ ID NO: 3 - DNA, 85 bp, Artificial Sequence
  Description of Artificial Sequence Synthetic oligonucleotide
caccagacagtgacgtcagctgccagatcccatggccgtcatactgtgacgtctttcagacaccccattgacgtcaatgggagaa

SEQ ID NO: 4 - DNA, 90 bp, Artificial Sequence
  Description of Artificial Sequence Synthetic oligonucleotide
ggaggaaaaactgtttcatacagaaggcgtggaggaaaaactgtttcatacagaaggcgtggaggaaaaactgtttcatacagaaggcgt

SEQ ID NO: 5 - DNA, 115 bp, Artificial Sequence
  Description of Artificial Sequence Synthetic polynucleotide
aggatgtccatattaggacatctaggatgtccatattaggacatctaggatgtccatattaggacatctaggatgtccatattaggacatctaggatgtccatattaggacatct

SEQ ID NO: 6 - DNA, 115 bp, Artificial Sequence
  Description of Artificial Sequence Synthetic polynucleotide
agtatgtccatattaggacatctaccatgtccatattaggacatctactatgtccatattaggacatcttgtatgtccatattaggacatctaaaatgtccatattaggacatct

SEQ ID NO: 7 - DNA, 48 bp, Artificial Sequence
  Description of Artificial Sequence Synthetic oligonucleotide
tgagtcagtgactcagtgagtcagtgactcagtgagtcagtgactcag

SEQ ID NO: 8 - DNA, 120 bp, Artificial Sequence
  Description of Artificial Sequence Synthetic polynucleotide
agatcaaagggtttaagatcaaagggcttaagatcaaagggtataagatcaaagggcctaagatcaaagggactaagatcaaagggtttaagatcaaagggcttaagatcaaagggccta

SEQ ID NO: 9 - DNA, 32 bp, Artificial Sequence
  Description of Artificial Sequence Synthetic oligonucleotide
gtctagacgtctagacgtctagacgtctagac

SEQ ID NO: 10 - DNA, 24 bp, Artificial Sequence
  Description of Artificial Sequence Synthetic oligonucleotide
cagacacagacacagacacagaca

SEQ ID NO: 11 - DNA, 65 bp, Artificial Sequence
  Description of Artificial Sequence Synthetic oligonucleotide
ggatccggtactcgagatctgcgatctaagtaagcttggcattccggtactgttggtaaagccac

SEQ ID NO: 12 - DNA, 588 bp, Artificial Sequence
  Description of Artificial Sequence Synthetic polynucleotide
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 13 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 544 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 15 - DNA, 399 bp, Artificial Sequence
  Description of Artificial Sequence Synthetic polynucleotide
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 16 - DNA, 511 bp, Artificial Sequence
  Description of Artificial Sequence Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 17 - DNA, 408 bp, Artificial Sequence
  Description of Artificial Sequence Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 18 - DNA, 344 bp, Artificial Sequence
  Description of Artificial Sequence Synthetic polynucleotide
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 19 - DNA, 1229 bp, Artificial Sequence
  Description of Artificial Sequence Synthetic polynucleotide
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 20 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 1718 bp, Artificial Sequence
  Description of Artificial Sequence Synthetic polynucleotide
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 212 bp, Artificial Sequence
  Description of Artificial Sequence Synthetic polynucleotide
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 23 - DNA, 2379 bp, Artificial Sequence
  Description of Artificial Sequence Synthetic polynucleotide
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 24 - DNA, 529 bp, Artificial Sequence
  Description of Artificial Sequence Synthetic polynucleotide
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 25 - DNA, 794 bp, Artificial Sequence
  Description of Artificial Sequence Synthetic polynucleotide
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 26 - DNA, 532 bp, Artificial Sequence
  Description of Artificial Sequence Synthetic polynucleotide
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 27 - DNA, 701 bp, Artificial Sequence
  Description of Artificial Sequence Synthetic polynucleotide
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 28 - DNA, 465 bp, Artificial Sequence
  Description of Artificial Sequence Synthetic polynucleotide
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 29 - DNA, 538 bp, Artificial Sequence
  Description of Artificial Sequence Synthetic polynucleotide
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 30 - DNA, 1091 bp, Artificial Sequence
  Description of Artificial Sequence Synthetic polynucleotide
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 31 - DNA, 66 bp, Homo sapiens
atgtgtcaccagcagctcgttatatcctggtttagtttggtgtttctcgcttcacccctggtggca

SEQ ID NO: 32 - DNA, 66 bp, Artificial Sequence
  Description of Artificial Sequence Synthetic oligonucleotide
atgtgccatcagcaactcgtcatctcctggttctcccttgtgttcctcgcttcccctctggtcgcc

SEQ ID NO: 33 - DNA, 42 bp, Artificial Sequence
  Description of Artificial Sequence Synthetic oligonucleotide
atgcaactgctgtcatgtatcgcactcatcctggcgctggta

SEQ ID NO: 34 - DNA, 60 bp, Homo sapiens
atgtatcggatgcaacttttgagctgcatcgcattgtctctggcgctggtgacaaattcc

SEQ ID NO: 35 - DNA, 45 bp, Homo sapiens
atgaatctcttgctcatacttacgtttgtcgctgctgccgttgcg

SEQ ID NO: 36 - DNA, 51 bp, Unknown
  Description of Unknown Gaussia Luciferase sequence
atgggcgtgaaggtcttgtttgcccttatctgcatagctgttgcggaggcg

SEQ ID NO: 37 - DNA, 72 bp, Homo sapiens
atgccgatggggagccttcaacctttggcaacgctttatcttctggggatgttggttgctagttgccttggg

SEQ ID NO: 38 - DNA, 63 bp, Mus sp.
atggaaactgacacgttgttgctgtgggtattgctcttgtgggtcccaggatctacgggcgac

SEQ ID NO: 39 - DNA, 66 bp, Homo sapiens
atggatatgagggttcccgcccagcttttggggctgcttttgttgtggcttcgaggggctcggtgt

SEQ ID NO: 40 - DNA, 48 bp, Unknown
  Description of Unknown VSV-G sequence
atgaagtgtctgttgtacctggcgtttctgttcattggtgtaaactgt

SEQ ID NO: 41 - DNA, 84 bp, Homo sapiens
atgaatatcaaaggaagtccgtggaagggtagtctcctgctgctcctcgtatctaaccttctcctttgtcaatccgtggcaccc

SEQ ID NO: 42 - DNA, 54 bp, Homo sapiens
atgaaatgggtaacattcatatcacttctctttctgttcagctctgcgtattct

SEQ ID NO: 43 - DNA, 57 bp, Homo sapiens
atgacaaggcttactgttttggctctcctcgctggactcttggcttcctcccgagca

SEQ ID NO: 44 - DNA, 51 bp, Homo sapiens
atgagggcttggattttttttctgctctgccttgccggtcgagccctggcg

SEQ ID NO: 45 - DNA, 48 bp, Homo sapiens
atgcctcttctgcttttgcttcctcttttgtgggcaggtgccctcgca

SEQ ID NO: 46 - DNA, 87 bp, Homo sapiens
atgaactctttctcaacctctgcgtttggtccggtcgctttctcccttgggctcctgcttgtcttgccagcagcgtttcctgcgcca

SEQ ID NO: 47 - DNA, 60 bp, Homo sapiens
atgacaagtaaactggcggtagccttgctcgcggcctttttgatttccgcagccctttgt

SEQ ID NO: 48 - DNA, 69 bp, Homo sapiens
atgaaggtaagtgcagcgttgctttgccttctcctcattgcagcgacctttattcctcaagggctggcc

SEQ ID NO: 49 - DNA, 78 bp, Homo sapiens
atgggagcggcagctagaacacttcgacttgcccttgggctcttgctccttgcaaccctccttagacctgccgacgca

SEQ ID NO: 50 - DNA, 63 bp, Homo sapiens
atgtcaccgttgttgcggagattgctgttggccgcacttttgcaactggctcctgctcaagcc

SEQ ID NO: 51 - DNA, 63 bp, Homo sapiens
atgaataacctgctctgttgtgcgctcgtgttcctggacatttctataaaatggacaacgcaa

SEQ ID NO: 52 - DNA, 69 bp, Homo sapiens
atgcaaatgtctcctgcccttacctgtctcgtacttggtcttgcgctcgtatttggagagggatcagcc

SEQ ID NO: 53 - DNA, 102 bp, Homo sapiens
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, Homo sapiens
atgaatgcaaaagtcgtggtcgtgctggttttggttctgacggcgttgtgtcttagtgatggg

SEQ ID NO: 55 - DNA, 66 bp, Artificial Sequence
  Description of Artificial Sequence Synthetic oligonucleotide
atggaacgcattgtgatctgcctgatggtcatcttcctgggcaccttagtgcacaagtcgagcagc

SEQ ID NO: 56 - DNA, 1626 bp, Homo sapiens
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagctag

SEQ ID NO: 57 - DNA, 1623 bp, Artificial Sequence
  Description of Artificial Sequence Synthetic polynucleotide
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 58 - DNA, 1635 bp, Mus sp.
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgtag

SEQ ID NO: 59 - DNA, 465 bp, Artificial Sequence
  Description of Artificial Sequence Synthetic polynucleotide
atggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 60 - DNA, 2163 bp, Artificial Sequence
  Description of Artificial Sequence Synthetic polynucleotide
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagtagacggaaacgcggaagcggagagggcagaggctcgctgcttacatgcggggacgtggaagagaaccccggtccgatggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 61 - DNA, 2103 bp, Homo sapiens
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagccggcgcaagaggggttccggagagggaaggggtagtctgctcacctgcggcgatgttgaagaaaatcctggtcccatggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctgccgaaaagatagaggcgcctcaaaaaccggaaaaaagggcaaaggttctaaaggatgtaagcggactgaacgctctcaaacgcctaaagggccgtag

SEQ ID NO: 62 - DNA, 2109 bp, Mus sp.
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgcggcgcaagaggggttccggagagggaaggggtagtctgctcacctgcggcgatgttgaagaaaatcctggtcccatggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggctag

SEQ ID NO: 63 - DNA, 2109 bp, Mus sp.
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggccggcgcaagaggggttccggagagggaaggggtagtctgctcacctgcggcgatgttgaagaaaatcctggtcccatgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgtag

SEQ ID NO: 64 - DNA, 465 bp, Mus sp.
atgtttcatgtgtccttcaggtacatatttggtatcccaccacttatattggtgctcttgcctgtaaccagctctgaatgtcatataaaagacaaggagggcaaagcatacgagtccgtattgatgatctcaatcgatgaacttgacaagatgacagggaccgattctaattgtccaaataacgagccaaacttctttcggaaacacgtgtgtgatgatacaaaagaagctgcttttcttaacagagctgccagaaaactcaagcagttcctcaagatgaatatatccgaggaatttaacgtgcatctcctcacagtatctcagggaactcaaacccttgtaaactgcacttctaaggaggagaagaatgtcaaagagcagaagaaaaatgatgcatgttttttgaaacggctgttgagggagatcaaaacatgctggaataaaatcctcaagggctcaatttag

SEQ ID NO: 65 - DNA, 1428 bp, Homo sapiens
atggaaacagacacattgctgctttgggtattgttgctctgggtgcctggatcaacaggaaactgggtaaacgtaatttcagatctgaagaagatcgaggaccttattcaatccatgcacatcgatgccactctctacaccgaaagcgacgttcacccatcttgcaaggtgaccgctatgaaatgtgaattgttggaacttcaggtaatttctctggagagcggcgatgcctcaatacatgacaccgttgaaaatcttatcatccttgctaatgattcactctctagtaatgggaacgtaacagagagcgggtgtaaggagtgtgaagaactggaggagaaaaacattaaggaatttttgcagtcattcgtccatatagtgcaaatgttcataaacacttccagaagaaagcgaggctctggggaggggcgaggctctctgctgacctgtggggatgtagaagagaatccaggtcccatggaccggctgaccagctcattcctgcttctgattgtgccagcctacgtgctctccatcacatgtcctcccccaatgagcgtcgagcatgctgacatctgggtgaagtcatactccttgtacagcagagagagatacatttgtaattccggattcaagcgcaaggccggcacctcctctctgacagagtgcgtccttaacaaagcaaccaacgtagcacattggaccacaccatccttgaagtgcatacgagaacctaaatcttgcgataagactcatacttgtccaccttgtccagccccagaactgcttggcggaccctcagtatttttgttcccaccaaagccaaaagacacactcatgatatccagaactcctgaggtgacctgtgtcgttgtagacgtttcccacgaagatcctgaagtaaaattcaactggtacgtggatggggtcgaagtccataacgccaagactaaaccaagggaggaacagtataactctacttaccgagtagtttctgtgttgaccgtgctgcaccaggactggttgaacgggaaggagtacaaatgcaaggtgagcaataaagctctgcccgcaccaatcgaaaagacaatatctaaggccaaggggcagccacgagagccccaggtatacacactgccaccctcacgcgatgaattgactaagaaccaggtttccctgacctgtcttgtaaaaggtttctacccttccgacatagctgttgagtgggaaagtaacgggcagccagagaacaattacaagacaactccacccgttcttgatagcgatggatcattttttctgtattccaaactcactgtcgataaaagtcgctggcagcaaggcaatgtttttagctgctcagtcatgcacgaagcactgcataatcactacacacaaaaaagtttgtcccttagccctggtaagtag

SEQ ID NO: 66 - DNA, 1080 bp, Homo sapiens
atgtactcaatgcagttggcctcctgtgtaacattgaccttggtcctcttggtcaacagcaattggatcgatgtacgctacgacttggagaagattgagtcccttatacagagtatacacatagatacaaccttgtatactgacagtgacttccatcccagctgtaaagtgactgcaatgaactgttttttgttggagttgcaagtaattctgcatgaatacagcaacatgaccctcaatgaaaccgttaggaatgtcctttatctcgcaaattctactctgagtagcaataagaatgttgccgaaagcggctgcaaggagtgcgaagaactggaggaaaaaactttcaccgagtttctccagagtttcatcagaattgtccaaatgttcattaatacaagtagtggtggtgggagcgggggtggaggcagtgggggaggtgggagcggaggtggagggtccggaggggggagccttcaaggcactacttgtcctccacccgtatccatcgagcacgccgatattcgagttaaaaattatagtgttaatagcagagaacgatacgtctgcaactcagggtttaagagaaaggccggaacttcaactctcatagaatgcgtgattaataagaatactaacgtcgcacattggactactcccagtctcaagtgcatacgcgatccatctctcgctcattactcaccagtacctacagtggttactcctaaggtgacctctcagcccgaatcaccatctcccagcgcaaaagagcctgaggccttttctcctaaatcagacactgctatgactacagaaacagccataatgccaggaagccggctgacaccatctcaaactaccagcgcaggcacaactgggactggctcccacaaaagctcacgcgcaccaagtctcgccgcaacaatgacattggagcctacagccagcacatctcttagaatcacagaaatttctccccacagtagcaagatgaccaaggtggcaattagtaccagcgtccttcttgtaggagctggagttgtgatggcatttttggcatggtatatcaaaagcaggtag

SEQ ID NO: 67 - DNA, 489 bp, Mus sp.
atgaagatcctcaagccatacatgcgaaacactagtattagctgttacttgtgttttctgctgaatagtcattttttgactgaagcaggaatccatgtatttatactcggttgtgtgtctgtaggtctgccaaagactgaggctaattggattgacgtgcgctatgatcttgaaaaaatagagtccttgattcaatcaatacacatcgataccactctctacaccgacagtgatttccatccttcctgcaaggtaacagctatgaattgcttcctcctggagctccaagtcattctccatgagtactccaacatgactttgaacgaaactgtaagaaacgtattgtatctggctaatagcaccttgtctagtaacaaaaatgtggcagagagcggctgcaaagaatgtgaagaattggaagagaaaacatttacagagttcctgcaatcctttattcgcatcgtccaaatgtttatcaatacctcttag

SEQ ID NO: 68 - DNA, 438 bp, Mus sp.
atgtattccatgcaacttgccagttgtgtaacccttactctcgtcctgctcgttaattccgctggtgctaactggatagatgttcgatacgatctggaaaagattgagtcccttatccaatccattcatatagataccaccctttatactgacagcgacttccatccttcttgcaaggtgaccgctatgaattgtttcctgctggaactccaagttattctgcatgaatactctaatatgacacttaacgagaccgtaagaaatgttctctatctcgctaatagtactttgagctcaaataagaacgtggccgagtctgggtgtaaggaatgcgaagagctggaagaaaagacattcaccgagtttctccagtctttcatacggattgtgcagatgtttatcaacacatcagattacaaagacgacgatgataagtag

SEQ ID NO: 69 - DNA, 579 bp, Mus sp.
atggcagccatgtctgaggactcttgtgtgaactttaaagaaatgatgttcatagacaatacactctactttatacctgaggagaatggagatttggaatctgacaactttggcaggctgcattgcactaccgcagttatccgaaacatcaacgatcaggtactgtttgttgataaaagacaacctgtattcgaggacatgaccgacatagatcagtctgcctcagagccccagactaggcttatcatctatatgtacaaggacagcgaagtacgaggcctggctgttacactctcagtcaaagactctaagatgagcaccctgtcatgcaagaacaaaattatcagttttgaggagatggacccacctgaaaacatagatgacattcagtcagacctcattttttttcaaaagcgggtaccaggacacaacaaaatggaatttgaatcatcactctacgaaggacatttccttgcatgccagaaagaggatgacgcattcaaattgatcctgaaaaaaaaggacgaaaatggtgataaatcagtcatgtttacattgaccaatcttcaccaaagttag

SEQ ID NO: 70 - DNA, 579 bp, Mus sp.
atggctgcaatgtctgaagatagctgtgtcaactttaaggagatgatgttcattgataatactttgtactttatacctgaagaaaatggagaccttgagtcagacaacttcgggagactgcactgcacaactgccgttatccgaaacataaatgatcaagtattgttcgtggacaaaagacaaccagtctttgaggatatgacagacatcgaccaatccgcatctgaacctcagactaggctgatcatctatatgtacgccgactccgaagtaagaggccttgctgtgacacttagtgttaaggatagtaagatgagcacactgtcctgtaagaataagattatatcttttgaagagatggaccctcccgagaacatagatgacatccagagcgacttgatcttctttcagaagcgagtgccaggccataacaagatggaatttgaatcatctctttatgaaggccatttcctcgcatgtcaaaaggaggacgatgccttcaagctcattctgaaaaaaaaagacgagaacggtgataagagcgtgatgttcactctgacaaatctgcaccagtcatag

SEQ ID NO: 71 - DNA, 534 bp, Homo sapiens
atgtatcgcatgcaactcctgtcctgcattgctctgagcttggctttggtaaccaactcatacttcgggaaactggagagtaaactctccgtaatcaggaatcttaatgaccaagtattgtttattgaccagggcaaccgcccgttgttcgaggatatgactgattctgactgtcgggataacgctccgagaactatctttatcatttcaatgtacaaggacagccaaccgcggggtatggctgtgacaatcagtgtcaaatgtgagaagatttccacgctgtcctgcgaaaacaagataatttctttcaaagaaatgaacccccctgacaatataaaggatacaaagagtgatatcatcttctttcagaggtccgtgcccggccacgataataagatgcaatttgaaagttcatcttatgaggggtactttttggcatgcgagaaagaaagggatctcttcaagttgatcctgaagaaggaggacgaattgggcgaccgctccatcatgttcacagtccagaacgaggactag

SEQ ID NO: 72 - DNA, 534 bp, Homo sapiens
atgtaccgcatgcagctcctgagttgtattgccctttccctcgctctcgttaccaattcttacttcggtaagcttgcctctaaactctctgttattaggaacttgaacgaccaagtccttttcatagaccaagggaacagaccactgtttgaagatatgacggatagcgattgccgagataatgcccctaggacgatttttatcattagtatgtatgcggactctcaaccgagggggatggccgttactataagtgtgaaatgcgagaaaatatcaacgctcagttgtgagaacaaaatcataagtttcaaggagatgaatccacctgataacatcaaagacactaagtctgatattatatttttccaacgaagtgttccgggacacgataacaaaatgcaatttgagagctcctcatacgagggctacttcctcgcgtgtgagaaagaaagggatttgtttaagcttatcctcaagaaagaggacgagttgggggatcggagcataatgtttaccgtacagaatgaggactag

SEQ ID NO: 73 - DNA, 441 bp, Mus sp.
atggagcggacactcgtgtgtcttgtcgtaatttttctcgggacagtcgcacacaagtcctcaccccagggtcctgatcgccttctcatacgcctccgacatttgatcgacattgtagagcagctcaaaatttacgagaatgacctcgatcccgagcttttgagtgctccccaagacgttaagggtcattgcgagcacgcagcttttgcttgcttccagaaggccaagttgaaaccaagcaaccctggtaataataagactttcatcatcgacttggtcgcccaactccgaaggaggctgcctgcccggcgcggaggaaaaaaacaaaagcatattgcaaagtgtccttcatgtgattcatacgaaaagcggactcccaaagagttcttggaaaggttgaaatggcttcttcagaagatgattcatcaacatttgtcatag

SEQ ID NO: 74 - DNA, 564 bp, Homo sapiens
atgaccaacaaatgccttttgcaaattgccctgcttttgtgttttagcactaccgcattgagcatgtcatataacctcctcggcttccttcagagatcatcaaactttcagtgtcagaaactgctttggcaacttaatggcaggctcgaatattgtctgaaagatcggatgaatttcgacattccagaagaaataaaacagcttcaacaattccagaaagaggacgccgccctgactatttacgagatgctccagaatatcttcgccattttccggcaggacagctcatccacggggtggaatgagactattgtagaaaatcttctggctaatgtgtaccatcaaattaatcacctcaaaacggtgcttgaggaaaaacttgaaaaggaagatttcacacggggcaagttgatgtcctccctgcaccttaaacgatactacggcaggattcttcattacttgaaggctaaggagtatagccattgcgcgtggacaattgtacgggtagaaatactgcgaaacttttatttcatcaaccggctcactggataccttagaaattag

SEQ ID NO: 75 - DNA, 549 bp, Mus sp.
atgaacaatcggtggatactccacgccgcatttctcctctgctttagcacgacggccctgtccatcaactacaaacagcttcagttgcaggagcggactaacataaggaagtgccaggaactgctggaacagcttaatggtaaaattaatcttacataccgagctgacttcaaaattcctatggaaatgaccgagaagatgcagaaatcctacacggcattcgccatccaggaaatgctccagaacgtatttctcgtgttccgcaataatttctcttctacgggttggaacgaaaccattgttgttagactgcttgacgaactgcatcagcaaaccgtgttccttaaaaccgtgcttgaggagaagcaggaggagcgcctgacttgggagatgtctagtaccgcacttcacttgaaatcctactactggcgcgttcagcggtatctgaagctgatgaagtataactcatacgcctggatggtagtgcgcgcagagatcttcagaaactttcttatcatccggcgactgacccgaaactttcagaattag

SEQ ID NO: 76 - DNA, 501 bp, Homo sapiens
atgaagtacactagctatatattggccttccagctttgcatcgtattgggtagcctcggatgctattgccaagacccgtatgtcaaagaagccgaaaatctcaaaaagtatttcaatgccggacactcagacgtcgcggataacggtacactgtttcttggcatcctgaaaaattggaaggaagagagtgacagaaaaataatgcagtcacaaatagtgtccttttactttaagctgttcaaaaatttcaaggatgaccaaagtatccagaagagtgttgaaactatcaaagaggacatgaatgtgaaattctttaacagtaataagaagaagcgcgatgacttcgagaaactcactaattacagcgtaacggatcttaacgtccaacgcaaggcaatccacgagcttatacaggtaatggctgagcttagtcccgcagccaagacagggaagagaaaaaggtctcaaatgctttttcggggccggcgagcttcacaatag

SEQ ID NO: 77 - DNA, 468 bp, Mus sp.
atgaacgctacgcattgcatcctcgcactccaattgttcctcatggctgtgtcagggtgttactgtcacggtactgtcatagaaagcctcgaatccctgaataactattttaacagtagcggtatagatgtagaagaaaagtctctctttcttgacatctggaggaattggcaaaaggatggagacatgaagattctccaatctcagattatatcattttacttgaggctttttgaggttctgaaggataaccaggcgatcagcaataatatcagcgtaattgaatctcaccttattacaacatttttctcaaattccaaggcaaagaaagatgctttcatgtctatcgcgaaatttgaggtgaacaatcctcaggtacaaaggcaagcctttaacgagctgattagagttgtacatcagttgttgcccgaaagtagtcttagaaaacgcaaacggagccgatgctag

SEQ ID NO: 78 - DNA, 570 bp, Mus sp.
atggcaaggttgtgcgcttttctcatggtactggctgtgctctcctattggcctacttgttctctgggatgcgacttgccacagacccacaatctgcggaataagagggctctgactctgctggtgcaaatgagacggctctctccacttagctgtttgaaagatagaaaggatttcgggttcccccaggagaaggtggatgcccagcagatcaagaaggcacaggctatccccgtcctttccgagctgacccagcaaattttgaacatctttacaagtaaggatagttcagctgcatggaataccacacttttggattctttttgtaacgatctgcatcagcagctgaacgatctccagggatgcctgatgcagcaagtcggcgtgcaagaatttccactcacccaggaggacgctctgctcgcagtgcgaaagtattttcaccgaattaccgtgtacctccgggagaaaaagcattcaccctgcgcttgggaagtagtcagggccgaagtatggagagcccttagtagctccgctaatgtactgggccggttgcgggaagagaaatag

SEQ ID NO: 79 - DNA, 405 bp, Homo sapiens
atggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctgccgaaaagatagaggcgcctcaaaaaccggaaaaaagggcaaaggttctaaaggatgtaagcggactgaacgctctcaaacgcctaaagggccgtag

SEQ ID NO: 80 - DNA, 402 bp, Mus sp.
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggctag

SEQ ID NO: 81 - DNA, 309 bp, Homo sapiens
atggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctag

SEQ ID NO: 82 - DNA, 309 bp, Mus sp.
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtag

SEQ ID NO: 83 - DNA, 327 bp, Mus sp.
atggcaccccgcgtcacacccttgcttgctttttctctgcttgtcctctggaccttccccgctcctacccttggaggagccaatgatgccgaggattgctgcctgagtgttacacaaaggccaataccagggaatatagtgaaggcattccggtatctgctcaatgaagatgggtgcagagtccccgcagttgtctttacaacattgcgaggttaccagctttgtgctcccccagaccagccttgggtagatcgcattattcgccggttgaagaagagctcagcaaagaataagggcaattccacacggagaagccccgtctcctag

SEQ ID NO: 84 - DNA, 381 bp, Mus sp.
atgaaatcagcagtccttttcttgctcgggattatttttctggaacaatgtggagtgaggggaacactcgtaataagaaacgctcggtgctcatgcatatcaacatcacggggcactatccactacaaatccctgaaggatctgaagcagttcgccccaagccctaactgtaacaagaccgaaattatcgcaactctcaaaaatggagatcagacttgtcttgacccagattcagcaaatgtcaagaagctgatgaaagagtgggaaaagaagatttcacaaaaaaaaaagcaaaaacgcggcaagaaacatcaaaagaacatgaaaaacaggaaacctaagactccccagtcaaggagaagatcccgcaagacaacctag

SEQ ID NO: 85 - DNA, 303 bp, Mus sp.
atgaacagaaaagttaccgctatagcacttgctgccataatatgggccaccgcagctcaagggttcctgatgttcaagcagggccgatgcctctgcattggccctggaatgaaggccgtgaaaatggccgaaatagaaaaagctagtgtcatatacccctctaacggttgcgataaagtcgaggttatagtcacaatgaaagctcataaacgccaacgctgcctcgacccccggtctaagcaggctaggctcataatgcaagcaatcgagaagaaaaactttcttagacggcaaaacatgtag

SEQ ID NO: 86 - DNA, 297 bp, Mus sp.
atgaacccatctgccgccgttattttctgtctgatactccttgggctgagtggcacacaaggcatacccctcgcccgcacagtccggtgtaattgtatacatattgacgacggccctgttagaatgcgggccatcggtaagctggagattataccagcaagccttagttgtcccagggttgaaatcatagcaactatgaaaaaaaacgacgaacaaagatgtttgaatcccgagagcaagacaatcaaaaaccttatgaaagcatttagtcaaaaacgctctaaacgcgctccatag

SEQ ID NO: 87 - DNA, 297 bp, Homo sapiens
atgaatcagacggcaatccttatatgctgccttatattccttactctctcagggatacaaggggtaccactttctcggactgttcgctgcacttgcatttcaatatctaaccaacctgtaaatccgcggagcctggaaaaattggagattatacctgcttctcaattctgccctcgggtggaaatcatcgccactatgaagaagaagggcgagaaaaggtgtctgaatccagagtcaaaggcaatcaaaaacctgctgaaagcggtgtcaaaggaacggtccaagagatcaccctag

SEQ ID NO: 88 - DNA, 534 bp, Mus sp.
atgaacaggaaagtaacagccattgcattggctgccatcatctgggccaccgcagcacagggttttctgatgtttaagcaagggcgctgtctctgtataggcccaggcatgaaggccgtgaagatggcagagattgagaaggcatctgtgatttatccttctaacgggtgcgataaagtcgaagttattgtgacaatgaaggcacacaaacgccaacggtgtttggacccacgatctaaacaggcaagattgattatgcaagccatcgagaaaaagaactttctccgaaggcaaaatatgatccctttggctcggacagtgcggtgtaactgtattcacatcgacgatgggccagtacggatgagagcaataggaaagctcgaaatcatacccgcctcattgtcttgtcccagggtggaaataatcgccactatgaaaaagaacgatgaacagaggtgtctcaacccagagagtaagactatcaagaaccttatgaaggcattcagtcagaagaggtcaaagcgagcaccatag

SEQ ID NO: 89 - DNA, 345 bp, Homo sapiens
atgagacttctcatattggcgcttctcgggatatgttctcttacggcatacatagttgagggggtgggatctgaggttagcgataaacgaacttgtgttagtcttacaacacagaggcttccagtctccaggataaaaacatatacgataactgagggatctctcagagcggtcatcttcataacgaagaggggcctgaaggtctgtgctgacccacaagcgacttgggtaagggacgttgtgcggagcatggacaggaagagcaatactcgcaacaacatgatccaaaccaaacctacgggcacccaacagtcaaccaatactgcggtaacattgacggggtag

SEQ ID NO: 90 - DNA, 345 bp, Mus sp.
atgcgcctccttctgctgacttttctgggtgtatgttgcctgacaccctgggtcgtagaaggagtaggaaccgaggttctggaagagtcctcatgtgtaaacttgcagacacaacgactccccgtccaaaaaatcaagacctatataatctgggagggggcaatgcgggccgtcattttcgtgactaaacgaggtctcaaaatctgcgccgaccccgaggctaagtgggtgaaggcagccattaagaccgtggatgggagagccagcaccagaaagaacatggccgaaacagtacctactggcgcacagcggtcaacctcaactgctataaccttgacaggatag

SEQ ID NO: 91 - DNA, 513 bp, Unknown
  Description of Unknown m_sCD40L #1 sequence
atggagactgacactctgcttctgtgggtgttgctgctgtgggtgcctggcagtacaggcgatatgcaacgaggtgacgaggaccctcaaatcgccgcccatgtagtctctgaagctaatagcaacgctgcatccgtcttgcagtgggcaaagaaaggctactatactatgaagtccaacttggtaatgcttgaaaacggcaagcagttgactgtcaagagagagggactttattacgtctatacccaagtcacattctgtagcaatcgagaaccctcctcacagaggccttttatagtgggactctggcttaaaccaagtagcggctctgagcgcatactgttgaaagccgcaaacacacacagctcttcccaactctgcgagcagcaatccgtgcatctcggtggagtatttgagcttcaagccggtgcctcagtgtttgtgaacgtcactgaggcctcccaggtcatacatcgagttgggttcagctccttcggcttgctcaagctctag

SEQ ID NO: 92 - DNA, 588 bp, Unknown
  Description of Unknown m_sCD40L #2 sequence
atggaaactgatacattgctgctctgggttttgctgctctgggtgcctgggagtacaggcgacatgaggaggcagttcgaggatctcgttaaggatattacccttaataaggaggagaagaaagaaaactcttttgagatgcaacgaggggacgaagatcctcagatcgctgctcacgtggtctctgaagctaacagcaacgccgcttctgtcctccagtgggccaagaaaggttattacaccatgaaatcaaaccttgtaatgcttgaaaacgggaaacagcttacagtgaagagggaaggtctttactacgtctatacccaggtaaccttctgctcaaacagagaaccatcaagccagaggccattcatagtggggctctggctcaaaccttccagtggcagcgagagaatcttgttgaaagctgctaatacacatagtagtagccagctttgcgagcaacagtcagtccacctcgggggggtgtttgagttgcaagcaggggcctcagtattcgtgaatgtcactgaggcttcccaggtaattcacagggtaggctttagttcattcggtttgctgaagctttag

SEQ ID NO: 93 - DNA, 849 bp, Unknown
  Description of Unknown m_sCD40L #3 sequence
atgcgaagaatgcagcttctgctccttattgctctgagtctcgcccttgtcaccaactccggggacagaatgaaacaaatcgaggacaaaattgaagaaatactgagtaaaatatatcacatcgaaaacgaaattgcacgcattaagaaattgattggcgaacgcaccagtggcggctctggtggcaccggaggttcaggcgggaccgggggctctgacaaagtcgaagaggaggttaaccttcatgaggactttgtgttcatcaagaagctgaaacggtgcaataaaggagaaggttctttgagcctccttaattgcgaagagatgcgacgacagttcgaggatctggttaaggacattacacttaataaggaagagaaaaaggagaactctttcgaaatgcagcgcggcgatgaagatccccagatagccgcccatgtcgtctctgaggccaactctaacgcagcatccgtcctccagtgggctaagaaaggatattatactatgaaaagcaatttggtcatgctcgaaaacggtaaacagctcactgttaagagagaaggcctctattacgtatatactcaagtaactttctgttctaatagggaaccctcctctcaaagaccttttatcgtaggactctggttgaaaccaagtagcggtagtgaaaggattctgctcaaagcagctaatactcactccagcagtcaactgtgcgaacaacaaagcgttcacctcgggggcgtctttgaacttcaggcaggtgccagtgttttcgtcaacgtaacagaagcatcccaggtaattcatcgagtagggttttctagctttggtttgctgaagctgtag

SEQ ID NO: 94 - DNA, 2160 bp, Unknown
  Description of Unknown anti-CD40_FGK4.5 sequence
atggaaactgatcgcctgttgctctgggtacttcttctgtgggtgcctgggtccactggtgacactgtacttacacaatcacccgctttggccgtttctcctggtgaacgggtcacaattagttgccgagcttccgattctgtatctactcttatgcattggtatcaacaaaaacctggtcagcagccaaaattgctcatttatcttgctagtcacttggagtccggcgtacctgctcgattcagcggtagtgggtctggcacagatttcactttgaccatagatcccgtggaggccgatgacactgcaacctactattgccagcaatcctggaacgacccttggactttcggcggcggcaccaagctggaactcaagcgagcagatgctgccccaaccgttagtatattcccaccctcaaccgaacaactcgccacaggaggcgctagtgtcgtgtgtcttatgaacaatttctatccacgagacattagcgtcaagtggaaaattgatgggacagaaaggcgagatggagttttggattcagtaacagaccaggattcaaaggattctacctatagcatgagctccaccttgagcctgaccaaagctgattatgaatctcataacctgtatacttgtgaagtggtgcataagacttctagctcaccagtggttaaatcttttaaccgcaacgaatgtcggcgcaagaggggttccggagagggaaggggtagtctgctcacctgcggcgatgttgaagaaaatcctggtcccatggacattcggctctctttggtattcctggtactttttataaagggggtgcaatgtgaagtccagctcgtggaaagcggtgggggcctggttcagcccggtcgcagccttaaacttagttgcgcagcatccggatttacattttctgactataacatggcctgggttcgacaggcacccaaaaaagggctggagtgggtcgcaactatcatatacgatggttcccggacatactatagagattcagtgaaggggcgctttacaataagcagggacaatgctaagtctaccttgtatcttcagatggactccctgaggagcgaagatacagcaacatattattgtgctacaaaccgctggttgctgcttcattatttcgactactggggtcagggcgtcatggtaactgtatcaagcgccgagaccacagccccttctgtatatccattggcaccaggtactgctctgaaatccaactcaatggtaacccttggatgtctggttaagggttattttcccgagcccgtcacagttacttggaactctggggccctttctagcggagtccatacctttcccgccgttttgcagagtggtctgtacacccttacctcaagcgtcacagttccatctagcacatggagctcccaggcagtaacttgtaatgtggcccatccagcctcctcaactaaggtagataaaaagatcgttcccagagaatgcaatccatgtggatgcaccgggtctgaggtcagcagtgtgttcattttcccacccaagactaaagatgtattgactattactcttacacccaaagtaacctgcgtggtggttgatattagtcaaaatgatcccgaggtacggttctcttggtttatcgacgacgtcgaagtacatacagctcagacacacgctcccgagaaacaaagcaattccactcttaggagcgtgtccgagttgccaatcgtacatagggattggcttaatggcaagacctttaagtgtaaggtcaattcaggggcattccccgcaccaatagagaagagtataagcaaacccgaggggacacccagaggtccacaggtctatacaatggctccccccaaggaagagatgacccaaagtcaagtctcaattacatgtatggtgaagggcttttatccacccgacatatacactgagtggaagatgaatggacagccccaagagaattataaaaacactccccctaccatggacaccgacgggtcctattttctttatagtaaattgaacgtgaaaaaggagacctggcaacaaggcaacactttcacctgctccgttcttcacgagggcctgcataatcatcataccgaaaagtctctcagtcattctccaggtaagtag

SEQ ID NO: 95 - DNA, 528 bp, Homo sapiens
atggaaacagatacgttgctgttgtgggtacttctcctttgggtccctggcagcacaggggacgagaatagtttcgaaatgcagaagggcgaccagaacccacagatcgcggctcacgttatatcagaagcaagtagtaagaccacttccgtacttcagtgggctgaaaaaggatattacaccatgtccaacaatctcgtgacactggagaacggtaaacaacttacggtgaaacgacagggcctctattacatctacgctcaggtgacattctgctcaaatagggaggcttctagtcaagcgcccttcatcgccagcctgtgcctcaaatctcccggccggttcgaacgaatcctgttgcgagcggccaatacccatagctcagctaaaccttgcggccagcagagtattcatcttggtggtgtgtttgaacttcagccgggagcatctgtgttcgtcaacgtaacggaccctagccaagtgtctcatgggacaggttttacatccttcggactcctcaagttgtag

SEQ ID NO: 96 - DNA, 540 bp, Homo sapiens
atgacagttctcgcgccagcttggagtcccaccacatacttgcttttgcttctgcttctgtcctctggcctgagtgggacccaagattgttcctttcaacattccccaattagttctgattttgcagtgaagattagagagctctcagactatctgctgcaagattatcctgtcacagtcgcttcaaacctgcaagacgaagagctctgcggtgccttgtggcggttggtcttggctcaaagatggatggagagactgaaaaccgtagcaggcagcaagatgcagggtctcctggaaagggtgaacacggaaatccattttgtgaccaagtgcgcgttccagcccccaccgagttgtctccggtttgttcaaacgaatatatcccggttgctccaggaaacctcagaacaactggtggctttgaaaccctggatcacaagacaaaactttagtcggtgcctcgaactccagtgccaaccagattcttctacacttccccccccgtggtccccgcgcccgttggaagcaacggccccatag

SEQ ID NO: 97 - DNA, 876 bp, Homo sapiens
atggcctggagtcctctgtttctgactcttataactcactgtgccggcagttgggctataccccctcatgtacagaagtctgtaaacaacgacatgattgtaaccgacaataatggcgcagtgaaattcccacaactgtgtaagttctgtgatgtacggtttagtacatgcgacaatcaaaaaagctgtatgtctaactgctctattacatccatatgtgaaaaacctcaggaggtgtgtgttgccgtttggcgaaaaaatgatgagaatatcacactggagacagtatgtcatgaccctaaactgccataccatgatttcatactggaggacgccgccagtcctaagtgcattatgaaagagaaaaagaaacccggtgaaacattctttatgtgctcttgtagctctgacgagtgtaacgacaacattatattcagcgaggagtacaatacaagcaaccccgatataccacctcacgtacaaaaaagtgtcaacaacgatatgattgttaccgacaataacggagctgttaagtttcctcagttgtgcaagttctgcgatgtacgattctctacctgcgacaaccaaaagtcatgtatgtctaactgttccataacctccatctgcgagaagccccaggaagtctgcgtcgccgtgtggcggaaaaacgacgagaatatcactcttgaaaccgtttgtcatgatcctaaactgccctatcacgactttattctggaagatgctgcttcccctaagtgtatcatgaaagaaaagaagaaacctggggagacattctttatgtgttcatgctcctccgatgagtgtaacgacaatatcatcttctctgaggaatacaacacttctaaccctgattag

SEQ ID NO: 98 - DNA, 2178 bp, Homo sapiens
atggcctggtcccctctttttctgaccctcatcacacactgtgcaggctcatgggctgagaccgtcttgacccagtccccaggaactttgtctctgtctcctggtgaaagagctacccttagttgtcgagcctctcagtcccttggttctagctatctcgcttggtaccagcaaaagccaggccaggccccacgactgctgatctacggagcatcttcacgggctcccggcattcccgatcgattttccggatctggtagtggtacagatttcacactgaccatatctcgcctggagcccgaggactttgctgtttattattgtcagcagtacgccgattctcctatcacttttggacagggaacccgcctggagattaagcgcacagtagcagctccatccgtctttatctttccaccatcagatgaacagctcaagagtgggaccgcaagtgtagtatgcctgctgaacaatttttaccctagagaggccaaagtgcagtggaaggtggataacgccctccagagtggcaatagtcaagaaagtgttactgagcaagatagtaaggactctacatactctttgagttctactttgaccctgtcaaaagcagattatgaaaaacataaggtgtatgcatgtgaagttacacaccaagggttgtcctctccagttacaaaatcttttaatagaggagagtgccgccgcaaacgcggtagtggagaaggtcgaggctcactcttgacctgtggcgacgtggaagaaaatcccggtcctatggattggacttggagggtattttgtcttttggcagtaacacctggagctcacccccaagtacagctcgtccaatctggtgccgaggttaaaaagcctggaagttcagtgaaggtctcttgcaaggcatctggatacaccttttcatctaacgtcatatcctgggtacggcaagccccaggacagggacttgagtggatgggaggggtcatccccatcgtggacattgctaattacgctcagcgattcaaagggcgggttactataactgccgacgagtctacctcaactacctacatggagttgtcctctctccgctccgaggacactgctgtatattactgtgccagcactctcgggttggtgttggatgccatggactattggggacaaggaaccctggtgacagttagctccgcaagcactaaaggcccttctgtttttcccttggcaccttgtagtaggtctacctctgagtctacagcagcacttggatgcttggttaaggactattttcccgagccagttacagtctcttggaacagtggtgccctcacaagtggggttcataccttccccgcagtcctccagagtagtggcctttacagcctctcatcagttgtgactgttcctagttcatcactcggtactaagacatatacatgtaacgtagaccacaagccaagcaacacaaaagtagacaaacgagtcgaatctaagtatggacccccttgtccctcctgtcctgctcccgagttccttgggggcccttccgtgttcttgtttcctcccaagcccaaggataccctcatgatctcacgaaccccagaggtaacatgtgtggttgttgacgtaagtcaggaagatcccgaagtgcaatttaattggtacgtggatggcgtcgaagtccataacgctaaaacaaaaccccgagaggaacaattcaattccacatatcgggtggtgagtgtattgaccgttcttcaccaagattggctgaacggcaaggagtataagtgtaaagtaagcaacaaaggtctgccaagtagcatagaaaaaacaatatctaaagctaagggccaaccaagggaaccacaagtatatacattgcccccctctcaggaagagatgacaaagaatcaagttagcctgacctgtttggtaaaggggttctatccctcagatatagcagtcgagtgggaatctaacggccagcccgagaataattataaaacaaccccccctgtgttggactcagacggcagcttctttctctattcacggctcactgttgataagtcccgatggcaggaggggaatgttttcagctgtagcgtgatgcacgaagctctccacaaccactatacacagaaaagtttgtctttgtcccttggaaaatag

SEQ ID NO: 99 - DNA, 1062 bp, Homo sapiens
atgagtacatcctttccagagctggatctggagaattttgagtatgacgacagtgccgaagcctgctacctcggggacatagtcgcattcgggacaatctttttgtctgtattttacgccctggtgtttacatttggcctggttggaaatctgttggtcgtactcgctctcaccaattcccgaaaacccaaaagtataacagacatatacctgttgaatctggcactgagtgaccttttgttcgtcgccacccttcctttttggacacactaccttatcagtcacgaggggcttcataatgctatgtgcaagctcactactgccttcttctttatcggattcttcgggggtatcttttttatcacagttattagcattgaccgataccttgccatagtgctcgcagccaactcaatgaacaaccgcaccgtgcagcatggagtgactatttccttgggtgtgtgggccgctgctatacttgtcgccagccctcaattcatgtttaccaaaaggaaagacaatgagtgcctcggagattaccctgaggtgttgcaagaaatgtggcctgtacttcgaaatagcgaagtgaatatactcggctttgctcttcctctgctcatcatgtcattctgttattttcgaataatccaaacattgttcagctgtaagaaccgaaagaaagcccgcgccgtacgcctgattctgctcgttgtgttcgccttttttctgttttggactccttacaacataatgatattcctggagactctcaaattctataacttttttccctcctgtgatatgaaaagggaccttagattggctctcagtgtcactgaaacagtagcctttagccattgttgtctcaaccctttcatatatgcatttgcaggggaaaagttccggcggtatctcggacatttgtatcggaagtgcttggccgtgttgtgtggtcatcctgtccataccggattctctcctgagagtcaacggagccgccaagattcaatcctgtccagtttcactcactatacttcagagggggatggcagccttctgctc

SEQ ID NO: 100 - DNA, 1458 bp, Unknown
  Description of Unknown Kynureinase #1 sequence
atggagaccgacactttgttgctgtgggtacttttgttgtgggtcccaggatctaccggggatatggaaccctctcctcttgaactgccagtagacgccgtgcgccgcattgcagccgagttgaattgcgatccaacagatgaacgcgttgccctgaggctcgacgaagaggataaattgtcacatttcaggaactgcttttacattccaaagatgagggatcttccatccatagatcttagcctcgtgtccgaggatgacgatgccatatattttcttgggaacagtcttgggttgcagccaaaaatggtacggacatatctcgaagaggagctggacaaatgggctaaaatgggtgcttacggccacgacgtgggaaaacgcccctggatagttggcgacgaatctatcgtgagtcttatgaaagatatagttggagcacatgagaaagaaattgcactgatgaatgcccttactatcaatctgcatctcctcttgctttcattctttaagcccactcctaaacgccacaaaatacttttggaagcaaaagcctttccaagcgaccactacgctattgagtcacaaatacaactccatggacttgatgtggaaaagtctatgcggatggtaaaaccacgcgaaggcgaggagacccttcgaatggaggacatacttgaggtcatcgaagaagaaggagatagtatagcagttatccttttcagcgggctgcacttctacacaggtcaactctttaacattccagctattactaaggcaggccacgctaaaggatgcttcgtgggctttgaccttgcacacgcagtaggaaacgtagagctccgcttgcacgattggggcgttgatttcgcctgctggtgttcatataagtatcttaactcaggagctggtgggttggcaggcgcattcgtacacgagaaacacgctcataccgtaaagcctgcactggtagggtggttcggacacgatctctctacccgcttcaatatggataataaactccagcttatacctggcgccaatggattcaggatctcaaatcctcctattttgctcgtttgcagtttgcacgcatctcttgaggtgttccagcaggctaccatgactgcactccgccggaagtcaatccttttgaccggatacttggagtatatgctgaaacattatcactcaaaagataacactgagaataagggccccatagtaaacattatcactccatctcgggctgaagagcgcggctgccaactcacattgactttttccattcccaagaagtcagtgttcaaagagttggagaaacggggggttgtatgtgataagcgggagccagatggaatccgcgttgccccagtccccctctataattcttttcacgatgtatacaagtttattagactgctgacaagtatcttggactcatctgagcgatcttag

SEQ ID NO: 101 - DNA, 1395 bp, Unknown
  Description of Unknown Kynureinase #2 sequence
atggaaccctctcctcttgaactgccagtagacgccgtgcgccgcattgcagccgagttgaattgcgatccaacagatgaacgcgttgccctgaggctcgacgaagaggataaattgtcacatttcaggaactgcttttacattccaaagatgagggatcttccatccatagatcttagcctcgtgtccgaggatgacgatgccatatattttcttgggaacagtcttgggttgcagccaaaaatggtacggacatatctcgaagaggagctggacaaatgggctaaaatgggtgcttacggccacgacgtgggaaaacgcccctggatagttggcgacgaatctatcgtgagtcttatgaaagatatagttggagcacatgagaaagaaattgcactgatgaatgcccttactatcaatctgcatctcctcttgctttcattctttaagcccactcctaaacgccacaaaatacttttggaagcaaaagcctttccaagcgaccactacgctattgagtcacaaatacaactccatggacttgatgtggaaaagtctatgcggatggtaaaaccacgcgaaggcgaggagacccttcgaatggaggacatacttgaggtcatcgaagaagaaggagatagtatagcagttatccttttcagcgggctgcacttctacacaggtcaactctttaacattccagctattactaaggcaggccacgctaaaggatgcttcgtgggctttgaccttgcacacgcagtaggaaacgtagagctccgcttgcacgattggggcgttgatttcgcctgctggtgttcatataagtatcttaactcaggagctggtgggttggcaggcgcattcgtacacgagaaacacgctcataccgtaaagcctgcactggtagggtggttcggacacgatctctctacccgcttcaatatggataataaactccagcttatacctggcgccaatggattcaggatctcaaatcctcctattttgctcgtttgcagtttgcacgcatctcttgaggtgttccagcaggctaccatgactgcactccgccggaagtcaatccttttgaccggatacttggagtatatgctgaaacattatcactcaaaagataacactgagaataagggccccatagtaaacattatcactccatctcgggctgaagagcgcggctgccaactcacattgactttttccattcccaagaagtcagtgttcaaagagttggagaaacggggggttgtatgtgataagcgggagccagatggaatccgcgttgccccagtccccctctataattcttttcacgatgtatacaagtttattagactgctgacaagtatcttggactcatctgagcgatcttag

SEQ ID NO: 102 - DNA, 573 bp, Unknown
  Description of Unknown VEGF sequence
atgaatttcttgctgagctgggtgcattggacactcgcattgttgctgtacttgcaccatgccaagtggtcccaggctgcacccactactgagggcgagcaaaagtctcatgaggtgattaaatttatggacgtttaccaacgatcatactgtcggccaatcgaaaccctcgtagatatattccaggagtacccagacgagatcgaatacattttcaagccctcatgtgtcccattgatgcgatgtgctgggtgctgtaacgacgaagcacttgaatgtgtccccacctccgagagtaacatcacaatgcaaataatgagaatcaagccccaccaatcccaacatatcggtgaaatgtcattccttcagcattcccgctgcgagtgccggcctaagaaggaccgcaccaaaccagagaaccattgtgaaccctgttctgagagacggaagcacttgttcgtacaggaccctcaaacatgcaagtgcagctgtaagaataccgactcacggtgtaaagctaggcaactggagcttaatgaaaggacctgccgatgcgataaacccaggaggtaa

SEQ ID NO: 103 - DNA, 426 bp, Unknown
  Description of Unknown GM-CSF sequence
atgtggttgcagaatttgctcttcctggggattgtggtctacagcctctccgcacctacccgctctcctatcacagttacaagaccctggaaacatgtggaggccattaaagaagcattgaatttgttggacgatatgcccgtcaccctgaatgaagaagtagaagttgtttctaatgagttcagctttaaaaaattgacctgtgtgcagacacggcttaaaatttttgaacagggacttagaggaaactttactaagctgaagggggcacttaacatgacagcttcttattatcagacctattgtcctccaacacctgaaaccgactgtgaaacacaggtaaccacttacgccgattttattgattctttgaaaacattcctcaccgatataccatttgagtgtaagaagccaggccaaaagtag

SEQ ID NO: 104 - DNA, 822 bp, Unknown
  Description of Unknown Anti-PD1 sequence
atggaaactgacacacttcttctgtgggtcttgctcctgtgggtcccaggctctactggtgacagtcctgataggccatggaacccacctacctttagtccagccttgctcgtcgtaaccgaaggggacaacgctacattcacctgctcttttagcaatacttctgagagttttcatgtagtctggcatcgggagagtccatccggacaaacagatactttggccgcttttccagaggataggtctcaacctgggcaagacgcaaggtttcgagtcacacagcttcctaacgggagagattttcacatgtctgtagttcgggcacgccgaaatgattctggcacatatgtttgcggtgtgatctcacttgctccaaagattcaaataaaggagagccttcgcgccgagttgcgggtgactgagcgggagcccaagtcctgcgacaaaacccatacttgtccaccctgtggcggcgggtcatccggtggcgggtctggggggcaaccaagagagccacaggtatatactcttccccccagcagagaagaaatgacaaaaaaccaagtgtccctgacatgtctggttaaaggattttatcccagtgacattgctgtagaatgggaatccaatggtcaacccgagaataactacaaaaccactcctccagtattggacagtgacggttccttcttcctctattccaaacttacagtggataaatcccgctggcagcaagggaatgtattcagctgtagtgtcatgcacgaagctcttcataaccattatacacagaaatctctttccctgagcccaggtaaatag

SEQ ID NO: 105 - DNA, 1116 bp, Mus sp.
atggagactgatacacttttgctctgggttttgctcttgtgggtaccagggtctactggagatgcacaaactcctgcattcaacaagcctaaggtagagcttcatgtccatttggacggagccataaaacctgaaaccatactctatttcggcaagaaacggggtatagcacttcccgctgataccgtggaagagttgagaaatatcattggcatggacaaacctcttagcctgcctggctttcttgcaaagttcgactactatatgccagttatagcagggtgtagagaagcaataaagcgaatcgcctatgagttcgttgagatgaaggctaaagaaggagttgtttacgtggaagtccggtactcacctcatctgcttgctaatagcaaggtggacccaatgccatggaatcaaactgaaggtgatgtaacccctgacgatgtggtcgatttggtcaatcaaggtctccaagaaggcgagcaggctttcggcattaaggtaagaagtatattgtgctgtatgcgacatcaaccttcatggtccctggaggtcctcgaattgtgcaaaaagtacaatcaaaaaacagtggtcgcaatggatctcgctggagatgagaccatagaaggttcctctcttttccccggtcatgtcgaagcatatgaaggggctgtcaaaaatggtatccaccgcaccgtccacgcaggggaagtagggtccccagaagtagtcagggaagccgttgacattttgaaaacagaaagagtcgggcatggctaccatacaatagaggacgaagccttgtacaatcgacttttgaaagaaaatatgcacttcgaggtctgtccctggagttcatatctcaccggagcatgggaccccaaaacaacccacgccgtcgtacgcttcaagaatgataaggcaaactacagtttgaatacagatgatccactgatattcaagtcaacacttgacactgactaccagatgacaaaaaaagatatgggtttcaccgaagaagagttcaagagattgaacattaacgcagcaaaaagctccttcctgccagaggaagagaaaaaagaattgcttgaaaggttgtatcgagaataccaa

SEQ ID NO: 106 - DNA, 1056 bp, Mus sp.
atggcacaaactccagcttttaataagcccaaagtggaacttcatgttcatctggatggggcaattaagcccgaaactatattgtactttggcaaaaagaggggtattgccctgccagcagataccgttgaggagcttcgcaacatcattgggatggacaagcccctctctctgccaggttttctcgctaaattcgattattatatgcctgttattgctggttgccgggaggccatcaagaggatagcctacgagtttgttgagatgaaggccaaagagggcgtggtgtacgtagaggtcagatacagccctcacctgcttgccaacagcaaggtggacccaatgccctggaaccaaaccgagggggatgtcactcccgacgacgttgtagacctcgtaaatcagggccttcaagagggcgagcaggcatttggcataaaagtccggtctatactctgctgtatgaggcaccaaccctcctggtctttggaggtacttgagttgtgtaagaaatacaatcaaaagactgtagtcgccatggatcttgcaggcgatgaaaccatcgagggtagctccttgttccctggacatgttgaagcctacgagggggccgtaaaaaatgggatacacaggactgtccacgctggtgaagtcggaagcccagaggtggtaagggaggcagttgacatactcaagacagagcgggttggacacggataccacacaattgaggacgaggccctgtataaccgcctcctcaaagagaacatgcattttgaggtgtgtccttggtccagctacctgactggtgcttgggaccctaaaacaactcacgccgtggtccggttcaagaacgataaagccaattactctttgaataccgacgaccccctcatattcaaatcaacattggataccgactaccaaatgaccaaaaaggatatggggtttactgaagaggagttcaagaggctcaacataaatgccgctaaatcctcctttctccccgaggaagaaaaaaaagaactccttgagcggctgtatagggagtatcaa

SEQ ID NO: 107 - DNA, 966 bp, Mus sp.
atggaaacagatacactcttgctctgggtactgcttctgtgggtccccggctctactggggatgaagatgatgtaactactacagaagaactcgctcccgctcttgtccccccacccaagggtacctgcgccggttggatggctggcatcccaggacatccaggtcacaacggtacccccggaagagatggtcgggatggaactcccggcgagaagggcgaaaaaggggatgcagggcttctgggacctaaaggtgaaacaggggacgttggaatgactggtgcagaagggcctcgcggctttcctggcacccctgggaggaaaggagagcccggagagctccagagaactgaacctcggcctgcactcactataactacttcccctaatcttgggacccgcgagaacaacgccgatcaggttacacctgtaagccatatcgggtgccccaatactacccagcaagggagtcccgtgttcgcaaagcttttggctaaaaaccaagcatccctgtgtaacactactcttaattggcattcacaagacggtgctggtagctcttatctttctcaggggctgcggtacgaagaagataagaaggaattggttgtggattctccaggactctattatgtctttctcgaattgaagctcagtcccaccttcacaaacactggacacaaagtccagggctgggtaagtctggtactccaagcaaagccccaggttgacgatttcgacaatttggcactcaccgtagagcttttcccatgctccatggaaaataaacttgttgatcggtcatggtcacagctcttgctgcttaaggcagggcatcgcctctcagtgggtctgagagcttatttgcatggtgcacaagatgcttacagggattgggaattgtcctacccaaacactacaagtttcgggttgttccttgtcaaacctgataacccatgggagtag

SEQ ID NO: 108 - DNA, 900 bp, Mus sp.
atggaaactgatacactcctcctgtgggtccttcttttgtgggtgcccggatcaaccggcgatggctggatggcaggcatcccaggacacccaggacacaacggtactccaggtcgagacggtcgggatgggactcctggggagaaaggcgagaaaggggacgctggtttgctcggtcctaagggggaaaccggggatgtaggaatgacaggggctgaagggcctcggggatttcctgggacaccaggcaggaagggtgaaccaggggaggccctccagcgcaccgagccacggccagctctgaccataacaacaagtccaaacctgggcacacgcgaaaacaatgctgaccaggtgactcctgtaagtcacatcggatgccctaacactacacaacagggctctcctgtatttgcaaagcttctcgcaaaaaatcaagcatcactttgtaatacaaccctgaactggcattctcaggacggagcagggtcctcttatttgtctcaagggctccgctacgaagaagataaaaaggaattggttgttgacagtccaggtttgtattatgtgtttttggaacttaagctgtcaccaaccttcactaacaccggccacaaggtccaaggctgggttagtcttgttttgcaagccaaacctcaagtggatgattttgacaatctggctttgactgttgagctttttccatgcagtatggagaataaactggttgatcggtcatggtcacagctccttctgctcaaggccggacataggctgagtgtgggacttcgggcctacttgcacggcgcccaggacgcataccgagactgggaactcagctaccctaacacaacttcttttgggttgttccttgtcaaacccgataatccttgggaatag

SEQ ID NO: 109 - DNA, 870 bp, Mus sp.
atggagactgatactttgctcctgtgggttcttctcctgtgggttcctggttccacaggggatatgcatgtcaatggcaaggtagcactcgtgactggggctgcacagggtatcgggaaagcttttgccgaggccctgttgctgcatggcgccaaggtcgctttggtagattggaacttggaggctggagttaaatgcaaagctgcactcgacgaacaatttgagcctcaaaaaaccctctttgtgcagtgtgacgttgctgaccaaaagcaactcagggacacattcaggaaggtcgtagaccatttcggacgcctcgatatactcgttaataatgccggggtaaacaacgaaaagaactgggaacaaacattgcaaatcaacctggtaagtgtcattagcggaacttatctgggtcttgattatatgagcaagcagaacgggggcgagggcgggatcattatcaacatgtcaagtcttgccggattgatgccagttgctcagcagcctgtttactgtgccagcaagcacggtattattgggtttacccggagtgccgccatggccgcaaatcttatgaagagtggggtaagactgaatgttatctgcccaggtttcgtagataccccaatcctggagagcatcgagaaggaggaaaatatgggacaatacattgaatataaagatcaaatcaaggctatgatgaagttctacggggttctgcatccatccacaattgccaacgggctcattaatctgattgaggacgacgccttgaacggagctataatgaaaatcacagcttccaaaggcattcacttccaagattatgatatatcacccttgcttgtcaaggctcctctgacaagt

SEQ ID NO: 110 - DNA, 807 bp, Mus sp.
atgcatgtcaatggcaaggtagcactcgtgactggggctgcacagggtatcgggaaagcttttgccgaggccctgttgctgcatggcgccaaggtcgctttggtagattggaacttggaggctggagttaaatgcaaagctgcactcgacgaacaatttgagcctcaaaaaaccctctttgtgcagtgtgacgttgctgaccaaaagcaactcagggacacattcaggaaggtcgtagaccatttcggacgcctcgatatactcgttaataatgccggggtaaacaacgaaaagaactgggaacaaacattgcaaatcaacctggtaagtgtcattagcggaacttatctgggtcttgattatatgagcaagcagaacgggggcgagggcgggatcattatcaacatgtcaagtcttgccggattgatgccagttgctcagcagcctgtttactgtgccagcaagcacggtattattgggtttacccggagtgccgccatggccgcaaatcttatgaagagtggggtaagactgaatgttatctgcccaggtttcgtagataccccaatcctggagagcatcgagaaggaggaaaatatgggacaatacattgaatataaagatcaaatcaaggctatgatgaagttctacggggttctgcatccatccacaattgccaacgggctcattaatctgattgaggacgacgccttgaacggagctataatgaaaatcacagcttccaaaggcattcacttccaagattatgatatatcacccttgcttgtcaaggctcctctgacaagt

SEQ ID NO: 111 - DNA, 6029 bp, Artificial Sequence
  Description of Artificial Sequence Synthetic polynucleotide
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaatcaaaattttatctcgacatggtggcgaccggtagcgctagcggatcgataagcttgatatcgcctgcagccgaattccttgacttgggatccgcgtcaagtggagcaaggcaggtggacagtcctgcaggcatgcgtgactgactgaggccgcgactctagtttaaactgcgtgactgactctagaagatccggcagtgcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 112 - protein, 22 aa, Homo sapiens
MCHQQLVISWFSLVFLASPLVA

SEQ ID NO: 113 - protein, 14 aa, Artificial Sequence
  Description of Artificial Sequence Synthetic peptide
MQLLSCIALILALV

SEQ ID NO: 114 - protein, 20 aa, Homo sapiens
MYRMQLLSCIALSLALVTNS

SEQ ID NO: 115 - protein, 15 aa, Homo sapiens
MNLLLILTFVAAAVA

SEQ ID NO: 116 - protein, 17 aa, Unknown
  Description of Unknown Gaussia Luciferase sequence
MGVKVLFALICIAVAEA

SEQ ID NO: 117 - protein, 24 aa, Homo sapiens
MPMGSLQPLATLYLLGMLVASCLG

SEQ ID NO: 118 - protein, 21 aa, Mus sp.
METDTLLLWVLLLWVPGSTGD

SEQ ID NO: 119 - protein, 22 aa, Homo sapiens
MDMRVPAQLLGLLLLWLRGARC

SEQ ID NO: 120 - protein, 16 aa, Unknown
  Description of Unknown VSV-G sequence
MKCLLYLAFLFIGVNC

SEQ ID NO: 121 - protein, 28 aa, Homo sapiens
MNIKGSPWKGSLLLLLVSNLLLCQSVAP

SEQ ID NO: 122 - protein, 18 aa, Homo sapiens
MKWVTFISLLFLFSSAYS

SEQ ID NO: 123 - protein, 19 aa, Homo sapiens
MTRLTVLALLAGLLASSRA

SEQ ID NO: 124 - protein, 17 aa, Homo sapiens
MRAWIFFLLCLAGRALA

SEQ ID NO: 125 - protein, 16 aa, Homo sapiens
MPLLLLLPLLWAGALA

SEQ ID NO: 126 - protein, 29 aa, Homo sapiens
MNSFSTSAFGPVAFSLGLLLVLPAAFPAP

SEQ ID NO: 127 - protein, 20 aa, Homo sapiens
MTSKLAVALLAAFLISAALC

SEQ ID NO: 128 - protein, 23 aa, Homo sapiens
MKVSAALLCLLLIAATFIPQGLA

SEQ ID NO: 129 - protein, 26 aa, Homo sapiens
MGAAARTLRLALGLLLLATLLRPADA

SEQ ID NO: 130 - protein, 21 aa, Homo sapiens
MSPLLRRLLLAALLQLAPAQA

SEQ ID NO: 131 - protein, 21 aa, Homo sapiens
MNNLLCCALVFLDISIKWTTQ

SEQ ID NO: 132 - protein, 23 aa, Homo sapiens
MQMSPALTCLVLGLALVFGEGSA

SEQ ID NO: 133 - protein, 34 aa, Homo sapiens
MARAALSAAPSNPRLLRVALLLLLLVAAGRRAAG

SEQ ID NO: 134 - protein, 21 aa, Homo sapiens
MNAKVVVVLVLVLTALCLSDG

SEQ ID NO: 135 - protein, 22 aa, Homo sapiens
MERIVICLMVIFLGTLVHKSSS

SEQ ID NO: 136 - DNA, 1557 bp, Artificial Sequence
  Description of Artificial Sequence Synthetic polynucleotide
atttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 137 - protein, 519 aa, Artificial Sequence
  Description of Artificial Sequence Synthetic polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 138 - protein, 541 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 139 - DNA, 75 bp, Artificial Sequence
  Description of Artificial Sequence Synthetic oligonucleotide
agacggaaacgcggaagcggagagggcagaggctcgctgcttacatgcggggacgtggaagagaaccccggtccg

SEQ ID NO: 140 - protein, 25 aa, Artificial Sequence
  Description of Artificial Sequence Synthetic peptide
RRKRGSGEGRGSLLTCGDVEENPGP

SEQ ID NO: 141 - DNA, 399 bp, Artificial Sequence
  Description of Artificial Sequence Synthetic polynucleotide
cagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 142 - protein, 133 aa, Homo sapiens [sequence omitted]

SEQ ID NO: 143 - protein, 155 aa, Homo sapiens
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 144 - DNA, 2598 bp, Artificial Sequence
  Description of Artificial Sequence Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgcggaattcgccgccaccatgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagtagacggaaacgcggaagcggagagggcagaggctcgctgcttacatgcggggacgtggaagagaaccccggtccgatggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattcatga

SEQ ID NO: 145 - DNA, 7213 bp, Artificial Sequence
  Description of Artificial Sequence Synthetic polynucleotide
tgactcctgcgcagtccaaaaaaaaaggctccaaaaggagcctttaattgtatcggtgggcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttaaacaaatagtcaaaagcctccggcgactagtcggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgggtctctctggttagaccagatttgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactgcagagtacgccaaaattttgactagcggaggctagaaggagagagatgggtgcgagagcgtcagtattaagcgggggaaaatagcggccgccacaattttaaaagaaaaggggggattggggggtacagtgcaggggaaagaatagtagacataatagcaacagacatacaaactaaagaattacaaaaacaaattacaaaaattcaaattttcgggggatccgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgcggaattcgccgccaccatgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagtagacggaaacgcggaagcggagagggcagaggctcgctgcttacatgcggggacgtggaagagaaccccggtccgatggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattcatgaggttagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcttagtactggtacctttaagaccaatgacttacaaggcagctgtagatcttagccactttttaaaagaaaaggggggactggaagggctaattcactcccaacgaagacaagattccggaatttatttgtgaaatttgtgatgctattgctttatttgtaaaccggtgcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatctagagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggctagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctagccgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcc